SEQUENCE LISTING (72 sequences)

SEQ ID NO: 1 - protein, 217 aa, Artificial Sequence
  Synthetic Sequence
APEFLGGPSVFLFPPKPKDTLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSSWQEGNVFSCSVMHEALHNHYTQKSLSLSLGK

SEQ ID NO: 2 - protein, 217 aa, Artificial Sequence
  Synthetic Sequence
APEFLGGPSVFLFPPKPKDQLMISRTPEVTCVVVDVSQEDPEVQFNWYVDGVEVHNAKTKPREEQFNSTYRVVSVLTTPHSDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSSWQEGNVFSCSVLHEALHNHYTQKSLSLSLGK

SEQ ID NO: 3 - protein, 217 aa, Artificial Sequence
  Synthetic Sequence
APEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGK

SEQ ID NO: 4 - protein, 11 aa, Artificial Sequence
  Synthetic Sequence
SKYGPPCPSCP

SEQ ID NO: 5 - protein, 11 aa, Artificial Sequence
  Synthetic Sequence
SKYGPPCPPCP

SEQ ID NO: 6 - protein, 6 aa, Artificial Sequence
  Synthetic Sequence
SKYGPP

SEQ ID NO: 7 - protein, 6 aa, Artificial Sequence
  Synthetic Sequence
IEGRMD

SEQ ID NO: 8 - protein, 9 aa, Artificial Sequence
  Synthetic Sequence
GGGVPRDCG

SEQ ID NO: 9 - protein, 15 aa, Artificial Sequence
  Synthetic Sequence
IEGRMDGGGGAGGGG

SEQ ID NO: 10 - protein, 8 aa, Artificial Sequence
  Synthetic Sequence
GGGSGGGS

SEQ ID NO: 11 - protein, 12 aa, Artificial Sequence
  Synthetic Sequence
GGGSGGGGSGGG

SEQ ID NO: 12 - protein, 14 aa, Artificial Sequence
  Synthetic Sequence
EGKSSGSGSESKST

SEQ ID NO: 13 - protein, 4 aa, Artificial Sequence
  Synthetic Sequence
GGSG

SEQ ID NO: 14 - protein, 12 aa, Artificial Sequence
  Synthetic Sequence
GGSGGGSGGGSG

SEQ ID NO: 15 - protein, 15 aa, Artificial Sequence
  Synthetic Sequence
EAAAKEAAAKEAAAK

SEQ ID NO: 16 - protein, 20 aa, Artificial Sequence
  Synthetic Sequence
EAAAREAAAREAAAREAAAR

SEQ ID NO: 17 - protein, 17 aa, Artificial Sequence
  Synthetic Sequence
GGGGSGGGGSGGGGSAS

SEQ ID NO: 18 - protein, 9 aa, Artificial Sequence
  Synthetic Sequence
GGGGAGGGG

SEQ ID NO: 19 - protein, 2 aa, Artificial Sequence
  Synthetic Sequence
  misc_feature: SEQ ID NO 19 can be GS or GGS or LE
GS

SEQ ID NO: 20 - protein, 6 aa, Artificial Sequence
  Synthetic Sequence
GSGSGS

SEQ ID NO: 21 - protein, 10 aa, Artificial Sequence
  Synthetic Sequence
GSGSGSGSGS

SEQ ID NO: 22 - protein, 7 aa, Artificial Sequence
  Synthetic Sequence
GGGGSAS

SEQ ID NO: 23 - protein, 20 aa, Artificial Sequence
  Synthetic Sequence
APAPAPAPAPAPAPAPAPAP

SEQ ID NO: 24 - protein, 4 aa, Artificial Sequence
  Synthetic Sequence
CPPC

SEQ ID NO: 25 - protein, 5 aa, Artificial Sequence
  Synthetic Sequence
GGGGS

SEQ ID NO: 26 - protein, 10 aa, Artificial Sequence
  Synthetic Sequence
GGGGSGGGGS

SEQ ID NO: 27 - protein, 15 aa, Artificial Sequence
  Synthetic Sequence
GGGGSGGGGSGGGGS

SEQ ID NO: 28 - protein, 20 aa, Artificial Sequence
  Synthetic Sequence
GGGGSGGGGSGGGGSGGGGS

SEQ ID NO: 29 - protein, 25 aa, Artificial Sequence
  Synthetic Sequence
GGGGSGGGGSGGGGSGGGGSGGGGS

SEQ ID NO: 30 - protein, 30 aa, Artificial Sequence
  Synthetic Sequence
GGGGSGGGGSGGGGSGGGGSGGGGSGGGGS

SEQ ID NO: 31 - protein, 35 aa, Artificial Sequence
  Synthetic Sequence
GGGGSGGGGSGGGGSGGGGSGGGGSGGGGSGGGGS

SEQ ID NO: 32 - protein, 40 aa, Artificial Sequence
  Synthetic Sequence
GGGGSGGGGSGGGGSGGGGSGGGGSGGGGSGGGGSGGGGS

SEQ ID NO: 33 - protein, 16 aa, Artificial Sequence
  Synthetic Sequence
GGSGGSGGGGSGGGGS

SEQ ID NO: 34 - protein, 8 aa, Artificial Sequence
  Synthetic Sequence
GGGGGGGG

SEQ ID NO: 35 - protein, 6 aa, Artificial Sequence
  Synthetic Sequence
GGGGGG

SEQ ID NO: 36 - protein, 5 aa, Artificial Sequence
  Synthetic Sequence
EAAAK

SEQ ID NO: 37 - protein, 10 aa, Artificial Sequence
  Synthetic Sequence
EAAAKEAAAK

SEQ ID NO: 38 - protein, 15 aa, Artificial Sequence
  Synthetic Sequence
EAAAKEAAAKEAAAK

SEQ ID NO: 39 - protein, 12 aa, Artificial Sequence
  Synthetic Sequence
AEAAAKEAAAKA

SEQ ID NO: 40 - protein, 17 aa, Artificial Sequence
  Synthetic Sequence
AEAAAKEAAAKEAAAKA

SEQ ID NO: 41 - protein, 22 aa, Artificial Sequence
  Synthetic Sequence
AEAAAKEAAAKEAAAKEAAAKA

SEQ ID NO: 42 - protein, 27 aa, Artificial Sequence
  Synthetic Sequence
AEAAAKEAAAKEAAAKEAAAKEAAAKA

SEQ ID NO: 43 - protein, 46 aa, Artificial Sequence
  Synthetic Sequence
AEAAAKEAAAKEAAAKEAAAKALEAEAAAKEAAAKEAAAKEAAAKA

SEQ ID NO: 44 - protein, 5 aa, Artificial Sequence
  Synthetic Sequence
PAPAP

SEQ ID NO: 45 - protein, 18 aa, Artificial Sequence
  Synthetic Sequence
KESGSVSSEQLAQFRSLD

SEQ ID NO: 46 - protein, 12 aa, Artificial Sequence
  Synthetic Sequence
GSAGSAAGSGEF

SEQ ID NO: 47 - protein, 5 aa, Artificial Sequence
  Synthetic Sequence
GGGSE

SEQ ID NO: 48 - protein, 5 aa, Artificial Sequence
  Synthetic Sequence
GSESG

SEQ ID NO: 49 - protein, 5 aa, Artificial Sequence
  Synthetic Sequence
GSEGS

SEQ ID NO: 50 - protein, 35 aa, Artificial Sequence
  Synthetic Sequence
GEGGSGEGSSGEGSSSEGGGSEGGGSEGGGSEGGS

SEQ ID NO: 51 - protein, 234 aa, Artificial Sequence
  Synthetic Sequence
SKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSSWQEGNVFSCSVMHEALHNHYTQKSLSLSLGKIEGRMD

SEQ ID NO: 52 - protein, 234 aa, Artificial Sequence
  Synthetic Sequence
SKYGPPCPSCPAPEFLGGPSVFLFPPKPKDQLMISRTPEVTCVVVDVSQEDPEVQFNWYVDGVEVHNAKTKPREEQFNSTYRVVSVLTTPHSDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSSWQEGNVFSCSVLHEALHNHYTQKSLSLSLGKIEGRMD

SEQ ID NO: 53 - protein, 234 aa, Artificial Sequence
  Synthetic Sequence
SKYGPPCPSCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMD

SEQ ID NO: 54 - protein, 234 aa, Artificial Sequence
  Synthetic Sequence
SKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSSWQEGNVFSCSVMHEALHNHYTQKSLSLSLGKIEGRMD

SEQ ID NO: 55 - protein, 234 aa, Artificial Sequence
  Synthetic Sequence
SKYGPPCPPCPAPEFLGGPSVFLFPPKPKDQLMISRTPEVTCVVVDVSQEDPEVQFNWYVDGVEVHNAKTKPREEQFNSTYRVVSVLTTPHSDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSSWQEGNVFSCSVLHEALHNHYTQKSLSLSLGKIEGRMD

SEQ ID NO: 56 - protein, 234 aa, Artificial Sequence
  Synthetic Sequence
SKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMD

SEQ ID NO: 57 - protein, 343 aa, Artificial Sequence
  Synthetic polymer
EEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIY

SEQ ID NO: 58 - protein, 215 aa, Artificial Sequence
  Synthetic polymer
HRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 59 - protein, 792 aa, Artificial Sequence
  Synthetic polymer
EEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMDHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 60 - DNA, 2436 bp, Artificial Sequence
  Synthetic polymer
atggaatggagctgggtgttcttgttcttcctgtccgtgaccaccggcgtgcactcggaggaggagctccaggtcatccagccggacaagtcggtgctcgtggccgccggagaaactgccaccctgaggtgcaccgcgacctcgctgattcccgtgggcccgattcagtggttccggggggccgggcctggcagagaactgatctacaaccagaaggaaggccatttccctcgcgtgactactgtgtccgatcttactaagcggaacaacatggacttcagcattaggatcggcaacatcacccctgctgacgcgggaacctactactgcgtcaagttcaggaaaggaagcccggacgacgtggagttcaagagcggggcgggcaccgaactgtccgtgcgcgccaagccatccgcgcccgtggtgtccggacccgcagccagagcaactccgcagcacaccgtgtcgttcacttgcgaatcacacggattctccccgcgcgatatcacgcttaagtggttcaagaacgggaacgaactgagcgacttccagaccaacgtggaccccgtcggagaaagcgtcagctactccattcactcgaccgccaaagtggtgctgaccagggaggacgtgcatagccaagtgatctgcgaggtcgcccacgtcactctgcaaggagatccgctgcggggaacagccaacctgtccgaaactatccgcgtgcctcccaccctggaagtgacccagcagcccgtccgagcggagaatcaagtcaatgtgacctgtcaagtccggaaattctaccctcaacggctccagctgacctggctggaaaacggaaacgtgtcccgcacggaaaccgcctcgaccgtgaccgagaacaaggacggcacctacaactggatgtcctggctcttggtgaacgtgtcagcccaccgggacgatgtcaagctgacttgccaagtggaacatgatgggcagccagctgtcagcaagagccacgacctgaaggtgtccgcgcacccgaaggaacagggttcgaatactgccgccgaaaacactggtagcaacgaacggaacatctactctaagtacggcccaccttgccctccctgcccggcacctgaatttctgggtggaccctccgtgtttcttttcccgcccaagccaaaggaccagttgatgatctcccgcactccggaagtgacatgcgtggtggtggacgtgtcccaggaagatccggaagtgcagttcaattggtacgtggatggcgtggaggtccataacgccaagactaagccgcgcgaggaacagttcaattccacctaccgggtggtgtccgtgctgaccgtgctgcatcaggactggctctccggcaaagagtacaagtgcaaggtgtcatccaagggtctgccgtcgtcaatcgaaaagaccatttccaatgccactgggcagcccagagaacctcaagtctacaccctcccaccgtcccaagaggaaatgaccaagaaccaagtctcgctgacgtgtctcgtgaagggattctacccatccgacattgctgtggaatgggagtccaacggccagcccgagaacaactacaagactacccctcccgtcctggactccgacggttccttcttcctttactctcgcctcaccgtggataagtcgcggtggcaggaggggaacgtgttctcctgctccgtcctgcacgaagcattgcacaaccactacacccagaagtccctgtcactgtccctgggaaagattgagggtcggatggatcatcggcgcctggacaagatcgaggacgagcggaacctccacgaggatttcgtgttcatgaaaaccatccagagatgcaacaccggagagagaagcctgtccctgctcaactgcgaggaaatcaagtcccagtttgaaggatttgtgaaggacattatgctgaacaaggaagagactaagaaggaaaactccttcgagatgcagaagggcgatcagaacccacagatcgcggcccacgtgatctccgaggcctcgtcaaagaccacttcagtgctccaatgggccgagaagggttactataccatgagcaacaaccttgtgaccctggagaacggaaagcagctcaccgtgaaaagacagggactgtactatatctatgcccaagtcaccttctgttcgaaccgcgaggctagcagccaggccccgttcatcgcctccctctgtttgaagtcgccggggcggtttgaaaggattctgctgagagctgcgaatacccattcgtccgccaagccttgcggacagcagtcaatccacctggggggagtgttcgagctgcagcctggcgcgagcgtgttcgtcaacgtgaccgacccctcccaagtgtctcacggcaccggattcacttcgtttggcctgctgaagctgtaa

SEQ ID NO: 61 - protein, 811 aa, Artificial Sequence
  Synthetic polymer
MEWSWVFLFFLSVTTGVHSEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMDHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 62 - protein, 342 aa, Artificial Sequence
  Synthetic polymer
KELKVTQPEKSVSVAAGDSTVLNCTLTSLLPVGPIRWYRGVGPSRLLIYSFAGEYVPRIRNVSDTTKRNNMDFSIRISNVTPADAGIYYCVKFQKGSSEPDTEIQSGGGTEVYVLAKPSPPEVSGPADRGIPDQKVNFTCKSHGFSPRNITLKWFKDGQELHPLETTVNPSGKNVSYNISSTVRVVLNSMDVNSKVICEVAHITLDRSPLRGIANLSNFIRVSPTVKVTQQSPTSMNQVNLTCRAERFYPEDLQLIWLENGNVSRNDTPKNLTKNTDGTYNYTSLFLVNSSAHREDVVFTCQVKHDQQPAITRNHTVLGFAHSSDQGSMQTFPDNNATHNWN

SEQ ID NO: 63 - protein, 227 aa, Artificial Sequence
  Synthetic polymer
VPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 64 - protein, 214 aa, Artificial Sequence
  Synthetic polymer
HRRLDKVEEEVNLHEDFVFIKKLKRCNKGEGSLSLLNCEEMRRQFEDLVKDITLNKEEKKENSFEMQRGDEDPQIAAHVVSEANSNAASVLQWAKKGYYTMKSNLVMLENGKQLTVKREGLYYVYTQVTFCSNREPSSQRPFIVGLWLKPSSGSERILLKAANTHSSSQLCEQQSVHLGGVFELQAGASVFVNVTEASQVIHRVGFSSFGLLKL

SEQ ID NO: 65 - protein, 343 aa, Artificial Sequence; identical to SEQ ID NO: 57
  Synthetic polymer

SEQ ID NO: 66 - protein, 229 aa, Artificial Sequence
  Synthetic polymer
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 67 - DNA, 37 bp, Artificial Sequence
  Synthetic polymer
gcggcgctcgaggccaccatgtggcccctggtagcgg

SEQ ID NO: 68 - DNA, 45 bp, Artificial Sequence
  Synthetic polymer
actagcggtaccccatcacttcacttcagttattccacaaatttc

SEQ ID NO: 69 - DNA, 35 bp, Artificial Sequence
  Synthetic polymer
gcggcgctcgaggccaccatgtggcccttggcggc

SEQ ID NO: 70 - DNA, 41 bp, Artificial Sequence
  Synthetic polymer
actagcggtacctcacctattcctaggaggttggatagtcc

SEQ ID NO: 71 - DNA, 39 bp, Artificial Sequence
  Synthetic polymer
gcggcgctcgaggccaccatggtgtctttgcctcggctg

SEQ ID NO: 72 - DNA, 31 bp, Artificial Sequence
  Synthetic polymer
actagcggtacctcagaccaggggcctcaag

(19) United States
(12) Patent Application Publication
(10) Pub. No.: US 2025/0320268 A1
(43) Pub. Date: Oct. 16, 2025

(54) CLINICAL DOSING OF SIRP1A CHIMERIC PROTEIN

(71) Applicant: Shattuck Labs, Inc., Austin, TX (US)

(72) Inventors: Arundathy PANDITE, Austin, TX (US); Fatima RANGWALA, Austin, TX (US); Tom LAMPKIN, Austin, TX (US); Taylor SCHREIBER, Austin, TX (US); George FROMM, Austin, TX (US); Suresh DE SILVA, Austin, TX (US)

(21) Appl. No.: 18/026,391

(22) PCT Filed: Sep. 17, 2021
(86) PCT No.: PCT/US21/50888

§ 371 Date: Mar. 15, 2023

Related U.S. Application Data
(60) Provisional application No. 63/079,982, filed on Sep. 17, 2020.
(60) Provisional application No. 63/229,244, filed on Aug. 4, 2021.
(60) Provisional application No. 63/231,578, filed on Aug. 10, 2021.

(51) Int. Cl. C07K 14/705; A61K 38/00; A61P 35/00

(52) U.S. Cl. CPC C07K 14/70503; A61P 35/00; C07K 14/70575; A61K 38/00; C07K 2319/30

(57) ABSTRACT

The present disclosure relates, inter alia, to methods of treating cancer with chimeric proteins comprising an extracellular domain of human signal regulatory protein α (CD172a (SIRPα)) and an extracellular domain of human CD40 ligand (CD40L), including doses and regimens.

### PRIORITY

This application is a U.S. National Stage Application under 37 U.S.C. § 371 of International Application No. PCT/US2021/050888, filed Sep. 19, 2021, which claims the benefit of, and priority to, U.S. Provisional Application Nos. 63/231,578, filed Aug. 10, 2021; 63/229,244, filed Aug. 4, 2021; and 63/079,982, filed Sep. 17, 2020, the contents of each of which are hereby incorporated by reference in its entirety.

### TECHNICAL FIELD

The present disclosure relates to, inter alia, compositions and methods, including chimeric proteins that find use in the treatment of disease, such as immunotherapies for cancer comprising doses, dosing regimens that including biphasic dosing or dosing regimens comprising three cycles.

### INCORPORATION BY REFERENCE OF MATERIAL SUBMITTED ELECTRONICALLY

The contents of the text file named “SHK-034_Sequence_Listing_ST25”, which was created on Mar. 15, 2023 and is 65,465 bytes in size, are hereby incorporated herein by reference in their entireties.

### BACKGROUND

The field of cancer immunotherapy has grown tremendously over the past several years. This has been largely driven by the clinical efficacy of antibodies targeting the family of checkpoint molecules (e.g., CTLA-4 and PD-1) in many tumor types. However, despite this success, clinical response to these agents as monotherapy occurs in a minority of patients (10-45% in various solid tumors), and these therapies are hindered by side effects.

Discovery of the proper dose and regimen of such agents is crucial to efficacious treatment of cancers. Developing novel treatment strategies, including dosing and regimens, remains a formidable task given the complexity of the human immune system, the high cost, and the potential for toxicity which may result from such interventions.

### SUMMARY

In various aspects, the present disclosure provides for compositions and methods that are useful for cancer immunotherapy. For instance, the present disclosure, in part, relates to doses and treatment regimens of specific chimeric proteins that simultaneously block immune inhibitory signals and stimulate immune activating signals. Importantly, inter alia, the present disclosure provides for improved chimeric proteins that can maintain a stable and reproducible multimeric state. Accordingly, the present compositions and methods overcome various deficiencies in producing bi-specific agents. Using this approach, combination immunotherapy can be achieved by a single chimeric protein, having superior preclinical activity compared to the separate administration of two individual antibodies against each of the identical targets. Further, the present disclosure allows for treatment of human cancer patients with amounts of the present chimeric proteins to yield successful therapy.

The present disclosure relates to chimeric proteins comprising an extracellular domain of human signal regulatory protein α (CD172a (SIRPα)) and an extracellular domain of human CD40 ligand (CD40L). CD172a (SIRPα) is Type I transmembrane protein, which binds, at least, CD47 on the surface of human tumor cells; this binding blocks an inhibitory signal produced by the tumor cell, or other cells in the tumor microenvironment. Thus, the CD172a (SIRPα) end of a chimeric protein disrupts, blocks, reduces, inhibits and/or sequesters the transmission of immune inhibitory signals, e.g., originating from a cancer cell that is attempting to avoid its detection and/or destruction. CD40L is a Type II transmembrane that binds a CD40 receptor (e.g., CD40) on the surface of primary peripheral blood mononuclear cells (PBMCs), as well as tissue-resident antigen presenting cells; this binding provides immune stimulatory properties upon anti-cancer immune cells. Thus, the CD40L end of a chimeric protein enhances, increases, and/or stimulates the transmission of an immune stimulatory signal to the CD40 expressing immune cell. Together, chimeric proteins of the present disclosure are capable of treating cancer via two distinct mechanisms.

In a chimeric protein of the present disclosure, the extracellular domain of human CD172a (SIRPα) (a Type I transmembrane protein) is located at the chimeric protein's amino terminus (see, by way of non-limiting example, FIG. 1A, left protein), whereas the extracellular domain of human CD40L (a Type II transmembrane protein), is located at the chimeric protein's carboxy terminus (see, by way of non-limiting example, FIG. 1A, right protein). The extracellular domain of CD172a (SIRPα) contains the functional domains that are responsible for interacting with other binding partners (either ligands or receptors) in the extracellular environment (see, FIG. 1B, left protein) and the extracellular domain of CD40L contains the functional domains that are responsible for interacting with other binding partners (either ligands or receptors) in the extracellular environment (see, FIG. 1B, right protein).

An aspect of the present disclosure is a method for treating a cancer in a human subject. The method comprising a step of administering to the human subject a chimeric protein having a general structure of: N terminus-(a)-(b)-(c)-C terminus, in which (a) is a first domain comprising an extracellular domain of human signal regulatory protein α (CD172a (SIRPα)), (b) is a linker adjoining the first and second domains, wherein the linker comprises at least one cysteine residue capable of forming a disulfide bond and/or comprises a hinge-CH2-CH3 Fc domain, and (c) is a second domain comprising an extracellular domain of human CD40 ligand (CD40L). See, by way of non-limiting examples, FIG. 1C and FIG. 1D. See, also, FIG. 3A.

In embodiments, the dose of the chimeric protein administered is at least about 0.0001 mg/kg, e.g., between about 0.0001 mg/kg and about 10.0 mg/kg. The chimeric protein may be administered at an initial dose (e.g., at one of about 0.0001, about 0.001, about 0.003, about 0.01, about 0.03, about 0.1, about 0.3, about 1, about 2, about 3, about 4, about 6 or about 10.0 mg/kg) and the chimeric protein is administered in one or more subsequent administrations (e.g., at one or more of about 0.0001, about 0.001, about 0.003, about 0.01, about 0.03, about 0.1, about 0.3, about 1.0, about 2, about 3, about 4, about 6, about 8, and about 10 mg/kg). In embodiments, the initial dose is less than the dose for at least one of the subsequent administrations (e.g. each of the subsequent administrations) or the initial dose is the same as the dose for at least one of the subsequent administrations (e.g., each of the subsequent administrations). In embodiments, the starting dose and/or the subsequent doses is the maximum tolerated dose or less than the maximum tolerated dose. In embodiments, the chimeric protein is administered at least about one time a month, e.g., at least about two times a month, at least about three times a month, and at least about four times a month. In embodiments, the chimeric protein is first administered once a week for three weeks and the chimeric protein is then administered about once every three weeks or once every four weeks; alternately, the chimeric protein is first administered once a week for three weeks and the chimeric protein is then administered about two times per month, e.g., once a week for three weeks and the chimeric protein is then administered about once every two weeks. In embodiments, the chimeric protein exhibits a linear dose response in the dose range of e.g., about 0.3 mg/kg to about 3 mg/kg, or about 0.5 mg/kg to about 3 mg/kg, or about 0.7 mg/kg to about 3 mg/kg, or about 1 mg/kg to about 3 mg/kg, or about 1.5 mg/kg to about 3 mg/kg, or about 2 mg/kg to about 3 mg/kg, or about 2.5 mg/kg to about 3 mg/kg, or about 0.3 mg/kg to about 2.5 mg/kg, or about 0.3 mg/kg to about 2 mg/kg, or about 0.3 mg/kg to about 1.5 mg/kg, or about 0.3 mg/kg to about 1.0 mg/kg, or about 0.3 mg/kg to about 0.5 mg/kg. In embodiments, the chimeric protein does not exhibit a bell-shaped dose response.

In embodiments, the administration is intravenous. In embodiments, the administration is intratumoral. In embodiments, the administration is by injection. In embodiments, the administration is by infusion. In embodiments, the administration is performed by an intravenous infusion. In embodiments, the administration is performed by an intratumoral injection.

In embodiments, the cancer is selected from ovarian cancer, fallopian tube cancer, peritoneal cancer, cutaneous squamous cell carcinoma (CSCC), and squamous cell carcinoma of the head and neck (SCCHN). In embodiments, the cancer comprises an advanced solid tumor (local and/or metastatic) or advanced lymphoma. In embodiments, the cancer is a solid cancer. In embodiments, the cancer is a solid tumor. In embodiments, the cancer is a metastatic cancer. In embodiments, the cancer is a hematological cancer. In embodiments, the cancer expresses CD47.

In one aspect, the present disclosure relates to a method for treating a cancer in a human subject comprising: (i) administering to the human subject a chimeric protein having a general structure of: N terminus-(a)-(b)-(c)-C terminus, wherein: (a) is a first domain comprising an extracellular domain of human signal regulatory protein α (CD172a (SIRPα)), (b) is a linker adjoining the first and second domains, wherein the linker comprises at least one cysteine residue capable of forming a disulfide bond and/or comprises a hinge-CH2-CH3 Fc domain, and (c) is a second domain comprising an extracellular domain of human CD40 ligand (CD40L); and (ii) administering a second therapeutic agent. In embodiments, the dose of the chimeric protein administered is at least about 0.3 mg/kg, e.g., at least about 0.3 mg/kg, or about 1.0 mg/kg, or about 2 mg/kg, or about 3, about 4 mg/kg, or about 6 mg/kg, or about 8 mg/kg, or about 10 mg/kg. In embodiments, the dose of the chimeric protein administered is at least about 1 mg/kg, e.g., at least about 1.0 mg/kg, or about 2 mg/kg, or about 3, about 4 mg/kg, or about 6 mg/kg, or about 8 mg/kg, or about 10 mg/kg. In embodiments, the chimeric protein exhibits a linear dose response in the dose range of e.g., about 0.3 mg/kg to about 3 mg/kg, or about 0.5 mg/kg to about 3 mg/kg, or about 0.7 mg/kg to about 3 mg/kg, or about 1 mg/kg to about 3 mg/kg, or about 1.5 mg/kg to about 3 mg/kg, or about 2 mg/kg to about 3 mg/kg, or about 2.5 mg/kg to about 3 mg/kg, or about 0.3 mg/kg to about 2.5 mg/kg, or about 0.3 mg/kg to about 2 mg/kg, or about 0.3 mg/kg to about 1.5 mg/kg, or about 0.3 mg/kg to about 1.0 mg/kg, or about 0.3 mg/kg to about 0.5 mg/kg. In embodiments, the chimeric protein does not exhibit a bell-shaped dose response.

In one aspect, the present disclosure relates to a method for treating a cancer in a human subject comprising administering to a subject in need thereof: a chimeric protein of a general structure of N terminus-(a)-(b)-(c)-C terminus, wherein: (a) is a first domain comprising an extracellular domain of human signal regulatory protein α (CD172a (SIRPα)), (b) is a linker adjoining the first and second domains, wherein the linker comprises at least one cysteine residue capable of forming a disulfide bond and/or comprises a hinge-CH2-CH3 Fc domain, and (c) is a second domain comprising an extracellular domain of human CD40 ligand (CD40L); wherein: the subject is undergoing or has undergone treatment with a second therapeutic agent. In embodiments, the chimeric protein exhibits a linear dose response. In embodiments, the chimeric protein does not exhibit a bell-shaped dose response.

In one aspect, the present disclosure relates to a method for treating a cancer in a human subject comprising administering to a subject in need thereof a second anticancer therapeutic agent, wherein the subject is undergoing or has undergone treatment with a chimeric protein of a general structure of N terminus-(a)-(b)-(c)-C terminus, wherein: (a) is a first domain comprising an extracellular domain of human Signal regulatory protein α (CD172a (SIRPα)), (b) is a linker adjoining the first and second domains, wherein the linker comprises at least one cysteine residue capable of forming a disulfide bond and/or comprises a hinge-CH2-CH3 Fc domain, and (c) is a second domain comprising an extracellular domain of human CD40 ligand (CD40L). In embodiments, the chimeric protein exhibits a linear dose response in the dose range of e.g., about 0.3 mg/kg to about 3 mg/kg, or about 0.5 mg/kg to about 3 mg/kg, or about 0.7 mg/kg to about 3 mg/kg, or about 1 mg/kg to about 3 mg/kg, or about 1.5 mg/kg to about 3 mg/kg, or about 2 mg/kg to about 3 mg/kg, or about 2.5 mg/kg to about 3 mg/kg, or about 0.3 mg/kg to about 2.5 mg/kg, or about 0.3 mg/kg to about 2 mg/kg, or about 0.3 mg/kg to about 1.5 mg/kg, or about 0.3 mg/kg to about 1.0 mg/kg, or about 0.3 mg/kg to about 0.5 mg/kg. In embodiments, the chimeric protein does not exhibit a bell-shaped dose response.

In embodiments, the chimeric protein is administered before the second therapeutic agent. In embodiments, the second therapeutic agent is administered before the chimeric protein. In embodiments, the second therapeutic agent and the chimeric protein are administered substantially together.

In embodiments, the second therapeutic agent is selected from an antibody, and a chemotherapeutic agent. In embodiments, the antibody is capable of antibody-dependent cellular cytotoxicity (ADCC). In embodiments, the antibody is selected from cetuximab, rituximab, obinutuzumab, Hul4.18K322A, Hu3F8, dinituximab, and trastuzumab. In embodiments, the antibody is capable of antibody-dependent cellular phagocytosis (ADCP). In embodiments, the antibody is selected from cetuximab, daratumumab, rituximab, and trastuzumab. In embodiments, the antibody is capable of binding a molecule selected from carcinoembryonic antigen (CEA), EGFR, HER-2, epithelial cell adhesion molecule (EpCAM), and human epithelial mucin-1, CD20, CD30, CD38, CD40, and CD52. In embodiments, the antibody is capable of binding EGFR. In embodiments, the antibody is selected from Mab A13, AMG595, cetuximab (Erbitux, C225), panitumumab (ABX-EGF, Vectibix), depatuxizumab (ABT 806), depatuxizumab, mafodotin, duligotuzumab (MEHD7945A, RG7597), Futuximab (Sym004), GC1118, imgatuzumab (GA201), matuzumab (EMD 72000), necitumumab (Portrazza), nimotuzumab (h-R3), anitumumab (Vectibix, ABX-EGF), zalutumumab, humMR1, and tomuzotuximab. In embodiments, the antibody is cetuximab.

In embodiments, the chemotherapeutic agent is an anthracycline. In embodiments, the anthacycline is selected from doxorubicin, daunorubicin, epirubicin and idarubicin, and pharmaceutically acceptable salts, acids or derivatives thereof. In embodiments, the chemotherapeutic agent is doxorubicin.

In embodiments, the chemotherapeutic agent is an antimetabolite chemotherapeutic. In embodiments, the anti-metabolite chemotherapeutic is selected from 5-fluorouracil, methotrexate, capecitabine, azacitidine, 6-diazo-5-oxo-L-norleucine (DON), azaserine, acivicin, and pharmaceutically acceptable salts, acids or derivatives thereof. In embodiments, the chemotherapeutic agent is azacitidine.

In embodiments, the chemotherapeutic agent is a B-cell lymphoma-2 (Bcl-2) inhibitor. In embodiments, the chemotherapeutic agent is a BH3-mimetic. In embodiments, the Bcl-2 inhibitor and/or the BH3-mimetic is venetoclax, ABT-737, or navitoclax. In embodiments, the chemotherapeutic agent is venetoclax.

In embodiments, the dose of the chimeric protein administered is at least about 0.0001 mg/kg, e.g., between about 0.0001 mg/kg and about 10 mg/kg. The chimeric protein may be administered at an initial dose (e.g., at one of about 0.0001, about 0.001, about 0.003, about 0.01, about 0.03, about 0.1, about 0.3, about 1, about 2, about 3, about 4, about 6 or about 10.0 mg/kg) and the chimeric protein is administered in one or more subsequent administrations (e.g., at one or more of about 0.0001, about 0.001, about 0.003, about 0.01, about 0.03, about 0.1, about 0.3, about 1, about 2, about 3, about 4, about 6, about 8, and about 10 mg/kg). In embodiments, the dose of the chimeric protein administered is at least about 0.3 mg/kg, e.g., at least about 0.3 mg/kg, or about 1.0 mg/kg, or about 2 mg/kg, or about 3, about 4 mg/kg, or about 6 mg/kg, or about 8 mg/kg, or about 10 mg/kg. In embodiments, the dose of the chimeric protein administered is at least about 1 mg/kg, e.g., at least about 1.0 mg/kg, or about 2 mg/kg, or about 3, about 4 mg/kg, or about 6 mg/kg, or about 8 mg/kg, or about 10 mg/kg. In embodiments, the initial dose is less than the dose for at least one of the subsequent administrations (e.g. each of the subsequent administrations) or the initial dose is the same as the dose for at least one of the subsequent administrations (e.g., each of the subsequent administrations). In embodiments, the starting dose and/or the subsequent doses is the maximum tolerated dose or less than the maximum tolerated dose. In embodiments, the chimeric protein is administered at least about one time a month, e.g., at least about two times a month, at least about three times a month, and at least about four times a month. In embodiments, the chimeric protein is first administered once a week for three weeks and the chimeric protein is then administered about once every three weeks or once every four weeks; alternately, the chimeric protein is first administered once a week for three weeks and the chimeric protein is then administered about two times per month, e.g., once a week for three weeks and the chimeric protein is then administered about once every two weeks.

In embodiments, the cancer comprises an advanced solid tumor (local and/or metastatic) or a lymphoma. In embodiments, the cancer is selected from ovarian cancer, fallopian tube cancer, peritoneal cancer, cutaneous squamous cell carcinoma (CSCC), and squamous cell carcinoma of the head and neck (SCCHN). In embodiments, the cancer comprises an advanced solid tumor (local and/or metastatic) or advanced lymphoma. In embodiments, the cancer is a solid cancer. In embodiments, the cancer is a solid tumor. In embodiments, the cancer is a metastatic cancer. In embodiments, the cancer is a hematological cancer. In embodiments, the cancer expresses CD47.

Accordingly, in one aspect, the present disclosure relates to a method for treating a cancer in a human subject in need thereof the method comprising a step of administering to the human subject an effective amount of a chimeric protein having a general structure of: N terminus-(a)-(b)-(c)-C terminus, wherein: (a) is a first domain comprising an extracellular domain of human signal regulatory protein α (CD172a (SIRPα)), (b) is a linker adjoining the first and second domains, wherein the linker comprises a hinge-CH2-CH3 Fc domain, and (c) is a second domain comprising an extracellular domain of human CD40 ligand (CD40L). In embodiments, the chimeric protein is administered at a dose between about 0.0001 mg/kg and about 10 mg/kg. In embodiments, the chimeric protein exhibits a linear dose response in the dose range of e.g., about 0.3 mg/kg to about 3 mg/kg, or about 0.5 mg/kg to about 3 mg/kg, or about 0.7 mg/kg to about 3 mg/kg, or about 1 mg/kg to about 3 mg/kg, or about 1.5 mg/kg to about 3 mg/kg, or about 2 mg/kg to about 3 mg/kg, or about 2.5 mg/kg to about 3 mg/kg, or about 0.3 mg/kg to about 2.5 mg/kg, or about 0.3 mg/kg to about 2 mg/kg, or about 0.3 mg/kg to about 1.5 mg/kg, or about 0.3 mg/kg to about 1.0 mg/kg, or about 0.3 mg/kg to about 0.5 mg/kg. In embodiments, the chimeric protein does not exhibit a bell-shaped dose response.

In one aspect, the present disclosure relates to a method for evaluating the efficacy of cancer treatment in a subject in need thereof, wherein the subject is suffering from a cancer, the method comprising the steps of: (i) administering a dose of a chimeric protein, wherein the dose of from about 0.03 mg/kg to 10 mg/kg; wherein the chimeric protein has a general structure of: N terminus-(a)-(b)-(c)-C terminus, wherein: (a) is a first domain comprising an extracellular domain of human programmed cell death protein 1 (CD172a (SIRPα)), (b) is a linker adjoining the first and second domains, wherein the linker comprises a hinge-CH2-CH3 Fc domain, and (c) is a second domain comprising an extracellular domain of human CD40 ligand (CD40L); (ii) obtaining a biological sample from the subject; (iii) performing an assay on the biological sample to determine level and/or activity of a cytokine selected from CCL2, CXCL9, CXCL10, IFNα, IL15, IL23, IL-12, MCP-1, MIP-1β, MIP-1α, and MDC; and (iv) continuing administration of the chimeric protein if the subject has an increase in the level and/or activity of at least one cytokine selected from CCL2, CXCL9, CXCL10, IFNα, IL15, IL23, IL-12, MCP-1, MIP-1β, MIP-1α, and MDC.

In one aspect, the present disclosure relates to a method for evaluating the efficacy of cancer treatment in a subject in need thereof, wherein the subject is suffering from a cancer, the method comprising the steps of: (i) administering a dose of a chimeric protein, wherein the dose of from about 0.03 mg/kg to 10 mg/kg; wherein the chimeric protein has a general structure of: N terminus-(a)-(b)-(c)-C terminus, wherein: (a) is a first domain comprising an extracellular domain of human programmed cell death protein 1 (CD172a (SIRPα)), (b) is a linker adjoining the first and second domains, wherein the linker comprises a hinge-CH2-CH3 Fc domain, and (c) is a second domain comprising an extracellular domain of human CD40 ligand (CD40L); (ii) obtaining a biological sample from the subject; (iii) performing an assay on the biological sample to determine level and/or activity of a cytokine selected from CCL2, CXCL9, CXCL10, IFNα, IL6, IL15, IL23, and TNFα; and (iv) continuing administration of the chimeric protein if the subject has an increase in the level and/or activity of at least one cytokine selected from CCL2, CXCL9, CXCL10, IFNα, IL15, IL23, IL-12, MCP-1, MIP-1β, MIP-1α, and MDC, and/or if the subject has a lack of substantial increase in the level and/or activity of IL6 and/or TNFα.

In one aspect, the present disclosure relates to a method of evaluating the efficacy of a cancer treatment in a subject in need thereof comprising, the method comprising the steps of: obtaining a biological sample from the subject that has received a dose of a chimeric protein, wherein the dose of from about 0.03 mg/kg to 10 mg/kg, wherein the chimeric protein has a general structure of: N terminus-(a)-(b)-(c)-C terminus, wherein: (a) is a first domain comprising an extracellular domain of human signal regulatory protein α (CD172a (SIRPα)), (b) is a linker adjoining the first and second domains, wherein the linker comprises a hinge-CH2-CH3 Fc domain, and (c) is a second domain comprising an extracellular domain of human CD40 ligand (CD40L); performing an assay on the biological sample to determine level and/or activity of a cytokine selected from CCL2, CXCL9, CXCL10, IFNα, IL15, IL23, IL-12, MCP-1, MIP-1β, MIP-1α, and MDC; and administering the chimeric protein to the subject if the subject has an increase in the level and/or activity of at least one cytokine selected from CCL2, CXCL9, CXCL10, IFNα, IL15, IL23, IL-12, MCP-1, MIP-1β, MIP-1α, and MDC.

In one aspect, the present disclosure relates to a method of evaluating the efficacy of a cancer treatment in a subject in need thereof comprising, the method comprising the steps of: obtaining a biological sample from the subject that has received a dose of a chimeric protein, wherein the dose of from about 0.03 mg/kg to 10 mg/kg, wherein the chimeric protein has a general structure of: N terminus-(a)-(b)-(c)-C terminus, wherein: (a) is a first domain comprising an extracellular domain of human signal regulatory protein α (CD172a (SIRPα)), (b) is a linker adjoining the first and second domains, wherein the linker comprises a hinge-CH2-CH3 Fc domain, and (c) is a second domain comprising an extracellular domain of human CD40 ligand (CD40L); performing an assay on the biological sample to determine level and/or activity of a cytokine selected from CCL2, CXCL9, CXCL10, IFNα, IL6, IL15, IL23, and TNFα; and administering the chimeric protein to the subject if the subject has an increase in the level and/or activity of at least one cytokine selected from CCL2, CXCL9, CXCL10, IFNα, IL6, IL15, and IL23, and/or if the subject has a lack of substantial increase in the level and/or activity of IL6 and/or TNFα.

In one aspect, the present disclosure relates to a method of selecting a subject for treatment with a therapy for a cancer, the method comprising the steps of: (i) administering a dose of a chimeric protein, wherein the dose of from about 0.03 mg/kg to 10 mg/kg; wherein the chimeric protein has a general structure of: N terminus-(a)-(b)-(c)-C terminus, wherein: (a) is a first domain comprising an extracellular domain of human programmed cell death protein 1 (CD172a (SIRPα)), (b) is a linker adjoining the first and second domains, wherein the linker comprises a hinge-CH2-CH3 Fc domain, and (c) is a second domain comprising an extracellular domain of human CD40 ligand (CD40L); (ii) obtaining a biological sample from the subject; (iii) performing an assay on the biological sample to determine level and/or activity of a cytokine selected from CCL2, CXCL9, CXCL10, IFNα, IL15, IL23, IL-12, MCP-1, MIP-13, MIP-1α, and MDC; and (iv) selecting the subject for treatment with the chimeric protein if the subject has an increase in the level and/or activity of at least one cytokine selected from CCL2, CXCL9, CXCL10, IFNα, IL15, IL23, IL-12, MCP-1, MIP-1β, MIP-1α, and MDC.

In one aspect, the present disclosure relates to a method of selecting a subject for treatment with a therapy for a cancer, the method comprising the steps of: (i) administering a dose of a chimeric protein, wherein the dose of from about 0.03 mg/kg to 10 mg/kg; wherein the chimeric protein has a general structure of: N terminus-(a)-(b)-(c)-C terminus, wherein: (a) is a first domain comprising an extracellular domain of human programmed cell death protein 1 (CD172a (SIRPα)), (b) is a linker adjoining the first and second domains, wherein the linker comprises a hinge-CH2-CH3 Fc domain, and (c) is a second domain comprising an extracellular domain of human CD40 ligand (CD40L); (ii) obtaining a biological sample from the subject; (iii) performing an assay on the biological sample to determine level and/or activity of a cytokine selected from CCL2, CXCL9, CXCL10, IFNα, IL6, IL15, IL23 and TNFα; and (iv) selecting the subject for treatment with the chimeric protein if the subject has an increase in the level and/or activity of at least one cytokine selected from CCL2, CXCL9, CXCL10, IFNα, IL15, IL23, IL-12, MCP-1, MIP-1β, MIP-1α, and MDC, and/or if the subject has a lack of substantial increase in the level and/or activity of IL6 and/or TNFα.

In one aspect, the present disclosure relates to a method of selecting a subject for treatment with a therapy for cancer, the method comprising the steps of: obtaining a biological sample from the subject that has received a dose of a chimeric protein, wherein the dose of from about 0.03 mg/kg to 10 mg/kg, wherein the chimeric protein has a general structure of: N terminus-(a)-(b)-(c)-C terminus, wherein: (a) is a first domain comprising an extracellular domain of human signal regulatory protein α (CD172a (SIRPα)), (b) is a linker adjoining the first and second domains, wherein the linker comprises a hinge-CH2-CH3 Fc domain, and (c) is a second domain comprising an extracellular domain of human CD40 ligand (CD40L); performing an assay on the biological sample to determine level and/or activity of a cytokine selected from CCL2, CXCL9, CXCL10, IFNα, IL15, IL23, IL-12, MCP-1, MIP-1β, MIP-1α, and MDC; and selecting the subject for treatment with the chimeric protein if the subject has an increase in the level and/or activity of at least one cytokine selected from CCL2, CXCL9, CXCL10, IFNα, IL15, IL23, IL-12, MCP-1, MIP-13, MIP-1α, and MDC.

In one aspect, the present disclosure relates to a method of selecting a subject for treatment with a therapy for cancer, the method comprising the steps of: obtaining a biological sample from the subject that has received a dose of a chimeric protein, wherein the dose of from about 0.03 mg/kg to 10 mg/kg, wherein the chimeric protein has a general structure of: N terminus-(a)-(b)-(c)-C terminus, wherein: (a) is a first domain comprising an extracellular domain of human signal regulatory protein α (CD172a (SIRPα)), (b) is a linker adjoining the first and second domains, wherein the linker comprises a hinge-CH2-CH3 Fc domain, and (c) is a second domain comprising an extracellular domain of human CD40 ligand (CD40L); performing an assay on the biological sample to determine level and/or activity of a cytokine selected from CCL2, CXCL9, CXCL10, IFNα, IL6, IL15, IL23, and TNFα; and selecting the subject for treatment with the chimeric protein if the subject has an increase in the level and/or activity of at least one cytokine selected from CCL2, CXCL9, CXCL10, IFNα, IL6, IL15, and IL23; and/or if the subject has a lack of substantial increase in the level and/or activity of IL6 and/or TNFα.

In embodiments, the cancer comprises an advanced solid tumor (local and/or metastatic) or a lymphoma. In some embodiments of any of the aspects disclosed herein, the cancer is selected from ovarian cancer, fallopian tube cancer, peritoneal cancer, cutaneous squamous cell carcinoma (CSCC), and squamous cell carcinoma of the head and neck (SCCHN).

### BRIEF DESCRIPTION OF THE DRAWINGS

FIG. 1A to FIG. 1D show schematic illustrations of Type I transmembrane proteins (FIG. 1A and FIG. 1B, left proteins) and Type II transmembrane proteins (FIG. 1A and FIG. 1B, right proteins). A Type I transmembrane protein and a Type II transmembrane protein may be engineered such that their transmembrane and intracellular domains are omitted and the transmembrane proteins' extracellular domains are adjoined using a linker sequence to generate a single chimeric protein. As shown in FIG. 1C and FIG. 1D, the extracellular domain of a Type I transmembrane protein, e.g., CD172a (SIRPα), and the extracellular domain of a Type II transmembrane protein, e.g., CD40L, are combined into a single chimeric protein. FIG. 1C depicts the linkage of the Type I transmembrane protein and the Type II transmembrane protein by omission of the transmembrane and intracellular domains of each protein, and where the liberated extracellular domains from each protein have been adjoined by a linker sequence. The extracellular domains in this depiction may include the entire amino acid sequence of the Type I protein (e.g., CD172a (SIRPα)) and/or Type II protein (e.g., CD40L) which is typically localized outside the cell membrane, or any portion thereof which retains binding to the intended receptor or ligand. Moreover, the chimeric protein comprises sufficient overall flexibility and/or physical distance between domains such that a first extracellular domain (shown at the left end of the chimeric protein in FIG. 1C and FIG. 1D) is sterically capable of binding its receptor/ligand and/or a second extracellular domain (shown at the right end of the chimeric protein in FIG. 1C and FIG. 1D) is sterically capable of binding its receptor/ligand. FIG. 1D depicts adjoined extracellular domains in a linear chimeric protein wherein each extracellular domain of the chimeric protein is facing “outward”.

FIG. 2 shows immune inhibitory and immune stimulatory signaling that is relevant to the present disclosure (from Mahoney, Nature Reviews Drug Discovery 2015:14; 561-585), the entire contents of which are hereby incorporated by reference.

FIG. 3A to FIG. 3E illustrate that the SIRPα-Fc-CD40L chimeric protein (also referred to herein as agonist redirected checkpoint (ARC) fusion protein) retained proper folding and binding to CD47 and CD40. FIG. 3A (top pane) I shows the predicted tertiary structure of human SIRPα-Fc-CD40L (RaptorX; University of Chicago, Chicago, IL). FIG. 3A (lower panel) shows the western blot analysis of SIRPα-Fc-CD40L performed by probing purified protein with human anti-SIRPα, anti-Fc, and anti-CD40L, under nonreducing and reducing conditions, and ± the deglycosylase PNGase F. FIG. 3B shows the electron microscopy indicating hexameric structure of the SIRPα-Fc-CD40L drug substance. Scale is shown, and lighter arrows correspond with each identified monomer. A schematic of the hexamer species is shown to the right, depicting dimerization of the Fc domain (red lines=disulfide bonds) and trimerization of the CD40L domain. FIG. 3C shows the functional dual ELISA using capture with recombinant hCD40, followed by detection with recombinant hCD47-His and then anti-His-HRP. FIG. 3D shows the flow cytometry-based binding of SIRPα-Fc-CD40L to CHOK1 cells engineered to stably express human CD47 or human CD40. MFI, mean fluorescence intensity. FIG. 3E shows the competition ELISA in which the disruption of binding of recombinant hSIRPα-Fc to plate-bound hCD47 was assessed in the presence or absence of hSIRPα-Fc-CD40L or a human CD47-blocking antibody (clone CC2C6).

FIG. 4A to FIG. 4F illustrate that the SIRPα domain of the SIRPα-Fc-CD40L chimeric protein stimulated phagocytosis in vitro and activation of DCs in vivo. FIG. 4A shows the human macrophage (red)/lymphoma (Toledo; green) coculture analysis of phagocytosis using immunofluorescence (IF). DAPI was used to label nuclei (blue), and merged channels and phase images are shown to the right. The human IgG negative control was used at 0.06 μmol/L, rituximab at 0.06 μmol/L, and hSIRPα-Fc-CD40L at 1 μmol/L. FIG. 4B shows the number of tumor cells engulfed per total number of macrophages was quantitated using 7 to 13 distinct IF images obtained for each of the treatment conditions. ImageJ software was used for the analysis. FIG. 4C shows the human macrophages and Toledo lymphoma cell coculture phagocytosis assessed by flow cytometry; with an IgG negative control, monotherapy hSIRPα-Fc-CD40L and rituximab, or the combination of these agents; analyzed after 2 hours. Identical assays were set up where macrophages were preincubated for 1 hour with 20 μg/mL of a commercially available Fc block cocktail, with 20 μg/mL of a CD40 blocking antibody or with 5 μg/mL of a calreticulin blocking peptide. FIG. 4D shows the analysis of phagocytosis. IncuCyte phRodo Red prelabeled Toledo cells were cocultured with human macrophages, and phagocytosis was assessed using time-lapse fluorescent microscopy over a course of 5 hours. Cocultures were treated with hSIRPα-Fc-CD40L (1 μmol/L), rituximab (1 μg/mL), anti-CD47 (clone CC2C6; 33 μg/mL), or with combinations of either SIRPα-Fc-CD40L and rituximab or anti-CD47 and rituximab. FIG. 4E shows the analysis of phagocytosis as assessed by flow cytometry. The indicated human tumor cell lines were treated with monotherapy with SIRPα-Fc-CD40L at 1 μmol/L, cetuximab/trastuzumab monotherapy at 0.06 μmol/L, or the combination of the agents. The human tumor cell lines were verified to express the given targets. K562 cells were used as a negative control for phagocytosis. FIG. 4F shows the analysis of various cell types in mice treated with a single intravenous dose of sheep RBCs (1×10⁷ cells; as a positive control), blocking antibodies to CD47 and SIRPα (100 μg each), or mSIRPα-Fc-CD40L (at 100 or 300 μg). After 6 or 24 hours, mice were euthanized and spleens excised, dissociated, and assessed by flow cytometry for populations of activated CD4+ (left) or CD8+ DCs (right); also positive for MHC-II (I-Ab), CD11c, and DC1R2. See corresponding data in FIG. 10D to FIG. 10F.

FIG. 5A to FIG. 5F illustrate that the CD40L domain of the SIRPα-Fc-CD40L chimeric protein induced NFκB signaling, antigen-independent PBMC activation, and a type I IFN response. FIG. 5A shows that the human SIRPα-Fc-CD40L stimulated canonical NFκB signaling in CHO-K1/hCD40/NFκB reporter cells. Shown is luminescence after 6 hours. Recombinant human CD40L-His serves as a positive control. FIG. 5B shows the bioluminescence in the noncanonical NIK/NFκB reporter U2OS cells (expressing human CD40). The NIK/NFκB reporter U2OS cells were cultured with a titration of recombinant hCD40L-Fc, an agonist hCD40 antibody, or SIRPα-Fc-CD40L. Shown is luminescence after 6 hours. CD8-depleted PBMCs from 33 to 50 distinct human donors were cultured with media only, the neoantigen positive control KLH, the clinical stage nonactivating control exenatide, or 0.3, 3, 30, or 300 nmol/L of hSIRPα-Fc-CD40L. FIG. 5C shows the proliferation on days 5, 6, and 7, as assessed via [³H]-Thymidine incorporation. FIG. 5D shows the IL2-positive cells on day 8 as assessed by ELISpot. FIG. 5E shows the IFNα1, IFNβ1, CD80, and CD86 gene expression from macrophages harvested from macrophage:Toledo lymphoma cell cocultures in phagocytosis assays in the presence of rituximab (0.06 μmol/L), hSIRPα-Fc-CD40L (1 μmol/L), or the combination of both agents. FIG. 5F shows the type I IFN-induced luminescence in RAW 264.7-Lucia ISG cells, which were cocultured with A20 lymphoma cells in the presence of 50 μg/mL of mSIRPα-Fc-CD40L, recombinant Fc-mCD40L, mSIRPα-Fc, or their combination or 1 μg/mL anti-mCD20, or the combination of mSIRPα-Fc-CD40L and anti-mCD20. After 24 hours, type I IFN-induced luminescence was assessed using a luminometer. The maximum signal of luminescence across all experiments was set to 1, and all other values were normalized accordingly.

FIG. 6A to FIG. 6E illustrate the antitumor efficacy of the murine SIRPα-Fc-CD40L chimeric protein surrogate. FIG. 6A shows the CT26 tumor growth curves of mice treated with two doses of vehicle (PBS; n=21), anti-CD40 (clone FGK4.5; n=8), anti-CD47 (clone MIAP301; n=8), the combination of both antibodies (n=9; 100 μg per antibody, per dose), or mSIRPα-Fc-CD40L (150-300 μg per dose; n=10). STV stands for “starting tumor volume” on the day that treatment began. FIG. 6B shows the percentage of AH antigen-specific CD8+ T cells in the spleens and tumors using tetramer reagents. FIG. 6C shows the CT26 tumor growth curves in mice predepleted of CD4, CD8, or both CD4 and CD8 cells prior to the initiation of treatment with vehicle (PBS; n=10) or the mSIRPα-Fc-CD40L chimeric protein (n=10; 300 μg per dose) on days 7, 9, and 11. Tumor growth curves of WEHI3 (FIG. 6D) or A20 tumors (FIG. 6E) in mice treated with vehicle (PBS; n=11 mice in each tumor model), anti-CD20 (100 μg per dose; n=9 in A20 and n=10 in WEHI3), mSIRPα-Fc-CD40L (300 μg per dose; n=11 in A20 and n=10 in WEHI3), or the combination of mSIRPα-Fc-CD40L and anti-CD20 (n=10 in A20 and n=9 in WEHI3) on days 7, 9, and 11 (WEHI3) or days 10, 12, and 14 (A20); when tumors were established and reached approximately 65 to 70 mm³. Also shown are tumor growth curves in mice predepleted of IFNAR1 cells prior to the initiation of treatment. Groups included anti-CD20 (n=10 in both tumor models), mSIRPα-Fc-CD40L (n=12 in A20 and n=11 in WEHI3), or the combination of the two agents (n=10 in A20 and n=8 in WEHI3).

FIG. 7A to FIG. 7C illustrate that the mSIRPα-Fc-CD40L exhibited improved antitumor efficacy when combined with anti-CTLA4 or anti-PD-1. FIG. 7A and FIG. 7B show the tumor growth curves of mice bearing larger CT26 tumors [mean starting tumor volume (STV) 89.76 mm³] after treatment with anti-CTLA4 (clone 9D9; A) and anti-PD-1 (clone RMP1-14; B) either dosed before mSIRPα-Fc-CD40L (on days 7, 9, and 11 with the SIRPα-Fc-CD40L chimeric protein on days 12, 14, and 16), after mSIRPα-Fc-CD40L (on days 12, 14, and 16 with the SIRPα-Fc-CD40L chimeric protein on days 7, 9, and 11), or simultaneously with mSIRPα-Fc-CD40L (on days 7, 9, and 11). Experimental replicates are shown in FIG. 13A and FIG. 13C. FIG. 7C shows Day 11 CT26 tumor and infiltrating leukocyte phenotyping by flowcytometry in mice treated with two intraperitoneal doses of 100 mg of either anti-CTLA4 or anti-PD-1 given on days 7 and 9. Cell populations were assessed for CD40+ DCs (CD11c+), B cells (CD19+), and T cells (CD3+; top) and MHC-1, MHC-II, and CD47+ cells (bottom) based on cell counts and tumor weights taken at harvest.

FIG. 8A to FIG. 8I illustrate the safety of the hSIRPα-Fc-CD40L chimeric protein in cynomolgus macaques and overall mechanism of action of the safety of the hSIRPα-Fc-CD40L chimeric protein (without wishing to be bound by theory). FIG. 8A shows the blood chemistry analysis as assessed by the peripheral erythrocyte counts, hemoglobin levels, and hematocrit. Cynomolgus macaques were treated with vehicle or 0.1, 1, 10, and 40 mg/kg of SIRPα-Fc-CD40L peripheral erythrocyte counts, hemoglobin levels, and hematocrit were measured at the indicated times. FIG. 8B shows the CD47 receptor occupancy as assessed by flow cytometry following the first dose. Plotted is the inverse of the Free-CD47 flow cytometry signal, since the loss in detectable CD47 signal with increasing dose is directly proportional to the percentage of cells whose CD47 receptor is already bound by hSIRPα-Fc-CD40L. FIG. 8C shows the fold change in peripheral lymphocyte count from predose to 24 hours postdose. FIG. 8D shows the levels of cytokines CCL2, IL-8 and CXCL9 in serum after dosing compared to the background levels prior to dosing. FIG. 8E shows the staining of Ki67 positive cells in lymph nodes after dosing compared to the background levels prior to dosing. FIG. 8F to FIG. 8I show the proposed mechanism of action of SIRPα-Fc-CD40L (without wishing to be bound by theory). FIG. 8F shows that tumor-expressed CD47 can provide a “do not eat me” signal to APCs through the binding of SIRPα. FIG. 8G shows that the SIRPα-Fc-CD40L chimeric protein can relieve this inhibitory signal while simultaneously providing an “eat me” signal via costimulation of CD40 by CD40L. This enhances tumor phagocytosis, APC activation, increased antigen processing/presentation, and induction of an antitumor antigen-specific CD8+ T-cell response. FIG. 8H and FIG. 8I show that combining SIRPα-Fc-CD40L with targeted ADCP-competent antibodies potentiates their phagocytosis activity.

FIG. 9A to FIG. 9G show further characterization of human SIRPα-Fc-CD40L and the murine mSIRPα-Fc-CD40L surrogate. FIG. 9A shows the single-sided ELISA detection of hSIRPα-Fc-CD40L using recombinant Fc, CD47, and CD40 capture. FIG. 9B shows the verification of human CD47 and human CD40 expression in CHO-K1 cells used to assess binding to hSIRPα-Fc-CD40L. FIG. 9C shows the exemplary flow cytometry gating from FIG. 3E, depicting binding of SIRPα-Fc-CD40L to CHO-K1 parental cells, or CHO-K1 cells engineered to overexpress human CD47 or CD40. In this example, cells were incubated with 10 g/mL of human SIRPα-Fc-CD40L. To generate the binding curves in FIG. 3E, cells were incubate with a dose titration of SIRPα-Fc-CD40L. FIG. 9D shows the western blot analysis of the murine SIRPα-Fc-CD40L surrogate with antibodies detecting mSIRPα, mFc, and mCD40L under non-reducing, reducing, and PNGase F/reducing conditions. FIG. 9E shows the dual functional ELISA of the murine SIRPα-Fc-CD40L surrogate, demonstrating simultaneous binding to recombinant mouse CD47 and CD40. FIG. 9F shows the murine version of the phagocytosis assay using bone marrow derived macrophages co-cultured with A20 lymphoma or WEHI3 leukemia cells, in the presence of mSIRPα-Fc-CD40L or anti-CD47. FIG. 9G shows the results of the murine version of the NFκB-luciferase reporter assay in CHO-K1 cells developed to express murine CD40 and an NFκB-luciferase reporter.

FIG. 10A to FIG. 10F show that the supportive phagocytosis data and SIRPα-driven activation of dendritic cells in vivo. FIG. 10A shows the phagocytosis quantitation of Raji cells by human macrophages using flow cytometry in the presence of hSIRPα-Fc-CD40L+/−Rituximab. FIG. 10B shows the flow cytometry phenotyping of surface expressed EGFR, HER2, CD40, and CD47 in the human tumor cell lines used for phagocytosis assays in FIG. 4E. FIG. 10C shows the exemplary gating for flow cytometry based phagocytosis assays in FIG. 4C, FIG. 4E, and FIG. 9F. FIG. 10D shows a schematic and FIG. 10E shows the quantitation of in vivo dendritic cell activation; corresponding to FIG. 4F. Shown is the absolute percentage of CD4+ and CD8+ dendritic cells; also gated on CD11c and DC1R2. FIG. 10F shows the exemplary gating for flow cytometry in FIG. 4F, FIG. 10C, and FIG. 10D. The same gating strategy was used for CD4+ DCs as is shown for CD8+ DCs.

FIG. 11A and FIG. 11B illustrate that the type I interferon expression in macrophages co-cultured with Raji cells. FIG. 11A shows the exemplary gating for CD8 depletion flow cytometry from human PBMCs presented in FIG. 5C-FIG. 5D. FIG. 11B shows the supportive data for FIG. 5E. Fold-change in gene expression relative to ACTB and the untreated control was assessed in CD11b+ sorted macrophages following 2 h co-culture with Raji cells treated with h SIRPα-Fc-CD40L+/−Rituximab (Ritux).

FIG. 12A to FIG. 12E illustrate that the blockade of CD4, CD8, and IFNAR1. FIG. 12A shows the exemplary gating for flow cytometry in FIG. 6B. FIG. 12B shows the peripheral blood analysis by flow cytometry of CD4 (left), CD8 (middle), and IFNAR1 (right) following depleting antibody treatment corresponding to FIG. 6C-FIG. 6E. Samples were normalized to untreated animals. FIG. 12C shows the exemplary flow cytometry gating for CD4, CD8, and IFNAR1 depletion shown in FIG. 12B. FIG. 12D shows that the CD4 and CD8 cells were depleted from CT26 tumor bearing mice on days 9, 11, and 15 of the time-course; after treatment with SIRPα-Fc-CD40L began (treatment on days 7, 9, and 11). This strategy is referred to as ‘late’ depletion and correlates with the ‘early’ CD4/CD8 depletion shown in FIG. 6C. FIG. 12E shows the effect of the IFNAR1+ cell depletion. IFNAR1+ cells were depleted from WEHI3 (left) and A20 (right) bearing mice on days 9, 11, and 13 in the WEHI3 animals and on days 12, 14, and 16 in the A20 animals after treatment with SIRPα-Fc-CD40L began. As in FIG. 6D-FIG. 6E, mSIRPα-Fc-CD40L was given on days 7, 9, and 11 in WEHI3 bearing mice and on days 10, 12, and 14 in A20 bearing mice. This strategy is referred to as ‘late’ depletion and correlates with the ‘early’ IFNAR1 depletion shown in FIG. 6D-FIG. 6E.

FIG. 13A to FIG. 13E illustrate the CT26 combination experiment with various sequencing of mSIRPα-Fc-CD40L and anti-CTLA4 or anti-PD1. FIG. 13A and FIG. 13C show the combinations with anti-CTLA4 or anti-PD1 respectively; number of mice in each treatment group, number of mice that rejected the primary CT26 tumor, number of mice that rejected the secondary CT26 tumor re-challenge (without subsequent retreatment). FIG. 13B and FIG. 13D show the Mantle Cox survival and statistical analysis between monotherapy and combination groups. ‘d’ represents ‘day’ on which the therapies were administered. FIG. 13E shows the exemplary flow cytometry gating for FIG. 7C.

FIG. 14A to FIG. 14F illustrate that the hemolysis assessment and pharmacodynamic decrease in peripheral B cells following treatment with SIRPα-Fc-CD40L. FIG. 14A shows the exemplary flow cytometry gating for FIG. 8B. FIG. 14B shows the in vitro hemolysis assay using human donor RBCs treated with a titration of the positive control Triton X-100, a CD47 blocking antibody previously shown to induce RBClysis (clone CC2C6), and a titration of 3 separate lots of SIRPα-Fc-CD40L. FIG. 14C shows a decrease in overall CD45+ peripheral lymphocytes was observed 24 h following a single IP injection of mSIRPα-Fc-CD40L (300 μg). FIG. 14D shows the peripheral blood was isolated from mice receiving 3 IP doses (300 μg) of the murine SIRPα-Fc-CD40L surrogate (arrows). Cell populations were assessed by flow cytometry and included CD20+ B cells, CD11C+, CD4+/CD11cc+, and CD8+/CD11c+ dendritic cells. No significant differences were observed in mice treated with an interferon alpha receptor 1 depleting antibody (anti-IFNAR1). FIG. 14E shows a decrease in peripheral CD20+ B cells was observed 24 h following a single IP injection of a dose range of mSIRPα-Fc-CD40L. FIG. 14F shows the exemplary flow cytometry gating for FIG. 14C-FIG. 14E.

FIG. 15A and FIG. 15B show lymphocyte margination caused by SL-172154 in nonhuman primates. FIG. 15A shows the post-dose lymphocyte margination from day 15 to day 16. Cynomolgus monkeys were treated with SL-172154 on Day 1, 8 and 15 at the indicated dose. Pre- and post-dose lymphocyte counts were obtained on day 15 prior to the third dose, and on day 16 approximately 24 hours after the third dose. The number of peripheral blood lymphocytes was observed to decrease in a dose-dependent manner following the Day 15 dose, and is plotted as the (100−((#of lymphocytes on Day 16)/(#of lymphocytes on Day 15)×100). Each data point indicates an individual animal. FIG. 15B shows the histochemical analysis of spleens of from untreated and SL-172154-treated monkeys illustrating the migration of lymphocytes. Cynomolgus monkeys were administered 5 consecutive weekly doses SL-172154. Illustrative spleen section from a control and SL-172154-treated animal are shown.

FIG. 16 shows a schematic of the design of the Phase 1 clinical trial of SL-172154. The Phase 1 clinical trial is a first in human, open label, multi-center, dose escalation and dose expansion study in subjects with advanced solid tumors or lymphomas. The primary objective of this study is to evaluate the safety, tolerability of SL-172154. The secondary objective of this study is to evaluate the recommended phase II dose (RP2D), pharmacokinetic (PK), anti-tumor activity and pharmacodynamic effects of SL-172154. The exploratory objective of this study is to evaluate the pharmacodynamic (PD) markers in blood and tumor. The study design consists of Dose Escalation Cohorts and PD Cohorts, shown in the left and middle panel, respectively. The dose levels (DL) used in this study were DL1 through DL5, ranging from 0.1 mg/kg to 10.0 mg/kg. Right panel illustrates the recommended phase II dose (RP2D) decision based on the totality of study data, including safety, PK, PD and anti-tumor activity. The abbreviations used include: D=Day; q2wks=Every two weeks; PK=pharmacokinetics; PD=pharmacodynamics; and DLT=dose limiting toxicity.

FIG. 17 shows a schematic of the initial clinical development strategy of SL-172154 in Ovarian Cancer. The dose levels (DL) used in this study were DL1 through DL5, ranging from 0.1 mg/kg to 10.0 mg/kg.

FIG. 18 shows a schematic of the design of the Phase 1 clinical trial of SL-172154. In the dose escalation portion of the study, three or more patients will be enrolled through each of four dose levels, ranging from 0.003 mg to 0.1 mg.

FIG. 19 shows a schematic of the initial clinical development strategy of SL-172154 in CSCC and HNSCC. In the dose escalation portion of the study, three or more patients will be enrolled through each of four dose levels, ranging from 0.003 mg to 0.1 mg.

FIG. 20 shows a schematic of the initial clinical development strategy of SL-172154 in hematological cancers acute myeloid leukemia (AML), including TP53 mutant AML, and high-risk myelodysplastic syndromes (HR-MDS).

### DETAILED DESCRIPTION

The present disclosure is based, in part, on the discovery of certain doses of chimeric proteins for anti-cancer safety and efficacy in human patients, the chimeric proteins being engineered from the extracellular, or effector, regions of human signal regulatory protein α (CD172a (SIRPα)) and human CD40 ligand (CD40L). In addition, the present disclosure is based, in part, on the discovery that the maximal tolerated dose of the present chimeric proteins in humans is more than about 1 mg/kg. Further, the present disclosure is based, in part, on the discovery that the present chimeric proteins exhibit a linear dose response, as opposed to a bell-shaped response expected for this kind of agent.

Stimulation of CD40/CD40L signaling is a very appealing approach for experimental cancer therapy. However, multiple therapies that have been used in human patients have exhibited maximum-tolerated doses (MTD) in the range of 0.1 to 0.2 mg/kg. For example, the recombinant human CD40 ligand (rhuCD40L) (Avrend; Immunex Corp, Seattle, WA), exhibited an MTD of 0.1 mg/kg. Vonderheide et al, Phase I study of recombinant human CD40 ligand in cancer patients, J Clin Oncol 19(13): 3280-3287 (2001). The transient grade 3-4 liver function test abnormalities found in this study turned out to be a class effect of CD40 agonists. See Vonderheide, CD40 Agonist Antibodies in Cancer Immunotherapy, Annu. Rev. Med. 71:47-58 (2020). Likewise, CD40 agonist monoclonal antibodies like CP-870,893 (Selicrelumab, Pfizer) and APX005M (Sotigalimab, Apexigen) demonstrated MTDs in the range of 0.1 mg/kg to 0.2 mg/kg. Nowak et al, A phase 1b clinical trial of the CD40-activating antibody CP-870,893 in combination with cisplatin and pemetrexed in malignant pleural mesothelioma, Annals of Oncology 26(12): 2483-2490 (2015); Vonderheide et al, Phase I study of the CD40 agonist antibody CP-870,893 combined with carboplatin and paclitaxel in patients with advanced solid tumors, Oncoimmunology 2(1): e23033 (2013); Li and Wang, Characteristics and clinical trial results of agonistic anti-CD40 antibodies in the treatment of malignancies (Review), Oncology Letters 20: 176 (2020). In contrast to those studies, as the data presented herein in, e.g., Example 8, even a 3 mg/kg dose level of the SIRPα-Fc-CD40L chimeric protein—about 10 times higher than that of prior CD40 agonists—did not produce dose-limiting toxicities.

Accordingly, in aspects the present disclosure relates to a method for treating a cancer in a human subject, the method comprising a step of administering to the human subject a chimeric protein having a general structure of: N terminus-(a)-(b)-(c)-C terminus, in which (a) is a first domain comprising an extracellular domain of human signal regulatory protein α (CD172a (SIRPα)), (b) is a linker adjoining the first and second domains, wherein the linker comprises at least one cysteine residue capable of forming a disulfide bond and/or comprises a hinge-CH2-CH3 Fc domain, and (c) is a second domain comprising an extracellular domain of human CD40 ligand (CD40L). In embodiments, the dose of the chimeric protein administered is greater than about 0.2 mg/kg. In embodiments, the dose of the chimeric protein administered is at least about 1 mg/kg, e.g., at least about 1.0 mg/kg, or about 2 mg/kg, or about 3, about 4 mg/kg, or about 6 mg/kg, or about 8 mg/kg, or about 10 mg/kg.

In aspects, the present disclosure relates to a method for treating a cancer in a human subject, the method comprising a step of administering to the human subject a chimeric protein having a general structure of: N terminus-(a)-(b)-(c)-C terminus, in which (a) is a first domain comprising an extracellular domain of human signal regulatory protein α (CD172a (SIRPα)), (b) is a linker adjoining the first and second domains, wherein the linker comprises at least one cysteine residue capable of forming a disulfide bond and/or comprises a hinge-CH2-CH3 Fc domain, and (c) is a second domain comprising an extracellular domain of human CD40 ligand (CD40L). In embodiments, the dose of the chimeric protein administered is at least about 0.3 mg/kg, e.g., at least about 0.3 mg/kg, or about 1.0 mg/kg, or about 2 mg/kg, or about 3, about 4 mg/kg, or about 6 mg/kg, or about 8 mg/kg, or about 10 mg/kg.

A bell-shaped dose-response refers to the phenomenon where an agent exerts a therapeutic effect (e.g. stimulatory effect) at low doses, which is diminished at higher doses. Instead, at higher doses, an inhibitory effect is observed. For example, previous studies have demonstrated a bell-shaped curve for the pharmacodynamic biomarker response to CD40 agonist antibodies in the circulation. See Smith et al., Rationale and clinical development of CD40 agonistic antibodies for cancer immunotherapy, Expert Opinion on Biological Therapy 17:1-12 (2021). In contrast, as disclosed herein, the present chimeric proteins display a linear dose response.

The present chimeric proteins provide advantages including, without limitation, ease of use and ease of production. This is because two distinct immunotherapy agents are combined into a single product which may allow for a single manufacturing process instead of two independent manufacturing processes. In addition, administration of a single agent instead of two separate agents allows for easier administration and greater patient compliance. Further, in contrast to, for example, monoclonal antibodies, which are large multimeric proteins containing numerous disulfide bonds and post-translational modifications such as glycosylation, the present chimeric proteins are easier and more cost effective to manufacture. Furthermore, while individual immunotherapy agents may or may not exert therapeutic effects in the place, at the same time, a single agent instead of two separate agents ensures their concerted action at the same microenvironment at the same time.

Another advantage the SIRPα-Fc-CD40L chimeric protein (e.g. SEQ ID NO: 59 or SEQ ID NO: 61) offers is that despite targeting does not cause an anemia or another cytopenia in the patient. This is because although the CD47/SIRPα interaction plays a key role in the lysis of RBCs, as shown herein, the SIRPα-Fc-CD40L chimeric protein does not cause lysis of RBCs. Accordingly, the present methods are less likely to cause anemia or another cytopenia in than, e.g. an anti-CD47 Ab. Yet another advantage is that the doses of the SIRPα-Fc-CD40L chimeric protein are not limited by anemia or another cytopenia effects and are therefore higher than doses are allowed compared to certain other therapeutics (e.g. anti-CD47 antibodies or SIRPalphaFc fusion protein). Further, in embodiments, a low dose priming is not needed.

Importantly, since a chimeric protein of the present disclosure (via binding of the extracellular domain of CD172a (SIRPα) to its receptor/ligand on a cancer cell) disrupts, blocks, reduces, inhibits, and/or sequesters the transmission of immune inhibitory signals, e.g., originating from a cancer cell that is attempting to avoid its phagocytosis and/or destruction, and (via binding of CD40L to its receptor) enhances, increases, and/or stimulates the transmission of an immune stimulatory signal to an anti-cancer immune cell, it can provide an anti-tumor effect by two distinct pathways; this dual-action is more likely to provide any anti-tumor effect in a patient and/or to provide an enhanced anti-tumor effect in a patient. Furthermore, since such chimeric proteins can act via two distinct pathways, they can be efficacious, at least, in patients who respond poorly to treatments that target one of the two pathways. Thus, a patient who is a poor responder to treatments acting via one of the two pathway, can receive a therapeutic benefit by targeting the other pathway.

### Chimeric Proteins

The chimeric proteins of the present disclosure comprise an extracellular domain of CD172a (SIRPα) and an extracellular domain of CD40L which together can simultaneously block immune inhibitory signals and stimulate immune activating signals.

Aspects of the present disclosure provide a chimeric protein comprising a general structure of: N terminus-(a)-(b)-(c)-C terminus, where (a) is a first domain comprising an extracellular domain of CD172a (SIRPα), (b) is a linker adjoining the first domain and the second domain, e.g., the linker comprising at least one cysteine residue capable of forming a disulfide bond and/or comprising a hinge-CH2-CH3 Fc domain, and (c) is a second domain comprising an extracellular domain of CD40L; wherein the linker connects the first domain and the second domain.

In embodiments, the first domain comprises substantially all of the extracellular domain of CD172a (SIRPα). In embodiments, the first domain is capable of binding a CD172a (SIRPα) ligand. In embodiments, the first domain is capable of binding a CD172a (SIRPα) ligand (e.g. CD47) expressed on cancer cell surface. In embodiments, the first domain is capable of inhibiting the binding of a CD172a (SIRPα) ligand (e.g. CD47) to the CD172a (SIRPα) protein located on myeloid and hematopoietic stem cells and neurons. In embodiments, the first domain is capable of inhibiting an immunosuppressive signal. In embodiments, the first domain is capable of inhibiting an immunosuppressive signal. In embodiments, the first domain is capable of inhibiting a macrophage checkpoint or “do not eat me” signal. In embodiments the therapy with the SIRPα-Fc-CD40L chimeric protein (e.g. SEQ ID NO: 59 or SEQ ID NO: 61) stimulates macrophages to phagocytize tumor cells and effectively present the tumor antigens of phagocytized tumor cells to T cells.

In embodiments, the second domain is capable of binding a CD40 receptor. In embodiments, the second domain comprises substantially all of the extracellular domain of CD40L. In embodiments, the second domain is capable of activating an immune stimulatory signal.

In embodiments, the chimeric protein is a recombinant fusion protein, e.g., a single polypeptide having the extracellular domains disclosed herein. For example, in embodiments, the chimeric protein is translated as a single unit in a prokaryotic cell, a eukaryotic cell, or a cell-free expression system.

In embodiments, the present chimeric protein is producible in a mammalian host cell as a secretable and fully functional single polypeptide chain.

In embodiments, chimeric protein refers to a recombinant protein of multiple polypeptides, e.g., multiple extracellular domains disclosed herein, that are combined (via covalent or non-covalent bonding) to yield a single unit, e.g., in vitro (e.g., with one or more synthetic linkers disclosed herein).

In embodiments, the chimeric protein is chemically synthesized as one polypeptide or each domain is chemically synthesized separately and then combined. In embodiments, a portion of the chimeric protein is translated and a portion is chemically synthesized.

In embodiments, an extracellular domain refers to a portion of a transmembrane protein which is capable of interacting with the extracellular environment. In embodiments, an extracellular domain refers to a portion of a transmembrane protein which is sufficient for binding to a ligand or receptor and is effective in transmitting a signal to a cell. In embodiments, an extracellular domain is the entire amino acid sequence of a transmembrane protein which is normally present at the exterior of a cell or of the cell membrane. In embodiments, an extracellular domain is that portion of an amino acid sequence of a transmembrane protein which is external of a cell or of the cell membrane and is needed for signal transduction and/or ligand binding as may be assayed using methods know in the art (e.g., in vitro ligand binding and/or cellular activation assays).

Transmembrane proteins typically consist of an extracellular domain, one or a series of transmembrane domains, and an intracellular domain. Without wishing to be bound by theory, the extracellular domain of a transmembrane protein is responsible for interacting with a soluble receptor or ligand or membrane-bound receptor or ligand (i.e., a membrane of an adjacent cell). Without wishing to be bound by theory, the trans-membrane domain(s) is responsible for localizing the transmembrane protein to the plasma membrane. Without wishing to be bound by theory, the intracellular domain of a transmembrane protein is responsible for coordinating interactions with cellular signaling molecules to coordinate intracellular responses with the extracellular environment (or visa-versa).

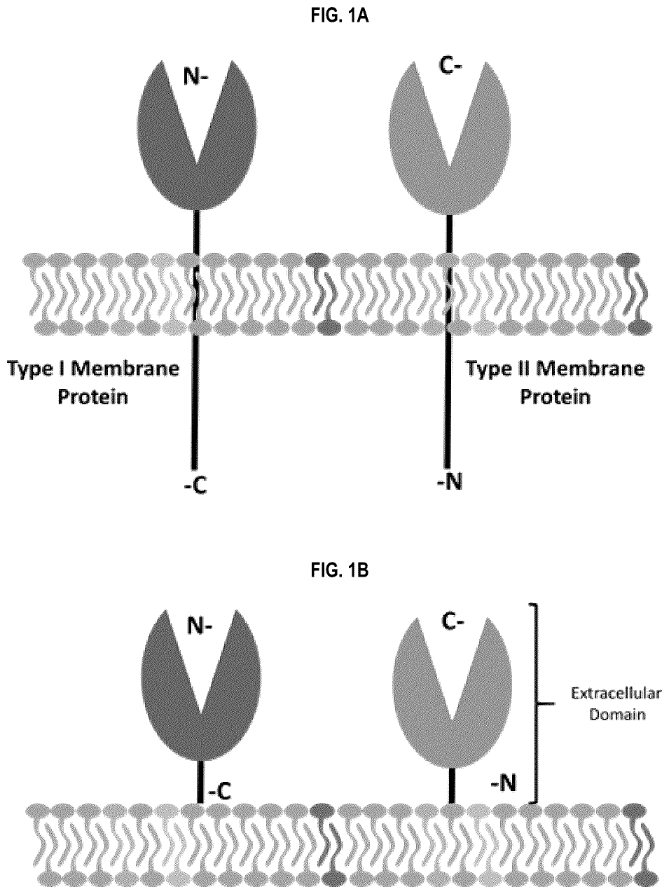

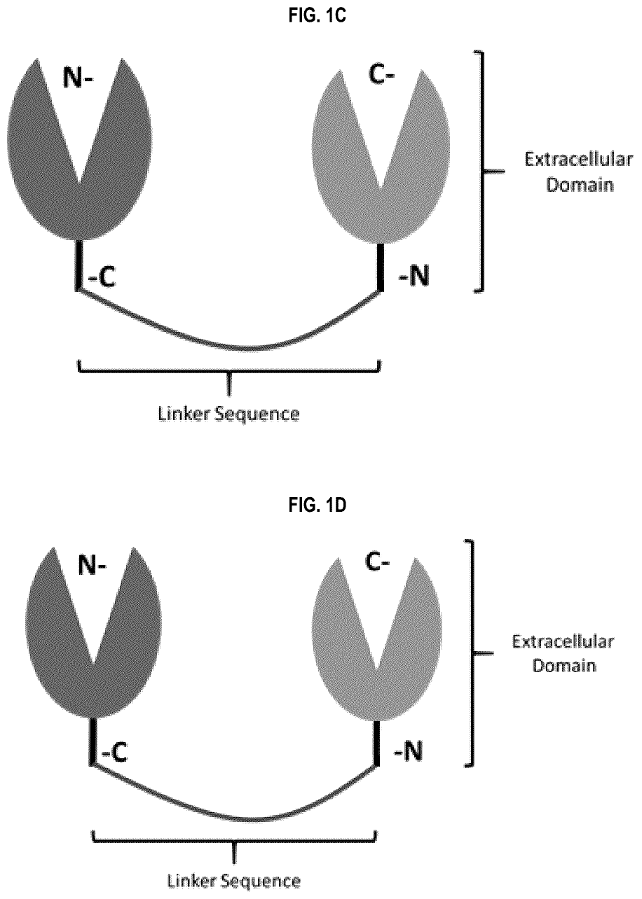

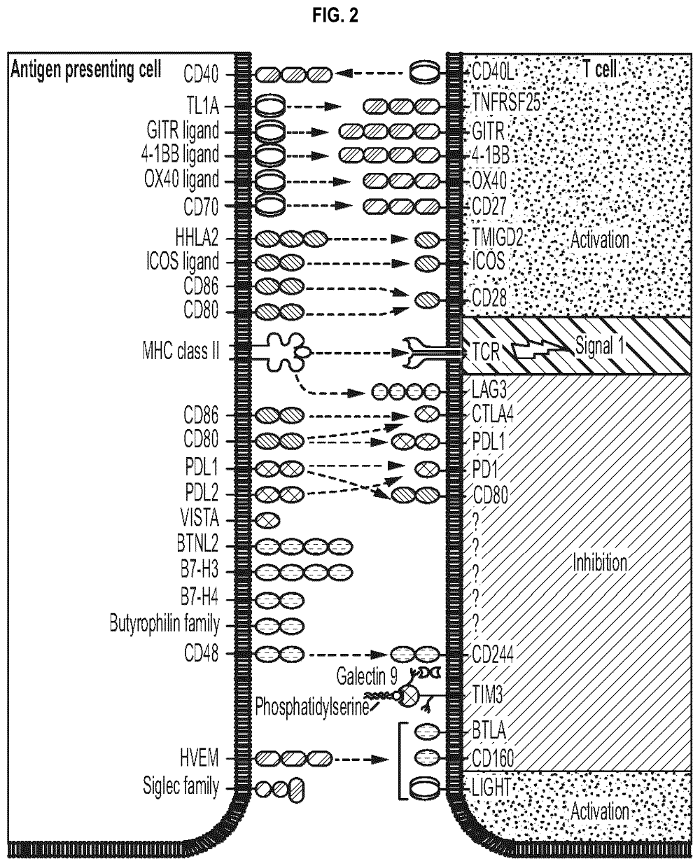

There are generally two types of single-pass transmembrane proteins: Type I transmembrane proteins which have an extracellular amino terminus and an intracellular carboxy terminus (see, FIG. 1A, left protein) and Type II transmembrane proteins which have an extracellular carboxy terminus and an intracellular amino terminus (see, FIG. 1A, right protein). Type I and Type II transmembrane proteins can be either receptors or ligands. For Type I transmembrane proteins (e.g., CD172a (SIRPα)), the amino terminus of the protein faces outside the cell, and therefore contains the functional domains that are responsible for interacting with other binding partners (either ligands or receptors) in the extracellular environment (see, FIG. 1B, left protein). For Type II transmembrane proteins (e.g., CD40L), the carboxy terminus of the protein faces outside the cell, and therefore contains the functional domains that are responsible for interacting with other binding partners (either ligands or receptors) in the extracellular environment (see, FIG. 1B, right protein). Thus, these two types of transmembrane proteins have opposite orientations to each other relative to the cell membrane.

The description of CD47 as a “do not eat me” signal in a broad range of cancers stimulated exploration of what combinations of “eat me” signals may enhance antitumor immunity in the setting of CD47 blockade. Willingham et al., The CD47-signal regulatory protein alpha (SIRPα) interaction is a therapeutic target for human solid tumors. Proc Natl Acad Sci USA 109: 6662-6667 (2012); Jaiswal et al., CD47 is upregulated on circulating hematopoietic stem cells and leukemia cells to avoid phagocytosis. Cell 138:271-285 (2009); Weiskopf et al., Engineered SIRPalpha variants as immunotherapeutic adjuvants to anticancer antibodies. Science 341:88-91 (2013). Preclinical combinations of CD47 blockade and ADCP-competent antibodies, including rituximab and trastuzumab, enhance tumor phagocytosis. Kauder et al, ALX148 blocks CD47 and enhances innate and adaptive antitumor immunity with a favorable safety profile. PLoS One 13: e0201832 (2018); Chao et al, Anti-CD47 antibody synergizes with rituximab to promote phagocytosis and eradicate non-Hodgkin lymphoma. Cell 142:699-713 (2010); Chao et al, Calreticulin is the dominant pro-phagocytic signal on multiple human cancers and is counterbalanced by CD47. Sci Transl Med 2:63ra94 (2010); Advani et al, CD47 Blockade by Hu5F9-G4 and rituximab in non-Hodgkin's lymphoma. N Engl J Med 379:1711-1721 (2018); Zhao et al., CD47-signal regulatory protein-alpha (SIRPaIpha) interactions form a barrier for antibody-mediated tumor cell destruction. Proc Natl Acad Sci USA 108:18342-18347 (2011). At least 50% of patients with relapsed or refractory diffuse large B-cell lymphoma or follicular lymphoma treated with Hu5F9-G4, a humanized, IgG4 isotype, CD47-blocking mAb, in combination with rituximab demonstrate objective responses. Advani et al., CD47 Blockade by Hu5F9-G4 and rituximab in non-Hodgkin's lymphoma. N Engl J Med 379:1711-1721 (2018). CD47 blockade enhances antigen presentation in immune-neglected tumors (Tseng et al., Anti-CD47 antibody-mediated phagocytosis of cancer by macrophages primes an effective antitumor T-cell response. Proc Natl Acad Sci USA 110:11103-11108 (2013)), yet only sporadic clinical responses have been observed using CD47/SIRPα blocking therapeutics as monotherapy or in combination with PD-1/L1-blocking antibodies.

Disrupting the binding of CD47 to SIRPα has emerged as a promising immunotherapeutic strategy for advanced cancers by potentiating antibody-dependent cellular phagocytosis (ADCP) of targeted antibodies. Preclinically, CD47/SIRPα blockade induces antitumor activity by increasing the phagocytosis of tumor cells by macrophages and enhancing the cross-presentation of tumor antigens to CD8+ T cells by dendritic cells; both of these processes are potentiated by CD40 signaling. Here a novel, two-sided fusion protein incorporating the extracellular domains of SIRPα and CD40L, adjoined by a central Fc domain, termed SIRPα-Fc-CD40L was generated. As shown herein, the SIRPα-Fc-CD40L chimeric protein bound CD47 and CD40 with high affinity and activated CD40 signaling in the absence of Fc receptor cross-linking. No evidence of hemolysis, hemagglutination, or thrombocytopenia was observed in vitro or in cynomolgus macaques. Further, as shown herein, the SIRPα-Fc-CD40L chimeric protein outperformed CD47 blocking and CD40 agonist antibodies in murine CT26 tumor models and synergized with immune checkpoint blockade of PD-1 and CTLA4. SIRPα-Fc-CD40L activated a type I interferon response in macrophages and potentiated the activity of ADCP-competent targeted antibodies both in vitro and in vivo. These data illustrated that whereas CD47/SIRPα inhibition could potentiate tumor cell phagocytosis, CD40-mediated activation of a type I interferon response provided a bridge between macrophage- and T-cell-mediated immunity that significantly enhanced durable tumor control and rejection.

Chimeric proteins of the present disclosure comprise an extracellular domain of CD172a (SIRPα) and an extracellular domain of CD40L. Thus, a chimeric protein of the present disclosure comprises, at least, a first domain comprising the extracellular domain of CD172a (SIRPα), which is connected—directly or via a linker—to a second domain comprising the extracellular domain of CD40L. As illustrated in FIG. 1C and FIG. 1D, when the domains are linked in an amino-terminal to carboxy-terminal orientation, the first domain is located on the “left’” side of the chimeric protein and is “outward facing” and the second domain is located on “right” side of the chimeric protein and is “outward facing”.

Other configurations of first and second domains are envisioned, e.g., the first domain is outward facing and the second domain is inward facing, the first domain is inward facing and the second domain is outward facing, and the first and second domains are both inward facing. When both domains are “inward facing”, the chimeric protein would have an amino-terminal to carboxy-terminal configuration comprising an extracellular domain of CD40L, a linker, and an extracellular domain of CD172a (SIRPα). In such configurations, it may be necessary for the chimeric protein to include extra “slack”, as described elsewhere herein, to permit binding domains of the chimeric protein to one or both of its receptors/ligands.

Constructs could be produced by cloning of the nucleic acids encoding the three fragments (the extracellular domain of CD172a (SIRPα), followed by a linker sequence, followed by the extracellular domain of CD40L) into a vector (plasmid, viral or other) wherein the amino terminus of the complete sequence corresponded to the ‘left’ side of the molecule containing the extracellular domain of CD172a (SIRPα) and the carboxy terminus of the complete sequence corresponded to the ‘right’ side of the molecule containing the extracellular domain of CD40L. In some embodiments of chimeric proteins having one of the other configurations, as described above, a construct would comprise three nucleic acids such that the translated chimeric protein produced would have the desired configuration, e.g., a dual inward-facing chimeric protein. Accordingly, In embodiments, the present chimeric proteins are engineered as such.

### CD172a (SIRPα)-Fc-CD40L Chimeric Protein

In embodiments, the chimeric protein is capable of contemporaneously binding the human CD172a (SIRPα) ligand and the human CD40 receptor, wherein the CD172a (SIRPα) ligand is CD47 and the CD40L receptor is CD40.

The chimeric protein has a general structure of: N terminus-(a)-(b)-(c)-C terminus, in which (a) is a first domain comprising an extracellular domain of human signal regulatory protein α (CD172a (SIRPα)), (b) is a linker adjoining the first and second domains, wherein the linker comprises a hinge-CH2-CH3 Fc domain, and (c) is a second domain comprising an extracellular domain of human CD40 ligand (CD40L). In embodiments, the linker comprises at least one cysteine residue capable of forming a disulfide bond.

Chimeric proteins of the present disclosure have a first domain which is sterically capable of binding its ligand/receptor and/or a second domain which is sterically capable of binding its ligand/receptor. This means that there is sufficient overall flexibility in the chimeric protein and/or physical distance between an extracellular domain (or portion thereof) and the rest of the chimeric protein such that the ligand/receptor binding domain of the extracellular domain is not sterically hindered from binding its ligand/receptor. This flexibility and/or physical distance (which is herein referred to as “slack”) may be normally present in the extracellular domain(s), normally present in the linker, and/or normally present in the chimeric protein (as a whole). Alternately, or additionally, the chimeric protein may be modified by including one or more additional amino acid sequences (e.g., the joining linkers described below) or synthetic linkers (e.g., a polyethylene glycol (PEG) linker) which provide additional slack needed to avoid steric hindrance.

In embodiments, the chimeric proteins of the present disclosure comprise variants of the extracellular domain of CD172a (SIRPα). As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the known amino acid sequence of CD172a (SIRPα), e.g., human CD172a (SIRPα).

In embodiments, the extracellular domain of CD172a (SIRPα) has the following amino acid sequence:

(SEQ ID NO: 57)
EEELQVIQPDKSVLVAAGETATLRCTATSLIPVGPIQWFRGAGPGRELIY
NQKEGHFPRVTTVSDLTKRNNMDFSIRIGNITPADAGTYYCVKFRKGSPD
DVEFKSGAGTELSVRAKPSAPVVSGPAARATPQHTVSFTCESHGFSPRDI
TLKWFKNGNELSDFQTNVDPVGESVSYSIHSTAKVVLTREDVHSQVICEV
AHVTLQGDPLRGTANLSETIRVPPTLEVTQQPVRAENQVNVTCQVRKFYP
QRLQLTWLENGNVSRTETASTVTENKDGTYNWMSWLLVNVSAHRDDVKLT
CQVEHDGQPAVSKSHDLKVSAHPKEQGSNTAAENTGSNERNIY

In embodiments, a chimeric protein comprises a variant of the extracellular domain of CD172a (SIRPα). As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 57.

In embodiments, the first domain of a chimeric protein comprises an amino acid sequence that is at least 90%, or 93%, or 95%, or 97%, or 98%, or 99% identical to the amino acid sequence of SEQ ID NO: 57. In embodiments, the first domain of a chimeric protein comprises an amino acid sequence that is at least 95% identical to the amino acid sequence of SEQ ID NO: 57. In embodiments, the first domain of a chimeric protein comprises an amino acid sequence that is at least 97% identical to the amino acid sequence of SEQ ID NO: 57. In embodiments, the first domain of a chimeric protein comprises an amino acid sequence that is at least 98% identical to the amino acid sequence of SEQ ID NO: 57. In embodiments, the first domain of a chimeric protein comprises an amino acid sequence that is at least 99% identical to the amino acid sequence of SEQ ID NO: 57. In embodiments, the first domain of a chimeric protein comprises an amino acid sequence that is identical to the amino acid sequence of SEQ ID NO: 57.

One of ordinary skill may select variants of the known amino acid sequence of CD172a (SIRPα) by consulting the literature, e.g. Hatherley et al., “Paired receptor specificity explained by structures of signal regulatory proteins alone and complexed with CD47.” Mol Cell 31: 266-277 (2008); Hatherley et al., “The Structure of the Macrophage Signal Regulatory Protein Alpha (Sirpalpha) Inhibitory Receptor Reveals a Binding Face Reminiscent of that Used by T Cell Receptors.” J Biol Chem 282: 14567 (2007); Hatherley et al, “Structure of Signal-Regulatory Protein Alpha: A Link to Antigen Receptor Evolution.” J Biol Chem 284: 26613 (2009); Hatherley et al., “Polymorphisms in the Human Inhibitory Signal-Regulatory Protein Alpha Do not Affect Binding to its Ligand Cd47.” J Biol Chem 289: 10024 (2014); Ring et al., “Anti-SIRP alpha antibody immunotherapy enhances neutrophil and macrophage antitumor activity.” Proc Natl Acad Sci USA 114: E10578-E10585 (2017), each of which is incorporated by reference in its entirety.

In embodiments, the chimeric proteins of the present disclosure comprise variants of the extracellular domain of CD40L. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the known amino acid sequence of CD40L, e.g., human CD40L.

In embodiments, the extracellular domain of CD40L has the following amino acid sequence:

(SEQ ID NO: 58)
HRRLDKIEDERNLHEDFVFMKTIQRCNTGERSLSLLNCEEIKSQFEGFVK
DIMLNKEETKKENSFEMQKGDQNPQIAAHVISEASSKTTSVLQWAEKGYY
TMSNNLVTLENGKQLTVKRQGLYYIYAQVTFCSNREASSQAPFIASLCLK
SPGRFERILLRAANTHSSAKPCGQQSIHLGGVFELQPGASVFVNVTDPSQ
VSHGTGFTSFGLLKL.

In embodiments, a chimeric protein comprises a variant of the extracellular domain of CD40L. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 58.

In embodiments, the second domain of a chimeric protein comprises an amino acid sequence that is at least 90%, or 93%, or 95%, or 97%, or 98%, or 99% identical to the amino acid sequence of SEQ ID NO: 58. In embodiments, the second domain of a chimeric protein comprises an amino acid sequence that is at least 95% identical to the amino acid sequence of SEQ ID NO: 58. In embodiments, the second domain of a chimeric protein comprises an amino acid sequence that is at least 97% identical to the amino acid sequence of SEQ ID NO: 58. In embodiments, the second domain of a chimeric protein comprises an amino acid sequence that is at least 98% identical to the amino acid sequence of SEQ ID NO: 58. In embodiments, the second domain of a chimeric protein comprises an amino acid sequence that is at least 99% identical to the amino acid sequence of SEQ ID NO: 58. In embodiments, the second domain of a chimeric protein comprises an amino acid sequence that is identical to the amino acid sequence of SEQ ID NO: 58.

One of ordinary skill may select variants of the known amino acid sequence of CD40L by consulting the literature, e.g. Karpusas et al., “2 A crystal structure of an extracellular fragment of human CD40 ligand.” Structure 3: 1031-1039 (1995); Karpusas et al, “Structure of CD40 ligand in complex with the Fab fragment of a neutralizing humanized antibody.” Structure 9: 321-329 (2001); Silvian et al, “Small Molecule Inhibition of the TNF Family Cytokine CD40 Ligand through a Subunit Fracture Mechanism.” ACS Chem Biol 6: 636-647 (2011); An et al., “Crystallographic and mutational analysis of the CD40-CD154 complex and its implications for receptor activation.” J Biol Chem 286: 11226-11235 (2011); Karnell et al, “A CD40L-targeting protein reduces autoantibodies and improves disease activity in patients with autoimmunity.” Sci Transl Med 11 (2019), each of which is incorporated by reference in its entirety.

In embodiments, the linker of a chimeric protein comprises an amino acid sequence that is at least 90%, or 93%, or 95%, or 97%, or 98%, or 99% identical to the amino acid sequence of SEQ ID NO: 1, SEQ ID NO: 2, or SEQ ID NO: 3. In embodiments, the linker of a chimeric protein comprises an amino acid sequence that is at least 95% identical to the amino acid sequence of SEQ ID NO: 1, SEQ ID NO: 2, or SEQ ID NO: 3. In embodiments, the linker of a chimeric protein comprises an amino acid sequence that is at least 97% identical to the amino acid sequence of SEQ ID NO: 1, SEQ ID NO: 2, or SEQ ID NO: 3. In embodiments, the linker of a chimeric protein comprises an amino acid sequence that is at least 98% identical to the amino acid sequence of SEQ ID NO: 1, SEQ ID NO: 2, or SEQ ID NO: 3. In embodiments, the linker of a chimeric protein comprises an amino acid sequence that is at least 99% identical to the amino acid sequence of SEQ ID NO: 1, SEQ ID NO: 2, or SEQ ID NO: 3. In embodiments, the linker of a chimeric protein comprises an amino acid sequence that is identical to the amino acid sequence of SEQ ID NO: 1, SEQ ID NO: 2, or SEQ ID NO: 3.

In embodiments, a chimeric protein of the present disclosure comprises: (1) a first domain comprising the amino acid sequence that is at least 90%, or 93%, or 95%, or 97%, or 98%, or 99% identical to SEQ ID NO: 57, (b) a second domain comprises the amino acid sequence that is at least 90%, or 93%, or 95%, or 97%, or 98%, or 99% identical to SEQ ID NO: 58, and (c) a linker comprises an amino acid sequence that is that is at least 90%, or 93%, or 95%, or 97%, or 98%, or 99% identical to the amino acid sequence of SEQ ID NO: 1, SEQ ID NO: 2, or SEQ ID NO: 3.

In embodiments, a chimeric protein of the present disclosure comprises: (1) a first domain comprising the amino acid sequence that is at least 95% identical to SEQ ID NO: 57, (b) a second domain comprises the amino acid sequence that is at least 95% identical to SEQ ID NO: 58, and (c) a linker comprises an amino acid sequence that is that is at least 95% identical to the amino acid sequence of SEQ ID NO: 1, SEQ ID NO: 2, or SEQ ID NO: 3. In embodiments, a chimeric protein of the present disclosure comprises: (1) a first domain comprising the amino acid sequence that is at least 97% identical to SEQ ID NO: 57, (b) a second domain comprises the amino acid sequence that is at least 97% identical to SEQ ID NO: 58, and (c) a linker comprises an amino acid sequence that is that is at least 97% identical to the amino acid sequence of SEQ ID NO: 1, SEQ ID NO: 2, or SEQ ID NO: 3. In embodiments, a chimeric protein of the present disclosure comprises: (1) a first domain comprising the amino acid sequence that is at least 98% identical to SEQ ID NO: 57, (b) a second domain comprises the amino acid sequence that is at least 98% identical to SEQ ID NO: 58, and (c) a linker comprises an amino acid sequence that is that is at least 98% identical to the amino acid sequence of SEQ ID NO: 1, SEQ ID NO: 2, or SEQ ID NO: 3. In embodiments, a chimeric protein of the present disclosure comprises: (1) a first domain comprising the amino acid sequence that is at least 99% identical to SEQ ID NO: 57, (b) a second domain comprises the amino acid sequence that is at least 99% identical to SEQ ID NO: 58, and (c) a linker comprises an amino acid sequence that is that is at least 99% identical to the amino acid sequence of SEQ ID NO: 1, SEQ ID NO: 2, or SEQ ID NO: 3. In embodiments, a chimeric protein of the present disclosure comprises: (1) a first domain comprising the amino acid sequence of SEQ ID NO: 57, (b) a second domain comprises the amino acid sequence of SEQ ID NO: 58, and (c) a linker comprises an amino acid sequence that is at least 95% identical to SEQ ID NO: 1, SEQ ID NO: 2, or SEQ ID NO: 3. In embodiments, a chimeric protein of the present disclosure comprises: (1) a first domain comprising the amino acid sequence identical to SEQ ID NO: 57, (b) a second domain comprises the amino acid sequence that is to SEQ ID NO: 58, and (c) a linker comprises an amino acid sequence that is that is identical to the amino acid sequence of SEQ ID NO: 1, SEQ ID NO: 2, or SEQ ID NO: 3.

In embodiments, a CD172a (SIRPα)-Fc-CD40L chimeric protein of the present disclosure has the following amino acid sequence (the extracellular domain of CD172a (SIRPα) is shown in a boldface font, the extracellular domain of CD40L is indicated by underline, Fc domain is shown in italic:

(SEQ ID NO: 59)
EEELQVIQPDKSVLVAAGETATLRCTATSLIPVGPIQWFRGAGPGRELIY
NQKEGHFPRVTTVSDLTKRNNMDFSIRIGNITPADAGTYYCVKFRKGSPD
DVEFKSGAGTELSVRAKPSAPVVSGPAARATPQHTVSFTCESHGFSPRDI
TLKWFKNGNELSDFQTNVDPVGESVSYSIHSTAKVVLTREDVHSQVICEV
AHVTLQGDPLRGTANLSETIRVPPTLEVTQQPVRAENQVNVTCQVRKFYP
QRLQLTWLENGNVSRTETASTVTENKDGTYNWMSWLLVNVSAHRDDVKLT
CQVEHDGQPAVSKSHDLKVSAHPKEQGSNTAAENTGSNERNIYSKYGPPC
PPCPAPEFLGGPSVFLFPPKPKDQLMISRTPEVTCVVVDVSQEDPEVQFN
WYVDGVEVHNAKTKPREEQFNSTYRVVSVLTVLHQDWLSGKEYKCKVSSK
GLPSSIEKTISNATGQPREPQVYTLPPSQEEMTKNQVSLTCLVKGFYPSD
IAVEWESNGQPENNYKTTPPVLDSDGSFFLYSRLTVDKSRWQEGNVFSCS
VLHEALHNHYTQKSLSLSLGKIEGRMDHRRLDKIEDERNLHEDFVFMKTI
QRCNTGERSLSLLNCEEIKSQFEGFVKDIMLNKEETKKENSFEMQKGDQN
PQIAAHVISEASSKTTSVLQWAEKGYYTMSNNLVTLENGKQLTVKRQGLY
YIYAQVTFCSNREASSQAPFIASLCLKSPGRFERILLRAANTHSSAKPCG
QQSIHLGGVFELQPGASVFVNVTDPSQVSHGTGFTSFGLLKL.

The 792 amino acid sequence of the CD172a (SIRPα)-Fc-CD40L chimeric protein (SL-172154) (not including the leader sequence) is shown above. The CD172a (SIRPα)-Fc-CD40L chimeric protein exists as a profile of oligomeric forms. There are 17 cysteines in the amino acid sequence with 8 likely disulfide pairs. Both N and O-linked glycosylation have been identified.

In embodiments, the chimeric protein of the present disclosure comprises at least 1, 2, 3, 4, 5, 6, 7, 8, 9, or 10 potential N glycosylation sites. In embodiments, the chimeric protein of the present disclosure comprises at least two potential N glycosylation sites. In embodiments, the chimeric protein of the present disclosure comprises at least four potential N glycosylation sites. In embodiments, the chimeric protein of the present disclosure comprises at least six potential N glycosylation sites. In embodiments, the chimeric protein of the present disclosure comprises at least eight potential N glycosylation sites. In embodiments, the chimeric protein of the present disclosure comprises at least ten potential N glycosylation sites. In embodiments, the chimeric protein of the present disclosure comprises at least 1, 2, 3, 4, 5, 6, 7, or 8 potential O glycosylation sites. In embodiments, the chimeric protein of the present disclosure comprises at least two potential O glycosylation sites. In embodiments, the chimeric protein of the present disclosure comprises at least four potential O glycosylation sites. In embodiments, the chimeric protein of the present disclosure comprises at least six potential O glycosylation sites. In embodiments, the chimeric protein of the present disclosure comprises at least eight potential O glycosylation sites. In embodiments, the chimeric protein of the present disclosure comprises at least two potential N glycosylation sites, and at least two potential O glycosylation sites. In embodiments, the chimeric protein of the present disclosure comprises at least four potential N glycosylation sites, and at least four potential O glycosylation sites. In embodiments, the chimeric protein of the present disclosure comprises at least six potential N glycosylation sites, and at least six potential O glycosylation sites. In embodiments, the chimeric protein of the present disclosure comprises at least eight potential N glycosylation sites, and at least eight potential O glycosylation sites. In embodiments, the chimeric protein of the present disclosure comprises at least ten potential N glycosylation sites, and at least eight potential O glycosylation sites. In embodiments, the chimeric protein expressed in Chinese Hamster Ovary (CHO) cells is glycosylated.

There are 17 cysteines present in the SL-172154 chimeric protein. In some embodiments, the SL-172154 chimeric protein has no disulfide bonds. In some embodiments, the SL-172154 chimeric protein has at least one, or at least two, or at least 3, or at least 4, or at least 5, or at least 6, or at least 7, or at least 8, or at least 9, or at least 10 disulfide bonds. In some embodiments, the SL-172154 chimeric protein has at least one, or at least two interchain disulfide bonds. In some embodiments, the SL-172154 chimeric protein has at least one, or at least two, or at least 3, or at least 4, or at least 5, or at least 6, or at least 7, or 8 intrachain disulfide bonds. In some embodiments, the SL-172154 chimeric protein has a C350=C350 interchain disulfide bond.

In some embodiments, the SL-172154 chimeric protein has a C353=C353 interchain disulfide bond. In some embodiments, the SL-172154 chimeric protein has a C153=C153 interchain disulfide bond. In some embodiments, the SL-172154 chimeric protein has a C25=C91 disulfide bond. In some embodiments, the SL-172154 chimeric protein has a C140=C198 disulfide bond. In some embodiments, the SL-172154 chimeric protein has a C243=C301 disulfide bond. In some embodiments, the SL-172154 chimeric protein has a C385=C445 disulfide bond. In some embodiments, the SL-172154 chimeric protein has a C491=C549 disulfide bond. In some embodiments, the SL-172154 chimeric protein has a C603=C615 disulfide bond. In some embodiments, the SL-172154 chimeric protein has a C709=C725 disulfide bond. In some embodiments, the SL-172154 chimeric protein has a C140=C243=C709/C725 scrambled disulfide bond. In some embodiments, the SL-172154 chimeric protein has a C615 (chain1)=C615 (chain2) scrambled disulfide bond.

In some embodiments, the CD172a (SIRPα)-Fc-CD40L chimeric protein of the present disclosure is encoded by the following nucleotide sequence (leader sequence is shown by a bold-underlined font):

(SEQ ID NO: 60)
ATGGAATGGAGCTGGGTGTTCTTGTTCTTCCTGTCCGTGACCACCGGCGTGCACTCGGAGGAGGAG
CTCCAGGTCATCCAGCCGGACAAGTCGGTGCTCGTGGCCGCCGGAGAAACTGCCACCCTGAGGTGC
ACCGCGACCTCGCTGATTCCCGTGGGCCCGATTCAGTGGTTCCGGGGGGCCGGGCCTGGCAGAGAA
CTGATCTACAACCAGAAGGAAGGCCATTTCCCTCGCGTGACTACTGTGTCCGATCTTACTAAGCGGAA
CAACATGGACTTCAGCATTAGGATCGGCAACATCACCCCTGCTGACGCGGGAACCTACTACTGCGTCA
AGTTCAGGAAAGGAAGCCCGGACGACGTGGAGTTCAAGAGCGGGGGGGGCACCGAACTGTCCGTGC
GCGCCAAGCCATCCGCGCCCGTGGTGTCCGGACCCGCAGCCAGAGCAACTCCGCAGCACACCGTGT
CGTTCACTTGCGAATCACACGGATTCTCCCCGCGCGATATCACGCTTAAGTGGTTCAAGAACGGGAAC
GAACTGAGCGACTTCCAGACCAACGTGGACCCCGTCGGAGAAAGCGTCAGCTACTCCATTCACTCGA
CCGCCAAAGTGGTGCTGACCAGGGAGGACGTGCATAGCCAAGTGATCTGCGAGGTCGCCCACGTCA
CTCTGCAAGGAGATCCGCTGCGGGGAACAGCCAACCTGTCCGAAACTATCCGCGTGCCTCCCACCCT
GGAAGTGACCCAGCAGCCCGTCCGAGCGGAGAATCAAGTCAATGTGACCTGTCAAGTCCGGAAATTC
TACCCTCAACGGCTCCAGCTGACCTGGCTGGAAAACGGAAACGTGTCCCGCACGGAAACCGCCTCGA
CCGTGACCGAGAACAAGGACGGCACCTACAACTGGATGTCCTGGCTCTTGGTGAACGTGTCAGCCCA
CCGGGACGATGTCAAGCTGACTTGCCAAGTGGAACATGATGGGCAGCCAGCTGTCAGCAAGAGCCAC
GACCTGAAGGTGTCCGCGCACCCGAAGGAACAGGGTTCGAATACTGCCGCCGAAAACACTGGTAGCA
ACGAACGGAACATCTACTCTAAGTACGGCCCACCTTGCCCTCCCTGCCCGGCACCTGAATTTCTGGGT
GGACCCTCCGTGTTTCTTTTCCCGCCCAAGCCAAAGGACCAGTTGATGATCTCCCGCACTCCGGAAGT
GACATGCGTGGTGGTGGACGTGTCCCAGGAAGATCCGGAAGTGCAGTTCAATTGGTACGTGGATGGC
GTGGAGGTCCATAACGCCAAGACTAAGCCGCGCGAGGAACAGTTCAATTCCACCTACCGGGTGGTGT
CCGTGCTGACCGTGCTGCATCAGGACTGGCTCTCCGGCAAAGAGTACAAGTGCAAGGTGTCATCCAA
GGGTCTGCCGTCGTCAATCGAAAAGACCATTTCCAATGCCACTGGGCAGCCCAGAGAACCTCAAGTCT
ACACCCTCCCACCGTCCCAAGAGGAAATGACCAAGAACCAAGTCTCGCTGACGTGTCTCGTGAAGGG
ATTCTACCCATCCGACATTGCTGTGGAATGGGAGTCCAACGGCCAGCCCGAGAACAACTACAAGACTA
CCCCTCCCGTCCTGGACTCCGACGGTTCCTTCTTCCTTTACTCTCGCCTCACCGTGGATAAGTCGCGG
TGGCAGGAGGGGAACGTGTTCTCCTGCTCCGTCCTGCACGAAGCATTGCACAACCACTACACCCAGA
AGTCCCTGTCACTGTCCCTGGGAAAGATTGAGGGTCGGATGGATCATCGGCGCCTGGACAAGATCGA
GGACGAGCGGAACCTCCACGAGGATTTCGTGTTCATGAAAACCATCCAGAGATGCAACACCGGAGAG
AGAAGCCTGTCCCTGCTCAACTGCGAGGAAATCAAGTCCCAGTTTGAAGGATTTGTGAAGGACATTAT
GCTGAACAAGGAAGAGACTAAGAAGGAAAACTCCTTCGAGATGCAGAAGGGCGATCAGAACCCACAG
ATCGCGGCCCACGTGATCTCCGAGGCCTCGTCAAAGACCACTTCAGTGCTCCAATGGGCCGAGAAGG
GTTACTATACCATGAGCAACAACCTTGTGACCCTGGAGAACGGAAAGCAGCTCACCGTGAAAAGACAG
GGACTGTACTATATCTATGCCCAAGTCACCTTCTGTTCGAACCGCGAGGCTAGCAGCCAGGCCCCGTT
CATCGCCTCCCTCTGTTTGAAGTCGCCGGGGCGGTTTGAAAGGATTCTGCTGAGAGCTGCGAATACC
CATTCGTCCGCCAAGCCTTGCGGACAGCAGTCAATCCACCTGGGGGGAGTGTTCGAGCTGCAGCCTG
GCGCGAGCGTGTTCGTCAACGTGACCGACCCCTCCCAAGTGTCTCACGGCACCGGATTCACTTCGTT
TGGCCTGCTGAAGCTGTAA

In some embodiments, the SEQ ID NO: 60 encodes for a precursor of the CD172a (SIRPα)-Fc-CD40L chimeric protein of the present disclosure having following amino acid sequence (leader sequence is shown by an italic font):

(SEQ ID NO: 61)
MEWSWVFLFFLSVTTGVHSEEELQVIQPDKSVLVAAGETATLRCTATSLI
PVGPIQWFRGAGPGRELIYNQKEGHFPRVTTVSDLTKRNNMDFSIRIGNI
TPADAGTYYCVKFRKGSPDDVEFKSGAGTELSVRAKPSAPVVSGPAARAT
PQHTVSFTCESHGFSPRDITLKWFKNGNELSDFQTNVDPVGESVSYSIHS
TAKVVLTREDVHSQVICEVAHVTLQGDPLRGTANLSETIRVPPTLEVTQQ
PVRAENQVNVTCQVRKFYPQRLQLTWLENGNVSRTETASTVTENKDGTYN
WMSWLLVNVSAHRDDVKLTCQVEHDGQPAVSKSHDLKVSAHPKEQGSNTA
AENTGSNERNIYSKYGPPCPPCPAPEFLGGPSVFLFPPKPKDQLMISRTP
EVTCVVVDVSQEDPEVQFNWYVDGVEVHNAKTKPREEQFNSTYRVVSVLT
VLHQDWLSGKEYKCKVSSKGLPSSIEKTISNATGQPREPQVYTLPPSQEE
MTKNQVSLTCLVKGFYPSDIAVEWESNGQPENNYKTTPPVLDSDGSFFLY
SRLTVDKSRWQEGNVFSCSVLHEALHNHYTQKSLSLSLGKIEGRMDHRRL
DKIEDERNLHEDFVFMKTIQRCNTGERSLSLLNCEEIKSQFEGFVKDIML
NKEETKKENSFEMQKGDQNPQIAAHVISEASSKTTSVLQWAEKGYYTMSN
NLVTLENGKQLTVKRQGLYYIYAQVTFCSNREASSQAPFIASLCLKSPGR
FERILLRAANTHSSAKPCGQQSIHLGGVFELQPGASVFVNVTDPSQVSHG
TGFTSFGLLKL

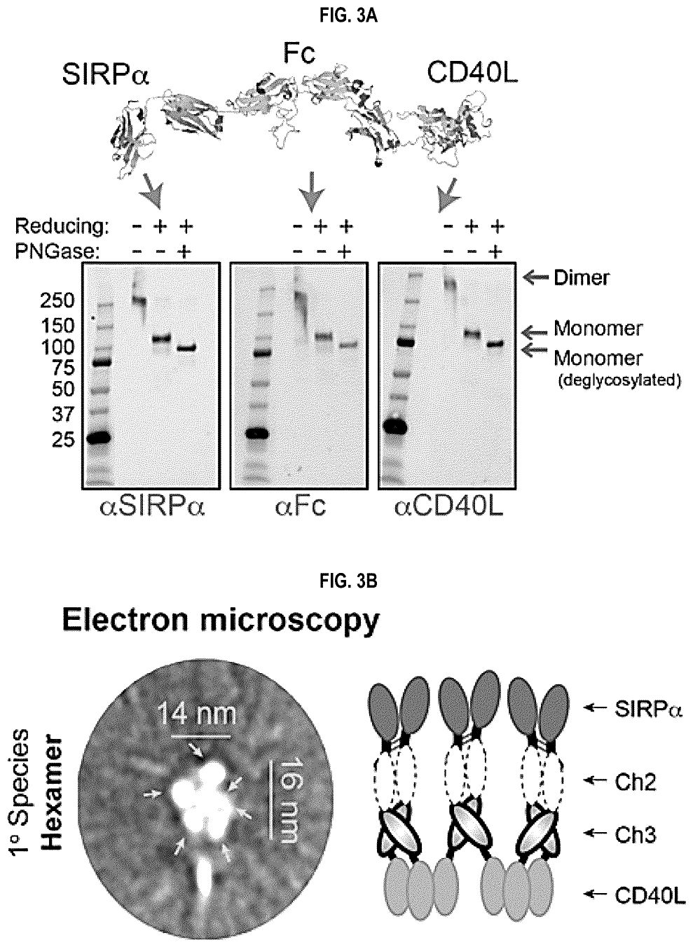

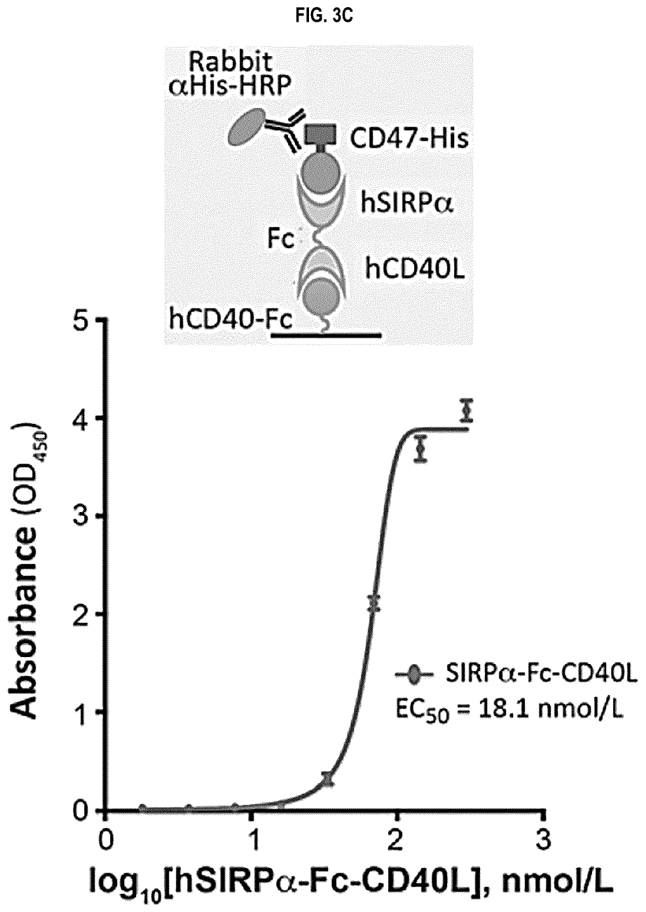

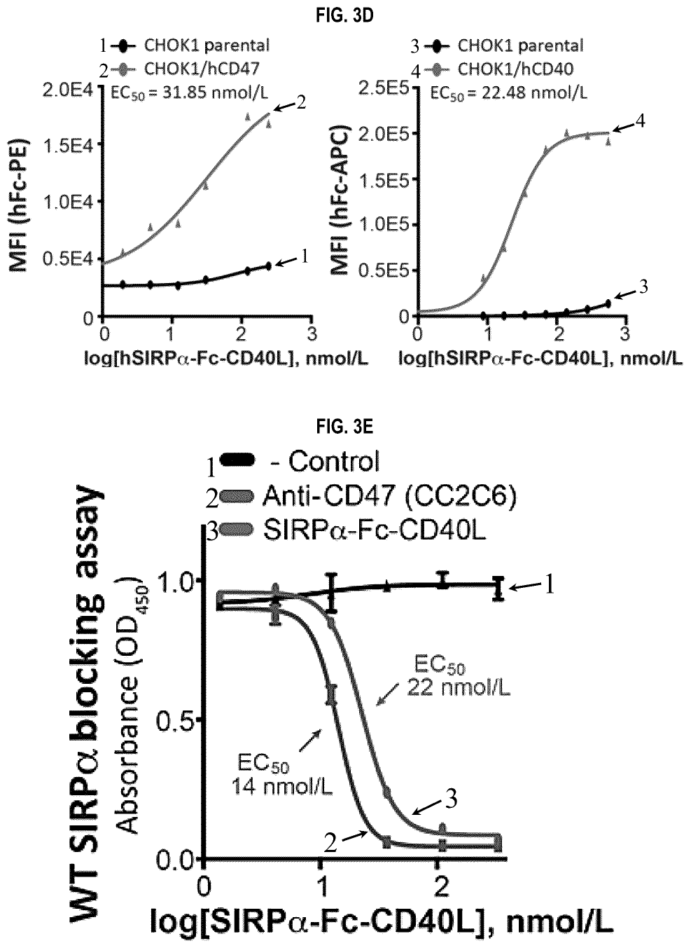

The chimeric protein of SEQ ID NO: 59 (also referred to herein as SL-172154) is a recombinant fusion glycoprotein comprising the extracellular domain of human CD172a (SIRPα) (PDCD1, CD272a), a central domain including the hinge-CH2-CH3 region from human immunoglobulin constant gamma 4 (Inhibitory receptor SHPS-1, IgG4), and the extracellular domain of human CD40L (TNFSF5, TRAP, CD154). The linear configuration of SL-172154 is CD172a (SIRPα)-Fc-CD40L. The tertiary structure of SL-172154, predicted by RaptorX, and without wishing to be bound by theory, is shown in FIG. 3A.

The predicted molecular weight for the monomeric chimeric protein of SEQ ID NO: 59 is 88.1 kDa. The predicted molecular weight for the glycosylated monomeric chimeric protein of SEQ ID NO: 59 is about 115 kDa.

The dual-sided nature of the chimeric proteins disclosed herein, such as the CD172a (SIRPα)-Fc-CD40L chimeric protein (e.g. SEQ ID NO: 59 or SEQ ID NO: 61), is designed to intercept one of the key immunosuppressive pathways within the tumor microenvironment (TME): the CD172a (SIRPα)-CD47 macrophage checkpoint.

Tumor cells may express CD47 on their cell surface, which can bind to CD172a (SIRPα) expressed by a macrophage to suppress phagocytosis of the tumor cells. Thus, the CD172a (SIRPα)-Fc-CD40L chimeric protein (e.g. SEQ ID NO: 59 or SEQ ID NO: 61) can bind to CD47 expressed on the surface of tumor, with the CD172a (SIRPα) domain of the CD172a (SIRPα)-Fc-CD40L chimeric proteins disclosed herein intended to provide competitive inhibition of CD47, and to replace the CD47 inhibitory signal with functionally trimerized/hexamerized CD40L, resulting in an incoming T cell experiencing co-stimulation via engagement through its CD40 receptor instead of suppression through CD172a (SIRPα) interactions. In other words, because the extracellular domains (ECDs) of CD172a (SIRPα) and CD40L are physically linked to one another and localized to the TME, tumor infiltrating T cells will receive co-stimulation at the same time they recognize a tumor antigen via its T cell receptor (TCR). Importantly, because the ECDs of CD172a (SIRPα) and CD40L are physically linked to one another, and localized to the TME, tumor infiltrating T cells will receive costimulation at the same time they recognize a tumor antigen via the T cell receptor. Together, these would result in replacement of an inhibitory CD47 signal with a co-stimulatory CD40L signal to enhance the anti-tumor activity of T cells.

The three constituent components of the chimeric proteins disclosed herein, including the CD172a (SIRPα)-Fc-CD40L chimeric protein (e.g. SEQ ID NO: 59 or SEQ ID NO: 61), have unique attributes that facilitate dimerization or oligomerization. The extracellular domain of CD172a (SIRPα) normally exists as a monomer and is not known to form higher-order homomeric complexes. The central Fc domain contains cysteine residues that are capable of disulfide bonding to form a dimeric structure. In embodiments, the chimeric proteins disclosed herein, including the CD172a (SIRPα)-Fc-CD40L chimeric protein (e.g. SEQ ID NO: 59 or SEQ ID NO: 61), contains an S228P mutation in the hinge region of the Fc domain to prevent Fab arm exchange. The CD40L domain is known to form homotrimeric complexes, which are stabilized through noncovalent, electrostatic interactions. Although the chimeric proteins disclosed herein, including the CD172a (SIRPα)-Fc-CD40L chimeric protein (e.g. SEQ ID NO: 59 or SEQ ID NO: 61), are expressed as a continuous monomeric protein by production cell lines, the resulting monomeric proteins self-assemble into higher-order species based on these disulfide and charge-based interactions of CD40L (creating a trimer) and the combined influence of these attractive forces, resulting in a hexamer (dimer of trimers). The majority (>80%) of the CD172a (SIRPα)-Fc-CD40L chimeric protein (e.g. SEQ ID NO: 59 or SEQ ID NO: 61) comprises the hexamer and trimer forms, which have similar activity. Importantly, because the CD172a (SIRPα)-Fc-CD40L chimeric protein (e.g. SEQ ID NO: 59 or SEQ ID NO: 61), are comprised of hexamers and trimers, they stimulate CD40 signaling in the absence of cross-linking by Fc receptors or any other cross-linking agent. The predicted tertiary structures of the CD172a (SIRPα)-Fc-CD40L chimeric protein (e.g. SEQ ID NO: 59 or SEQ ID NO: 61) as a monomer and in various oligomeric states, based on disulfide (Fc) and charge-based (CD40L) interactions are illustrated in FIG. 3A. FIG. 3B shows visualization by electron microscopy of the CD172a (SIRPα)-Fc-CD40L chimeric protein (e.g. SEQ ID NO: 59 or SEQ ID NO: 61) hexamers (top two images) and the CD172a (SIRPα)-Fc-CD40L chimeric protein (e.g. SEQ ID NO: 59 or SEQ ID NO: 61) trimers (bottom two images). Accordingly, the CD172a (SIRPα)-Fc-CD40L chimeric protein (e.g. SEQ ID NO: 59 or SEQ ID NO: 61) forms trimers/hexamers and activates CD40 without the need for cross-linking. It is noteworthy that, unlike monoclonal antibodies, Fc receptor cross-linking is not required for functional activity of the CD172a (SIRPα)-Fc-CD40L chimeric protein (e.g. SEQ ID NO: 59 or SEQ ID NO: 61).

In embodiments, a chimeric protein comprises a variant of the CD172a (SIRPα)-Fc-CD40L chimeric protein (e.g. SEQ ID NO: 59 or SEQ ID NO: 61). As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99%, or at least about 99.2%, or at least about 99.4%, or at least about 99.6%, or at least about 99.8% sequence identity with SEQ ID NO: 59 or SEQ ID NO: 61.

In embodiments, the first domain of a chimeric protein comprises an amino acid sequence that is at least 90%, or 93%, or 95%, or 97%, or 98%, or 99% identical to the amino acid sequence of SEQ ID NO: 59 or SEQ ID NO: 61. In embodiments, the first domain of a chimeric protein comprises an amino acid sequence that is at least 95% identical to the amino acid sequence of SEQ ID NO: 59 or SEQ ID NO: 61. In embodiments, the first domain of a chimeric protein comprises an amino acid sequence that is at least 97% identical to the amino acid sequence of SEQ ID NO: 59 or SEQ ID NO: 61. In embodiments, the first domain of a chimeric protein comprises an amino acid sequence that is at least 98% identical to the amino acid sequence of SEQ ID NO: 59 or SEQ ID NO: 61. In embodiments, the first domain of a chimeric protein comprises an amino acid sequence that is at least 99% identical to the amino acid sequence of SEQ ID NO: 59 or SEQ ID NO: 61. In embodiments, the first domain of a chimeric protein comprises an amino acid sequence that is identical to the amino acid sequence of SEQ ID NO: 59 or SEQ ID NO: 61.

In embodiments, the first domain is capable of binding a CD172a (SIRPα) ligand.

In embodiments, the first domain comprises substantially all of the extracellular domain of CD172a (SIRPα).

In embodiments, the second domain is capable of binding a CD40 receptor.

In embodiments, the second domain comprises substantially all of the extracellular domain of CD40L.

In embodiments, the linker comprises a hinge-CH2-CH3 Fc domain derived from IgG4, e.g., human IgG4.

In embodiments, the linker comprises an amino acid sequence that is at least 95% identical to the amino acid sequence of SEQ ID NO: 1, SEQ ID NO: 2, or SEQ ID NO: 3. In embodiments, the linker comprises an amino acid sequence that is at least 90%, or 93%, or 95%, or 97%, or 98%, or 99% identical to the amino acid sequence of SEQ ID NO: 1, SEQ ID NO: 2, or SEQ ID NO: 3. In embodiments, the linker comprises an amino acid sequence that is at least 95% identical to the amino acid sequence of SEQ ID NO: 1, SEQ ID NO: 2, or SEQ ID NO: 3. In embodiments, the linker comprises an amino acid sequence that is at least 97% identical to the amino acid sequence of SEQ ID NO: 1, SEQ ID NO: 2, or SEQ ID NO: 3. In embodiments, the linker comprises an amino acid sequence that is at least 98% identical to the amino acid sequence of SEQ ID NO: 1, SEQ ID NO: 2, or SEQ ID NO: 3. In embodiments, the linker comprises an amino acid sequence that is at least 99% identical to the amino acid sequence of SEQ ID NO: 1, SEQ ID NO: 2, or SEQ ID NO: 3. In embodiments, the linker comprises an amino acid sequence that is identical to the amino acid sequence of SEQ ID NO: 1, SEQ ID NO: 2, or SEQ ID NO: 3.

In embodiments, the first domain comprises an amino acid sequence that is at least 95% identical to the amino acid sequence of SEQ ID NO: 57. In embodiments, the first domain of a chimeric protein comprises an amino acid sequence that is at least 90%, or 93%, or 95%, or 97%, or 98%, or 99% identical to the amino acid sequence of SEQ ID NO: 57. In embodiments, the first domain of a chimeric protein comprises an amino acid sequence that is at least 95% identical to the amino acid sequence of SEQ ID NO: 57. In embodiments, the first domain of a chimeric protein comprises an amino acid sequence that is at least 97% identical to the amino acid sequence of SEQ ID NO: 57. In embodiments, the first domain of a chimeric protein comprises an amino acid sequence that is at least 98% identical to the amino acid sequence of SEQ ID NO: 57. In embodiments, the first domain of a chimeric protein comprises an amino acid sequence that is at least 99% identical to the amino acid sequence of SEQ ID NO: 57. In embodiments, the first domain of a chimeric protein comprises an amino acid sequence that is identical to the amino acid sequence of SEQ ID NO: 57.

In embodiments, the second domain comprises an amino acid sequence that is at least 95% identical to the amino acid sequence of SEQ ID NO: 58. In embodiments, the second domain of a chimeric protein comprises an amino acid sequence that is at least 90%, or 93%, or 95%, or 97%, or 98%, or 99% identical to the amino acid sequence of SEQ ID NO: 58. In embodiments, the second domain of a chimeric protein comprises an amino acid sequence that is at least 95% identical to the amino acid sequence of SEQ ID NO: 58. In embodiments, the second domain of a chimeric protein comprises an amino acid sequence that is at least 97% identical to the amino acid sequence of SEQ ID NO: 58. In embodiments, the second domain of a chimeric protein comprises an amino acid sequence that is at least 98% identical to the amino acid sequence of SEQ ID NO: 58. In embodiments, the second domain of a chimeric protein comprises an amino acid sequence that is at least 99% identical to the amino acid sequence of SEQ ID NO: 58. In embodiments, the second domain of a chimeric protein comprises an amino acid sequence that is identical to the amino acid sequence of SEQ ID NO: 58.

In embodiments, the (a) the first domain comprises the amino acid sequence of SEQ ID NO: 57, (b) the second domain comprises the amino acid sequence of SEQ ID NO: 58, and (c) the linker comprises an amino acid sequence that is at least 95% identical to SEQ ID NO: 1, SEQ ID NO: 2, or SEQ ID NO: 3.

In embodiments, the chimeric protein further comprises an amino acid sequence that is at least 90%, or 93%, or 95%, or 97%, or 98%, or 99% identical to the amino acid sequence of SEQ ID NO: 5 and/or SEQ ID NO: 7. In embodiments, the chimeric protein further comprises an amino acid sequence that is at least 95% identical to the amino acid sequence of SEQ ID NO: 5 and/or SEQ ID NO: 7. In embodiments, the chimeric protein further comprises an amino acid sequence that is at least 98% identical to the amino acid sequence of SEQ ID NO: 5 and/or SEQ ID NO: 7. In embodiments, the chimeric protein further comprises the amino acid sequence of SEQ ID NO: 5 and/or SEQ ID NO: 7.

In embodiments, the chimeric protein comprises an amino acid sequence that is at least about 95% identical to SEQ ID NO: 59 or SEQ ID NO: 61, e.g., at least about 98% identical to SEQ ID NO: 59 or SEQ ID NO: 61, at least about 99% identical to SEQ ID NO: 59 or SEQ ID NO: 61, at least about 99.2% identical to SEQ ID NO: 59 or SEQ ID NO: 61, at least about 99.4% identical to SEQ ID NO: 59 or SEQ ID NO: 61, at least about 99.6% identical to SEQ ID NO: 59 or SEQ ID NO: 61, or at least about 99.8% identical to SEQ ID NO: 59 or SEQ ID NO: 61. In embodiments, the chimeric protein comprises the amino acid sequence of SEQ ID NO: 59 or SEQ ID NO: 61.

In any herein-disclosed aspect and embodiment, the chimeric protein may comprise an amino acid sequence having one or more amino acid mutations relative to any of the protein sequences disclosed herein. In embodiments, the one or more amino acid mutations may be independently selected from substitutions, insertions, deletions, and truncations.

In embodiments, the amino acid mutations are amino acid substitutions, and may include conservative and/or non-conservative substitutions. “Conservative substitutions” may be made, for instance, on the basis of similarity in polarity, charge, size, solubility, hydrophobicity, hydrophilicity, and/or the amphipathic nature of the amino acid residues involved. The 20 naturally occurring amino acids can be grouped into the following six standard amino acid groups: (1) hydrophobic: Met, Ala, Val, Leu, Ile; (2) neutral hydrophilic: Cys, Ser, Thr; Asn, Gln; (3) acidic: Asp, Glu; (4) basic: His, Lys, Arg; (5) residues that influence chain orientation: Gly, Pro; and (6) aromatic: Trp, Tyr, Phe. As used herein, “conservative substitutions” are defined as exchanges of an amino acid by another amino acid listed within the same group of the six standard amino acid groups shown above. For example, the exchange of Asp by Glu retains one negative charge in the so modified polypeptide. In addition, glycine and proline may be substituted for one another based on their ability to disrupt α-helices. As used herein, “non-conservative substitutions” are defined as exchanges of an amino acid by another amino acid listed in a different group of the six standard amino acid groups (1) to (6) shown above.

In embodiments, the substitutions may also include non-classical amino acids (e.g., selenocysteine, pyrrolysine, N-formylmethionine β-alanine, GABA and 6-Aminolevulinic acid, 4-aminobenzoic acid (PABA), D-isomers of the common amino acids, 2,4-diaminobutyric acid, α-amino isobutyric acid, 4-aminobutyric acid, Abu, 2-amino butyric acid, γ-Abu, ε-Ahx, 6-amino hexanoic acid, Aib, 2-amino isobutyric acid, 3-amino propionic acid, ornithine, norleucine, norvaline, hydroxyproline, sarcosme, citrulline, homocitrulline, cysteic acid, t-butylglycine, t-butylalanine, phenylglycine, cyclohexylalanine, β-alanine, fluoro-amino acids, designer amino acids such as β methyl amino acids, C α-methyl amino acids, N α-methyl amino acids, and amino acid analogs in general).

Mutations may also be made to the nucleotide sequences of the chimeric proteins by reference to the genetic code, including taking into account codon degeneracy.

In embodiments, a chimeric protein is capable of binding human ligand(s)/receptor(s).

In embodiments, each extracellular domain (or variant thereof) of the chimeric protein binds to its cognate receptor or ligand with a K_{D} of about 1 nM to about 5 nM, for example, about 1 nM, about 1.5 nM, about 2 nM, about 2.5 nM, about 3 nM, about 3.5 nM, about 4 nM, about 4.5 nM, or about 5 nM. In embodiments, the chimeric protein binds to a cognate receptor or ligand with a K_{D} of about 5 nM to about 15 nM, for example, about 5 nM, about 5.5 nM, about 6 nM, about 6.5 nM, about 7 nM, about 7.5 nM, about 8 nM, about 8.5 nM, about 9 nM, about 9.5 nM, about 10 nM, about 10.5 nM, about 11 nM, about 11.5 nM, about 12 nM, about 12.5 nM, about 13 nM, about 13.5 nM, about 14 nM, about 14.5 nM, or about 15 nM.

In embodiments, each extracellular domain (or variant thereof) of the chimeric protein binds to its cognate receptor or ligand with a K_{D} of less than about 1 μM, about 900 nM, about 800 nM, about 700 nM, about 600 nM, about 500 nM, about 400 nM, about 300 nM, about 200 nM, about 150 nM, about 130 nM, about 100 nM, about 90 nM, about 80 nM, about 70 nM, about 60 nM, about 55 nM, about 50 nM, about 45 nM, about 40 nM, about 35 nM, about 30 nM, about 25 nM, about 20 nM, about 15 nM, about 10 nM, or about 5 nM, or about 1 nM (as measured, for example, by surface plasmon resonance or biolayer interferometry).

In embodiments, the chimeric protein binds to human CD47 with a K_{D} of about 1 nM to about 5 nM, for example, about 1 nM, about 1.5 nM, about 2 nM, about 2.5 nM, about 3 nM, about 3.5 nM, about 4 nM, about 4.5 nM, or about 5 nM. In embodiments, the chimeric protein binds to human CD47 with a K_{D} of less than about 3 nM, about 2 nM, about 1 nM, about 900 pM, about 800 pM, about 700 pM, about 600 pM, about 500 pM, about 400 pM, about 300 pM, about 200 pM, about 100 pM, about 90 pM, about 80 pM, about 70 pM, about 60 pM about 55 pM about 50 pM about 45 pM, about 40 pM, about 35 pM, about 30 pM, about 25 pM, about 20 pM, about 15 pM, or about 10 pM, or about 1 pM (as measured, for example, by surface plasmon resonance or biolayer interferometry).

In embodiments, the chimeric protein binds to human CD40 with a K_{D} of less than about 1 nM, about 900 pM, about 800 pM, about 700 pM, about 600 pM, about 500 pM, about 400 pM, about 300 pM, about 200 pM, about 100 pM, about 90 pM, about 80 pM, about 70 pM, about 60 pM about 55 pM about 50 pM about 45 pM, about 40 pM, about 35 pM, about 30 pM, about 25 pM, about 20 pM, about 15 pM, or about 10 pM, or about 1 pM (as measured, for example, by surface plasmon resonance or biolayer interferometry).

As used herein, a variant of an extracellular domain is capable of binding the receptor/ligand of a native extracellular domain. For example, a variant may include one or more mutations in an extracellular domain which do not affect its binding affinity to its receptor/ligand; alternately, the one or more mutations in an extracellular domain may improve binding affinity for the receptor/ligand; or the one or more mutations in an extracellular domain may reduce binding affinity for the receptor/ligand, yet not eliminate binding altogether. In embodiments, the one or more mutations are located outside the binding pocket where the extracellular domain interacts with its receptor/ligand. In embodiments, the one or more mutations are located inside the binding pocket where the extracellular domain interacts with its receptor/ligand, as long as the mutations do not eliminate binding altogether. Based on the skilled artisan's knowledge and the knowledge in the art regarding receptor-ligand binding, s/he would know which mutations would permit binding and which would eliminate binding.

In embodiments, the chimeric protein exhibits enhanced stability and protein half-life.

A chimeric protein of the present disclosure may comprise more than two extracellular domains. For example, the chimeric protein may comprise three, four, five, six, seven, eight, nine, ten, or more extracellular domains. A second extracellular domain may be separated from a third extracellular domain via a linker, as disclosed herein. Alternately, a second extracellular domain may be directly linked (e.g., via a peptide bond) to a third extracellular domain. In embodiments, a chimeric protein includes extracellular domains that are directly linked and extracellular domains that are indirectly linked via a linker, as disclosed herein.

### Linkers

In embodiments, the chimeric protein comprises a linker.

In embodiments, the linker comprising at least one cysteine residue capable of forming a disulfide bond. The at least one cysteine residue is capable of forming a disulfide bond between a pair (or more) of chimeric proteins. Without wishing to be bound by theory, such disulfide bond forming is responsible for maintaining a useful multimeric state of chimeric proteins. This allows for efficient production of the chimeric proteins; it allows for desired activity in vitro and in vivo.

In a chimeric protein of the present disclosure, the linker is a polypeptide selected from a flexible amino acid sequence, an IgG hinge region, or an antibody sequence.

In embodiments, the linker is derived from naturally-occurring multi-domain proteins or is an empirical linker as described, for example, in Chichili et al., Protein Sci. 22(2):153-167 (2013); Chen et al., Adv Drug Deliv Rev. 65(10):1357-1369 (2013), the entire contents of which are hereby incorporated by reference. In embodiments, the linker may be designed using linker designing databases and computer programs such as those described in Chen et al, Adv Drug Deliv Rev. 65(10):1357-1369 (2013); and Crasto et al, Protein Eng. 13(5):309-312 (2000), the entire contents of which are hereby incorporated by reference.

In embodiments, the linker comprises a polypeptide. In embodiments, the polypeptide is less than about 500 amino acids long, about 450 amino acids long, about 400 amino acids long, about 350 amino acids long, about 300 amino acids long, about 250 amino acids long, about 200 amino acids long, about 150 amino acids long, or about 100 amino acids long. For example, the linker may be less than about 100, about 95, about 90, about 85, about 80, about 75, about 70, about 65, about 60, about 55, about 50, about 45, about 40, about 35, about 30, about 25, about 20, about 19, about 18, about 17, about 16, about 15, about 14, about 13, about 12, about 11, about 10, about 9, about 8, about 7, about 6, about 5, about 4, about 3, or about 2 amino acids long.

In embodiments, the linker is flexible.

In embodiments, the linker is rigid.

In embodiments, the linker is substantially comprised of glycine and serine residues (e.g., about 30%, or about 40%, or about 50%, or about 60%, or about 70%, or about 80%, or about 90%, or about 95%, or about 97%, or about 98%, or about 99%, or about 100% glycines and serines).

In embodiments, the linker comprises a hinge region of an antibody (e.g., of IgG, IgA, IgD, and IgE, inclusive of subclasses (e.g., IgG1, IgG2, IgG3, and IgG4, and IgA1, and IgA2)). The hinge region, found in IgG, IgA, IgD, and IgE class antibodies, acts as a flexible spacer, allowing the Fab portion to move freely in space. In contrast to the constant regions, the hinge domains are structurally diverse, varying in both sequence and length among immunoglobulin classes and subclasses. For example, the length and flexibility of the hinge region varies among the IgG subclasses. The hinge region of IgG1 encompasses amino acids 216-231 and, because it is freely flexible, the Fab fragments can rotate about their axes of symmetry and move within a sphere centered at the first of two inter-heavy chain disulfide bridges. IgG2 has a shorter hinge than IgG1, with 12 amino acid residues and four disulfide bridges. The hinge region of IgG2 lacks a glycine residue, is relatively short, and contains a rigid poly-proline double helix, stabilized by extra inter-heavy chain disulfide bridges. These properties restrict the flexibility of the IgG2 molecule. IgG3 differs from the other subclasses by its unique extended hinge region (about four times as long as the IgG1 hinge), containing 62 amino acids (including 21 prolines and 11 cysteines), forming an inflexible poly-proline double helix. In IgG3, the Fab fragments are relatively far away from the Fc fragment, giving the molecule a greater flexibility. The elongated hinge in IgG3 is also responsible for its higher molecular weight compared to the other subclasses. The hinge region of IgG4 is shorter than that of IgG1 and its flexibility is intermediate between that of IgG1 and IgG2. The flexibility of the hinge regions reportedly decreases in the order IgG3>IgG1>IgG4>IgG2. In embodiments, the linker may be derived from human IgG4 and contain one or more mutations to enhance dimerization (including S228P) or FcRn binding.

According to crystallographic studies, the immunoglobulin hinge region can be further subdivided functionally into three regions: the upper hinge region, the core region, and the lower hinge region. See Shin et al., Immunological Reviews 130:87 (1992). The upper hinge region includes amino acids from the carboxyl end of CH1 to the first residue in the hinge that restricts motion, generally the first cysteine residue that forms an interchain disulfide bond between the two heavy chains. The length of the upper hinge region correlates with the segmental flexibility of the antibody. The core hinge region contains the inter-heavy chain disulfide bridges, and the lower hinge region joins the amino terminal end of the CH2 domain and includes residues in C_{H2}. Id. The core hinge region of wild-type human IgG1 contains the sequence CPPC (SEQ ID NO: 24) which, when dimerized by disulfide bond formation, results in a cyclic octapeptide believed to act as a pivot, thus conferring flexibility. In embodiments, the present linker comprises, one, or two, or three of the upper hinge region, the core region, and the lower hinge region of any antibody (e.g., of IgG, IgA, IgD, and IgE, inclusive of subclasses (e.g., IgG1, IgG2, IgG3, and IgG4, and IgA1 and IgA2)). The hinge region may also contain one or more glycosylation sites, which include a number of structurally distinct types of sites for carbohydrate attachment. For example, IgA1 contains five glycosylation sites within a 17-amino-acid segment of the hinge region, conferring resistance of the hinge region polypeptide to intestinal proteases, considered an advantageous property for a secretory immunoglobulin. In embodiments, the linker of the present disclosure comprises one or more glycosylation sites.

In embodiments, the linker comprises an Fc domain of an antibody (e.g., of IgG, IgA, IgD, and IgE, inclusive of subclasses (e.g., IgG1, IgG2, IgG3, and IgG4, and IgA1 and IgA2)).

In a chimeric protein of the present disclosure, the linker comprises a hinge-CH2-CH3 Fc domain derived from IgG4. In embodiments, the linker comprises a hinge-CH2-CH3 Fc domain derived from a human IgG4. In embodiments, the linker comprises an amino acid sequence that is at least 90%, or 93%, or 95%, or 97%, or 98%, or 99% identical to the amino acid sequence of any one of SEQ ID NO: 1 to SEQ ID NO: 3. In embodiments, the linker comprises an amino acid sequence that is at least 95% identical to the amino acid sequence of any one of SEQ ID NO: 1 to SEQ ID NO: 3 (e.g., at least 95% identical to the amino acid sequence of SEQ ID NO: 2.). In embodiments, the linker comprises one or more joining linkers, such joining linkers independently selected from SEQ ID NOs: 4-50 (or a variant thereof). In embodiments, the linker comprises two or more joining linkers each joining linker independently selected from SEQ ID NOs: 4-50 (or a variant thereof); wherein one joining linker is N terminal to the hinge-CH2-CH3 Fc domain and another joining linker is C terminal to the hinge-CH2-CH3 Fc domain.

In embodiments, the linker comprises a hinge-CH2-CH3 Fc domain derived from a human IgG1 antibody. In embodiments, the Fc domain exhibits increased affinity for and enhanced binding to the neonatal Fc receptor (FcRn). In embodiments, the Fc domain includes one or more mutations that increases the affinity and enhances binding to FcRn. Without wishing to be bound by theory, it is believed that increased affinity and enhanced binding to FcRn increases the in vivo half-life of the present chimeric proteins.

In embodiments, the Fc domain in a linker contains one or more amino acid substitutions at amino acid residue 250, 252, 254, 256, 308, 309, 311, 416, 428, 433, or 434 (in accordance with Kabat numbering, as in as in Kabat, et al, Sequences of Proteins of Immunological Interest, 5th Ed. Public Health Service, National Institutes of Health, Bethesda, Md. (1991) expressly incorporated herein by reference), or equivalents thereof. In embodiments, the amino acid substitution at amino acid residue 250 is a substitution with glutamine. In embodiments, the amino acid substitution at amino acid residue 252 is a substitution with tyrosine, phenylalanine, tryptophan or threonine. In embodiments, the amino acid substitution at amino acid residue 254 is a substitution with threonine. In embodiments, the amino acid substitution at amino acid residue 256 is a substitution with serine, arginine, glutamine, glutamic acid, aspartic acid, or threonine. In embodiments, the amino acid substitution at amino acid residue 308 is a substitution with threonine. In embodiments, the amino acid substitution at amino acid residue 309 is a substitution with proline. In embodiments, the amino acid substitution at amino acid residue 311 is a substitution with serine. In embodiments, the amino acid substitution at amino acid residue 385 is a substitution with arginine, aspartic acid, serine, threonine, histidine, lysine, alanine or glycine. In embodiments, the amino acid substitution at amino acid residue 386 is a substitution with threonine, proline, aspartic acid, serine, lysine, arginine, isoleucine, or methionine. In embodiments, the amino acid substitution at amino acid residue 387 is a substitution with arginine, proline, histidine, serine, threonine, or alanine. In embodiments, the amino acid substitution at amino acid residue 389 is a substitution with proline, serine or asparagine. In embodiments, the amino acid substitution at amino acid residue 416 is a substitution with serine. In embodiments, the amino acid substitution at amino acid residue 428 is a substitution with leucine. In embodiments, the amino acid substitution at amino acid residue 433 is a substitution with arginine, serine, isoleucine, proline, or glutamine. In embodiments, the amino acid substitution at amino acid residue 434 is a substitution with histidine, phenylalanine, or tyrosine.

In embodiments, the Fc domain linker (e.g., comprising an IgG constant region) comprises one or more mutations such as substitutions at amino acid residue 252, 254, 256, 433, 434, or 436 (in accordance with Kabat numbering, as in as in Kabat, et al., Sequences of Proteins of Immunological Interest, 5th Ed. Public Health Service, National Institutes of Health, Bethesda, Md. (1991) expressly incorporated herein by reference). In embodiments, the IgG constant region includes a triple M252Y/S254T/T256E mutation or YTE mutation. In embodiments, the IgG constant region includes a triple H433K/N434F/Y436H mutation or KFH mutation. In embodiments, the IgG constant region includes an YTE and KFH mutation in combination.

In embodiments, the linker comprises an IgG constant region that contains one or more mutations at amino acid residues 250, 253, 307, 310, 380, 428, 433, 434, and 435 (in accordance with Kabat numbering, as in as in Kabat, et al., Sequences of Proteins of Immunological Interest, 5th Ed. Public Health Service, National Institutes of Health, Bethesda, Md. (1991) expressly incorporated herein by reference). Illustrative mutations include T250Q, M428L, T307A, E380A, 1253A, H310A, M428L, H433K, N434A, N434F, N434S, and H435A. In embodiments, the IgG constant region comprises a M428L/N434S mutation or LS mutation. In embodiments, the IgG constant region comprises a T250Q/M428L mutation or QL mutation. In embodiments, the IgG constant region comprises an N434A mutation. In embodiments, the IgG constant region comprises a T307A/E380A/N434A mutation or AAA mutation. In embodiments, the IgG constant region comprises an 1253A/H310A/H435A mutation or IHH mutation. In embodiments, the IgG constant region comprises a H433K/N434F mutation. In embodiments, the IgG constant region comprises a M252Y/S254T/T256E and a H433K/N434F mutation in combination.

Additional exemplary mutations in the IgG constant region are described, for example, in Robbie, et al., Antimicrobial Agents and Chemotherapy 57(12):6147-6153 (2013); Dall'Acqua et al., Journal Biol Chem 281(33):23514-24 (2006); Dall'Acqua et al., Journal of Immunology 169:5171-80 (2002); Ko et al. Nature 514:642-645 (2014); Grevys et al. Journal of Immunology 194(11):5497-508 (2015); and U.S. Pat. No. 7,083,784, the entire contents of which are hereby incorporated by reference.

An illustrative Fc stabilizing mutant is S228P. Illustrative Fc half-life extending mutants are T250Q, M428L, V308T, L309P, and Q311S and the present linkers may comprise 1, or 2, or 3, or 4, or 5 of these mutants.

In embodiments, the chimeric protein binds to FcRn with high affinity. In embodiments, the chimeric protein may bind to FcRn with a K_{D} of about 1 nM to about 80 nM. For example, the chimeric protein may bind to FcRn with a K_{D} of about 1 nM, about 2 nM, about 3 nM, about 4 nM, about 5 nM, about 6 nM, about 7 nM, about 8 nM, about 9 nM, about 10 nM, about 15 nM, about 20 nM, about 25 nM, about 30 nM, about 35 nM, about 40 nM, about 45 nM, about 50 nM, about 55 nM, about 60 nM, about 65 nM, about 70 nM, about 71 nM, about 72 nM, about 73 nM, about 74 nM, about 75 nM, about 76 nM, about 77 nM, about 78 nM, about 79 nM, or about 80 nM. In embodiments, the chimeric protein may bind to FcRn with a K_{D} of about 9 nM. In embodiments, the chimeric protein does not substantially bind to other Fc receptors (i.e. other than FcRn) with effector function.

In embodiments, the Fc domain in a linker has the amino acid sequence of SEQ ID NO: 1 (see Table 1, below), or at least 90%, or 93%, or 95%, or 97%, or 98%, or 99% identity thereto. In embodiments, mutations are made to SEQ ID NO: 1 to increase stability and/or half-life. For instance, in embodiments, the Fc domain in a linker comprises the amino acid sequence of SEQ ID NO: 2 (see Table 1, below), or at least 90%, or 93%, or 95%, or 97%, or 98%, or 99% identity thereto. For instance, in embodiments, the Fc domain in a linker comprises the amino acid sequence of SEQ ID NO: 3 (see Table 1, below), or at least 90%, or 93%, or 95%, or 97%, or 98%, or 99% identity thereto.

Further, one or more joining linkers may be employed to connect an Fc domain in a linker (e.g., one of SEQ ID NO: 1, SEQ ID NO: 2, SEQ ID NO: 3 or at least 90%, or 93%, or 95%, or 97%, or 98%, or 99% identity thereto) and the extracellular domains. For example, any one of SEQ ID NO: 4, SEQ ID NO: 5, SEQ ID NO: 6, SEQ ID NO: 7, SEQ ID NO: 8, SEQ ID NO: 9, or variants thereof may connect an extracellular domain as disclosed herein and an Fc domain in a linker as disclosed herein. Optionally, any one of SEQ ID NOs: 4 to 50, or variants thereof are located between an extracellular domain as disclosed herein and an Fc domain as disclosed herein.

In embodiments, the present chimeric proteins may comprise variants of the joining linkers disclosed in Table 1, below. For instance, a linker may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the amino acid sequence of any one of SEQ ID NOs: 4 to 50.

In embodiments, the first and second joining linkers may be different or they may be the same.

Without wishing to be bound by theory, including a linker comprising at least a part of an Fc domain in a chimeric protein, helps avoid formation of insoluble and, likely, non-functional protein concatemers and/or aggregates. This is in part due to the presence of cysteines in the Fc domain which are capable of forming disulfide bonds between chimeric proteins.

In embodiments, a chimeric protein may comprise one or more joining linkers, as disclosed herein, and lack a Fc domain linker, as disclosed herein.

In embodiments, the first and/or second joining linkers are independently selected from the amino acid sequences of SEQ ID NOs: 4 to 50 and are provided in Table 1 below:

TABLE 1
Illustrative linkers (Fc domain linkers and joining linkers)

| SEQ | |
|---|---|
| ID | |
| NO. | Sequence |
| 1 | APEFLGGPSVFLFPPKPKDTLMISRTPEVTCVVVDVSQEDPEVQFNWYVDGVEVHNAKTKPR |
| | EEQFNSTYRVVSVLTVLHQDWLSGKEYKCKVSSKGLPSSIEKTISNATGQPREPQVYTLPPSQ |
| | EEMTKNQVSLTCLVKGFYPSDIAVEWESNGQPENNYKTTPPVLDSDGSFFLYSRLTVDKSSW |
| | QEGNVFSCSVMHEALHNHYTQKSLSLSLGK |
| 2 | APEFLGGPSVFLFPPKPKDQLMISRTPEVTCVVVDVSQEDPEVQFNWYVDGVEVHNAKTKPR |
| | EEQFNSTYRVVSVLTTPHSDWLSGKEYKCKVSSKGLPSSIEKTISNATGQPREPQVYTLPPSQ |
| | EEMTKNQVSLTCLVKGFYPSDIAVEWESNGQPENNYKTTPPVLDSDGSFFLYSRLTVDKSSW |
| | QEGNVFSCSVLHEALHNHYTQKSLSLSLGK |
| 3 | APEFLGGPSVFLFPPKPKDQLMISRTPEVTCVVVDVSQEDPEVQFNWYVDGVEVHNAKTKPR |
| | EEQFNSTYRVVSVLTVLHQDWLSGKEYKCKVSSKGLPSSIEKTISNATGQPREPQVYTLPPSQ |
| | EEMTKNQVSLTCLVKGFYPSDIAVEWESNGQPENNYKTTPPVLDSDGSFFLYSRLTVDKSRW |
| | QEGNVFSCSVLHEALHNHYTQKSLSLSLGK |
| 4 | SKYGPPCPSCP |
| 5 | SKYGPPCPPCP |
| 6 | SKYGPP |
| 7 | IEGRMD |
| 8 | GGGVPRDCG |
| 9 | IEGRMDGGGGAGGGG |
| 10 | GGGSGGGS |
| 11 | GGGSGGGGSGGG |
| 12 | EGKSSGSGSESKST |
| 13 | GGSG |
| 14 | GGSGGGSGGGSG |
| 15 | EAAAKEAAAKEAAAK |
| 16 | EAAAREAAAREAAAREAAAR |
| 17 | GGGGSGGGGSGGGGSAS |
| 18 | GGGGAGGGG |
| 19 | GS or GGS or LE |
| 20 | GSGSGS |
| 21 | GSGSGSGSGS |
| 22 | GGGGSAS |
| 23 | APAPAPAPAPAPAPAPAPAP |
| 24 | CPPC |
| 25 | GGGGS |
| 26 | GGGGSGGGGS |
| 27 | GGGGSGGGGSGGGGS |
| 28 | GGGGSGGGGSGGGGSGGGGS |
| 29 | GGGGSGGGGSGGGGSGGGGSGGGGS |
| 30 | GGGGSGGGGSGGGGSGGGGSGGGGSGGGGS |
| 31 | GGGGSGGGGSGGGGSGGGGSGGGGSGGGGSGGGGS |
| 32 | GGGGSGGGGSGGGGSGGGGGGGGSGGGGSGGGGSGGGGS |
| 33 | GGSGGSGGGGSGGGGS |
| 34 | GGGGGGGG |
| 35 | GGGGGG |
| 36 | EAAAK |
| 37 | EAAAKEAAAK |
| 38 | EAAAKEAAAKEAAAK |
| 39 | AEAAAKEAAAKA |
| 40 | AEAAAKEAAAKEAAAKA |
| 41 | AEAAAKEAAAKEAAAKEAAAKA |
| 42 | AEAAAKEAAAKEAAAKEAAAKEAAAKA |
| 43 | AEAAAKEAAAKEAAAKEAAAKALEAEAAAKEAAAKEAAAKEAAAKA |
| 44 | PAPAP |
| 45 | KESGSVSSEQLAQFRSLD |
| 46 | GSAGSAAGSGEF |
| 47 | GGGSE |
| 48 | GSESG |
| 49 | GSEGS |
| 50 | GEGGSGEGSSGEGSSSEGGGSEGGGSEGGGSEGGS |

In embodiments, the joining linker substantially comprises glycine and serine residues (e.g., about 30%, or about 40%, or about 50%, or about 60%, or about 70%, or about 80%, or about 90%, or about 95%, or about 97%, or about 98%, or about 99%, or about 100% glycines and serines). For example, in embodiments, the joining linker is (Gly₄Ser)ₙ, where n is from about 1 to about 8, e.g., 1, 2, 3, 4, 5, 6, 7, or 8 (SEQ ID NO: 25 to SEQ ID NO: 9, respectively). In embodiments, the joining linker sequence is GGSGGSGGGGSGGGGS (SEQ ID NO: 33). Additional illustrative joining linkers include, but are not limited to, linkers having the sequence LE, (EAAAK)ₙ (n=1-3) (SEQ ID NO: 36 to SEQ ID NO: 38), A(EAAAK)ₙA (n=2-5) (SEQ ID NO: 39 to SEQ ID NO: 42), A(EAAAK)₄ALEA(EAAAK)₄A (SEQ ID NO: 43), PAPAP (SEQ ID NO: 44), KESGSVSSEQLAQFRSLD (SEQ ID NO: 45), GSAGSAAGSGEF (SEQ ID NO: 46), and (XP)ₙ, with X designating any amino acid, e.g., Ala, Lys, or Glu. In embodiments, the joining linker is GGS. In embodiments, a joining linker has the sequence (Gly)ₙ where n is any number from 1 to 100, for example: (Gly)₈ (SEQ ID NO: 34) and (Gly)₆ (SEQ ID NO: 35).

In embodiments, the joining linker is one or more of GGGSE (SEQ ID NO: 47), GSESG (SEQ ID NO: 48), GSEGS (SEQ ID NO: 49), GEGGSGEGSSGEGSSSEGGGSEGGGSEGGGSEGGS (SEQ ID NO: 50), and a joining linker of randomly placed G, S, and E every 4 amino acid intervals.

In embodiments, the chimeric protein comprises a joining linker comprising the amino acid sequence of SEQ ID NO: 5 and/or SEQ ID NO: 7.

In embodiments, where a chimeric protein comprises an extracellular domain (ECD) of CD172a (SIRPα), one joining linker preceding an Fc domain, a second joining linker following the Fc domain, and an ECD of CD40L, the chimeric protein may comprise the following structure:

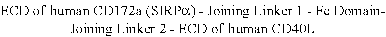

The combination of a first joining linker, an Fc Domain linker, and a second joining linker is referend to herein as a “modular linker”. In embodiments, a chimeric protein comprises a modular linker as shown in Table 2:

TABLE 2
Illustrative modular linkers

| | | | Modular Linker = |
|---|---|---|---|
| | | | Joining Linker |
| Joining | | Joining | 1 + Fc + |
| Linker 1 | Fc | Linker 2 | Joining Linker 2 |
| SKYGPPCPSCP | APEFLGGPSVFLFPPKPKDTL | IEGRMD | SKYGPPCPSCPAPEFLGGPSV |
| (SEQ ID | MISRTPEVTCVVVDVSQEDPE | (SEQ ID | FLFPPKPKDTLMISRTPEVTCV |
| NO: 4) | VQFNWYVDGVEVHNAKTKPR | NO: 7) | WVDVSQEDPEVQFNWYVDGV |
| | EEQFNSTYRVVSVLTVLHQDW | | EVHNAKTKPREEQFNSTYRVV |
| | LSGKEYKCKVSSKGLPSSIEKT | | SVLTVLHQDWLSGKEYKCKVS |
| | ISNATGQPREPQVYTLPPSQE | | SKGLPSSIEKTISNATGQPREP |
| | EMTKNQVSLTCLVKGFYPSDIA | | QVYTLPPSQEEMTKNQVSLTC |
| | VEWESNGQPENNYKTTPPVL | | LVKGFYPSDIAVEWESNGQPE |
| | DSDGSFFLYSRLTVDKSSWQE | | NNYKTTPPVLDSDGSFFLYSR |
| | GNVFSCSVMHEALHNHYTQK | | LTVDKSSWQEGNVFSCSVMH |
| | SLSLSLGK (SEQ ID NO: 1) | | EALHNHYTQKSLSLSLGKIEGR |
| | | | MD (SEQ ID NO: 51) |
| SKYGPPCPSCP | APEFLGGPSVFLFPPKPKDQL | IEGRMD | SKYGPPCPSCPAPEFLGGPSV |
| (SEQ ID | MISRTPEVTCVVVDVSQEDPE | (SEQ ID | FLFPPKPKDQLMISRTPEVTCV |
| NO: 4) | VQFNWYVDGVEVHNAKTKPR | NO: 7) | WDVSQEDPEVQFNWYVDGV |
| | EEQFNSTYRVVSVLTTPHSDW | | EVHNAKTKPREEQFNSTYRVV |
| | LSGKEYKCKVSSKGLPSSIEKT | | SVLTTPHSDWLSGKEYKCKVS |
| | ISNATGQPREPQVYTLPPSQE | | SKGLPSSIEKTISNATGQPREP |
| | EMTKNQVSLTCLVKGFYPSDIA | | QVYTLPPSQEEMTKNQVSLTC |
| | VEWESNGQPENNYKTTPPVL | | LVKGFYPSDIAVEWESNGQPE |
| | DSDGSFFLYSRLTVDKSSWQE | | NNYKTTPPVLDSDGSFFLYSR |
| | GNVFSCSVLHEALHNHYTQKS | | LTVDKSSWQEGNVFSCSVLHE |
| | LSLSLGK (SEQ ID NO: 2) | | ALHNHYTQKSLSLSLGKIEGR |
| | | | MD (SEQ ID NO: 52) |
| SKYGPPCPSCP | APEFLGGPSVFLFPPKPKDQL | IEGRMD | SKYGPPCPSCPAPEFLGGPSV |
| (SEQ ID | MISRTPEVTCVVVDVSQEDPE | (SEQ ID | FLFPPKPKDQLMISRTPEVTCV |
| NO: 4) | VQFNWYVDGVEVHNAKTKPR | NO: 7) | WDVSQEDPEVQFNWYVDGV |
| | EEQFNSTYRVVSVLTVLHQDW | | EVHNAKTKPREEQFNSTYRVV |
| | LSGKEYKCKVSSKGLPSSIEKT | | SVLTVLHQDWLSGKEYKCKVS |
| | ISNATGQPREPQVYTLPPSQE | | SKGLPSSIEKTISNATGQPREP |
| | EMTKNQVSLTCLVKGFYPSDIA | | QVYTLPPSQEEMTKNQVSLTC |
| | VEWESNGQPENNYKTTPPVL | | LVKGFYPSDIAVEWESNGQPE |
| | DSDGSFFLYSRLTVDKSRWQE | | NNYKTTPPVLDSDGSFFLYSR |
| | GNVFSCSVLHEALHNHYTQKS | | LTVDKSRWQEGNVFSCSVLHE |
| | LSLSLGK (SEQ ID NO: 3) | | ALHNHYTQKSLSLSLGKIEGR |
| | | | MD (SEQ ID NO: 53) |
| SKYGPPCPPCP | APEFLGGPSVFLFPPKPKDTL | IEGRMD | SKYGPPCPPCPAPEFLGGPSV |
| (SEQ ID | MISRTPEVTCVVVDVSQEDPE | (SEQ ID | FLFPPKPKDTLMISRTPEVTCV |
| NO: 5) | VQFNWYVDGVEVHNAKTKPR | NO: 7) | WDVSQEDPEVQFNWYVDGV |
| | EEQFNSTYRVVSVLTVLHQDW | | EVHNAKTKPREEQFNSTYRVV |
| | LSGKEYKCKVSSKGLPSSIEKT | | SVLTVLHQDWLSGKEYKCKVS |
| | ISNATGQPREPQVYTLPPSQE | | SKGLPSSIEKTISNATGQPREP |
| | EMTKNQVSLTCLVKGFYPSDIA | | QVYTLPPSQEEMTKNQVSLTC |
| | VEWESNGQPENNYKTTPPVL | | LVKGFYPSDIAVEWESNGQPE |
| | DSDGSFFLYSRLTVDKSSWQE | | NNYKTTPPVLDSDGSFFLYSR |
| | GNVFSCSVMHEALHNHYTQK | | LTVDKSSWQEGNVFSCSVMH |
| | SLSLSLGK (SEQ ID NO: 1) | | EALHNHYTQKSLSLSLGKIEGR |
| | | | MD (SEQ ID NO: 54) |
| SKYGPPCPPCP | APEFLGGPSVFLFPPKPKDQL | IEGRMD | SKYGPPCPPCPAPEFLGGPSV |
| (SEQ ID | MISRTPEVTCVVVDVSQEDPE | (SEQ ID | FLFPPKPKDQLMISRTPEVTCV |
| NO: 5) | VQFNWYVDGVEVHNAKTKPR | NO: 7) | WVDVSQEDPEVQFNWYVDGV |
| | EEQFNSTYRVVSVLTTPHSDW | | EVHNAKTKPREEQFNSTYRVV |
| | LSGKEYKCKVSSKGLPSSIEKT | | SVLTTPHSDWLSGKEYKCKVS |
| | ISNATGQPREPQVYTLPPSQE | | SKGLPSSIEKTISNATGQPREP |
| | EMTKNQVSLTCLVKGFYPSDIA | | QVYTLPPSQEEMTKNQVSLTC |
| | VEWESNGQPENNYKTTPPVL | | LVKGFYPSDIAVEWESNGQPE |
| | DSDGSFFLYSRLTVDKSSWQE | | NNYKTTPPVLDSDGSFFLYSR |
| | GNVFSCSVLHEALHNHYTQKS | | LTVDKSSWQEGNVFSCSVLHE |
| | LSLSLGK (SEQ ID NO: 2) | | ALHNHYTQKSLSLSLGKIEGR |
| | | | MD (SEQ ID NO: 55) |
| SKYGPPCPPCP | APEFLGGPSVFLFPPKPKDQL | IEGRMD | SKYGPPCPPCPAPEFLGGPSV |
| (SEQ ID | MISRTPEVTCVVVDVSQEDPE | (SEQ ID | FLFPPKPKDQLMISRTPEVTCV |
| NO: 5) | VQFNWYVDGVEVHNAKTKPR | NO: 7) | WDVSQEDPEVQFNWYVDGV |
| | EEQFNSTYRVVSVLTVLHQDW | | EVHNAKTKPREEQFNSTYRVV |
| | LSGKEYKCKVSSKGLPSSIEKT | | SVLTVLHQDWLSGKEYKCKVS |
| | ISNATGQPREPQVYTLPPSQE | | SKGLPSSIEKTISNATGQPREP |
| | EMTKNQVSLTCLVKGFYPSDIA | | QVYTLPPSQEEMTKNQVSLTC |
| | VEWESNGQPENNYKTTPPVL | | LVKGFYPSDIAVEWESNGQPE |
| | DSDGSFFLYSRLTVDKSRWQE | | NNYKTTPPVLDSDGSFFLYSR |
| | GNVFSCSVLHEALHNHYTQKS | | LTVDKSRWQEGNVFSCSVLHE |
| | LSLSLGK (SEQ ID NO: 3) | | ALHNHYTQKSLSLSLGKIEGR |
| | | | MD (SEQ ID NO: 56) |

In embodiments, the present chimeric proteins may comprise variants of the modular linkers disclosed in Table 2, above. For instance, a linker may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the amino acid sequence of any one of SEQ ID NOs: 51 to 56.

In embodiments, the linker may be flexible, including without limitation highly flexible. In embodiments, the linker may be rigid, including without limitation a rigid alpha helix. Characteristics of illustrative joining linkers is shown below in Table 3:

TABLE 3
Characteristics of illustrative joining linkers

| Joining Linker Sequence | Characteristics |
|---|---|
| SKYGPPCPPCP (SEQ ID NO: 5) | IgG4 Hinge Region |
| IEGRMD (SEQ ID NO: 7) | Linker |
| GGGVPRDCG (SEQ ID NO: 8) | Flexible |
| GGGSGGGS (SEQ ID NO: 10) | Flexible |
| GGGSGGGGSGGG (SEQ ID NO: 11) | Flexible |
| EGKSSGSGSESKST (SEQ ID NO: 12) | Flexible + soluble |
| GGSG (SEQ ID NO: 13) | Flexible |
| GGSGGGSGGGSG (SEQ ID NO: 14) | Flexible |
| EAAAKEAAAKEAAAK | Rigid Alpha Helix |
| (SEQ ID NO: 15) | |
| EAAAREAAAREAAAREAAAR | Rigid Alpha Helix |
| (SEQ ID NO: 16) | |
| GGGGSGGGGSGGGGSAS | Flexible |
| (SEQ ID NO: 17) | |
| GGGGAGGGG (SEQ ID NO: 18) | Flexible |
| GS (SEQ ID NO: 19) | Highly flexible |
| GSGSGS (SEQ ID NO: 20) | Highly flexible |
| GSGSGSGSGS (SEQ ID NO: 21) | Highly flexible |
| GGGGSAS (SEQ ID NO: 22) | Flexible |
| APAPAPAPAPAPAPAPAPAP | Rigid |
| (SEQ ID NO: 23) | |

In embodiments, the linker may be functional. For example, without limitation, the linker may function to improve the folding and/or stability, improve the expression, improve the pharmacokinetics, and/or improve the bioactivity of the present chimeric protein. In another example, the linker may function to target the chimeric protein to a particular cell type or location.

In embodiments, a chimeric protein comprises only one joining linkers.

In embodiments, a chimeric protein lacks joining linkers.

In embodiments, the linker is a synthetic linker such as polyethylene glycol (PEG).

In embodiments, a chimeric protein has a first domain which is sterically capable of binding its ligand/receptor and/or the second domain which is sterically capable of binding its ligand/receptor. Thus, there is enough overall flexibility in the chimeric protein and/or physical distance between an extracellular domain (or portion thereof) and the rest of the chimeric protein such that the ligand/receptor binding domain of the extracellular domain is not sterically hindered from binding its ligand/receptor. This flexibility and/or physical distance (which is referred to as “slack”) may be normally present in the extracellular domain(s), normally present in the linker, and/or normally present in the chimeric protein (as a whole). Alternately, or additionally, an amino acid sequence (for example) may be added to one or more extracellular domains and/or to the linker to provide the slack needed to avoid steric hindrance. Any amino acid sequence that provides slack may be added. In embodiments, the added amino acid sequence comprises the sequence (Gly)ₙ where n is any number from 1 to 100. Additional examples of addable amino acid sequence include the joining linkers described in Table 1 and Table 3. In embodiments, a polyethylene glycol (PEG) linker may be added between an extracellular domain and a linker to provide the slack needed to avoid steric hindrance. Such PEG linkers are well known in the art.

In embodiments, a chimeric protein of the present disclosure comprises the extracellular domain of human CD172a (SIRPα) (or a variant thereof), a linker, and the extracellular domain of human CD40L (or a variant thereof). In embodiments, the linker comprises a hinge-CH2-CH3 Fc domain, e.g., from an IgG1 or from IgG4, including human IgG1 or IgG4. Thus, in embodiments, a chimeric protein of the present disclosure comprises the extracellular domain of human CD172a (SIRPα) (or a variant thereof), linker comprising a hinge-CH2-CH3 Fc domain, and the extracellular domain of human CD40L (or a variant thereof). Such a chimeric protein may be referred to herein as “hCD172a (SIRPα)-Fc-CD40L” or “SL-172154”.

### Diseases, Methods of Treatment, and Mechanisms of Action

The chimeric proteins disclosed herein, including the CD172a (SIRPα)-Fc-CD40L chimeric protein (e.g. SEQ ID NO: 59 or SEQ ID NO: 61), finds use in methods for treating both advanced solid tumors and advanced lymphomas. These tumor types include: melanoma, non-small cell lung cancer (squamous, adeno, adeno-squamous), urothelial cancer, renal cell cancer, squamous cell cervical cancer, gastric or gastro-esophageal junction adenocarcinoma, squamous cell carcinoma of the anus, squamous cell carcinoma of the head and neck, squamous cell carcinoma of the skin, and microsatellite instability high or mismatch repair deficient solid tumors excluding central nervous system (CNS) tumors. Other tumors of interest include Hodgkin's lymphoma (HL), diffuse large B cell lymphoma, acute myeloid leukemia (AML) and high-risk myelodysplastic syndromes (HR-MDS).

In embodiments, the cancer comprises an advanced solid tumor (local and/or metastatic). In embodiments, the human subject has a cancer, wherein the cancer being treated is characterized by having macrophages in the tumor microenvironment and/or having tumor cells that are CD47+ cells in the tumor. In embodiments, the administration of the SIRPα-Fc-CD40L chimeric protein blocks the “don't eat me” signal of a tumor cell and/or stimulates an “eat me” signal. In embodiments the therapy with the SIRPα-Fc-CD40L chimeric protein (e.g. SEQ ID NO: 59 or SEQ ID NO: 61) stimulates macrophages to phagocytize tumor cells and effectively present the tumor antigens of phagocytized tumor cells to T cells.

In embodiments, the cancer is a solid cancer. In embodiments, the cancer is a solid tumor. In embodiments, the cancer is a metastatic cancer. In embodiments, the cancer is a hematological cancer. In embodiments, the cancer expresses CD47.

In embodiments, the cancer comprises an advanced lymphoma. In embodiments, the cancer comprises acute myeloid leukemia (AML). In embodiments, the cancer comprises p53 mutant AML. In embodiments, the cancer comprises a high-risk myelodysplastic syndrome (HR-MDS).

Aspects of the present disclosure provide methods of treating cancer. The methods comprise a step of administering to a subject in need thereof an effective amount of a chimeric protein, e.g., in a pharmaceutical composition, as disclosed herein.

It is often desirable to enhance immune stimulatory signal transmission to boost an immune response, for instance to enhance a patient's anti-tumor immune response.

In embodiments, the chimeric protein of the present disclosure comprises an extracellular domain of human CD172a (SIRPα), which disrupts, blocks, reduces, inhibits, and/or sequesters the transmission of immune inhibitory signals, e.g., originating from a cancer cell that is attempting to avoid its detection and/or destruction, and an extracellular domain of human CD40L, which enhances, increases, and/or stimulates the transmission of an immune stimulatory signal to an anti-cancer immune cell. Thus, the simultaneous binding of the extracellular domain of CD172a (SIRPα) to its ligand/receptor and the binding of the extracellular domain of CD40L with its receptor will prevent the transmission of an immunosuppressive signal from the cancer cell and will have stimulate immune activity in an immune system cell. In other words, chimeric proteins of the present disclosure are capable of treating cancer via two distinct mechanisms.

In embodiments, the present disclosure pertains to cancers and/or tumors; for example, the treatment or prevention of cancers and/or tumors. As disclosed elsewhere herein, the treatment of cancer involves, in embodiments, modulating the immune system with the present chimeric proteins to favor of increasing or activating immune stimulatory signals. In embodiments, the method reduces the amount or activity of regulatory T cells (Tregs) as compared to untreated subjects or subjects treated with antibodies directed to CD172a (SIRPα), CD40L, and/or their respective ligands or receptors. In embodiments, the method increases priming of effector T cells in draining lymph nodes of the subject as compared to untreated subjects or subjects treated with antibodies directed to CD172a (SIRPα), CD40L, and/or their respective ligands or receptors. In embodiments, the method causes an overall decrease in immunosuppressive cells and a shift toward a more inflammatory tumor environment as compared to untreated subjects or subjects treated with antibodies directed to the CD172a (SIRPα), CD40L, and/or their respective ligands or receptors.

In embodiments, the present chimeric proteins are capable of, or can be used in methods comprising, modulating the amplitude of an immune response, e.g. modulating the level of effector output. In embodiments, e.g. when used for the treatment of cancer, the present chimeric proteins alter the extent of immune stimulation as compared to immune inhibition to increase the amplitude of a T cell response, including, without limitation, stimulating increased levels of cytokine production, proliferation or target killing potential. In embodiments, the patient's T cells are activated and/or stimulated by the chimeric protein, with the activated T cells being capable of dividing and/or secreting cytokines.

Cancers or tumors refer to an uncontrolled growth of cells and/or abnormal increased cell survival and/or inhibition of apoptosis which interferes with the normal functioning of the bodily organs and systems. Included are benign and malignant cancers, polyps, hyperplasia, as well as dormant tumors or micrometastases. Also, included are cells having abnormal proliferation that is not impeded by the immune system (e.g., virus infected cells). The cancer may be a primary cancer or a metastatic cancer. The primary cancer may be an area of cancer cells at an originating site that becomes clinically detectable, and may be a primary tumor. In contrast, the metastatic cancer may be the spread of a disease from one organ or part to another non-adjacent organ or part. The metastatic cancer may be caused by a cancer cell that acquires the ability to penetrate and infiltrate surrounding normal tissues in a local area, forming a new tumor, which may be a local metastasis. The cancer may also be caused by a cancer cell that acquires the ability to penetrate the walls of lymphatic and/or blood vessels, after which the cancer cell is able to circulate through the bloodstream (thereby being a circulating tumor cell) to other sites and tissues in the body. The cancer may be due to a process such as lymphatic or hematogenous spread. The cancer may also be caused by a tumor cell that comes to rest at another site, re-penetrates through the vessel or walls, continues to multiply, and eventually forms another clinically detectable tumor. The cancer may be this new tumor, which may be a metastatic (or secondary) tumor.

The cancer may be caused by tumor cells that have metastasized, which may be a secondary or metastatic tumor. The cells of the tumor may be like those in the original tumor. As an example, if a breast cancer or colon cancer metastasizes to the liver, the secondary tumor, while present in the liver, is made up of abnormal breast or colon cells, not of abnormal liver cells. The tumor in the liver may thus be a metastatic breast cancer or a metastatic colon cancer, not liver cancer.

The cancer may have an origin from any tissue. The cancer may originate from melanoma, colon, breast, or prostate, and thus may be made up of cells that were originally skin, colon, breast, or prostate, respectively. The cancer may also be a hematological malignancy, which may be leukemia or lymphoma. The cancer may invade a tissue such as liver, lung, bladder, or intestinal.

In embodiments, the chimeric protein is used to treat a subject that has a treatment-refractory cancer. In embodiments, the chimeric protein is used to treat a subject that is refractory to one or more immune-modulating agents. For example, in embodiments, the chimeric protein is used to treat a subject that presents no response to treatment, or whose disease progresses, after 12 weeks or so of treatment. For instance, in embodiments, the subject is refractory to one or more CD172a (SIRPα) and/or CD47 agents, including, for example, Magrolimab (5F9), Hu5F9-G4, CC-90002, Ti-061, SRF231, TTI-621, TTI-622, or ALX148 refractory patients. For instance, in embodiments, the subject is refractory to an anti-CTLA-4 agent, e.g., ipilimumab (YERVOY)-refractory patients (e.g., melanoma patients). Accordingly, in embodiments the present disclosure provides methods of cancer treatment that rescue patients that are non-responsive to various therapies, including monotherapy of one or more immune-modulating agents.

In embodiments, the present disclosure provides chimeric proteins which target a cell or tissue within the tumor microenvironment. In embodiments, the cell or tissue within the tumor microenvironment expresses one or more targets or binding partners of the chimeric protein. The tumor microenvironment refers to the cellular milieu, including cells, secreted proteins, physiological small molecules, and blood vessels in which the tumor exists. In embodiments, the cells or tissue within the tumor microenvironment are one or more of: tumor vasculature; tumor-infiltrating lymphocytes; fibroblast reticular cells; endothelial progenitor cells (EPC); cancer-associated fibroblasts; pericytes; other stromal cells; components of the extracellular matrix (ECM); dendritic cells; antigen presenting cells; T-cells; regulatory T cells; macrophages; neutrophils; and other immune cells located proximal to a tumor. In embodiments, the present chimeric protein targets a cancer cell. In embodiments, the cancer cell expresses one or more of targets or binding partners of the chimeric protein.

The activation of regulatory T cells is critically influenced by costimulatory and co-inhibitory signals. Two major families of costimulatory molecules include the B7 and the tumor necrosis factor (TNF) families. These molecules bind to receptors on T cells belonging to the CD28 or TNF receptor families, respectively. Many well-defined co-inhibitors and their receptors belong to the B7 and CD28 families.

In embodiments, an immune stimulatory signal refers to a signal that enhances an immune response. For example, in the context of oncology, such signals may enhance antitumor immunity. For instance, without limitation, immune stimulatory signal may be identified by directly stimulating proliferation, cytokine production, killing activity, or phagocytic activity of leukocytes. For example, a chimeric protein may directly stimulate the proliferation and cytokine production of individual T cell subsets. Another example includes direct stimulation of an immune inhibitory cell with through a receptor that inhibits the activity of such an immune suppressor cell. This would include, for example, stimulation of CD4+FoxP3+ regulatory T cells, which would reduce the ability of those regulatory T cells to suppress the proliferation of conventional CD4+ or CD8+ T cells. In another example, this would include stimulation of CD40 on the surface of an antigen presenting cell, causing activation of antigen presenting cells including enhanced ability of those cells to present antigen in the context of appropriate native costimulatory molecules, including those in the B7 or TNF superfamily. In another example, the chimeric protein causes activation of the lymphoid cell and/or production of pro-inflammatory cytokines or chemokines to further stimulate an immune response, optionally within a tumor.

In embodiments, the present chimeric proteins are capable of, or find use in methods involving, enhancing, restoring, promoting and/or stimulating immune modulation. In embodiments, the present chimeric proteins described herein, restore, promote and/or stimulate the activity or activation of one or more immune cells against tumor cells including, but not limited to: T cells, cytotoxic T lymphocytes, T helper cells, natural killer (NK) cells, natural killer T (NKT) cells, anti-tumor macrophages (e.g. M1 macrophages), B cells, and dendritic cells. In embodiments, the present chimeric proteins enhance, restore, promote and/or stimulate the activity and/or activation of T cells, including, by way of a non-limiting example, activating and/or stimulating one or more T-cell intrinsic signals, including a pro-survival signal; an autocrine or paracrine growth signal; a p38 MAPK-, ERK-, STAT-, JAK-, AKT- or PI3K-mediated signal; an anti-apoptotic signal; and/or a signal promoting and/or necessary for one or more of: pro-inflammatory cytokine production or T cell migration or T cell tumor infiltration.

In embodiments, the present chimeric proteins are capable of, or find use in methods involving, causing an increase of one or more of T cells (including without limitation cytotoxic T lymphocytes, T helper cells, natural killer T (NKT) cells), B cells, natural killer (NK) cells, natural killer T (NKT) cells, dendritic cells, monocytes, and macrophages (e.g., one or more of M1 and M2) into a tumor or the tumor microenvironment. In embodiments, the chimeric protein enhances recognition of tumor antigens by CD8+ T cells, particularly those T cells that have infiltrated into the tumor microenvironment. In embodiments, the present chimeric protein induces CD19 expression and/or increases the number of CD19 positive cells (e.g., CD19 positive B cells). In embodiments, the present chimeric protein induces IL-15Rα expression and/or increases the number of IL-15Rα positive cells (e.g., IL-15Rα positive dendritic cells).

In embodiments, the present chimeric proteins are capable of, or find use in methods involving, inhibiting and/or causing a decrease in immunosuppressive cells (e.g., myeloid-derived suppressor cells (MDSCs), regulatory T cells (Tregs), tumor associated neutrophils (TANs), M2 macrophages, and tumor associated macrophages (TAMs)), and particularly within the tumor and/or tumor microenvironment (TME). In embodiments, the present therapies may alter the ratio of M1 versus M2 macrophages in the tumor site and/or TME to favor M1 macrophages. In embodiments, the SIRPα-Fc-CD40L chimeric protein suppresses/reduces/eliminates a “don't eat me” signal via Sipr1a/CD47 from being transmitted on tumor cells. In embodiments, the SIRPα-Fc-CD40L chimeric protein makes a tumor more likely to be attacked by the immune system of the subject. In embodiments, the SIRPα-Fc-CD40L chimeric protein makes a tumor more likely to be attacked by the innate immune system of the subject. In embodiments, the SIRPα-Fc-CD40L chimeric protein makes a tumor more likely to be attacked by the adaptive immune system of the subject. S In embodiments, the SIRPα-Fc-CD40L chimeric protein can suppress/reduce/eliminate binding of tumor-overexpressed CD47 with phagocyte-expressed SIRPα to permit phagocytic removal of cancer cells and/or immunogenic processing of tumor antigens by macrophages and/or dendritic cells. In embodiments, the administration of the SIRPα-Fc-CD40L chimeric protein blocks the “don't eat me” signal of a tumor cell and/or stimulates an “eat me” signal. In embodiments the therapy with the SIRPα-Fc-CD40L chimeric protein (e.g. SEQ ID NO: 59 or SEQ ID NO: 61) stimulates macrophages to phagocytize tumor cells and effectively present the tumor antigens of phagocytized tumor cells to T cells.

In embodiments, the present chimeric proteins are able to increase the serum levels of various cytokines including, but not limited to, one or more of IFNγ, TNFα, IL-2, IL-4, IL-5, IL-9, IL-10, IL-13, IL-17A, IL-17F, and IL-22. In embodiments, the presentchimeric proteins are capable of enhancing IL-2, IL-4, IL-5, IL-10, IL-13, IL-17A, IL-22, or IFNγ in the serum of a treated subject. In embodiments, the present chimeric proteins do not increase the serum levels of certain cytokines. In embodiments, the present chimeric proteins do not increase the serum levels of IL-6 and/or TNFα. In embodiments, the present chimeric proteins do not increase the serum levels of f IL-6 and/or TNFα in the serum of a treated subject. In embodiments, the present chimeric proteins do not increase the serum levels of f IL-6 and/or TNFα in the serum of a treated subject, while increasing the levels of other cytokines, including but not limited to, CCL2, IL-8 and CXCL9 in serum of a treated subject. Detection of such a cytokine response may provide a method to determine the optimal dosing regimen for the indicated chimeric protein.

In a chimeric protein of the present disclosure, the chimeric protein is capable of increasing or preventing a decrease in a sub-population of CD4+ and/or CD8+ T cells.

In a chimeric protein of the present disclosure, the chimeric protein is capable of enhancing tumor killing activity by T cells.

In embodiments, the chimeric protein activates the human subject's T cells when bound by the CD40L domain of the chimeric protein and (a) one or more tumor cells are prevented from transmitting an immunosuppressive signal when bound by the first domain of the chimeric protein, (b) a quantifiable cytokine response in the peripheral blood of the subject is achieved, and/or (c) tumor growth is reduced in the subject in need thereof as compared to a subject treated with CD40 agonist antibodies and/or CD47 blocking antibodies.

In embodiments, the present chimeric proteins inhibit, block and/or reduce cell death of an anti-tumor CD8+ and/or CD4+ T cell; or stimulate, induce, and/or increase cell death of a pro-tumor T cell. T cell exhaustion is a state of T cell dysfunction characterized by progressive loss of proliferative and effector functions, culminating in clonal deletion. Accordingly, a pro-tumor T cell refers to a state of T cell dysfunction that arises during many chronic infections, inflammatory diseases, and cancer. This dysfunction is defined by poor proliferative and/or effector functions, sustained expression of inhibitory receptors and a transcriptional state distinct from that of functional effector or memory T cells. Exhaustion prevents optimal control of infection and tumors. Illustrative pro-tumor T cells include, but are not limited to, Tregs, CD4+ and/or CD8+ T cells expressing one or more checkpoint inhibitory receptors, Th2 cells and Th17 cells. Checkpoint inhibitory receptors refer to receptors expressed on immune cells that prevent or inhibit uncontrolled immune responses. In contrast, an anti-tumor CD8+ and/or CD4+ T cell refers to T cells that can mount an immune response to a tumor.

In embodiments, the present chimeric proteins are capable of, and can be used in methods comprising, increasing a ratio of effector T cells to regulatory T cells. Illustrative effector T cells include ICOS⁺ effector T cells; cytotoxic T cells (e.g., αβ TCR, CD3⁺, CD8⁺, CD45RO⁺); CD4⁺ effector T cells (e.g., αβ TCR, CD3⁺, CD4⁺, CCR7⁺, CD62Lhi, IL⁻7R/CD127⁺); CD8⁺ effector T cells (e.g., αβ TCR, CD3⁺, CD8⁺, CCR7⁺, CD62Lhi, IL⁻7R/CD127⁺); effector memory T cells (e.g., CD62Llow, CD44⁺, TCR, CD3⁺, IL-7R/CD127⁺, IL-15R*, CCR7low); central memory T cells (e.g., CCR7⁺, CD62L⁺, CD27⁺; or CCR7hi, CD44⁺, CD62Lhi, TCR, CD3⁺, IL-7R/CD127⁺, IL-15R⁺); CD62L⁺ effector T cells; CD8⁺ effector memory T cells (TEM) including early effector memory T cells (CD27⁺ CD62L⁻) and late effector memory T cells (CD27⁻ CD62L⁻) (TemE and TemL, respectively); CD127(⁺)CD25(low/−) effector T cells; CD127(⁻)CD25(⁻) effector T cells; CD8⁺ stem cell memory effector cells (TSCM) (e.g., CD44(low)CD62L(high)CD122(high)sca(⁺)); TH1 effector T-cells (e.g., CXCR3⁺, CXCR6⁺ and CCR5⁺; or αβ TCR, CD3⁺, CD4⁺, IL-12R⁺, IFNγR⁺, CXCR3⁺), TH2 effector T cells (e.g., CCR3⁺, CCR4⁺ and CCR8⁺; or αβ TCR, CD3⁺, CD4⁺, IL-4R⁺, IL-33R⁺, CCR4⁺, IL-17RB⁺, CRTH2⁺); TH9 effector T cells (e.g., αβ TCR, CD3⁺, CD4⁺); TH17 effector T cells (e.g., αβ TCR, CD3⁺, CD4⁺, IL-23R⁺, CCR6⁺, IL-1R⁺); CD4⁺CD45RO⁺CCR7⁺ effector T cells, CD4⁺CD45RO⁺CCR7(−) effector T cells; and effector T cells secreting IL-2, IL-4 and/or IFN-γ. Illustrative regulatory T cells include ICOS⁺ regulatory T cells, CD4⁺CD25⁺FOXP3⁺ regulatory T cells, CD4⁺CD25⁺ regulatory T cells, CD4⁺CD25⁻ regulatory T cells, CD4⁺CD25high regulatory T cells, TIM-3⁺CD172a (SIRPα)⁺ regulatory T cells, lymphocyte activation gene-3 (LAG-3)⁺ regulatory T cells, CTLA-4/CD152⁺ regulatory T cells, neuropilin-1 (Nrp-1)⁺ regulatory T cells, CCR4⁺CCR8⁺ regulatory T cells, CD62L (L-selectin)⁺ regulatory T cells, CD45RBlow regulatory T cells, CD127low regulatory T cells, LRRC32/GARP⁺ regulatory T cells, CD39⁺ regulatory T cells, GITR⁺ regulatory T cells, LAP⁺ regulatory T cells, 1B11⁺ regulatory T cells, BTLA⁺ regulatory T cells, type 1 regulatory T cells (Tr1 cells), T helper type 3 (Th3) cells, regulatory cell of natural killer T cell phenotype (NKTregs), CD8⁺ regulatory T cells, CD8⁺CD28⁻ regulatory T cells and/or regulatory T-cells secreting IL-10, IL-35, TGF-β, TNF-α, Galectin-1, IFN-γ and/or MCP1.

In embodiments, the chimeric protein of the invention causes an increase in effector T cells (e.g., CD4+CD25− T cells).

In embodiments, the chimeric protein causes a decrease in regulatory T cells (e.g., CD4+CD25+ T cells).

In embodiments, the chimeric protein generates a memory response which may, e.g., be capable of preventing relapse or protecting the animal from a recurrence and/or preventing, or reducing the likelihood of, metastasis. Thus, an animal treated with the chimeric protein is later able to attack tumor cells and/or prevent development of tumors when rechallenged after an initial treatment with the chimeric protein. Accordingly, a chimeric protein of the present disclosure stimulates both active tumor destruction and also immune recognition of tumor antigens, which are essential in programming a memory response capable of preventing relapse.

In embodiments, the chimeric protein is capable of causing activation of antigen presenting cells. In embodiments, the chimeric protein is capable enhancing the ability of antigen presenting cells to present antigen.

In embodiments, the present chimeric proteins are capable of, and can be used in methods comprising, transiently stimulating effector T cells for longer than about 12 hours, about 24 hours, about 48 hours, about 72 hours or about 96 hours or about 1 week or about 2 weeks. In embodiments, the transient stimulation of effector T cells occurs substantially in a patient's bloodstream or in a particular tissue/location including lymphoid tissues such as for example, the bone marrow, lymph-node, spleen, thymus, mucosa-associated lymphoid tissue (MALT), non-lymphoid tissues, or in the tumor microenvironment.

In a chimeric protein of the present disclosure, the present chimeric protein unexpectedly provides binding of the extracellular domain components to their respective binding partners with slow off rates (Kd or K_{off}). In embodiments, this provides an unexpectedly long interaction of the receptor to ligand and vice versa. Such an effect allows for a longer positive signal effect, e.g., increase in or activation of immune stimulatory signals. For example, the present chimeric protein, e.g., via the long off rate binding allows sufficient signal transmission to provide immune cell proliferation, allow for anti-tumor attack, allows sufficient signal transmission to provide release of stimulatory signals, e.g., cytokines.

In a chimeric protein of the present disclosure, the chimeric protein is capable of forming a stable synapse between cells. The stable synapse of cells promoted by the chimeric proteins (e.g., between cells bearing negative signals) provides spatial orientation to favor tumor reduction—such as positioning the T cells to attack tumor cells and/or sterically preventing the tumor cell from delivering negative signals, including negative signals beyond those masked by the chimeric protein of the invention. In embodiments, this provides longer on-target (e.g., intratumoral) half-life (t_{1/2}) as compared to serum t_{1/2} of the chimeric proteins. Such properties could have the combined advantage of reducing off-target toxicities associated with systemic distribution of the chimeric proteins.

In embodiments, the chimeric protein is capable of providing a sustained immunomodulatory effect.

The present chimeric proteins provide synergistic therapeutic effects (e.g., anti-tumor effects) as it allows for improved site-specific interplay of two immunotherapy agents. In embodiments, the present chimeric proteins provide the potential for reducing off-site and/or systemic toxicity.

In embodiments, the present chimeric protein exhibit enhanced safety profiles. In embodiments, the present chimeric protein exhibit reduced toxicity profiles. For example, administration of the present chimeric proteins may result in reduced side effects such as one or more of diarrhea, inflammation (e.g., of the gut), or weight loss, which occur following administration of antibodies directed to the ligand(s)/receptor(s) targeted by the extracellular domains of the present chimeric proteins. In embodiments, the present chimeric protein provides improved safety, as compared to antibodies directed to the ligand(s)/receptor(s) targeted by the extracellular domains of the present chimeric proteins, yet, without sacrificing efficacy.

In embodiments, the present chimeric proteins provide reduced side-effects, e.g., GI complications, relative to current immunotherapies, e.g., antibodies directed to ligand(s)/receptor(s) targeted by the extracellular domains of the present chimeric proteins. Illustrative GI complications include abdominal pain, appetite loss, autoimmune effects, constipation, cramping, dehydration, diarrhea, eating problems, fatigue, flatulence, fluid in the abdomen or ascites, gastrointestinal (GI) dysbiosis, GI mucositis, inflammatory bowel disease, irritable bowel syndrome (IBS-D and IBS-C), nausea, pain, stool or urine changes, ulcerative colitis, vomiting, weight gain from retaining fluid, and/or weakness.

### Pharmaceutical Composition

Aspects of the present disclosure include a pharmaceutical composition comprising a therapeutically effective amount of a chimeric protein as disclosed herein.

Any chimeric protein disclosed herein may be used in a pharmaceutical composition.

In embodiments, a chimeric protein disclosed herein is provided as a sterile frozen solution in a vial or as a sterile liquid solution in a vial. A drug product comprising a chimeric protein disclosed herein comprises a sterile-filtered, formulated chimeric protein disclosed herein solution filled into a 10 mL single use glass vial stoppered with a Flurotec® rubber stopper and sealed with an aluminum flip off seal. In embodiments, a chimeric protein disclosed herein is formulated at between about 10 mg/mL to about 30 mg/mL, e.g., about 20 mg/mL in between about 30 mM to about 70 mM L-histidine, e.g., about 50 mM L-histidine and between about 125 mM and about 400 mM sucrose, e.g., about 250 mM sucrose in water for injection. In embodiments, each vial contains about 1 mL of drug product or about 20 mg of a chimeric protein disclosed herein.

The chimeric proteins disclosed herein, including the CD172a (SIRPα)-Fc-CD40L chimeric protein (e.g. SEQ ID NO: 59 or SEQ ID NO: 61), can possess a sufficiently basic functional group, which can react with an inorganic or organic acid, or a carboxyl group, which can react with an inorganic or organic base, to form a pharmaceutically acceptable salt. A pharmaceutically-acceptable acid addition salt is formed from a pharmaceutically acceptable acid, as is well known in the art. Such salts include the pharmaceutically acceptable salts listed in, for example, Journal of Pharmaceutical Science, 66, 2-19 (1977) and The Handbook of Pharmaceutical Salts; Properties, Selection, and Use. P. H. Stahl and C. G. Wermuth (eds.), Verlag, Zurich (Switzerland) 2002, which are hereby incorporated by reference in their entirety.

In embodiments, the compositions disclosed herein are in the form of a pharmaceutically acceptable salt.

Further, any chimeric protein disclosed herein can be administered to a subject as a component of a composition, e.g., pharmaceutical composition, that comprises a pharmaceutically acceptable carrier or vehicle. Such pharmaceutical compositions can optionally comprise a suitable amount of a pharmaceutically acceptable excipient so as to provide the form for proper administration. Pharmaceutical excipients can be liquids, such as water and oils, including those of petroleum, animal, vegetable, or synthetic origin, such as peanut oil, soybean oil, mineral oil, sesame oil and the like. The pharmaceutical excipients can be, for example, saline, gum acacia, gelatin, starch paste, talc, keratin, colloidal silica, urea and the like. In addition, auxiliary, stabilizing, thickening, lubricating, and coloring agents can be used. In embodiments, the pharmaceutically acceptable excipients are sterile when administered to a subject. Water is a useful excipient when any agent disclosed herein is administered intravenously. Saline solutions and aqueous dextrose and glycerol solutions can also be employed as liquid excipients, specifically for injectable solutions. Suitable pharmaceutical excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Any agent disclosed herein, if desired, can also comprise minor amounts of wetting or emulsifying agents, or pH buffering agents.

In embodiments, the compositions, e.g., pharmaceutical compositions, disclosed herein are resuspended in a saline buffer (including, without limitation TBS, PBS, and the like).

In embodiments, the chimeric proteins may by conjugated and/or fused with another agent to extend half-life or otherwise improve pharmacodynamic and pharmacokinetic properties. In embodiments, the chimeric proteins may be fused or conjugated with one or more of PEG, XTEN (e.g., as rPEG), polysialic acid (POLYXEN), albumin (e.g., human serum albumin or HAS), elastin-like protein (ELP), PAS, HAP, GLK, CTP, transferrin, and the like. In embodiments, each of the individual chimeric proteins is fused to one or more of the agents described in Strohl, BioDrugs 29(4):215-239 (2015), the entire contents of which are hereby incorporated by reference.

The present disclosure includes the disclosed chimeric protein in various formulations of pharmaceutical composition. Any chimeric protein disclosed herein can take the form of solutions, suspensions, emulsion, drops, tablets, pills, pellets, capsules, capsules containing liquids, powders, sustained-release formulations, suppositories, emulsions, aerosols, sprays, suspensions, or any other form suitable for use. DNA or RNA constructs encoding the protein sequences may also be used. In embodiments, the composition is in the form of a capsule (see, e.g., U.S. Pat. No. 5,698,155). Other examples of suitable pharmaceutical excipients are described in Remington's Pharmaceutical Sciences 1447-1676 (Alfonso R. Gennaro eds., 19th ed. 1995), incorporated herein by reference.

Where necessary, the pharmaceutical compositions comprising the chimeric protein (can also include a solubilizing agent. Also, the agents can be delivered with a suitable vehicle or delivery device as known in the art. Compositions for administration can optionally include a local anesthetic such as, for example, lignocaine to lessen pain at the site of the injection.

The pharmaceutical compositions comprising the chimeric protein of the present disclosure may conveniently be presented in unit dosage forms and may be prepared by any of the methods well known in the art of pharmacy. Such methods generally include the step of bringing therapeutic agents into association with a carrier, which constitutes one or more accessory ingredients. Typically, the pharmaceutical compositions are prepared by uniformly and intimately bringing therapeutic agent into association with a liquid carrier, a finely divided solid carrier, or both, and then, if necessary, shaping the product into dosage forms of the desired formulation (e.g., wet or dry granulation, powder blends, etc., followed by tableting using conventional methods known in the art)

In embodiments, any chimeric protein disclosed herein is formulated in accordance with routine procedures as a pharmaceutical composition adapted for a mode of administration disclosed herein.

### Administration, Dosing, and Treatment Regimens

In embodiments, a chimeric protein disclosed herein is presented as a sterile frozen solution at a concentration of about 20 mg/mL and a total volume of about 1 mL, optionally in a 10 mL glass vial. In embodiments, a chimeric protein disclosed herein is administered by intravenous (IV) infusion following dilution with normal saline. Starting dose, dose escalation schema and dose schedules of certain embodiments are presented below.

In embodiments, the dose of the chimeric protein administered is at least 0.0001 mg/kg, e.g., between about 0.0001 mg/kg and about 10 mg/kg. In embodiments, the dose of the chimeric protein administered is at least about 0.3 mg/kg, e.g., at least about 0.3 mg/kg, or about 1.0 mg/kg, or about 2 mg/kg, or about 3, about 4 mg/kg, or about 6 mg/kg, or about 8 mg/kg, or about 10 mg/kg. In embodiments, the dose of the chimeric protein administered is at least about 1 mg/kg, e.g., at least about 1.0 mg/kg, or about 2 mg/kg, or about 3, about 4 mg/kg, or about 6 mg/kg, or about 8 mg/kg, or about 10 mg/kg. In embodiments, the doses of the SIRPα-Fc-CD40L chimeric protein are not limited by anemia or another cytopenia effects and are therefore higher than doses are allowed compared to certain other therapeutics (e.g. anti-CD47 antibodies or SIRPalphaFc fusion protein). Further, in embodiments, a low dose priming is not needed.

In embodiments, the administration is intravenous. In embodiments, the administration is intratumoral. In embodiments, the administration is by injection. In embodiments, the administration is by infusion. In embodiments, the administration is performed by an intravenous infusion. In embodiments, the administration is performed by an intratumoral injection.

In embodiments, about the chimeric protein is administered at an initial dose (e.g., about one of about 0.0001, about 0.001, about 0.003, about 0.01, about 0.03, about 0.1, about 0.3, about 1, about 2, about 3, about 4, about 6, about 8 or about 10 mg/kg) and the chimeric protein is administered in one or more subsequent administrations. In embodiments, about the one or more subsequent administrations has a dose of one or more of about 0.0001, about 0.001, about 0.003, about 0.01, about 0.03, about 0.1, about 0.3, about 1, about 2, about 3, about 4, about 6, about 8, about and about 10 mg/kg.

In embodiments, the starting dose and/or the subsequent doses is the maximum tolerated dose or less than the maximum tolerated dose.

In embodiments, the dose escalates between one or more subsequent dose in log increments, e.g., 0.0001 mg/kg to 0.001 mg/kg, 0.001 mg/kg to 0.01 mg/kg, and 0.01 mg/kg to 0.1 mg/kg.

In embodiments, the dose escalates between one or more subsequent dose in about half log increments, e.g., 0.001 mg/kg to 0.003 mg/kg and 0.003 mg/kg to 0.01 mg/kg.

In embodiments, the human subject has failed platinum-based therapies, and optionally is ineligible for further platinum therapy. In embodiments, the human subject is not receiving a concurrent chemotherapy, immunotherapy, biologic or hormonal therapy, and/or wherein the human subject has received, been tolerant to, or is ineligible for standard therapy and/or the cancer has no approved therapy considered to be standard of care.

In embodiments, the initial dose is less than the dose for at least one of the subsequent administrations, e.g., each of the subsequent administrations.

In embodiments, the initial dose is the same as the dose for at least one of the subsequent administrations, e.g., each of the subsequent administrations.

In embodiments, the chimeric protein is administered at least about one time a month.

In embodiments, the chimeric protein is administered at least about two times a month.

In embodiments, the chimeric protein is administered at least about three times a month.

In embodiments, the chimeric protein is first administered once a week for three weeks and the chimeric protein is then administered about once every three weeks or once every four weeks.

In embodiments, the chimeric protein is first administered once a week for three weeks and the chimeric protein is then administered about two times per month. For example, the chimeric protein is first administered once a week for three weeks and the chimeric protein is then administered about once every two weeks.

In embodiments, the chimeric protein is administered at least about four times a month. For example, the chimeric protein is administered about once a week. In embodiments, the chimeric protein is administered once every week (once every seven days). in embodiments, the chimeric protein is administered once every two weeks.

In embodiments, the administration of the SIRPα-Fc-CD40L chimeric protein does not cause an anemia or another cytopenia in the patient. In embodiments, the administration of the does not cause lysis of RBCs. In embodiments, the administration of the SIRPα-Fc-CD40L chimeric protein is less likely to cause anemia or another cytopenia in than, e.g. an anti-CD47 Ab. In embodiments, the doses of the SIRPα-Fc-CD40L chimeric protein are not limited by anemia or another cytopenia effects and are therefore higher than doses are allowed compared to certain other therapeutics (e.g. anti-CD47 antibodies or SIRPalphaFc fusion protein). Further, in embodiments, a low dose priming is not needed.

Another advantage the SIRPα-Fc-CD40L chimeric protein (e.g. SEQ ID NO: 59 or SEQ ID NO: 61) offers is that despite targeting does not cause an anemia or another cytopenia in the patient. This is because although the CD47/SIRPα interaction plays a key role in the lysis of RBCs, as shown herein, the SIRPα-Fc-CD40L chimeric protein does not cause lysis of RBCs. Accordingly, the present methods are less likely to cause anemia or another cytopenia in than, e.g. an anti-CD47 Ab.

A chimeric protein may be administered intravenously by intravenous infusion or bolus injection into the bloodstream. A chimeric protein may be administered intravenously by intravenous infusion for patients suffering from advanced ovarian, fallopian tube and primary peritoneal cancers.

A chimeric protein may be administered an intratumoral injection. In embodiments, the therapeutic dose for intra-tumoral administration is equal or less than that of intravenous infusion. In embodiments, the therapeutic dose for intra-tumoral administration is equal to that of intravenous infusion. In embodiments, the therapeutic dose for intra-tumoral administration is less than that of intravenous infusion. In embodiments, the therapeutic dose for intra-tumoral administration for patients suffering from advanced or metastatic CSCC and HNSCC.

In embodiments, the present chimeric protein allows for a dual effect that provides less side effects than are seen in conventional immunotherapy (e.g., treatments with one or more of OPDIVO, KEYTRUDA, YERVOY, and TECENTRIQ). For example, the present chimeric proteins reduce or prevent commonly observed immune-related adverse events that affect various tissues and organs including the skin, the gastrointestinal tract, the kidneys, peripheral and central nervous system, liver, lymph nodes, eyes, pancreas, and the endocrine system; such as hypophysitis, colitis, hepatitis, pneumonitis, rash, and rheumatic disease.

Dosage forms suitable for intravenous administration include, for example, solutions, suspensions, dispersions, emulsions, and the like. They may also be manufactured in the form of sterile solid compositions (e.g., lyophilized composition), which can be dissolved or suspended in sterile injectable medium immediately before use. They may contain, for example, suspending or dispersing agents known in the art.

The dosage of any chimeric protein disclosed herein as well as the dosing schedule can depend on various parameters, including, but not limited to, the disease being treated, the subject's general health, and the administering physician's discretion.

In one aspect, the present disclosure relates to a method for treating a cancer in a human subject in need thereof, the method comprising a step of administering to the human subject an effective amount of a chimeric protein having a general structure of: N terminus-(a)-(b)-(c)-C terminus, wherein: (a) is a first domain comprising an extracellular domain of human signal regulatory protein α (CD172a (SIRPα)), (b) is a linker adjoining the first and second domains, wherein the linker comprises a hinge-CH2-CH3 Fc domain, and (c) is a second domain comprising an extracellular domain of human CD40 ligand (CD40L), wherein the step of administering comprises biphasic dosing. In embodiments, the first phase, and the second phase each independently comprise a dosing frequency of from about twice a week to about once every two months. In embodiments, the linker comprises at least one cysteine residue capable of forming a disulfide bond. In embodiments, the chimeric protein exhibits a linear dose response in the dose range of e.g., about 0.3 mg/kg to about 3 mg/kg, or about 0.5 mg/kg to about 3 mg/kg, or about 0.7 mg/kg to about 3 mg/kg, or about 1 mg/kg to about 3 mg/kg, or about 1.5 mg/kg to about 3 mg/kg, or about 2 mg/kg to about 3 mg/kg, or about 2.5 mg/kg to about 3 mg/kg, or about 0.3 mg/kg to about 2.5 mg/kg, or about 0.3 mg/kg to about 2 mg/kg, or about 0.3 mg/kg to about 1.5 mg/kg, or about 0.3 mg/kg to about 1.0 mg/kg, or about 0.3 mg/kg to about 0.5 mg/kg. In embodiments, the chimeric protein does not exhibit a bell-shaped dose response.

In embodiments, the dosing frequency of the first phase, and the dosing frequency of the second phase are the same. In other embodiments, the dosing frequency of the first phase, and the dosing frequency of the second phase are different.

In embodiments, the dosing frequency of the first phase is selected from about every three days, about twice a week, about every week, about every 10 days, about twice every 3 weeks, about every 2 weeks, about every 3 weeks, about every 4 weeks, about every month, about every 5 weeks, about every 6 weeks, about 7 seven weeks, about every 8 weeks and about every 2 months. In embodiments, the dosing frequency of the first phase is selected from about every 3 days to about every 10 days, about every week to about every 2 weeks, about every 10 days to about every 3 weeks, about every 2 weeks to about every 4 weeks, about every 3 weeks to about every 5 weeks, about every 4 weeks to about every 6 weeks, about every 5 weeks to about every 7 weeks, about every 6 weeks to about every 8 weeks, and, about every 6 weeks to about every 2 months.

In embodiments, the dosing frequency of the second phase is selected from about every three days, about twice a week, about every week, about every 10 days, about twice every 3 weeks, about every 2 weeks, about every 3 weeks, about every 4 weeks, about every month, about every 5 weeks, about every 6 weeks, about 7 seven weeks, about every 8 weeks and about every 2 months. In embodiments, the dosing frequency of the second phase is selected from about every 3 days to about every 10 days, about every week to about every 2 weeks, about every 10 days to about every 3 weeks, about every 2 weeks to about every 4 weeks, about every 3 weeks to about every 5 weeks, about every 4 weeks to about every 6 weeks, about every 5 weeks to about every 7 weeks, about every 6 weeks to about every 8 weeks, and, about every 6 weeks to about every 2 months.

In embodiments, the dosing frequency of the first phase is selected from from about every 3 days to about every 10 days, about every week to about every 2 weeks, about every 10 days to about every 3 weeks; and the frequency of the second phase is selected from from about every week to about every 2 weeks, about every 10 days to about every 3 weeks, about every 2 weeks to about every 4 weeks, about every 3 weeks to about every 5 weeks, about every 4 weeks to about every 6 weeks.

Additionally, or alternatively, in embodiments, the first phase, and the second phase each independently last from about two days to about 12 months. In embodiments, the first phase lasts from about two weeks to about 2 months; and the second phase lasts from about 2 weeks to about 12 months. In embodiments, the first phase lasts from about two weeks to about 1 month; and the second phase lasts from about 2 weeks to about 12 months. In embodiments, the first phase lasts from about two weeks to about 1 month; and the second phase lasts from about 4 weeks to about 12 months.

Additionally, or alternatively, in embodiments, the effective amount for the first phase, the second phase and the third phase each independently comprise about 0.01 mg/kg to about 10 mg/ml. In embodiments, the effective amount for the first phase, the second phase and the third phase each independently selected from about 0.03 mg/kg, about 0.1 mg/kg, about 0.3 mg/kg, about 1 mg/kg, about 3 mg/kg, about 10 mg/kg, and any range including and/or in between any two of the preceding values. In embodiments, the effective amount for the first phase, the second phase and the third phase each independently selected from from about 0.01 mg/kg to about 0.1 mg/kg, about 0.03 mg/kg to about 0.3 mg/kg, about 0.1 mg/kg to about 1 mg/kg, about 0.3 mg/kg to about 3 mg/kg, and about 1 mg/kg to about 10 mg/kg. In embodiments, the effective amount for the first phase, the second phase and the third phase are same. In embodiments, the effective amount for the first phase, the second phase and the third phase are different. In embodiments, the effective amount for the first phase is greater than the effective amount for the second phase. In embodiments, the effective amount for the first phase is from about 0.03 mg/kg to about 0.3 mg/kg, about 0.1 mg/kg to about 1 mg/kg, about 0.3 mg/kg to about 3 mg/kg, or about 1 mg/kg to about 10 mg/kg; and the effective amount for the second phase is from about 0.01 mg/kg to about 0.1 mg/kg, about 0.03 mg/kg to about 0.3 mg/kg, about 0.1 mg/kg to about 1 mg/kg, about 0.3 mg/kg to about 3 mg/kg, or about 1 mg/kg to about 10 mg/kg.

In embodiments, the chimeric proteins disclosed herein is the human CD172a (SIRPα)-Fc-CD40L chimeric protein.

In one aspect, the present disclosure relates to a method for treating a cancer in a human subject in need thereof the method comprising a step of administering to the human subject an effective amount of a chimeric protein having a general structure of: N terminus-(a)-(b)-(c)-C terminus, wherein: (a) is a first domain comprising an extracellular domain of human signal regulatory protein α (CD172a (SIRPα)), (b) is a linker adjoining the first and second domains, wherein the linker comprises a hinge-CH2-CH3 Fc domain, and (c) is a second domain comprising an extracellular domain of human CD40 ligand (CD40L), wherein the step of administration comprises a first cycle, a second cycle and a third cycle. In embodiments, the linker comprises at least one cysteine residue capable of forming a disulfide bond. In embodiments, the first cycle, the second cycle and the third cycle each independently comprise a dosing frequency of from about twice a week to about once every two months. In embodiments, the dosing frequency of the first cycle, the dosing frequency of the second cycle and the dosing frequency of the third cycle are the same. In embodiments, the dosing frequency of the first cycle, the dosing frequency of the second cycle and the dosing frequency of the third cycle are different. In embodiments, the dosing frequency of the first cycle is selected from about every three days, about twice a week, about every week, about every 10 days, about twice every 3 weeks, about every 2 weeks, about every 3 weeks, about every 4 weeks, about every month, about every 5 weeks, about every 6 weeks, about 7 seven weeks, about every 8 weeks and about every 2 months. In embodiments, the chimeric protein exhibits a linear dose response in the dose range of e.g., about 0.3 mg/kg to about 3 mg/kg, or about 0.5 mg/kg to about 3 mg/kg, or about 0.7 mg/kg to about 3 mg/kg, or about 1 mg/kg to about 3 mg/kg, or about 1.5 mg/kg to about 3 mg/kg, or about 2 mg/kg to about 3 mg/kg, or about 2.5 mg/kg to about 3 mg/kg, or about 0.3 mg/kg to about 2.5 mg/kg, or about 0.3 mg/kg to about 2 mg/kg, or about 0.3 mg/kg to about 1.5 mg/kg, or about 0.3 mg/kg to about 1.0 mg/kg, or about 0.3 mg/kg to about 0.5 mg/kg. In embodiments, the chimeric protein does not exhibit a bell-shaped dose response.

In embodiments, the dosing frequency of the first cycle is selected from about every 3 days to about every 10 days, about every week to about every 2 weeks, about every 10 days to about every 3 weeks, about every 2 weeks to about every 4 weeks, about every 3 weeks to about every 5 weeks, about every 4 weeks to about every 6 weeks, about every 5 weeks to about every 7 weeks, about every 6 weeks to about every 8 weeks, and, about every 6 weeks to about every 2 months. In embodiments, the dosing frequency of the second cycle is selected from about every three days, about twice a week, about every week, about every 10 days, about twice every 3 weeks, about every 2 weeks, about every 3 weeks, about every 4 weeks, about every month, about every 5 weeks, about every 6 weeks, about 7 seven weeks, about every 8 weeks and about every 2 months. In embodiments, the dosing frequency of the second cycle is selected from about every 3 days to about every 10 days, about every week to about every 2 weeks, about every 10 days to about every 3 weeks, about every 2 weeks to about every 4 weeks, about every 3 weeks to about every 5 weeks, about every 4 weeks to about every 6 weeks, about every 5 weeks to about every 7 weeks, about every 6 weeks to about every 8 weeks, and, about every 6 weeks to about every 2 months. In embodiments, the dosing frequency of the third cycle is selected from about every three days, about twice a week, about every week, about every 10 days, about twice every 3 weeks, about every 2 weeks, about every 3 weeks, about every 4 weeks, about every month, about every 5 weeks, about every 6 weeks, about 7 seven weeks, about every 8 weeks and about every 2 months. In embodiments, the dosing frequency of the third cycle is selected from about every 3 days to about every 10 days, about every week to about every 2 weeks, about every 10 days to about every 3 weeks, about every 2 weeks to about every 4 weeks, about every 3 weeks to about every 5 weeks, about every 4 weeks to about every 6 weeks, about every 5 weeks to about every 7 weeks, about every 6 weeks to about every 8 weeks, and, about every 6 weeks to about every 2 months. In embodiments, the dosing frequency of the first cycle is selected from from about every 3 days to about every 10 days, about every week to about every 2 weeks, about every 10 days to about every 3 weeks; and the frequency of the second cycle is selected from from about every week to about every 2 weeks, about every 10 days to about every 3 weeks, about every 2 weeks to about every 4 weeks, about every 3 weeks to about every 5 weeks, about every 4 weeks to about every 6 weeks.

Additionally, or alternatively, in embodiments, the first cycle, the second cycle and the third cycle each independently last from about two days to about 12 months. In embodiments, the first cycle lasts from about two weeks to about 2 months; and the second cycle lasts from about 2 weeks to about 12 months. In embodiments, the first cycle lasts from about two weeks to about 2 months; the second cycle lasts from about 2 weeks to about 12 months and the third cycle lasts from about 2 weeks to about 6 months.

Additionally, or alternatively, in embodiments, the effective amount for the first cycle, the second cycle and the third cycle each independently comprise about 0.01 mg/kg to about 10 mg/ml. In embodiments, the effective amount for the first cycle, the second cycle and the third cycle each independently selected from about 0.03 mg/kg, about 0.1 mg/kg, about 0.3 mg/kg, about 1 mg/kg, about 3 mg/kg, about 10 mg/kg, and any range including and/or in between any two of the preceding values. In embodiments, the effective amount for the first cycle, the second cycle and the third cycle each independently selected from from about 0.01 mg/kg to about 0.1 mg/kg, about 0.03 mg/kg to about 0.3 mg/kg, about 0.1 mg/kg to about 1 mg/kg, about 0.3 mg/kg to about 3 mg/kg, and about 1 mg/kg to about 10 mg/kg.

In embodiments, the effective amount for the first cycle, the second cycle and the third cycle are same. In other embodiments, the effective amount for the first cycle, the second cycle and the third cycle are different. In embodiments, the effective amount for the first cycle is greater than the effective amount for the second cycle. In other embodiments, the effective amount for the first cycle is lesser than the effective amount for the second cycle. In yet other embodiments, the effective amount for the first cycle and the effective amount for the second cycle are the same.

In embodiments, the effective amount for the first cycle is from about 0.03 mg/kg to about 0.3 mg/kg, about 0.1 mg/kg to about 1 mg/kg, about 0.3 mg/kg to about 3 mg/kg, or about 1 mg/kg to about 10 mg/kg; and the effective amount for the second cycle is from about 0.01 mg/kg to about 0.1 mg/kg, about 0.03 mg/kg to about 0.3 mg/kg, about 0.1 mg/kg to about 1 mg/kg, about 0.3 mg/kg to about 3 mg/kg, or about 1 mg/kg to about 10 mg/kg.

In embodiments, the chimeric proteins disclosed herein is the human CD172a (SIRPα)-Fc-CD40L chimeric protein.

In one aspect, the present disclosure relates to a method for treating a cancer in a human subject in need thereof the method comprising a step of administering to the human subject an effective amount of an effective amount of a chimeric protein having a general structure of: N terminus-(a)-(b)-(c)-C terminus, wherein: (a) is a first domain comprising an extracellular domain of human signal regulatory protein α (CD172a (SIRPα)), (b) is a linker adjoining the first and second domains, wherein the linker comprises a hinge-CH2-CH3 Fc domain, and (c) is a second domain comprising an extracellular domain of human CD40 ligand (CD40L) with a dosing regimen, wherein the dosing regimen comprises dosing with a frequency in the range of about every three days to about every 2 months. In embodiments, the linker comprises at least one cysteine residue capable of forming a disulfide bond. In embodiments, the dosing regimen is selected from about every three days, about twice a week, about every week, about every 10 days, about twice every 3 weeks, about every 2 weeks, about every 3 weeks, about every 4 weeks, about every month, about every 5 weeks, about every 6 weeks, about 7 seven weeks, about every 8 weeks and about every 2 months. In embodiments, the dosing regimen is selected from about every week, about every 10 days, about every 2 weeks, about every 3 weeks, about every 4 weeks, about every month, about every 5 weeks, about every 6 weeks, about 7 seven weeks, about every 8 weeks and about every 2 months. In embodiments, the dosing regimen is about every 2 weeks, about every 3 weeks, or about every 4 weeks.

In one aspect, the present disclosure relates to a method for treating a cancer in a human subject in need thereof the method comprising a step of administering to the human subject an effective amount of an effective amount of a chimeric protein having a general structure of: N terminus-(a)-(b)-(c)-C terminus, wherein: (a) is a first domain comprising an extracellular domain of human signal regulatory protein α (CD172a (SIRPα)), (b) is a linker adjoining the first and second domains, wherein the linker comprises a hinge-CH2-CH3 Fc domain, and (c) is a second domain comprising an extracellular domain of human CD40 ligand (CD40L) with a dosing regimen selected from about every 3 days to about every 10 days, about every week to about every 2 weeks, about every 10 days to about every 3 weeks, about every 2 weeks to about every 4 weeks, about every 3 weeks to about every 5 weeks, about every 4 weeks to about every 6 weeks, about every 5 weeks to about every 7 weeks, about every 6 weeks to about every 8 weeks, and, about every 6 weeks to about every 2 months. In embodiments, the linker comprises at least one cysteine residue capable of forming a disulfide bond. In embodiments, the dosing regimen is about every week to about every 2 weeks, about every 10 days to about every 3 weeks, or about every 2 weeks to about every 4 weeks. In embodiments, the chimeric protein exhibits a linear dose response in the dose range of e.g., about 0.3 mg/kg to about 3 mg/kg, or about 0.5 mg/kg to about 3 mg/kg, or about 0.7 mg/kg to about 3 mg/kg, or about 1 mg/kg to about 3 mg/kg, or about 1.5 mg/kg to about 3 mg/kg, or about 2 mg/kg to about 3 mg/kg, or about 2.5 mg/kg to about 3 mg/kg, or about 0.3 mg/kg to about 2.5 mg/kg, or about 0.3 mg/kg to about 2 mg/kg, or about 0.3 mg/kg to about 1.5 mg/kg, or about 0.3 mg/kg to about 1.0 mg/kg, or about 0.3 mg/kg to about 0.5 mg/kg. In embodiments, the chimeric protein does not exhibit a bell-shaped dose response.

In some embodiments of any of the aspects disclosed herein, the first domain is capable of binding a CD172a (SIRPα) ligand. In embodiments, the first domain comprises substantially all of the extracellular domain of CD172a (SIRPα). In embodiments, the second domain is capable of binding a CD40 receptor. In embodiments, the second domain comprises substantially all of the extracellular domain of CD40L. In embodiments, the linker comprises a hinge-CH2-CH3 Fc domain derived from IgG4, e.g., human IgG4. In embodiments, the linker comprises an amino acid sequence that is at least 90%, or 93%, or 95%, or 97%, or 98%, or 99% identical to the amino acid sequence of SEQ ID NO: 1, SEQ ID NO: 2, or SEQ ID NO: 3. In embodiments, the linker comprises an amino acid sequence that is at least 95% identical to the amino acid sequence of SEQ ID NO: 1, SEQ ID NO: 2, or SEQ ID NO: 3. In embodiments, the linker comprises an amino acid sequence that is at least 98% identical to the amino acid sequence of SEQ ID NO: 1, SEQ ID NO: 2, or SEQ ID NO: 3. In embodiments, the first domain comprises an amino acid sequence that is at least 90%, or 93%, or 95%, or 97%, or 98%, or 99% identical to the amino acid sequence of SEQ ID NO: 57. In embodiments, the first domain comprises an amino acid sequence that is at least 95% identical to the amino acid sequence of SEQ ID NO: 57. In embodiments, the first domain comprises an amino acid sequence that is at least 96% identical to the amino acid sequence of SEQ ID NO: 57. In embodiments, the first domain comprises an amino acid sequence that is at least 98% identical to the amino acid sequence of SEQ ID NO: 57. In embodiments, the first domain comprises an amino acid sequence that is at least 99% identical to the amino acid sequence of SEQ ID NO: 57. In embodiments, the first domain comprises an amino acid sequence that is identical to the amino acid sequence of SEQ ID NO: 57.

In some embodiments of any of the aspects disclosed herein, the second domain comprises an amino acid sequence that is at least 90%, or 93%, or 95%, or 97%, or 98%, or 99% identical to the amino acid sequence of SEQ ID NO: 58. In some embodiments of any of the aspects disclosed herein, the second domain comprises an amino acid sequence that is at least 95% identical to the amino acid sequence of SEQ ID NO: 58. In some embodiments of any of the aspects disclosed herein, the second domain comprises an amino acid sequence that is at least 96% identical to the amino acid sequence of SEQ ID NO: 58. In some embodiments of any of the aspects disclosed herein, the second domain comprises an amino acid sequence that is at least 97% identical to the amino acid sequence of SEQ ID NO: 58. In some embodiments of any of the aspects disclosed herein, the second domain comprises an amino acid sequence that is at least 98% identical to the amino acid sequence of SEQ ID NO: 58. In some embodiments of any of the aspects disclosed herein, the second domain comprises an amino acid sequence that is at least 99% identical to the amino acid sequence of SEQ ID NO: 58. In some embodiments of any of the aspects disclosed herein, the second domain comprises an amino acid sequence that is identical to the amino acid sequence of SEQ ID NO: 58. In embodiments, (a) the first domain comprises the amino acid sequence of SEQ ID NO: 57, (b) the second domain comprises the amino acid sequence of SEQ ID NO: 58, and (c) the linker comprises an amino acid sequence that is at least 95% identical to SEQ ID NO: 1, SEQ ID NO: 2, or SEQ ID NO: 3.

In embodiments, the chimeric protein further comprises the amino acid sequence of SEQ ID NO: 5 or SEQ ID NO: 7. In embodiments, the chimeric protein further comprises the amino acid sequence of SEQ ID NO: 5 and SEQ ID NO: 7. In embodiments, the chimeric protein comprises an amino acid sequence that is at least 90%, or 93%, or 95%, or 97%, or 98%, or 99% identical to SEQ ID NO: 59 or SEQ ID NO: 61. In embodiments, the chimeric protein comprises an amino acid sequence that is at least 95% identical to SEQ ID NO: 59 or SEQ ID NO: 61. In embodiments, the chimeric protein comprises an amino acid sequence that is at least 96% identical to SEQ ID NO: 59 or SEQ ID NO: 61. In embodiments, the chimeric protein comprises an amino acid sequence that is at least 97% identical to SEQ ID NO: 59 or SEQ ID NO: 61. In embodiments, the chimeric protein comprises an amino acid sequence that is at least 98% identical to SEQ ID NO: 59 or SEQ ID NO: 61. In embodiments, the chimeric protein comprises an amino acid sequence that is at least 99% identical to SEQ ID NO: 59 or SEQ ID NO: 61. In embodiments, the chimeric protein comprises an amino acid sequence that is identical to SEQ ID NO: 59 or SEQ ID NO: 61. In embodiments, the chimeric protein comprises an amino acid sequence that is at least about 98% identical to SEQ ID NO: 59 or SEQ ID NO: 61. In embodiments, the chimeric protein comprises an amino acid sequence that is at least about 99% identical to SEQ ID NO: 59 or SEQ ID NO: 61.

Additionally or alternatively, in embodiments, the chimeric protein comprises an amino acid sequence that is at least about 99.2% identical to SEQ ID NO: 59 or SEQ ID NO: 61. In embodiments, the chimeric protein comprises an amino acid sequence that is at least about 99.4% identical to SEQ ID NO: 59 or SEQ ID NO: 61. In embodiments, the chimeric protein comprises an amino acid sequence that is at least about 99.6% identical to SEQ ID NO: 59 or SEQ ID NO: 61. In embodiments, the chimeric protein comprises an amino acid sequence that is at least about 99.8% identical to SEQ ID NO: 59 or SEQ ID NO: 61. In embodiments, the chimeric protein comprises the amino acid sequence of SEQ ID NO: 59 or SEQ ID NO: 61. In embodiments, the human subject has received, been tolerant to, or is ineligible for standard therapy and/or the cancer has no approved therapy considered to be standard of care.

In one aspect, the present disclosure relates to a method for promoting the migration of lymphocytes from peripheral blood into secondary lymphoid organs (e.g. the lymph nodes and spleen in a human subject in need thereof, the method comprising a step of administering to the human subject an effective amount of a chimeric protein having a general structure of: N terminus-(a)-(b)-(c)-C terminus, wherein: (a) is a first domain comprising an extracellular domain of human signal regulatory protein α (CD172a (SIRPα)), (b) is a linker adjoining the first and second domains, wherein the linker comprises a hinge-CH2-CH3 Fc domain, and (c) is a second domain comprising an extracellular domain of human CD40 ligand (CD40L).

In some embodiments of any of the aspects disclosed herein, the human subject is not receiving a concurrent chemotherapy, immunotherapy, biologic or hormonal therapy.

In one aspect, the present disclosure relates to a chimeric protein for use in the method of any of the embodiments disclosed herein.

In one aspect, the present disclosure relates to a chimeric protein comprising an amino acid sequence that is at least about 98% identical to SEQ ID NO: 59 or SEQ ID NO: 61. In embodiments, the chimeric protein comprises an amino acid sequence that is at least about 99% identical to SEQ ID NO: 59 or SEQ ID NO: 61.

In embodiments, the chimeric protein comprises an amino acid sequence that is identical to SEQ ID NO: 59 or SEQ ID NO: 61.

The dosing frequency of the first phase, and the dosing frequency of the second phase may be same or different. In embodiments, the dosing frequency of the first phase and the dosing frequency of the second phase are each independently selected from about every three days, about twice a week, about every week, about every 10 days, about twice every 3 weeks, about every 2 weeks, about every 3 weeks, about every 4 weeks, about every month, about every 5 weeks, about every 6 weeks, about 7 seven weeks, about every 8 weeks and about every 2 months. In embodiments, the dosing frequency of the first phase is selected from about every 3 days to about every 10 days, about every week to about every 2 weeks, about every 10 days to about every 3 weeks, about every 2 weeks to about every 4 weeks, about every 3 weeks to about every 5 weeks, about every 4 weeks to about every 6 weeks, about every 5 weeks to about every 7 weeks, about every 6 weeks to about every 8 weeks, and, about every 6 weeks to about every 2 months.

In embodiments, the first phase, and the second phase each independently last from about two days to about 12 months. For example, In embodiments, the first phase lasts from about two weeks to about 2 months; and the second phase lasts from about 2 weeks to about 12 months. In embodiments, the first phase lasts from about two weeks to about 1 month; and the second phase lasts from about 2 weeks to about 12 months. In embodiments, the first phase lasts from about two weeks to about 1 month; and the second phase lasts from about 4 weeks to about 12 months.

The effective amount for the first phase, the second phase and the third phase may be same or different. In embodiments, the effective amount for the first phase, the second phase and the third phase each independently comprise about 0.01 mg/kg to about 10 mg/ml. In embodiments, the effective amount for the first phase is from about 0.03 mg/kg to about 0.3 mg/kg, about 0.1 mg/kg to about 1 mg/kg, about 0.3 mg/kg to about 3 mg/kg, or about 1 mg/kg to about 10 mg/kg; and the effective amount for the second phase is from about 0.01 mg/kg to about 0.1 mg/kg, about 0.03 mg/kg to about 0.3 mg/kg, about 0.1 mg/kg to about 1 mg/kg, about 0.3 mg/kg to about 3 mg/kg, or about 1 mg/kg to about 10 mg/kg. In embodiments, the chimeric proteins disclosed herein is the human CD172a (SIRPα)-Fc-CD40L chimeric protein.

In embodiments, the human CD172a (SIRPα)-Fc-CD40L chimeric protein is capable of providing a sustained immunomodulatory effect.

In embodiments, the linker comprises an amino acid sequence that is at least 90%, or 93%, or 95%, or 97%, or 98%, or 99% identical to the amino acid sequence of SEQ ID NO: 1, SEQ ID NO: 2, or SEQ ID NO: 3. In embodiments, the linker comprises an amino acid sequence that is at least 95% identical to the amino acid sequence of SEQ ID NO: 1, SEQ ID NO: 2, or SEQ ID NO: 3. In embodiments, the linker comprises an amino acid sequence that is at least 96% identical to the amino acid sequence of SEQ ID NO: 1, SEQ ID NO: 2, or SEQ ID NO: 3. In embodiments, the linker comprises an amino acid sequence that is at least 98% identical to the amino acid sequence of SEQ ID NO: 1, SEQ ID NO: 2, or SEQ ID NO: 3. In embodiments, the linker comprises an amino acid sequence that is at least 99% identical to the amino acid sequence of SEQ ID NO: 1, SEQ ID NO: 2, or SEQ ID NO: 3. In embodiments, the linker comprises an amino acid sequence that is identical to the amino acid sequence of SEQ ID NO: 1, SEQ ID NO: 2, or SEQ ID NO: 3.

In embodiments, the linker comprises hinge-CH2-CH3 Fc domain derived from IgG. In embodiments, the linker comprises hinge-CH2-CH3 Fc domain derived from an IgG selected from IgG1 and IgG4. In embodiments, the linker comprises hinge-CH2-CH3 Fc domain derived from human IgG1 or human IgG4. In embodiments, the linker comprises hinge-CH2-CH3 Fc domain derived from IgG4. In embodiments, the hinge-CH2-CH3 Fc domain is derived from human IgG4.

Additionally, or alternatively, in embodiments, the extracellular domain of human signal regulatory protein α (CD172a (SIRPα)) comprises an amino acid sequence that is at least 90%, or 93%, or 95%, or 97%, or 98%, or 99% identical to the amino acid sequence of SEQ ID NO: 57. In embodiments, the extracellular domain of human signal regulatory protein α (CD172a (SIRPα)) comprises an amino acid sequence that is at least 95% identical to the amino acid sequence of SEQ ID NO: 57. In embodiments, the extracellular domain of human signal regulatory protein α (CD172a (SIRPα)) comprises an amino acid sequence that is at least 96% identical to the amino acid sequence of SEQ ID NO: 57. In embodiments, the extracellular domain of human signal regulatory protein α (CD172a (SIRPα)) comprises an amino acid sequence that is at least 98% identical to the amino acid sequence of SEQ ID NO: 57. In embodiments, the extracellular domain of human signal regulatory protein α (CD172a (SIRPα)) comprises an amino acid sequence that is at least 99% identical to the amino acid sequence of SEQ ID NO: 57. In embodiments, the extracellular domain of human signal regulatory protein α (CD172a (SIRPα)) comprises an amino acid sequence that is identical to the amino acid sequence of SEQ ID NO: 57.

Additionally, or alternatively, in embodiments, the extracellular domain of human CD40 ligand (CD40L) comprises an amino acid sequence that is at least 90%, or 93%, or 95%, or 97%, or 98%, or 99% identical to the amino acid sequence of SEQ ID NO: 58. In embodiments, the extracellular domain of human CD40 ligand (CD40L) comprises an amino acid sequence that is at least 95% identical to the amino acid sequence of SEQ ID NO: 58. In embodiments, the extracellular domain of human CD40 ligand (CD40L) comprises an amino acid sequence that is at least 96% identical to the amino acid sequence of SEQ ID NO: 58. In embodiments, the extracellular domain of human CD40 ligand (CD40L) comprises an amino acid sequence that is at least 98% identical to the amino acid sequence of SEQ ID NO: 58. In embodiments, the extracellular domain of human CD40 ligand (CD40L) comprises an amino acid sequence that is at least 99% identical to the amino acid sequence of SEQ ID NO: 58. In embodiments, the extracellular domain of human CD40 ligand (CD40L) comprises an amino acid sequence that is identical to the amino acid sequence of SEQ ID NO: 58.

Additionally, or alternatively, in embodiments, the human CD172a (SIRPα)-Fc-CD40L chimeric protein comprises an amino acid sequence that is at least 90%, or 93%, or 95%, or 97%, or 98%, or 99% identical to SEQ ID NO: 59 or SEQ ID NO: 61. In embodiments, the human CD172a (SIRPα)-Fc-CD40L chimeric protein comprises an amino acid sequence that is at least 95% identical to SEQ ID NO: 59 or SEQ ID NO: 61. In embodiments, the human CD172a (SIRPα)-Fc-CD40L chimeric protein comprises an amino acid sequence that is at least 96% identical to SEQ ID NO: 59 or SEQ ID NO: 61. In embodiments, the human CD172a (SIRPα)-Fc-CD40L chimeric protein comprises an amino acid sequence that is at least 97% identical to SEQ ID NO: 59 or SEQ ID NO: 61. In embodiments, the human CD172a (SIRPα)-Fc-CD40L chimeric protein comprises an amino acid sequence that is at least 98% identical to SEQ ID NO: 59 or SEQ ID NO: 61. In embodiments, the human CD172a (SIRPα)-Fc-CD40L chimeric protein comprises an amino acid sequence that is at least 99% identical to SEQ ID NO: 59 or SEQ ID NO: 61. In embodiments, the human CD172a (SIRPα)-Fc-CD40L chimeric protein comprises an amino acid sequence that is identical to SEQ ID NO: 59 or SEQ ID NO: 61.

In one aspect, the present disclosure relates to a method for treating a cancer in a human subject comprising: (i) administering to the human subject a chimeric protein having a general structure of: N terminus-(a)-(b)-(c)-C terminus, wherein: (a) is a first domain comprising an extracellular domain of human signal regulatory protein α (CD172a (SIRPα)), (b) is a linker adjoining the first and second domains, wherein the linker comprises at least one cysteine residue capable of forming a disulfide bond and/or comprises a hinge-CH2-CH3 Fc domain, and (c) is a second domain comprising an extracellular domain of human CD40 ligand (CD40L); and (ii) administering a second therapeutic agent. In embodiments, the chimeric protein is administered at a dose between about 0.0001 mg/kg and about 10 mg/kg. In embodiments, the dose of the chimeric protein administered is at least about 0.3 mg/kg, e.g., at least about 0.3 mg/kg, or about 1.0 mg/kg, or about 2 mg/kg, or about 3, about 4 mg/kg, or about 6 mg/kg, or about 8 mg/kg, or about 10 mg/kg. In embodiments, the dose of the chimeric protein administered is at least about 1 mg/kg, e.g., at least about 1.0 mg/kg, or about 2 mg/kg, or about 3, about 4 mg/kg, or about 6 mg/kg, or about 8 mg/kg, or about 10 mg/kg. In embodiments, the chimeric protein exhibits a linear dose response in the dose range of e.g., about 0.3 mg/kg to about 3 mg/kg, or about 0.5 mg/kg to about 3 mg/kg, or about 0.7 mg/kg to about 3 mg/kg, or about 1 mg/kg to about 3 mg/kg, or about 1.5 mg/kg to about 3 mg/kg, or about 2 mg/kg to about 3 mg/kg, or about 2.5 mg/kg to about 3 mg/kg, or about 0.3 mg/kg to about 2.5 mg/kg, or about 0.3 mg/kg to about 2 mg/kg, or about 0.3 mg/kg to about 1.5 mg/kg, or about 0.3 mg/kg to about 1.0 mg/kg, or about 0.3 mg/kg to about 0.5 mg/kg. In embodiments, the chimeric protein does not exhibit a bell-shaped dose response.

In embodiments, the administration of the chimeric protein causes a CD47 receptor occupancy (RO) on leukocytes that is at least about 30%, or at least about 40%, or at least about 50%, or at least about 60%, or at least about 65%, or at least about 70%, or at least about 75%, or at least about 80%, or at least about 85%, or at least about 90%, or at least about 95% compared to the RO prior to administration of the chimeric protein, a second subject that is not administered the chimeric protein and/or an external control. In embodiments, the administration of the chimeric protein causes a CD47 receptor occupancy (RO) on B cells that is at least about 30%, or at least about 40%, or at least about 50%, or at least about 60%, or at least about 65%, or at least about 70%, or at least about 75%, or at least about 80%, or at least about 85%, or at least about 90%, or at least about 95% compared to the RO prior to administration of the chimeric protein, a second subject that is not administered the chimeric protein and/or an external control. In embodiments, the administration of the chimeric protein causes an increase in the amount or activity of one or more of IL-12, MCP-1, MIP-1β, MIP-1α, and MDC % compared to the RO prior to administration of the chimeric protein, a second subject that is not administered the chimeric protein and/or an external control.

In one aspect, the present disclosure relates to a method for treating a cancer in a human subject comprising administering to a subject in need thereof: a chimeric protein of a general structure of N terminus-(a)-(b)-(c)-C terminus, wherein: (a) is a first domain comprising an extracellular domain of human signal regulatory protein α (CD172a (SIRPα)), (b) is a linker adjoining the first and second domains, wherein the linker comprises at least one cysteine residue capable of forming a disulfide bond and/or comprises a hinge-CH2-CH3 Fc domain, and (c) is a second domain comprising an extracellular domain of human CD40 ligand (CD40L); wherein: the subject is undergoing or has undergone treatment with a second therapeutic agent. In embodiments, the chimeric protein is administered at a dose between about 0.0001 mg/kg and about 10 mg/kg. In embodiments, the dose of the chimeric protein administered is at least about 0.3 mg/kg, e.g., at least about 0.3 mg/kg, or about 1.0 mg/kg, or about 2 mg/kg, or about 3, about 4 mg/kg, or about 6 mg/kg, or about 8 mg/kg, or about 10 mg/kg. In embodiments, the dose of the chimeric protein administered is at least about 1 mg/kg, e.g., at least about 1.0 mg/kg, or about 2 mg/kg, or about 3, about 4 mg/kg, or about 6 mg/kg, or about 8 mg/kg, or about 10 mg/kg. In embodiments, the chimeric protein exhibits a linear dose response in the dose range of e.g., about 0.3 mg/kg to about 3 mg/kg, or about 0.5 mg/kg to about 3 mg/kg, or about 0.7 mg/kg to about 3 mg/kg, or about 1 mg/kg to about 3 mg/kg, or about 1.5 mg/kg to about 3 mg/kg, or about 2 mg/kg to about 3 mg/kg, or about 2.5 mg/kg to about 3 mg/kg, or about 0.3 mg/kg to about 2.5 mg/kg, or about 0.3 mg/kg to about 2 mg/kg, or about 0.3 mg/kg to about 1.5 mg/kg, or about 0.3 mg/kg to about 1.0 mg/kg, or about 0.3 mg/kg to about 0.5 mg/kg. In embodiments, the chimeric protein does not exhibit a bell-shaped dose response.

In one aspect, the present disclosure relates to a method for treating a cancer in a human subject comprising administering to a subject in need thereof a second anticancer therapeutic agent, wherein the subject is undergoing or has undergone treatment with a chimeric protein of a general structure of N terminus-(a)-(b)-(c)-C terminus, wherein: (a) is a first domain comprising an extracellular domain of human Signal regulatory protein α (CD172a (SIRPα)), (b) is a linker adjoining the first and second domains, wherein the linker comprises at least one cysteine residue capable of forming a disulfide bond and/or comprises a hinge-CH2-CH3 Fc domain, and (c) is a second domain comprising an extracellular domain of human CD40 ligand (CD40L). In embodiments, the chimeric protein is administered at a dose between about 0.0001 mg/kg and about 10 mg/kg. In embodiments, the chimeric protein exhibits a linear dose response in the dose range of e.g., about 0.3 mg/kg to about 3 mg/kg, or about 0.5 mg/kg to about 3 mg/kg, or about 0.7 mg/kg to about 3 mg/kg, or about 1 mg/kg to about 3 mg/kg, or about 1.5 mg/kg to about 3 mg/kg, or about 2 mg/kg to about 3 mg/kg, or about 2.5 mg/kg to about 3 mg/kg, or about 0.3 mg/kg to about 2.5 mg/kg, or about 0.3 mg/kg to about 2 mg/kg, or about 0.3 mg/kg to about 1.5 mg/kg, or about 0.3 mg/kg to about 1.0 mg/kg, or about 0.3 mg/kg to about 0.5 mg/kg. In embodiments, the chimeric protein does not exhibit a bell-shaped dose response.

In embodiments, the chimeric protein is administered before the second therapeutic agent. In embodiments, the second therapeutic agent is administered before the chimeric protein. In embodiments, the second therapeutic agent and the chimeric protein are administered substantially together.

In embodiments, the second therapeutic agent is selected from an antibody, and a chemotherapeutic agent. In embodiments, the antibody is capable of antibody-dependent cellular cytotoxicity (ADCC). In embodiments, the antibody is selected from cetuximab, rituximab, obinutuzumab, Hul4.18K322A, Hu3F8, dinituximab, and trastuzumab. In embodiments, the antibody is capable of antibody-dependent cellular phagocytosis (ADCP). In embodiments, the antibody is selected from cetuximab, daratumumab, rituximab, and trastuzumab. In embodiments, the antibody is capable of binding a molecule selected from carcinoembryonic antigen (CEA), EGFR, HER-2, epithelial cell adhesion molecule (EpCAM), and human epithelial mucin-1, CD20, CD30, CD38, CD40, and CD52. In embodiments, the antibody is capable of binding EGFR. In embodiments, the antibody is selected from Mab A13, AMG595, cetuximab (Erbitux, C225), panitumumab (ABX-EGF, Vectibix), depatuxizumab (ABT 806), depatuxizumab, mafodotin, duligotuzumab (MEHD7945A, RG7597), Futuximab (Sym004), GC1118, imgatuzumab (GA201), matuzumab (EMD 72000), necitumumab (Portrazza), nimotuzumab (h-R3), anitumumab (Vectibix, ABX-EGF), zalutumumab, humMR1, and tomuzotuximab. In embodiments, the antibody is cetuximab.

In embodiments, the chemotherapeutic agent is an anthracycline. In embodiments, the anthacycline is selected from doxorubicin, daunorubicin, epirubicin and idarubicin, and pharmaceutically acceptable salts, acids or derivatives thereof. In embodiments, the chemotherapeutic agent is doxorubicin.

In embodiments, the dose of the chimeric protein administered is at least about 0.0001 mg/kg, e.g., between about 0.0001 mg/kg and about 10.0 mg/kg. The chimeric protein may be administered at an initial dose (e.g., at one of about 0.0001, about 0.001, about 0.003, about 0.01, about 0.03, about 0.1, about 0.3, about 1, about 2, about 3, about 4, about 6 or about 10.0 mg/kg) and the chimeric protein is administered in one or more subsequent administrations (e.g., at one or more of about 0.0001, about 0.001, about 0.003, about 0.01, about 0.03, about 0.1, about 0.3, about 1, about 2, about 3, about 4, about 6, about 8, and about 10 mg/kg). In embodiments, the dose of the chimeric protein administered is at least about 0.3 mg/kg, e.g., at least about 0.3 mg/kg, or about 1.0 mg/kg, or about 2 mg/kg, or about 3, about 4 mg/kg, or about 6 mg/kg, or about 8 mg/kg, or about 10 mg/kg. In embodiments, the dose of the chimeric protein administered is at least about 1 mg/kg, e.g., at least about 1.0 mg/kg, or about 2 mg/kg, or about 3, about 4 mg/kg, or about 6 mg/kg, or about 8 mg/kg, or about 10 mg/kg. In embodiments, the initial dose is less than the dose for at least one of the subsequent administrations (e.g. each of the subsequent administrations) or the initial dose is the same as the dose for at least one of the subsequent administrations (e.g., each of the subsequent administrations). In embodiments, the starting dose and/or the subsequent doses is the maximum tolerated dose or less than the maximum tolerated dose. In embodiments, the chimeric protein is administered at least about one time a month, e.g., at least about two times a month, at least about three times a month, and at least about four times a month. In embodiments, the chimeric protein is first administered once a week for three weeks and the chimeric protein is then administered about once every three weeks or once every fourweeks; alternately, the chimeric protein is first administered once a week for three weeks and the chimeric protein is then administered about two times per month, e.g., once a week for three weeks and the chimeric protein is then administered about once every two weeks.

In embodiments, the cancer comprises an advanced solid tumor (local and/or metastatic) or a lymphoma. In embodiments, the cancer is selected from ovarian cancer, fallopian tube cancer, peritoneal cancer, cutaneous squamous cell carcinoma (CSCC), and squamous cell carcinoma of the head and neck (SCCHN). In embodiments, the cancer comprises an advanced solid tumor (local and/or metastatic) or advanced lymphoma.

### Methods of Selecting a Subject for Treatment and Evaluating the Efficacy of Cancer Treatment

In one aspect, the present disclosure relates to a method of evaluating the efficacy of a cancer treatment in a subject in need thereof comprising, the method comprising the steps of: obtaining a biological sample from the subject that has received a dose of a chimeric protein, wherein the dose of from about 0.03 mg/kg to 10 mg/kg, wherein the chimeric protein has a general structure of: N terminus-(a)-(b)-(c)-C terminus, wherein: (a) is a first domain comprising an extracellular domain of human signal regulatory protein α (CD172a (SIRPα)), (b) is a linker adjoining the first and second domains, wherein the linker comprises a hinge-CH2-CH3 Fc domain, and (c) is a second domain comprising an extracellular domain of human CD40 ligand (CD40L); performing an assay on the biological sample to determine level and/or activity of a cytokine selected from CCL2, CXCL9, CXCL10, IFNα, IL15, IL23, IL-12, MCP-1, MIP-13, MIP-1α, and MDC; and administering the chimeric protein to the subject if the subject has an increase in the level and/or activity of at least one cytokine selected from CCL2, CXCL9, CXCL10, IFNα, IL15, IL23, IL-12, MCP-1, MIP-1β, MIP-1α, and MDC.

In one aspect, the present disclosure relates to a method of evaluating the efficacy of a cancer treatment in a subject in need thereof comprising, the method comprising the steps of: obtaining a biological sample from the subject that has received a dose of a chimeric protein, wherein the dose of from about 0.03 mg/kg to 10 mg/kg, wherein the chimeric protein has a general structure of: N terminus-(a)-(b)-(c)-C terminus, wherein: (a) is a first domain comprising an extracellular domain of human signal regulatory protein α (CD172a (SIRPα)), (b) is a linker adjoining the first and second domains, wherein the linker comprises a hinge-CH2-CH3 Fc domain, and (c) is a second domain comprising an extracellular domain of human CD40 ligand (CD40L); performing an assay on the biological sample to determine level and/or activity of a cytokine selected from CCL2, CXCL9, CXCL10, IFNα, IL6, IL15, IL23, and TNFα; and administering the chimeric protein to the subject if the subject has an increase in the level and/or activity of at least one cytokine selected from CCL2, CXCL9, CXCL10, IFNα, IL6, IL15, and IL23, and/or if the subject has a lack of substantial increase in the level and/or activity of IL6 and/or TNFα.

In one aspect, the present disclosure relates to a method of selecting a subject for treatment with a therapy for a cancer, the method comprising the steps of: (i) administering a dose of a chimeric protein, wherein the dose of from about 0.03 mg/kg to 10 mg/kg; wherein the chimeric protein has a general structure of: N terminus-(a)-(b)-(c)-C terminus, wherein: (a) is a first domain comprising an extracellular domain of human signal regulatory protein α (CD172a (SIRPα)), (b) is a linker adjoining the first and second domains, wherein the linker comprises a hinge-CH2-CH3 Fc domain, and (c) is a second domain comprising an extracellular domain of human CD40 ligand (CD40L); (ii) obtaining a biological sample from the subject; (iii) performing an assay on the biological sample to determine level and/or activity of a cytokine selected from CCL2, CXCL9, CXCL10, IFNα, IL15, IL23, IL-12, MCP-1, MIP-11, MIP-1α, and MDC; and (iv) selecting the subject for treatment with the chimeric protein if the subject has an increase in the level and/or activity of at least one cytokine selected from CCL2, CXCL9, CXCL10, IFNα, IL15, IL23, IL-12, MCP-1, MIP-1β, MIP-1α, and MDC.

In one aspect, the present disclosure relates to a method of selecting a subject for treatment with a therapy for a cancer, the method comprising the steps of: (i) administering a dose of a chimeric protein, wherein the dose of from about 0.03 mg/kg to 10 mg/kg; wherein the chimeric protein has a general structure of: N terminus-(a)-(b)-(c)-C terminus, wherein: (a) is a first domain comprising an extracellular domain of human signal regulatory protein α (CD172a (SIRPα)), (b) is a linker adjoining the first and second domains, wherein the linker comprises a hinge-CH2-CH3 Fc domain, and (c) is a second domain comprising an extracellular domain of human CD40 ligand (CD40L); (ii) obtaining a biological sample from the subject; (iii) performing an assay on the biological sample to determine level and/or activity of a cytokine selected from CCL2, CXCL9, CXCL10, IFNα, IL6, IL15, IL23 and TNFα; and (iv) selecting the subject for treatment with the chimeric protein if the subject has an increase in the level and/or activity of at least one cytokine selected from CCL2, CXCL9, CXCL10, IFNα, IL15, IL23, IL-12, MCP-1, MIP-1β, MIP-1α, and MDC, and/or if the subject has a lack of substantial increase in the level and/or activity of IL6 and/or TNFα.

In one aspect, the present disclosure relates to a method of selecting a subject for treatment with a therapy for cancer, the method comprising the steps of: obtaining a biological sample from the subject that has received a dose of a chimeric protein, wherein the dose of from about 0.03 mg/kg to 10 mg/kg, wherein the chimeric protein has a general structure of: N terminus-(a)-(b)-(c)-C terminus, wherein: (a) is a first domain comprising an extracellular domain of human signal regulatory protein α (CD172a (SIRPα)), (b) is a linker adjoining the first and second domains, wherein the linker comprises a hinge-CH2-CH3 Fc domain, and (c) is a second domain comprising an extracellular domain of human CD40 ligand (CD40L); performing an assay on the biological sample to determine level and/or activity of a cytokine selected from CCL2, CXCL9, CXCL10, IFNα, IL15, IL23, IL-12, MCP-1, MIP-1β, MIP-1α, and MDC; and selecting the subject for treatment with the chimeric protein if the subject has an increase in the level and/or activity of at least one cytokine selected from CCL2, CXCL9, CXCL10, IFNα, IL15, IL23, IL-12, MCP-1, MIP-11, MIP-1α, and MDC.

In one aspect, the present disclosure relates to a method of selecting a subject for treatment with a therapy for cancer, the method comprising the steps of: obtaining a biological sample from the subject that has received a dose of a chimeric protein, wherein the dose of from about 0.03 mg/kg to 10 mg/kg, wherein the chimeric protein has a general structure of: N terminus-(a)-(b)-(c)-C terminus, wherein: (a) is a first domain comprising an extracellular domain of human signal regulatory protein α (CD172a (SIRPα)), (b) is a linker adjoining the first and second domains, wherein the linker comprises a hinge-CH2-CH3 Fc domain, and (c) is a second domain comprising an extracellular domain of human CD40 ligand (CD40L); performing an assay on the biological sample to determine level and/or activity of a cytokine selected from CCL2, CXCL9, CXCL10, IFNα, IL6, IL15, IL23, and TNFα; and selecting the subject for treatment with the chimeric protein if the subject has an increase in the level and/or activity of at least one cytokine selected from CCL2, CXCL9, CXCL10, IFNα, IL6, IL15, and IL23; and/or if the subject has a lack of substantial increase in the level and/or activity of IL6 and/or TNFα.

In one aspect, the present disclosure relates to a method for treating a cancer in a human subject comprising: (i) administering to the human subject a chimeric protein having a general structure of: N terminus-(a)-(b)-(c)-C terminus, wherein: (a) is a first domain comprising an extracellular domain of human signal regulatory protein α (CD172a (SIRPα)), (b) is a linker adjoining the first and second domains, wherein the linker comprises at least one cysteine residue capable of forming a disulfide bond and/or comprises a hinge-CH2-CH3 Fc domain, and (c) is a second domain comprising an extracellular domain of human CD40 ligand (CD40L); and (ii) administering a second therapeutic agent. In embodiments, the chimeric protein is administered at a dose between about 0.0001 mg/kg and about 10 mg/kg. In embodiments, the chimeric protein exhibits a linear dose response in the dose range of e.g., about 0.3 mg/kg to about 3 mg/kg, or about 0.5 mg/kg to about 3 mg/kg, or about 0.7 mg/kg to about 3 mg/kg, or about 1 mg/kg to about 3 mg/kg, or about 1.5 mg/kg to about 3 mg/kg, or about 2 mg/kg to about 3 mg/kg, or about 2.5 mg/kg to about 3 mg/kg, or about 0.3 mg/kg to about 2.5 mg/kg, or about 0.3 mg/kg to about 2 mg/kg, or about 0.3 mg/kg to about 1.5 mg/kg, or about 0.3 mg/kg to about 1.0 mg/kg, or about 0.3 mg/kg to about 0.5 mg/kg. In embodiments, the chimeric protein does not exhibit a bell-shaped dose response.

In one aspect, the present disclosure relates to a method for treating a cancer in a human subject comprising administering to a subject in need thereof: a chimeric protein of a general structure of N terminus-(a)-(b)-(c)-C terminus, wherein: (a) is a first domain comprising an extracellular domain of human signal regulatory protein α (CD172a (SIRPα)), (b) is a linker adjoining the first and second domains, wherein the linker comprises at least one cysteine residue capable of forming a disulfide bond and/or comprises a hinge-CH2-CH3 Fc domain, and (c) is a second domain comprising an extracellular domain of human CD40 ligand (CD40L); wherein: the subject is undergoing or has undergone treatment with a second therapeutic agent. In embodiments, the chimeric protein is administered at a dose between about 0.0001 mg/kg and about 10 mg/kg. In embodiments, the chimeric protein exhibits a linear dose response in the dose range of e.g., about 0.3 mg/kg to about 3 mg/kg, or about 0.5 mg/kg to about 3 mg/kg, or about 0.7 mg/kg to about 3 mg/kg, or about 1 mg/kg to about 3 mg/kg, or about 1.5 mg/kg to about 3 mg/kg, or about 2 mg/kg to about 3 mg/kg, or about 2.5 mg/kg to about 3 mg/kg, or about 0.3 mg/kg to about 2.5 mg/kg, or about 0.3 mg/kg to about 2 mg/kg, or about 0.3 mg/kg to about 1.5 mg/kg, or about 0.3 mg/kg to about 1.0 mg/kg, or about 0.3 mg/kg to about 0.5 mg/kg. In embodiments, the chimeric protein does not exhibit a bell-shaped dose response.

In one aspect, the present disclosure relates to a method for treating a cancer in a human subject comprising administering to a subject in need thereof a second anticancer therapeutic agent, wherein the subject is undergoing or has undergone treatment with a chimeric protein of a general structure of N terminus-(a)-(b)-(c)-C terminus, wherein: (a) is a first domain comprising an extracellular domain of human Signal regulatory protein α (CD172a (SIRPα)), (b) is a linker adjoining the first and second domains, wherein the linker comprises at least one cysteine residue capable of forming a disulfide bond and/or comprises a hinge-CH2-CH3 Fc domain, and (c) is a second domain comprising an extracellular domain of human CD40 ligand (CD40L). In embodiments, the chimeric protein is administered at a dose between about 0.0001 mg/kg and about 10 mg/kg. In embodiments, the chimeric protein exhibits a linear dose response in the dose range of e.g., about 0.3 mg/kg to about 3 mg/kg, or about 0.5 mg/kg to about 3 mg/kg, or about 0.7 mg/kg to about 3 mg/kg, or about 1 mg/kg to about 3 mg/kg, or about 1.5 mg/kg to about 3 mg/kg, or about 2 mg/kg to about 3 mg/kg, or about 2.5 mg/kg to about 3 mg/kg, or about 0.3 mg/kg to about 2.5 mg/kg, or about 0.3 mg/kg to about 2 mg/kg, or about 0.3 mg/kg to about 1.5 mg/kg, or about 0.3 mg/kg to about 1.0 mg/kg, or about 0.3 mg/kg to about 0.5 mg/kg. In embodiments, the chimeric protein does not exhibit a bell-shaped dose response.

In embodiments, the chimeric protein is administered before the second therapeutic agent. In embodiments, the second therapeutic agent is administered before the chimeric protein. In embodiments, the second therapeutic agent and the chimeric protein are administered substantially together.

In embodiments, the second therapeutic agent is selected from an antibody, and a chemotherapeutic agent. In embodiments, the antibody is capable of antibody-dependent cellular cytotoxicity (ADCC). In embodiments, the antibody is selected from cetuximab, rituximab, obinutuzumab, Hul4.18K322A, Hu3F8, dinituximab, and trastuzumab. In embodiments, the antibody is capable of antibody-dependent cellular phagocytosis (ADCP). In embodiments, the antibody is selected from cetuximab, daratumumab, rituximab, and trastuzumab. In embodiments, the antibody is capable of binding a molecule selected from carcinoembryonic antigen (CEA), EGFR, HER-2, epithelial cell adhesion molecule (EpCAM), and human epithelial mucin-1, CD20, CD30, CD38, CD40, and CD52. In embodiments, the antibody is capable of binding EGFR. In embodiments, the antibody is selected from Mab A13, AMG595, cetuximab (Erbitux, C225), panitumumab (ABX-EGF, Vectibix), depatuxizumab (ABT 806), depatuxizumab, mafodotin, duligotuzumab (MEHD7945A, RG7597), Futuximab (Sym004), GC1118, imgatuzumab (GA201), matuzumab (EMD 72000), necitumumab (Portrazza), nimotuzumab (h-R3), anitumumab (Vectibix, ABX-EGF), zalutumumab, humMR1, and tomuzotuximab. In embodiments, the antibody is cetuximab.

In embodiments, the chemotherapeutic agent is an anthracycline. In embodiments, the anthacycline is selected from doxorubicin, daunorubicin, epirubicin and idarubicin, and pharmaceutically acceptable salts, acids or derivatives thereof. In embodiments, the chemotherapeutic agent is doxorubicin.

In embodiments, the dose of the chimeric protein administered is at least about 0.0001 mg/kg, e.g., between about 0.0001 mg/kg and about 10.0 mg/kg. The chimeric protein may be administered at an initial dose (e.g., at one of about 0.0001, about 0.001, about 0.003, about 0.01, about 0.03, about 0.1, about 0.3, about 1, about 2, about 3, about 4, about 6 or about 10.0 mg/kg) and the chimeric protein is administered in one or more subsequent administrations (e.g., at one or more of about 0.0001, about 0.001, about 0.003, about 0.01, about 0.03, about 0.1, about 0.3, about 1, about 2, about 3, about 4, about 6, about 8, and about 10 mg/kg). In embodiments, the initial dose is less than the dose for at least one of the subsequent administrations (e.g. each of the subsequent administrations) or the initial dose is the same as the dose for at least one of the subsequent administrations (e.g., each of the subsequent administrations). In embodiments, the starting dose and/or the subsequent doses is the maximum tolerated dose or less than the maximum tolerated dose. In embodiments, the chimeric protein is administered at least about one time a month, e.g., at least about two times a month, at least about three times a month, and at least about four times a month. In embodiments, the chimeric protein is first administered once a week for three weeks and the chimeric protein is then administered about once every three weeks or once every four weeks; alternately, the chimeric protein is first administered once a week for three weeks and the chimeric protein is then administered about two times per month, e.g., once a week for three weeks and the chimeric protein is then administered about once every two weeks.

In embodiments, the cancer is selected from ovarian cancer, fallopian tube cancer, peritoneal cancer, cutaneous squamous cell carcinoma (CSCC), and squamous cell carcinoma of the head and neck (SCCHN).

In embodiments, the cancer comprises an advanced solid tumor (local and/or metastatic) or advanced lymphoma.

In one aspect, the present disclosure relates to a method for evaluating the efficacy of a cancer treatment in a subject in need thereof, wherein the subject is suffering from cancer, the method comprising the steps of: (i) administering a dose of a chimeric protein, wherein the dose of from about 0.03 mg/kg to 10 mg/kg; wherein the chimeric protein has a general structure of: N terminus-(a)-(b)-(c)-C terminus, wherein: (a) is a first domain comprising an extracellular domain of human signal regulatory protein α (CD172a (SIRPα)), (b) is a linker adjoining the first and second domains, wherein the linker comprises a hinge-CH2-CH3 Fc domain, and (c) is a second domain comprising an extracellular domain of human CD40 ligand (CD40L); (ii) obtaining a biological sample from the subject; (iii) performing an assay on the biological sample to determine a level and/or activity of a cytokine selected from CCL2, CXCL9, CXCL10, IFNα, IL15, IL23, IL-12, MCP-1, MIP-1β, MIP-1α, and MDC; and (iv) continuing administration of the chimeric protein if the subject has an increase in the level and/or activity of at least one cytokine selected from CCL2, CXCL9, CXCL10, IFNα, IL15, IL23, IL-12, MCP-1, MIP-13, MIP-1α, and MDC.

In one aspect, the present disclosure relates to a method for evaluating the efficacy of a cancer treatment in a subject in need thereof, wherein the subject is suffering from cancer, the method comprising the steps of: (i) administering a dose of a chimeric protein, wherein the dose of from about 0.03 mg/kg to 10 mg/kg; wherein the chimeric protein has a general structure of: N terminus-(a)-(b)-(c)-C terminus, wherein: (a) is a first domain comprising an extracellular domain of human signal regulatory protein α (CD172a (SIRPα)), (b) is a linker adjoining the first and second domains, wherein the linker comprises a hinge-CH2-CH3 Fc domain, and (c) is a second domain comprising an extracellular domain of human CD40 ligand (CD40L); (ii) obtaining a biological sample from the subject; (iii) performing an assay on the biological sample to determine a level and/or activity of a cytokine selected from CCL2, CXCL9, CXCL10, IFNα, IL6, IL15, IL23, and TNFα; and (iv) continuing administration of the chimeric protein if the subject has an increase in the level and/or activity of at least one cytokine selected from CCL2, CXCL9, CXCL10, IFNα, IL15, IL23, IL-12, MCP-1, MIP-1β, MIP-1α, and MDC, and/or if the subject has a lack of substantial increase in the level and/or activity of IL6 and/or TNFα.

In embodiments, the increase is calculated in comparison to the level and/or activity of the cytokine in another biological sample in the subject prior to administering the dose of the chimeric protein to the subject. In embodiments, the increase is calculated in comparison to a level and/or activity of the cytokine in another biological sample from a different subject that has not been administered the dose of the chimeric protein. In embodiments, the increase is calculated in comparison to a level and/or activity of the cytokine in a negative control. In embodiments, the negative control is devoid of the cytokine. In embodiments, the negative control contains the levels of the cytokine found in individuals that are not undergoing an inflammatory response. In embodiments, the increase occurs by a factor of at least about 0.1×, about 0.2×, about 0.3×, about 0.4×, about 0.5×, about 0.6×, about 0.7×, about 0.8×, about 0.9×, about 1×, about 1.1×, about 1.2×, about 1.3×, about 1.4×, about 1.5×, about 1.6×, about 1.7×, about 1.8×, about 1.9×, about 2×, about 2.1×, about 2.2×, about 2.3×, about 2.4×, about 2.5×, about 2.6×, about 2.7×, about 2.8×, about 2.9×, about 3×, about 3.1×, about 3.2×, about 3.3×, about 3.4×, about 3.5×, about 3.6×, about 3.7×, about 3.8×, about 3.9×, about 4×, about 4.1×, about 4.2×, about 4.3×, about 4.4×, about 4.5×, about 4.6×, about 4.7×, about 4.8×, about 4.9×, about 5×, about 5.1×, about 5.2×, about 5.3×, about 5.4×, about 5.5×, about 5.6×, about 5.7×, about 5.8×, about 5.9×, about 6×, about 6.1×, about 6.2×, about 6.3×, about 6.4×, about 6.5×, about 6.6×, about 6.7×, about 6.8×, about 6.9×, about 7×, about 7.1×, about 7.2×, about 7.3×, about 7.4×, about 7.5×, about 7.6×, about 7.7×, about 7.8×, about 7.9×, about 8×, about 8.1×, about 8.2×, about 8.3×, about 8.4×, about 8.5×, about 8.6×, about 8.7×, about 8.8×, about 8.9×, about 9×, about 9.1×, about 9.2×, about 9.3×, about 9.4×, about 9.5×, about 9.6×, about 9.7×, about 9.8×, about 9.9×, or about 10× compared to the negative control.

In embodiments, the increase is calculated in comparison to a level and/or activity of the cytokine in a positive control. In embodiments, the positive control comprises the cytokine. In embodiments, the positive control comprises the levels of the cytokine found in individuals that are undergoing an inflammatory response.

Additionally, or alternatively, in embodiments, the subject has a decrease in the level and/or activity of at least one cytokine selected from CCL2, CXCL9, CXCL10, IFNα, IL15, IL23, IL-12, MCP-1, MIP-13, MIP-1α, and MDC. In embodiments, the subject has a lack of substantial increase in the level and/or activity of IL6 and/or TNFα. In embodiments, the decrease is calculated in comparison to the level and/or activity of the cytokine in another biological sample in the subject prior to administering the dose of the chimeric protein to the subject. In embodiments, the decrease is calculated in comparison to a level and/or activity of the cytokine in another biological sample from a different subject that has not been administered the dose of the chimeric protein. In embodiments, the decrease is calculated in comparison to a level and/or activity of the cytokine in a negative control. In embodiments, the negative control is devoid of the cytokine. In embodiments, the negative control contains the levels of the cytokine found in individuals that are not undergoing an inflammatory response. In embodiments, the decrease occurs by a factor of at least about 0.1×, about 0.2×, about 0.3×, about 0.4×, about 0.5×, about 0.6×, about 0.7×, about 0.8×, about 0.9×, about 1×, about 1.1×, about 1.2×, about 1.3×, about 1.4×, about 1.5×, about 1.6×, about 1.7×, about 1.8×, about 1.9×, about 2×, about 2.1×, about 2.2×, about 2.3×, about 2.4×, about 2.5×, about 2.6×, about 2.7×, about 2.8×, about 2.9×, about 3×, about 3.1×, about 3.2×, about 3.3×, about 3.4×, about 3.5×, about 3.6×, about 3.7×, about 3.8×, about 3.9×, about 4×, about 4.1×, about 4.2×, about 4.3×, about 4.4×, about 4.5×, about 4.6×, about 4.7×, about 4.8×, about 4.9×, about 5×, about 5.1×, about 5.2×, about 5.3×, about 5.4×, about 5.5×, about 5.6×, about 5.7×, about 5.8×, about 5.9×, about 6×, about 6.1×, about 6.2×, about 6.3×, about 6.4×, about 6.5×, about 6.6×, about 6.7×, about 6.8×, about 6.9×, about 7×, about 7.1×, about 7.2×, about 7.3×, about 7.4×, about 7.5×, about 7.6×, about 7.7×, about 7.8×, about 7.9×, about 8×, about 8.1×, about 8.2×, about 8.3×, about 8.4×, about 8.5×, about 8.6×, about 8.7×, about 8.8×, about 8.9×, about 9×, about 9.1×, about 9.2×, about 9.3×, about 9.4×, about 9.5×, about 9.6×, about 9.7×, about 9.8×, about 9.9×, or about 10× compared to the negative control.

In embodiments, the decrease is calculated in comparison to a level and/or activity of the cytokine in a positive control. In embodiments, the positive control comprises the cytokine. In embodiments, the positive control comprises the levels of the cytokine found in individuals that are undergoing an inflammatory response.

In some embodiments of any of the aspects disclosed herein, the cancer is selected from ovarian cancer, fallopian tube cancer, peritoneal cancer, cutaneous squamous cell carcinoma (CSCC), and squamous cell carcinoma of the head and neck (SCCHN).

In embodiments, the biological sample is a body fluid, a sample of separated cells, a sample from a tissue or an organ, or a sample of wash/rinse fluid obtained from an outer or inner body surface of a subject. In embodiments, the biological sample is a body fluid selected from blood, plasma, serum, lacrimal fluid, tears, bone marrow, blood, blood cells, ascites, tissue or fine needle biopsy sample, cell-containing body fluid, free floating nucleic acids, sputum, saliva, urine, cerebrospinal fluid, peritoneal fluid, pleural fluid, feces, lymph, gynecological fluid, skin swab, vaginal swab, oral swab, nasal swab, washing or lavage such as a ductal lavage or broncheoalveolar lavage, aspirate, scraping, bone marrow specimen, tissue biopsy specimen, surgical specimen, feces, other body fluids, secretions, and/or excretions, and/or cells therefrom.

In embodiments, the biological sample is a fresh tissue sample, a frozen tumor tissue specimen, cultured cells, circulating tumor cells, or a formalin-fixed paraffin-embedded tumor tissue specimen. In embodiments, the biological sample is a tumor sample derived from a tumor selected from ovarian cancer, fallopian tube cancer, peritoneal cancer, cutaneous squamous cell carcinoma (CSCC), and squamous cell carcinoma of the head and neck (SCCHN).

In embodiments, the biological sample is obtained by a well-known technique including, but not limited to scrapes, swabs or biopsies. In embodiments, the biological sample is obtained by needle biopsy. In embodiments, the biological sample is obtained by use of brushes, (cotton) swabs, spatula, rinse/wash fluids, punch biopsy devices, puncture of cavities with needles or surgical instrumentation. In embodiments, the biological sample is or comprises cells obtained from an individual. In embodiments, the obtained cells are or include cells from an individual from whom the biological sample is obtained. In embodiments, a biological sample is a “primary sample” obtained directly from a source of interest by any appropriate means. For example, In embodiments, the biological sample is obtained by methods selected from the group consisting of biopsy (e.g., fine needle aspiration or tissue biopsy), surgery, collection of body fluid (e.g., blood, lymph, feces etc.), etc. In embodiments, the biological sample is originates from a tumor, blood, liver, the urogenital tract, the oral cavity, the upper aerodigestive tract the epidermis, or anal canal. It is to be understood that the biological sample may be further processed in order to carry out the method of the present disclosure. Such a “processed sample” may comprise, for example nucleic acids or proteins extracted from a sample or obtained by subjecting a primary sample to techniques such as amplification or reverse transcription of mRNA, isolation and/or purification of certain components, etc.

In embodiments, the level and/or activity of the cytokine is measured by one or more of RNA sequencing, immunohistochemical staining, western blotting, in cell western, immunofluorescent staining, ELISA, and fluorescent activating cell sorting (FACS).

In embodiments, the level and/or activity of the cytokine is measured by contacting the sample with an agent that specifically binds to one or more of the cytokines. In embodiments, the agent that specifically binds to one or more of the cytokines is an antibody or fragment thereof. In embodiments, the antibody is a recombinant antibody, a monoclonal antibody, a polyclonal antibody, or fragment thereof.

In embodiments, the level and/or activity of the cytokine is measured by contacting the sample with an agent that specifically binds to one or more of the nucleic acids. In embodiments, the agent that specifically binds to one or more of the nucleic acids is a nucleic acid primer or probe.

In embodiments, the evaluating comprises any one of diagnosis, prognosis, and response to treatment. In embodiments, the evaluating informs classifying the subject into a high or low risk group. In embodiments, the high risk classification comprises a high level of cancer aggressiveness, wherein the aggressiveness is characterizable by one or more of a high tumor grade, low overall survival, high probability of metastasis, and the presence of a tumor marker indicative of aggressiveness. In embodiments, the low risk classification comprises a low level of cancer aggressiveness, wherein the aggressiveness is characterizable by one or more of a low tumor grade, high overall survival, low probability of metastasis, and the absence and/or reduction of a tumor marker indicative of aggressiveness. In embodiments, the low risk or high risk classification is indicative of withholding of neoadjuvant therapy. In embodiments, the low risk or high risk classification is indicative of withholding of adjuvant therapy. In embodiments, the low risk or high risk classification is indicative of continuing of the administration of the chimeric protein. In embodiments, the low risk or high risk classification is indicative of withholding of the administration of the chimeric protein.

In embodiments, the evaluating is predictive of a positive response to and/or benefit from the administration of the chimeric protein. In embodiments, the evaluating is predictive of a negative or neutral response to and/or benefit from the administration of the chimeric protein. In embodiments, the evaluating informs continuing the administration or withholding of the administration of the chimeric protein. In embodiments, the evaluating informs continuing of the administration of the chimeric protein. In embodiments, the evaluating informs changing the dose of the chimeric protein. In embodiments, the evaluating informs increasing the dose of the chimeric protein. In embodiments, the evaluating informs decreasing the dose of the chimeric protein. In embodiments, the evaluating informs changing the regimen of administration of the chimeric protein. In embodiments, the evaluating informs increasing the frequency of administration of the chimeric protein.

In embodiments, the evaluating informs administration of neoadjuvant therapy. In embodiments, the evaluating informs administration of adjuvant therapy. In embodiments, the evaluating informs withholding of neoadjuvant therapy. In embodiments, the evaluating informs changing of neoadjuvant therapy. In embodiments, the evaluating informs changing of adjuvant therapy. In embodiments, the evaluating informs withholding of adjuvant therapy.

In embodiments, the evaluating is predictive of a positive response to and/or benefit from neoadjuvant chemotherapy or a non-responsiveness to and/or lack of benefit from neoadjuvant chemotherapy. In embodiments, the evaluating is predictive of a positive response to and/or benefit from adjuvant chemotherapy or a non-responsiveness to and/or lack of benefit from adjuvant chemotherapy. In embodiments, the evaluating is predictive of a negative or neutral response to and/or benefit from neoadjuvant chemotherapy or a non-responsiveness to and/or lack of benefit from neoadjuvant chemotherapy. In embodiments, the evaluating is predictive of a negative or neutral response to and/or benefit from adjuvant chemotherapy or a non-responsiveness to and/or lack of benefit from adjuvant chemotherapy.

In embodiments, the evaluating informs decreasing the frequency of administration of the chimeric protein. In embodiments, the neoadjuvant therapy and/or adjuvant therapy is a chemotherapeutic agent. In embodiments, the neoadjuvant therapy and/or adjuvant therapy is a cytotoxic agent. In embodiments, the neoadjuvant therapy and/or adjuvant therapy is checkpoint inhibitor.

In embodiments, the neoadjuvant therapy and/or adjuvant therapy is checkpoint inhibitor. In embodiments, the checkpoint inhibitor is an agent that targets one of TIM-3, BTLA, CTLA-4, B7-H4, GITR, galectin-9, HVEM, PD-L1, PD-L2, B7-H3, CD244, CD160, TIGIT, SIRPα, ICOS, CD172a, and TMIGD2.

Subjects and/or Animals

In embodiments, the subject and/or animal is a human. In embodiments, the human is a pediatric human. In embodiments, the human is an adult human. In embodiments, the human is a geriatric human. In embodiments, the human may be referred to as a patient.

In embodiments, the administration of the SIRPα-Fc-CD40L chimeric protein does not cause an anemia or another cytopenia in the patient. In embodiments, the administration of the does not cause lysis of RBCs. In embodiments, the administration of the SIRPα-Fc-CD40L chimeric protein is less likely to cause anemia or another cytopenia in than, e.g. an anti-CD47 Ab. Accordingly, the SIRPα-Fc-CD40L chimeric protein may be administered to individuals that are at risk of developing anemia or another cytopenia.

In embodiments, the human subject has received, been tolerant to, or is ineligible for standard therapy and/or the cancer has no approved therapy considered to be standard of care. Standard of care is the treatment that is accepted by medical experts as a proper treatment for a certain type of cancer and that is widely used by healthcare professionals, and also called best practice, standard medical care, and standard therapy. For example, radical surgery has been reported to be the standard of care for fit stage I non-small cell lung cancer (NSCLC) patients. Zarogoulidis et al., J Thorac Dis. 5(Suppl 4): S389-S396 (2013). In the curative setting, high-dose cisplatin concurrent with radiotherapy has been reported to be the standard of care, either as primary treatment or after surgery for head and neck squamous cell carcinoma (HNSCC). Oosting and Haddard, Front Oncol. 9: 815 (2019). Some cancers have no standard of care either because no treatment is accepted by medical experts as a proper treatment, or no treatment exists.

In embodiments, the human subject is ineligible for standard therapy may be exclusion criteria such as blood count, organ function, co-morbid conditions (e.g. heart disease, such as individuals with baseline abnormal electrocardiogram readings, uncontrolled diabetes, kidney disease, liver disease), women who are or may become pregnant, prior cancer treatments, exposure to certain medications, demographics, disease characteristics, overall illness burden, prior cancer history, and physiological reserve.

In embodiments, the human subject has received more than two prior checkpoint inhibitor-containing treatment regimens, e.g., as a monotherapy or as a combination immunotherapy.

In embodiments, the human subject has failed platinum-based therapies, and optionally is ineligible for further platinum therapy. In embodiments, the human subject is not receiving a concurrent chemotherapy, immunotherapy, biologic or hormonal therapy, and/or wherein the human subject has received, been tolerant to, or is ineligible for standard therapy and/or the cancer has no approved therapy considered to be standard of care.

In embodiments, the human subject is refractory to a prior checkpoint inhibitor therapy. For example, the subject is experiencing or has experienced disease progression within three months of treatment initiation of the prior checkpoint inhibitor therapy.

In embodiments, the human subject has a life expectancy of greater than 12 weeks.

In embodiments, the human subject has a measurable disease by iRECIST (solid tumors) or RECIL 2017 (lymphoma).

In embodiments, the human subject is not receiving a concurrent chemotherapy, immunotherapy, biologic or hormonal therapy. However, the human subject may be receiving concurrent hormonal therapy for non-cancer related conditions is acceptable.

In certain embodiments, the human has an age in a range of from about 0 months to about 6 months old, from about 6 to about 12 months old, from about 6 to about 18 months old, from about 18 to about 36 months old, from about 1 to about 5 years old, from about 5 to about 10 years old, from about 10 to about 15 years old, from about 15 to about 20 years old, from about 20 to about 25 years old, from about 25 to about 30 years old, from about 30 to about 35 years old, from about 35 to about 40 years old, from about 40 to about 45 years old, from about 45 to about 50 years old, from about 50 to about 55 years old, from about 55 to about 60 years old, from about 60 to about 65 years old, from about 65 to about 70 years old, from about 70 to about 75 years old, from about 75 to about 80 years old, from about 80 to about 85 years old, from about 85 to about 90 years old, from about 90 to about 95 years old or from about 95 to about 100 years old.

In embodiments, the human subject has a cancer, wherein the cancer being treated is characterized by having macrophages in the tumor microenvironment and/or having tumor cells that are CD47+ in the tumor.

Aspects of the present disclosure include use of a chimeric protein as disclosed herein in the manufacture of a medicament, e.g., a medicament for treatment of cancer.

Aspects of the present disclosure include use of a chimeric protein as disclosed herein in the treatment of cancer.

Any aspect or embodiment disclosed herein can be combined with any other aspect or embodiment as disclosed herein In embodiments, the chimeric proteins disclosed herein (e.g. a recombinant, chimeric glycoprotein comprising the extracellular domain of human CD172a (SIRPα), a central domain from the human immunoglobulin constant gamma 4 (IgG4), and the extracellular domain of human CD40L, i.e., hCD172a (SIRPα)-Fc-CD40L) binds to its cognate target molecules CD47 and CD40 with nanomolar affinity in a dose-dependent manner, both individually and simultaneously. The chimeric proteins disclosed herein displayed slower dissociation kinetics when bound to CD47 and CD40 compared to its interactions with control molecules suggesting that the fusion of CD172a (SIRPα) and CD40L via an Fc domain increases receptor-occupancy time, a beneficial characteristic in a tumor microenvironment.

Binding of the CD172a (SIRPα)-Fc-CD40L chimeric protein (e.g. SEQ ID NO: 59 or SEQ ID NO: 61) to CD40 was shown to increase NFκB signaling and increase secretion of IL2 from CD3+ T cells in the presence of tumor cells expressing high levels of CD47. It also was found to stimulate expression of Ki67 (an intracellular marker for cell proliferation) in CD4+ and CD8+ T cells and increase expression of IFNγ and TNFα in human CD8+ T cells.

In a Staphylococcus enterotoxin B (SEB) assay, the CD172a (SIRPα)-Fc-CD40L chimeric protein (e.g. SEQ ID NO: 59 or SEQ ID NO: 61) stimulated higher cytokine production from human PBMCs than its components alone or in combination, suggesting that the physical tethering of the CD172a (SIRPα) and CD40L domains by the Fc fragment provides a greater IL2 response than either ligand/receptor separately. The geometric mean for the EC₅₀ values of the CD172a (SIRPα)-Fc-CD40L chimeric protein (e.g. SEQ ID NO: 59 or SEQ ID NO: 61) following SEB super antigen stimulation, 50 ng/mL and 100 ng/mL, were 0.4866 nM and 0.5903 nM, respectively; however, because SEB is capable of activating a large proportion of TCRs present in a PBMC sample, these values likely over-estimate the minimal concentration at which an additional immune stimulating agent (such as the CD172a (SIRPα)-Fc-CD40L chimeric protein (e.g. SEQ ID NO: 59 or SEQ ID NO: 61)) may enhance immune responses in patients. As tumor-antigen specific immune responses in human cancer patients comprise only a small proportion of the overall T cell-mediated immune response repertoire, it is likely that the estimated EC₅₀ values for the CD172a (SIRPα)-Fc-CD40L chimeric protein (e.g. SEQ ID NO: 59 or SEQ ID NO: 61) from the SEB assay provide a conservative estimate for the dose level at which similar responses could be seen in human cancer patients. In similar experiments with PBMCs from cynomolgus monkeys, the species utilized in the below-disclosed non-human primate studies, IL-2 secretion was observed at similar concentrations of the CD172a (SIRPα)-Fc-CD40L chimeric protein (e.g. SEQ ID NO: 59 or SEQ ID NO: 61) to that of the human PBMCs. In comparison to commercial antibodies, the CD172a (SIRPα)-Fc-CD40L chimeric protein (e.g. SEQ ID NO: 59 or SEQ ID NO: 61) also induced higher expression of IL2, further supporting the expectation that the bispecific actions of the CD172a (SIRPα)-Fc-CD40L chimeric protein (e.g. SEQ ID NO: 59 or SEQ ID NO: 61) will improve upon the success rate of immune checkpoint or CD40 single-targeted therapies currently available in the clinical setting.

In summary, the CD172a (SIRPα)-Fc-CD40L chimeric protein (e.g. SEQ ID NO: 59 or SEQ ID NO: 61) selectively and specifically binds to its intended targets of CD47 and CD40 with high affinity. The CD172a (SIRPα)-Fc-CD40L chimeric protein (e.g. SEQ ID NO: 59 or SEQ ID NO: 61) has exhibited functional activity associated with the binding of both targets in a variety of in vitro assays including in vitro anti-tumor models. In vivo anti-tumor activity of a murine version of the protein was demonstrated in mouse tumor models. Minimal cross-reactivity with non-specific targets was observed in human tissues. The minimum anticipated biological effect level (MABEL) based on the EC₅₀ of the SEB super antigen stimulation assay was determined to be 0.587 nM or 33.8 ng/mL.

The invention will be further described in the following examples, which do not limit the scope of the invention described in the claims.

### EXAMPLES

The examples herein are provided to illustrate advantages and benefits of the present disclosure and to further assist a person of ordinary skill in the art with preparing or using the chimeric proteins of the present disclosure. The examples herein are also presented in order to more fully illustrate the preferred aspects of the present disclosure. The examples should in no way be construed as limiting the scope of the present disclosure, as defined by the appended claims. The examples can include or incorporate any of the variations, aspects or embodiments of the present disclosure described above. The variations, aspects or embodiments described above may also further each include or incorporate the variations of any or all other variations, aspects or embodiments of the present disclosure.

### Example 1: Materials and Methods

### Construct Generation and Protein Purification.

The coding sequences of both human (h) and mouse (m) SIRPα-Fc-CD40L were codon-optimized and directionally cloned into the pcDNA3.4 TOPO TA (Thermo Fisher Scientific, catalog no. A14697) mammalian expression vector and nucleotide sequences were verified by Sanger sequencing methodology (performed off site by GENEWIZ). The SIRPα-Fc-CD40L Sequences are provided in the Table below:

| | |
|---|---|
| mSIRPα | KELKVTQPEKSVSVAAGDSTVLNCTLTSLLPVGPIRWYRGVGPSRLLIYSFAGE |
| ECD | YVPRIRNVSDTTKRNNMDFSIRISNVTPADAGIYYCVKFQKGSSEPDTEIQSGG |
| | GTEVYVLAKPSPPEVSGPADRGIPDQKVNFTCKSHGFSPRNITLKWFKDGQEL |
| | HPLETTVNPSGKNVSYNISSTVRVVLNSMDVNSKVICEVAHITLDRSPLRGIANL |
| | SNFIRVSPTVKVTQQSPTSMNQVNLTCRAERFYPEDLQLIWLENGNVSRNDTP |
| | KNLTKNTDGTYNYTSLFLVNSSAHREDVVFTCQVKHDQQPAITRNHTVLGFAH |
| | SSDQGSMQTFPDNNATHNWN (SEQ ID NO: 62) |
| mhinge- | VPRDCGCKPCICTVPEVSSVFIFPPKPKDVLTITLTPKVTCVVVDISKDDPEVQF |
| CD2-CD3 | SWFVDDVEVHTAQTQPREEQFNSTFRSVSELPIMHQDWLNGKEFKCRVNSAA |
| | FPAPIEKTISKTKGRPKAPQVYTIPPPKEQMAKDKVSLTCMITDFFPEDITVEWQ |
| | WNGQPAENYKNTQPIMNTNGSYFVYSKLNVQKSNWEAGNTFTCSVLHEGLHN |
| | HHTEKSLSHSPGK (SEQ ID NO: 63) |
| mCD40L | HRRLDKVEEEVNLHEDFVFIKKLKRCNKGEGSLSLLNCEEMRRQFEDLVKDITL |
| ECD | NKEEKKENSFEMQRGDEDPQIAAHVVSEANSNAASVLQWAKKGYYTMKSNLV |
| | MLENGKQLTVKREGLYYVYTQVTFCSNREPSSQRPFIVGLWLKPSSGSERILLK |
| | AANTHSSSQLCEQQSVHLGGVFELQAGASVFVNVTEASQVIHRVGFSSFGLLK |
| | L (SEQ ID NO: 64) |
| hSIRPα | EEELQVIQPDKSVLVAAGETATLRCTATSLIPVGPIQWFRGAGPGRELIYNQKE |
| ECD | GHFPRVTTVSDLTKRNNMDFSIRIGNITPADAGTYYCVKFRKGSPDDVEFKSGA |
| | GTELSVRAKPSAPVVSGPAARATPQHTVSFTCESHGFSPRDITLKWFKNGNEL |
| | SDFQTNVDPVGESVSYSIHSTAKVVLTREDVHSQVICEVAHVTLQGDPLRGTAN |
| | LSETIRVPPTLEVTQQPVRAENQVNVTCQVRKFYPQRLQLTWLENGNVSRTET |
| | ASTVTENKDGTYNWMSWLLVNVSAHRDDVKLTCQVEHDGQPAVSKSHDLKVS |
| | AHPKEQGSNTAAENTGSNERNIY (SEQ ID NO: 65) |
| hIgG4 | ESKYGPPCPSCPAPEFLGGPSVFLFPPKPKDTLMISRTPEVTCVVVDVSQEDP |
| hinge- | EVQFNWYVDGVEVHNAKTKPREEQFNSTYRVVSVLTVLHQDWLNGKEYKCKV |
| CH2-CH3 | SNKGLPSSIEKTISKAKGQPREPQVYTLPPSQEEMTKNQVSLTCLVKGFYPSDI |
| | AVEWESNGQPENNYKTTPPVLDSDGSFFLYSRLTVDKSRWQEGNVFSCSVMH |
| | EALHNHYTQKSLSLSLGK (SEQ ID NO: 66) |
| hCD40L | HRRLDKIEDERNLHEDFVFMKTIQRCNTGERSLSLLNCEEIKSQFEGFVKDIMLN |
| ECD | KEETKKENSFEMQKGDQNPQIAAHVISEASSKTTSVLQWAEKGYYTMSNNLVT |
| | LENGKQLTVKRQGLYYIYAQVTFCSNREASSQAPFIASLCLKSPGRFERILLRAA |
| | NTHSSAKPCGQQSIHLGGVFELQPGASVFVNVTDPSQVSHGTGFTSFGLLKL |
| | (SEQ ID NO:58) |

For transient transfection-based protein production runs, human or mouse SIRPα-Fc-CD40L-expressing vectors were transfected into Expi293 cells using the ExpiFectamine 293 transfection kit (Thermo Fisher Scientific, catalog no. A14524) and cell culture media containing SIRPα-Fc-CD40L was harvested on day 6 posttransfection. In addition, the hSIRPα-Fc-CD40L vector was stably transfected into CHO cells and a final clone expressing high levels of hSIRPα-Fc-CD40L was selected for large scale protein production and purification (Selexis SA). Briefly, SIRPα-Fc-CD40L containing cell culture media were harvested by centrifugation at 5,000 rpm for 10 minutes followed by filtration through a 0.2-μm filter. The clarified supernatant was bound to a HighTrap Protein A HP column, washed and eluted under standard conditions. The eluted protein was dialyzed into 1×PBS and the concentration was determined by absorption at 280 nm using a NanoDrop spectrophotometer (Thermo Fisher Scientific).

### Western Blot Analysis

Human (h) and mouse (m) SIRPα-Fc-CD40L proteins were treated with or without the deglycosylase PNGase F (NEB, catalog no. P0704) for 1 hour at 37° C. according to manufacturer's recommendations, and then with or without the reducing agent β-mercaptoethanol, and diluted in SDS loading buffer prior to separation by SDS-PAGE. Primary and secondary antibodies were used for probing hSIRPα-Fc-CD40L (anti-SIRPα; Cell Signaling Technology, catalog no. 43248S, anti-human IgG; Jackson ImmunoResearch, catalog no. 109-005-098, anti-CD40L; R&D Systems, catalog no. AF1054), and mSIRPα-Fc-CD40L (anti-SIRPα; BioLegend, catalog no. 144001, anti-mouse IgG; Jackson ImmunoResearch, catalog no. 115-005-068, anti-CD40L; R&D Systems, catalog no. AF1163). Secondary anti-goat IgG and anti-rabbit IgG were obtained from LI-COR; catalog numbers 925-32214 and 925-32211, respectively.

### Electron Microscopy

Samples were diluted in PBS (30- to 140-fold) and imaged over a layer of continuous carbon supported by nitrocellulose on a 400-mesh copper grid. Electron microscopy was performed using an FEI Tecnai T12 electron microscope operating at 120 keV equipped with an FEI Eagle 4k×4k CCD camera (NanoImaging Services). Negative stain grids were transferred into the electron microscope using a room temperature stage. Images of each grid were acquired at multiple scales to assess the overall distribution of the specimen. After identifying suitable target areas for imaging, high magnification images were acquired at nominal magnifications of 110,000× and 67,000×. The images were acquired at a nominal underfocus of −1.6 μmol/L to −0.9 μmol/L and electron doses of approximately 25 e⁻/A². Two-dimensional averaging analysis was performed using automated picking protocols followed by reference-free alignment based on the XMIPP processing package.

### Functional ELISA

For characterization of the human or mouse SIRPα-Fc-CD40L chimeric proteins, high-binding ELISA plates (Corning) were coated overnight at 4° C. with 5 μg/mL of either anti-hFc, anti-mFc, recombinant hCD47-Fc, hCD40-His, mCD47-Fc or mCD40-His, in PBS (reagents were obtained from Jackson ImmunoResearch Laboratories, Sino Biologics, Inc., AcroBiosystems, and R&D Systems). Plates were then blocked with casein buffer for 1 hour at room temperature and then probed with serial dilutions of the human or mouse SIRPα-Fc-CD40L, along with the appropriate standards (human and mouse; IgG, SIRPα-Fc, and Fc-CD40L) for 1 hour at room temperature. Plates were washed with TBS-T (Tris-buffered saline containing 0.1% Tween 20) and then detection antibody was added for 1 hour at room temperature in the dark. Detection antibodies included anti-hIgG-HRP, anti-mIgG-HRP, anti-hCD40L, anti-mCD40L, and then anti-Goat-HRP (all antibodies were obtained from Jackson ImmunoResearch Laboratories or R&D Systems). Plates were washed again and SureBlue TMB Microwell Peroxidase Substrate (KPL; purchased from VWR, catalog no. 95059-282) was added to each well and allowed to incubate at room temperature in the dark. To stop the reaction, 1N sulfuric acid was added to each well and absorbance at 450 nm was read immediately on a BioTek plate reader. Samples were run at a minimum in triplicate and at multiple dilutions.

For SIRPα-blocking ELISA, hCD47-Fc (Sino Biological Inc.) was used to coat a high binding ELISA plate as described above. The following day, binding was detected using a recombinant hSIRPα-Biotin protein (Acro Biosystems) in combination with increasing concentrations of either hSIRPα-Fc-CD40L (to compete with SIRPα-Biotin binding for CD47) or an anti-CD47 blocking antibody (clone CC2C6, BioLegend), which served as a positive control. Following this incubation, plates were washed as described above and probed with an avidin-HRP detection antibody (BioLegend) for 1 hour at room temperature in the dark. Plates were then washed and analyzed as above.

### Surface Plasmon Resonance

Direct binding of human or mouse SIRPα-Fc-CD40L fusion protein to recombinant protein targets was performed using a Bio-Rad ProteOn XPR36 protein interaction array instrument. To determine the on-rates (Kₐ), off-rates (K_{d}), and binding affinities (K_{D}) of hSIRPα-Fc-CD40L to its intended binding targets (referred to as “Ligands”), histidine-tagged versions of the human recombinant targets-CD47 (AcroBiosystems), CD40 (AcroBiosystems), FcγR1A (Sino Biological Inc.), FcγR2B (Sino Biological Inc.), FcγR3B (Sino Biological Inc.), and FcRn (R&D Systems) were immobilized to a Ni-sulfate activated ProteOn HTG sensor chip (Bio-Rad). Increasing concentrations of the hSIRPα-Fc-CD40L fusion protein (referred to as “Analyte”) diluted in PBS/Tween (0.005%) buffer pH 7.0 were injected for 3 minutes followed by a dissociation phase of 5 minutes at a flow rate of 80 μL/minute. hSIRPα-Fc and hCD40L-Fc (AcroBiosystems) recombinant proteins were used as positive control analytes for binding to their partners (CD47 and CD40, respectively) and human IgG was used as a positive control for binding to Fcγ receptors and FcRn. To assess analyte binding to FcRn ligand, the pH of the buffer was reduced to pH 5.5.

### Cell Culture

CHO-K1, CT26, A20, WEHI3, Toledo, Raji, Ramos, A431, HCC827, K562, HCC1954, and MCF7 cells were obtained from ATCC (between 2017 and 2019) and cultured according to their guidelines; maintained at 37° C. in 5% C02. All parental cell lines in active culture are tested monthly using the Venor GeM Mycoplasma Detection Kit (Sigma). All transfected cell lines are tested an additional two times, separated by at least 2 weeks, posttransfection, and confirmed to remain negative for Mycoplasma. Research cell banks (RCB) of all purchased cell lines were generated within 5 passages of initial cell line thawing. All cells used for experimentation were used within 10 passages of vial thawing. Expression from receptor/ligand overexpressing cells lines was verified by flow cytometry prior to generating the RCB, and then periodically checked again when new vials were thawed and put into culture.

### In Vitro Cell Line Generation

Stable cells lines were generated to assess in vitro binding of the human and mouse SIRPα-Fc-CD40L chimeric protein proteins. To generate the CHO-K1/hCD47, CHO-K1/mCD47, and CHO-K1/mCD40 cell lines, total RNA was extracted from CD3/CD28 activated human PBMCs and mouse splenocytes (1×10⁶ cells) using the RNeasy mini kit (Qiagen, catalog no. 74104) and 1 μg of RNA was used to generate first strand cDNA using a commercial kit (Origene, catalog no. 11801-025). One microliter of the resulting first strand cDNA was used to PCR amplify each of the targets using Platinum Taq DNA polymerase (Thermo Fisher Scientific, catalog no. 10966034) and the following primer pairs with restriction enzyme sites appended to the 5′ ends for subcloning the PCR fragment into the pcDNA3.1(−) expression vector (Life Technologies) using standard molecular biology techniques-hCD47 forward 5′ GCGGCGCTCGAGGCCACCATGTGGCCCCTGGTAGCGG (SEQ ID NO: 67); hCD47 reverse 5′ ACTAGCGGTACCCCATCACTTCACTTCAGTTATTCCACAAATTTC (SEQ ID NO: 68); mCD47 forward 5′ GCGGCGCTCGAGGCCACCATGTGGCCCTTGGCGGC (SEQ ID NO: 69), mCD47 reverse 5′ ACTAGCGGTACCTCACCTATTCCTAGGAGGTTGGATAGTCC (SEQ ID NO: 70); mCD40 forward 5′ GCGGCGCTCGAGGCCACCATGGTGTCTTTGCCTCGGCTG (SEQ ID NO: 71); mCD47 reverse 5′ ACTAGCGGTACCTCAGACCAGGGGCCTCAAG (SEQ ID NO: 72). The CHOK1-hCD40 cell line was generated by cloning the hCD40 cDNA (R&D Systems #RD1325) into pcDNA3.1(−) vector. The nucleotide sequences of the subcloned cDNAs in the pcDNA3.1(−) vector were confirmed by sanger sequencing (GENEWIZ). Parental CHO-K1 cells were nucleofected with each of the target cDNA expressing pcDNA3.1(−) vectors using the 4D-Nucleofector and Cell Line Nucleofector Kit SE (Lonza, catalog no. V4XC-1012) according to manufacturer's directions. Two days postnucleofection the cells were placed under G418 selection (0.5 mg/mL) for two weeks and the stable pool was subsequently single-cell-cloned using limiting dilution to isolate clones that expressed high amounts of the target receptors, which was confirmed by flow cytometry using the following APC-conjugated fluorescent antibodies from BioLegend-anti-hCD40 (catalog no. 334310), anti-hCD47 (catalog no. 323124), anti-mouse CD40 (catalog no. 124612), and anti-mouse CD47 (catalog no. 127514). CHO-K1/mCD40 and CHO-K1/hCD40 cells were also subsequently stably nucloefected with pGL4.32[luc2P/NF-κB-RE/Hygro] (Promega) reporter plasmid, to generate CD40-driven NFκB reporter cells.

### Flow Cytometry

Briefly, isolated cells were washed one time with 1×PBS, followed by centrifugation at 400×g for 5 minutes. Cells were then stained with antibodies for 30 minutes on ice in the dark. Indicated antibodies were purchased from Sony, BioLegend, or Abcam, and used at their recommended concentrations, and diluted in FACS buffer [1×PBS buffer containing 1% BSA, 0.02% sodium azide, and 2 mmol/L EDTA]. The AH1-tetramer reagent was purchased from MBL International (catalog no. TB-M521-2), and was incubated with cells for 1 hour on ice in the dark before adding the rest of the antibody cocktail. Following this incubation period, stained cells were washed by adding 0.5 mL of FACS buffer, and then centrifuged at 400×g for 5 minutes. The supernatant above the pellet was then aspirated and the resulting cells were resuspended in FACS buffer and flow cytometry was performed on a BD LSRII Fortessa according to manufacturer's recommendations, and data were analyzed by FlowJo.

### In Vitro Functional Assays

### Phagocytosis Assay.

Frozen vials of peripheral blood mononuclear cells (PBMC) from healthy human donors were purchased from STEMCELL Technologies and monocytes were isolated using a commercial kit according to the manufacturer's protocol (STEMCELL, catalog no. 19059) and were confirmed to be CD14⁺ by flow cytometry postisolation using an APC-conjugated anti-human CD14 (BioLegend, catalog no. 367118) and the appropriate isotype control antibody. Following the isolation of monocytes, a live cell count was performed using Trypan blue staining 4×10⁵ live and monocytes were seeded in Iscove modified Dulbecco media (IMDM) containing 10% FBS and 50 ng/mL of human macrophage colony-stimulating factor (M-CSF; BioLegend) in 24-well plates (Corning). Cells were incubated in 37° C./5% CO₂ for 7 days to differentiate into macrophages. Media were changed on day 4 and supplemented with M-CSF. On day 7, macrophages were polarized to an M1 state by replacing the media with fresh media containing 10 ng/mL lipopolysaccharide (LPS-EB; InvivoGen) and 50 ng/mL human IFNγ (STEMCELL Technologies). Cells were incubated for another 48 hours and M1 polarization status was confirmed by flow cytometry for CD11b, HLA-DR, CD80, and CD206 (BioLegend). Macrophages were confirmed to be M1 polarized if they stained positive for CD11b, HLA-DR, and CD80 and negative for CD206. On day 9, the media containing LPS and IFNγ was replaced with fresh media (IMDM+10% FBS), and macrophage:tumor cocultures were initiated.

Flow cytometry-based analysis: Tumor cells were harvested and incubated with SIRPα-Fc-CD40L either alone at 1 μmol/L test concentration or in combination with select antibody-dependent cellular phagocytosis (ADCP)-competent antibodies (based on the type of tumor cell used in the assay) at 0.06 μmol/L test concentration for 37° C. for 1 hour. Following the incubation, cells were washed with Dulbecco PBS (DPBS) and stained with a CellTracker Green CMFDA dye (Thermo Fisher Scientific/Invitrogen, catalog no. C2925) according to the manufacturer guidelines. Cells were washed twice with DPBS and resuspended in serum-free media. The stained tumor cells were cocultured with M1-polarized macrophages for 2 hours at 37° C. at a tumor:macrophage ratio of 5:1. When appropriate, Fc receptors and CD40 receptors were blocked on macrophages using commercial antibodies (20 μg/mL; BioLegend and R&D Systems, respectively) and calreticulin was blocked on tumor cells using a calreticulin blocking peptide (10 μg/mL; MBL International Corporation) for 1 hour at 37° C. prior to coculture initiation. After the coculture incubation period, unbound/unengulfed tumor cells were removed and the macrophages were rinsed in DPBS and harvested using a nonenzymatic cell stripping solution (Thermo Fisher Scientific/Corning, catalog no. 25-056-C1). Harvested macrophage Fc receptors were blocked using a commercial human Fc blocking reagent (BioLegend). Thereafter, the Fc-receptor blocked macrophages were stained with an anti-human CD11b antibody conjugated to PE/Cy7 (BioLegend), washed, and analyzed on an LSRII Fortessa flow cytometer (BD Biosciences) to detect macrophages that stained positive for both CD11b and the CellTracker Green CMFDA dye following a phagocytic event. Cells were pregated on CD11b⁺ macrophages. Samples were analyzed on a LSRII Fortessa and flow cytometry standard (FCS) files were analyzed using the FlowJo software version 10. The percentage of CD11b⁺ Green dye⁺ macrophages were plotted in GraphPad Prism 8. A phagocytosis index was calculated by setting the maximum response across the treatment groups to 1 and calculating the fold change for each of the treatment groups relative to the maximum response. A similar flow-based methodology was used to study phagocytosis of mouse tumor cells (WEHI or A20) by bone marrow-derived macrophages (BMDM) with mouse SIRPα-Fc-CD40L or mouse anti-CD47 (BioXCell, clone MIAP301). BMDMs were isolated and grown in IMDM with 10% FBS supplemented with mouse M-CSF (50 ng/mL) for 7 days, and activated with LPS (10 ng/mL) and mIFNγ (50 ng/mL) for 2 days.

Fluorescence microscopy-based analysis: Human monocytes (CD14+) were plated in 16-well chamber glass slides (Thermo Fisher Scientific) to differentiate into macrophages and were polarized to an M1 state using LPS and IFNγ as described above. On the day of the experiment, tumor cells (Toledo) were prepared and cocultured with the M1 macrophages in the chamber slides in the presence of SIRPα-Fc-CD40L either alone or in combination with select ADCP-competent antibodies for 2 hours at 37° C. After the incubation period the unbound/unengulfed tumor cells were removed from the wells and the macrophages were rinsed four times in 1×DPBS. Subsequent blocking and staining steps were performed in the chamber slide. The Fc receptors on macrophages were blocked as described previously and subsequently stained with an anti-human CD11b antibody conjugated to Alexa Fluor 594 (BioLegend) overnight at 4° C. protected from light. Macrophages were rinsed twice in DPBS to remove unbound stain and coverslips were mounted in ProLong Diamond anti-fade mountant containing DAPI (Invitrogen) and allowed to dry overnight at room temperature protected from light. Once dry, the edges of the coverslips were sealed with clear nail polish and fluorescent imaging was performed on a Zeiss 800 confocal microscope. Four representative images were acquired per well in the chamber slide under 10× and 60× magnifications and ImageJ software (NIH, Bethesda, MD) was used to process the raw images and quantify the number of engulfed tumor cells (labeled green) per total macrophages (labeled red) based on the pixel density. The compiled data included one representative image per condition and the quantification data incorporated at least four images per well and two wells per condition.

Time-lapse microscopy-based analysis: Human monocytes (CD14+) were plated in a 96-well plate (Millipore Sigma), differentiated into macrophages, and polarized to an M1 state using LPS and IFNγ as described above. On the day of the experiment, Toledo tumor cells were labeled with the IncuCyte phRodo Red cell labeling kit (Sartorius, catalog no. 4649) according to the manufacturer's guidelines and then were cocultured with the M1 macrophages at a 5:1 ratio in the presence of SIRPα-Fc-CD40L (1 μmol/L), anti-CD20 (rituximab; 1 μg/mL), anti-CD47 (BioLegend clone CC2C6; 33 μg/mL); or the combination of anti-CD20 with either SIRPα-Fc-CD40L or anti-CD47. The 96-well plate was placed in the IncuyCyte S3 and time-lapse imaging was performed over the course of 5 hours at 37° C. Five images per well, in duplicate, using two distinct donors were acquired under 10× magnification for each treatment group and the red object integrated intensity per image was calculated using the IncuCyte S3 software. The phagocytosis index was calculated by taking the maximum signal observed from each experiment and setting that as the 100% point. Each data point from a given experiment was normalized to the maximum value for that experiment (i.e., fluorescence or luminescence). These calculations were repeated for each experimental replicate, and then all combined data was plotted using GraphPad Prism 8.

### In Vivo DC Activation Assay.

BALB/C mice were obtained from Jackson Laboratories, and treated with a single intravenous injection of 1×10⁷ PBS washed sheep red blood cells (RBC diluted in PBS; Rockland Immunochemicals), anti-CD47 (clone MIAP301), anti-SIRPα (clone P84), or mSIRPα-Fc-CD40L. Antibodies were given at a total dose of 100 μg and the SIRPα-Fc-CD40L chimeric protein at 300 μg; all in a volume of 100 μL diluted in PBS. After 6 or 24 hours, mice were humanely euthanized and splenocytes were isolated for flow cytometry-based immune profiling of DCs, by removing the spleens from treated animals, mechanically dissociating them using the flat ends of two razors followed by repeated pipetting to break up larger pieces, and then passing the cells through a 100-μm cell strainer. RBCs were lysed with RBC lysis buffer according to manufacturer's recommendations (BioLegend, catalog no. 420301) and the remaining mononuclear cells were stained with fluorescent-conjugated antibodies to CD11c, DC1R2, I-A^{b} (MHC-II), and either CD4 or CD8. After 30 minutes on ice in the dark, cells were washed, and then analyzed on an LSR II Fortessa flow cytometer. CD4⁺ or CD8⁺ DCs were pregated on CD11c⁺/DC1R2⁺ populations, and activated DCs were also positive for I-A^{b}. Additional pharmacodynamic activity was assessed in the peripheral blood, 24 hours following single intraperitoneal injections of a dose titration of mSIRPα-Fc-CD40L. A small amount of peripheral blood was collected from the tail and RBCs were lysed as described above. Populations of CD20⁺ cells were assessed by flow cytometry.

### NFκB-Luciferase Reporter Assay.

CHOK1/mCD40/NFκB-luciferase cells were treated with a dose titration of mSIRPα-Fc-CD40L, recombinant Fc-mCD40L protein (Sino Biologics), or anti-mCD40 (clone FGK4.5; BioXcell). CHOK1/hCD40/NFκB-luciferase cells, were treated with a dose titration of SIRPα-Fc-CD40L and recombinant hCD40L-His protein (Sino Biologics). Bright-Glo luciferase reagent and a GloMax Navigator luminometer were used to assess activation of NFκB signaling. The expression vector, luciferase reagent (catalog no. E2650), and luminometer (catalog no. GM2000) are all from Promega and were used according to manufacturer suggestions. Briefly, 10,000 CHO-K1/CD40/NFκB-luciferase cells were plated in each well of a 96-well plate±the SIRPα-Fc-CD40L chimeric protein or other test agents. Plates were incubated at 37° C./5% C02 for 6 hours, then the Bright-Glo reagent was added and luminescence was assessed on the luminometer.

### NIK/NFκB Reporter Assay.

U2OS/NIK/NFκB reporter cells expressing CD40 were purchased from Eurofins/DiscoverX (catalog no. 93-1059C3) and cultured according to their recommendations. On the day of the assay, 10,000 U2OS/NIK/NFκB reporter cells were plated into each well of a 96-well plate with a dose titration of either SIRPα-Fc-CD40L, recombinant Fc-hCD40L protein (Sino Biologics), or an anti-CD40 agonist antibody (clone HB14; BioLegend). After 6 hours in culture, luminescence activity was assessed on a luminometer (Promega) as described above.

### AIMV Activation Assay.

Proliferation: PBMCs were isolated from 50 healthy donor buffy coats and CD8⁺ T cells were depleted using CD8⁺ RosetteSep (STEMCELL Technologies). Cell depletion was confirmed by flow cytometry and was >90% for all donors. Cells were plated at a density of 4-6 million cells per mL in AIM-V medium (Gibco). On days 5, 6, and 7, cells were gently resuspended in 3×100 μL aliquots and transferred to each well of a round-bottom 96-well plate. Cultures were pulsed with 0.75 μCi [3H]-Thymidine (PerkinElmer) in 100 μL AIM-V culture medium, and incubated for a further 18 hours, before harvesting onto filter mats (PerkinElmer), using a TomTec Mach III cell harvester. Counts per minute (cpm) for each well were determined by Meltilex (PerkinElmer) scintillation counting on a 1450 Microbeta Wallac Trilux Liquid Scintillation Counter (PerkinElmer), with low background counting.

IL2 ELISpot: PBMCs were isolated, CD8 cells depleted, and cultured in AIM-V as described above, and on day 8, cells were assessed. Briefly, ELISpot plates (Millipore) were prewetted and coated overnight with 100 μL/well IL2 capture antibody (R&D Systems) in PBS. Cells were plated at a density of 4-6 million cells/mL in a volume of 100 μL per well, in sextuplicate. After 8 days, ELISpot plates were developed by sequential washing in dH₂O and PBS (×3), prior to the addition of 100 μL filtered biotinylated detection antibody (R&D Systems). Following incubation at 37° C. for 1.5 hours, plates were further washed with PBS and 1% BSA, and incubated with 100 μL filtered streptavidin-AP (R&D Systems) for 1 hour; and then washed again. One-hundred microliters BCIP/NBT substrate (R&D Systems) was added to each well for 30 minutes at room temperature. Spot development was stopped by washing wells three times with dH₂O. Dried plates were scanned on an Immunoscan Analyzer and spots per well (SPW) were determined using Immunoscan Version 5 software. Samples included human SIRPα-Fc-CD40L (at 0.3, 3, 30, and 300 nmol/L), the neoantigen KLH (Keyhole limpet hemocyanin; 300 nmol/L) as a positive control, and exenatide (Bydureon, 20 μmol/L) as a clinical benchmark control; representing a negative control in this assay. For ELISpot, a mitogen-positive control (PHA at 8 μg/mL) was included on each plate as an internal test for ELISpot function and cell viability.

### Type I IFN Response

### Gene Expression Analysis.

Human macrophage:Toledo/Raji tumor cocultures were set up as described above. After 2 hours of coculture and treatment with either SIRPα-Fc-CD40L (1 μmol/L), anti-human CD20/rituximab (0.06 μmol/L), or the combination of both agents; cells were collected from the plate using a nonenzymatic cell stripping solution (Thermo Fisher Scientific/Corning, catalog no. 25-056-C1), washed with PBS, and stained with a fluorescently conjugated antibody for CD11b, after Fc blocking. After a 30-minute incubation on ice in the dark, cells were washed and CD11b⁺ populations were sorted using a FACS Melody (BD Biosciences). Sorted macrophages were lysed with RLT lysis buffer (Qiagen RNeasy Micro Kit, catalog no. 74004) containing 5% 2-mercaptoethanol and processed according to manufacturer's directions; including on-column DNase I digestion. RNA was quantitated using a Nanodrop and 250 ng of resulting RNA was reverse transcribed (Origene First-strand cDNA Synthesis kit) according to manufacturer's recommendations. The resulting cDNA was diluted with nuclease-free water and the equivalent of 10 ng of starting RNA served as the template for each qPCR reaction. Gene expression was assessed using SYBR Green and the CFX96 Touch Real-Time PCR Detection System (Bio-Rad). Validated qPCR primers for IFNα1, IFNβ1, CD80, CD86 and R-Actin (ACTB) as a housekeeping control, were purchased from Origene. Fold change in gene expression was determined using the ΔΔCₜ method compared with untreated samples. Data generated were from a minimum of three experimental replicates all run in at least triplicate. Error bars represent SEM and significance was determined using one-way ANOVA.

### ISG Reporter Cells.

RAW264.7-Lucia ISG cells were obtained from InvivoGen and cultured according to their recommendations. RAW-Lucia ISG cells were cultured directly with A20 tumor cells, and 50 μg/mL of mSIRPα-Fc-CD40L, recombinant mFc-CD40L, recombinant mSIRPα-Fc (both at 50 μg/mL), the combination of mFc-CD40L and mSIRPα-Fc, anti-mCD20 (BioXcell clone AIS1B12; 1 μg/mL), or the combination of mSIRPα-Fc-CD40L and anti-CD20. Supernatants from cultures were collected 24 hours after culture, incubated with QUANTI-Luc reagent (InvivoGen, catalog no. rep-qlc1; according to their recommendations), and read on a luminometer (Promega).

### Tumor Model Systems

For CT26, A20, and WEHI3 studies, BALB/C mice were subcutaneously implanted with 5×10⁵ (CT26 and A20) or 1×10⁶ (WEHI3) tumor cells in 100 μL of PBS, into the rear flank, respectively, on day 0. On treatment days (treatment schematics shown in figures and described in figure legends), tumor bearing mice were randomized and either untreated or treated via intraperitoneal injection with the mSIRPα-Fc-CD40L chimeric protein, or anti-CD40 (clone FGK4.5), anti-CD47 (clone MIAP301), anti-CD20 (clone AIS1B12), anti-PD-1 (clone RMP1-14), or anti-CTLA4 (clone 9D9). All test agents were diluted in PBS and injected in volumes between 100 and 200 μL. All therapeutic antibodies are from BioXcell. Tumor volume (mm³) and overall survival was assessed throughout the time-course. Survival criteria included total tumor volume less than 1,700 mm³ with no sign of tumor ulceration. Complete responders, in which tumors established and were subsequently rejected, are listed in the appropriate figures. Cohorts of CT26 experimental mice were euthanized between days 11 and 13 for immune profiling in splenocytes and tumor tissue using flow cytometry. Tumors were excised from these mice and dissociated using a tumor dissociation kit (Miltenyi Biotec, catalog no. 130-096-730). Tumors were excised, combined with the dissociation reagent, and then minced with a razor. The resulting slurry was transferred to a 1.5 mL Eppendorf tube and placed in a 37° C. shaker for 30 minutes. During this period, the slurry was pipetted up and down every 10 minutes to break up larger pieces. Dissociated cells were homogenized through a 100-μm strainer to isolate tumor cells and infiltrating immune cells. Experimental group sizes are described in each figure legend and come from a minimum of two independent experiments.

For CD4, CD8, and IFNAR1 depletion experiments, mice were treated via intraperitoneal injection of 100 μg of anti-CD4 (clone GK1.5), 100 μg of anti-CD8 (clone 2.43), or 500 μg of anti-IFNAR1 (clone MAR1-5A3) on the schedules described in the appropriate figures (all antibodies are from BioXcell). Antibodies were diluted in PBS and injected in volumes of 100 μL. CD4, CD8, and IFNAR1 populations in the peripheral blood were assessed at several time points to verify depletion and were normalized to untreated mice. Peripheral blood from WEHI3-bearing mice treated on days 7, 9, and 11 with 300 μg SIRPα-Fc-CD40L±anti-IFNAR1 was collected, the RBCs lysed, and the resulting mononuclear cells (MNC) assessed by flow cytometry with fluorescently conjugated antibodies to CD45, CD11c, CD20, CD4, and CD8 (antibodies from BioLegend).

### Safety Studies

### Nonhuman Primate Studies.

Naïve cynomolgus macaques (2-4 years of age) were given SIRPα-Fc-CD40L by intravenous infusion every week, for 5 consecutive weeks, at doses of 0.1, 1, 10, and 40 mg/kg. Hematology and clinical chemistry parameters were collected by venipuncture before and after each dose. CD47 expression on circulating RBCs was assessed by flow cytometry. MNCs and serum were isolated from whole blood using Ficoll gradient separation. The resulting erythroid pellet was stained with anti-CD47 and pregated using forward/side scatter to isolate a majority RBC population. All studies were conducted at Charles River Laboratories in accordance with Institutional Animal Care and Use Committee (IACUC) guidelines.

### Experimental Animal Guidelines

All animal studies have been conducted in accordance with, and with the approval of an IACUC and reviewed and approved by a licensed veterinarian. Experimental mice were monitored daily and euthanized by CO₂ asphyxiation and cervical dislocation prior to any signs of distress.

### Statistical Analysis

GraphPad Prism was used to plot and generate all graphs throughout; as well as automatically calculate error and significance. Experimental replicates (N) are shown in figures and figure legends. Unless noted otherwise, values plotted represent the mean from a minimum of three distinct experiments and error is SEM.

Statistical significance (P value) was determined using unpaired t tests or one-way ANOVA with multiple comparisons. Significant P values are labeled with one or more “*”, denoting *, P<0.05; **, P<0.01; ***, P<0.001; and ****, P<0.0001. Mantel-Cox statistical tests were used to determine the significance between the survival curves. P values are noted in the legends to these figures.

### Example 2: Production and Characterization of the SIRPα-Fc-CD40L Chimeric Protein

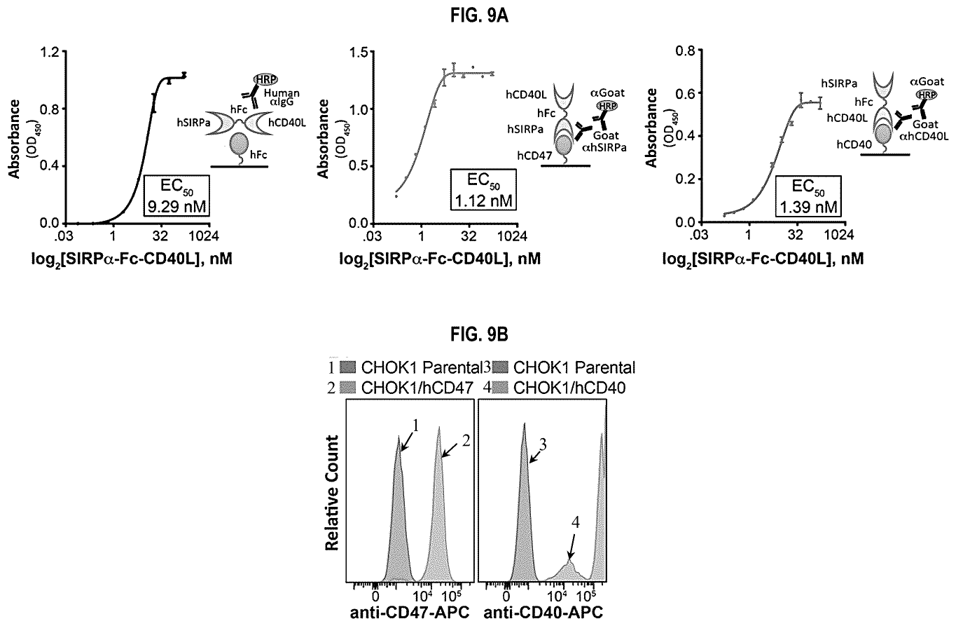

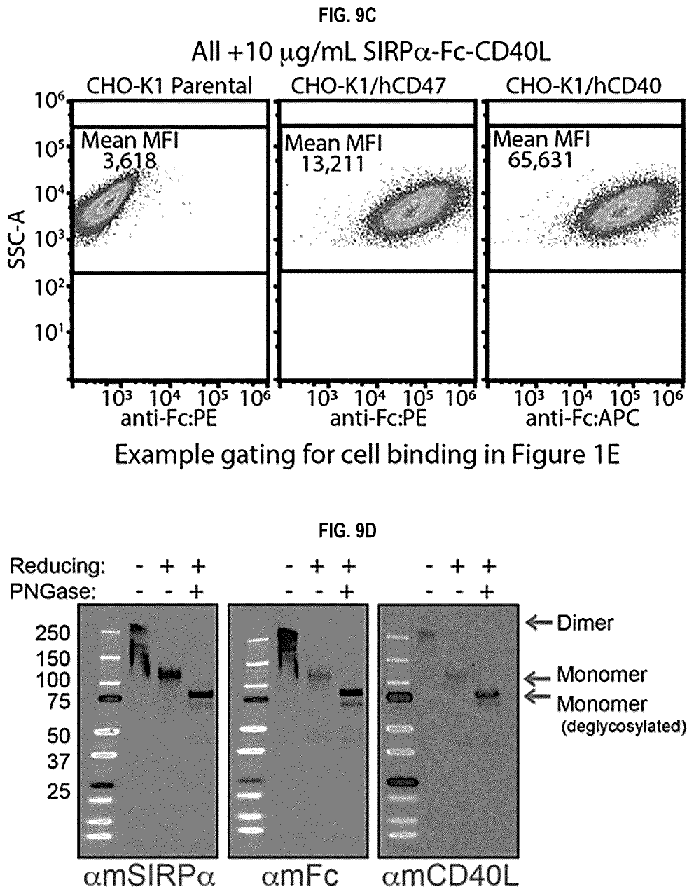

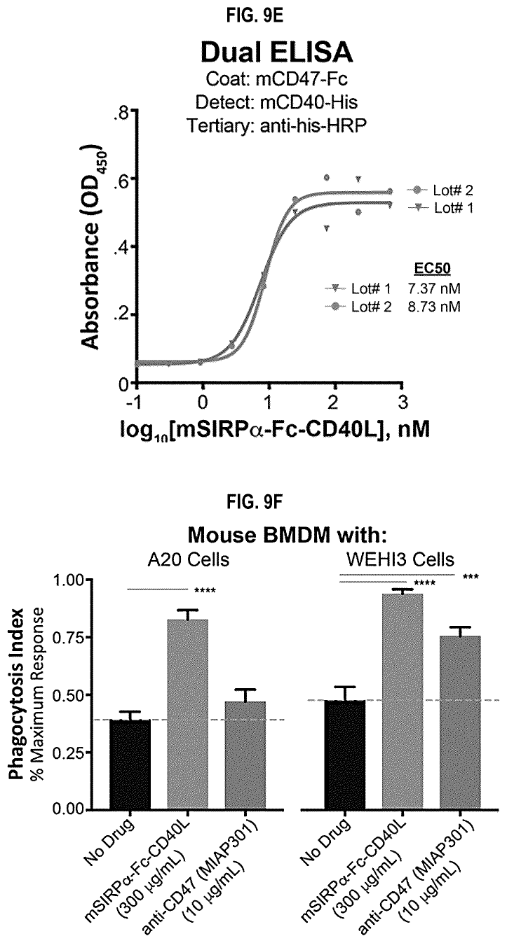

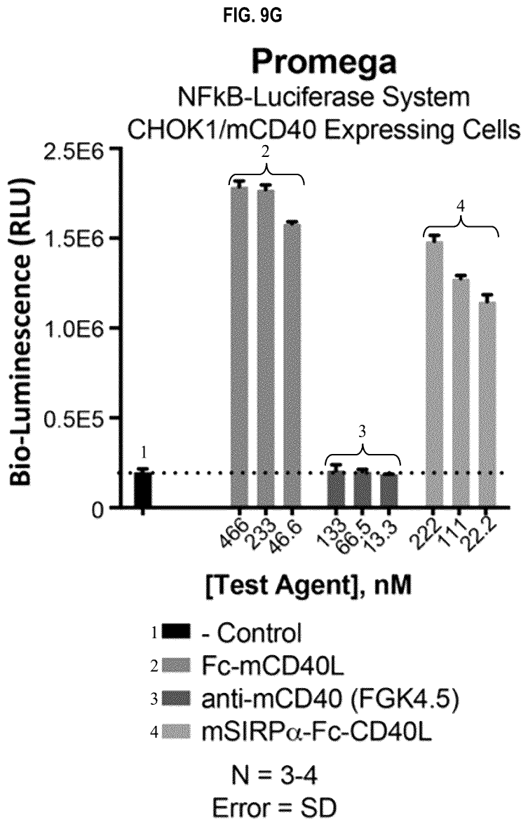

The ECDs of SIRPα and CD40L were fused via an antibody Fc domain for both human and mouse to generate SIRPα_{ECD}-Fc-CD40L_{ECD}; hereafter referred to as the SIRPα-Fc-CD40L chimeric protein. In silico structural modeling predicted that each individual domain of the adjoined construct would fold in accordance with the native molecules, suggesting preservation of both binding functions (FIG. 3A, top panel). Purified the SIRPα-Fc-CD40L chimeric protein was then analyzed for the presence of each individual domain by Western blotting using anti-SIRPα, anti-Fc, and anti-CD40L (FIG. 3A, bottom panel), revealing a glycosylated protein that formed a multimer under nonreducing conditions at the predicted monomeric molecular weight of 88.1 kDa. To further characterize the native state of the SIRPα-Fc-CD40L chimeric protein in the absence of detergents, electron microscopy was performed, demonstrating that the major peak fraction contained a hexameric species (≥60% for both mouse and human), consistent to what had been previously described for TNF ligand fusion proteins (FIG. 3B). Oberst et al. Potent immune modulation by MEDI6383, an engineered human OX40 ligand IgG4P Fc fusion protein. Mol Cancer Ther 17:1024-38 (2018). A minor peak fraction (˜30%-35%) was also present, which comprised a tetrameric protein complex that had equivalent activity in the dual-binding ELISA (hexamer EC50=24.21 nmol/L, tetramer EC50=33.3 nmol/L, reference=18.1 nmol/L). A dual-binding ELISA assay was developed to quantitatively demonstrate simultaneous binding of SIRPα to recombinant CD47 and CD40L to recombinant CD40 (FIG. 3C). Individual ELISAs also confirmed binding to recombinant human CD47, Fc, and CD40 (FIG. 9A). Binding affinity studies using surface plasmon resonance (SPR) were performed as described in Example 1. The results of these studies are shown in the Table below:

| | | | Ka | Kd | KD | |
|---|---|---|---|---|---|---|
| | | | (on-rate; | (off-rate; | (binding; | |
| | | Sample | 1/Ms) | 1/s) | M) | |
| SPR | CD47 | SIRPα-Fc | 1.31 E+6 | 2.28 E−2 | 17.6 | nM |
| Binding: | | SIRPα-Fc-CD40L | 2.92 E+5 | 1.84 E−4 | .628 | nM |
| | CD40 | CD40L-Fc | 3.04 E+5 | 3.51 E−4 | 1.15 | nM |
| | | SIRPα-Fc-CD40L | 8.18 E+4 | 4.57 E−4 | 4.74 | nM |
| | FcγR1A | IgG1 | 1.84 E+4 | 1.56 E−4 | 8.42 | nM |
| | | SIRPα-Fc-CD40L | ND | ND | ND | |
| | FcRn | IgG1 | 1.78 E+5 | 4.67 E−3 | 2.62 | nM |
| | | SIRPα-Fc-CD40L | 1.45 E+5 | 3.38 E−3 | 2.33 | nM |

As shown in the Table above, the binding affinity studies using SPR indicated that human the SIRPα-Fc-CD40L chimeric protein bound with 0.628 nmol/L affinity to recombinant human CD47, 4.74 nmol/L affinity to recombinant human CD40, had undetectable binding to FcγR1a, -2b and -3b, while having preserved 2.33 nmol/L binding affinity to FcRn (FIG. 3D). To justify testing of the human SIRPα-Fc-CD40L chimeric protein in nonhuman primates, high-affinity binding to recombinant cynomolgus macaque CD40 (3.24 nmol/L) and CD47 (1.7 nmol/L) were also confirmed. Finally, to confirm that the SIRPα-Fc-CD40L chimeric protein interacted with native CD47 and CD40 in a similar manner to recombinant CD47 and CD40, CHOK1-hCD47, and CHOK1-hCD40 reporter cell lines were developed (FIG. 9B and FIG. 9C). Flow cytometry studies using these reporter cell lines confirmed that the SIRPα-Fc-CD40L chimeric protein bound to CHOK1-CD47 cells (31.85 nmol/L EC₅₀) and CHOK1-CD40 cells (22.48 nmol/L EC₅₀), but not to parental CHOK1 cells (FIG. 3D, FIG. 9B and FIG. 9C). A functional ELISA demonstrated that the SIRPα-Fc-CD40L chimeric protein outcompeted a commercially available single-sided SIRPα-Fc control for binding to recombinant CD47, generating an EC50 of 22 nmol/L, comparable with the 14 nmol/L EC₅₀ produced by a commercial CD47-blocking antibody (FIG. 3E). FIG. 9D shows the western blot analysis of the murine SIRPα-Fc-CD40L surrogate with antibodies detecting mSIRPα, mFc, and mCD40L under non-reducing, reducing, and PNGase F/reducing conditions. FIG. 9E shows the dual functional ELISA of the murine SIRPα-Fc-CD40L surrogate, demonstrating simultaneous binding to recombinant mouse CD47 and CD40.

These results demonstrate that mouse and human the SIRPα-Fc-CD4L chimeric proteins were constructed. These protein specifically bound to their cognate receptor/ligands, and that the human SIRPα-Fc-CD40L chimeric protein bound to in nonhuman primates (cynomolgus macaque) CD40 and CD47 with high-affinities.

The 792 amino acid sequence of SL-172154 (not including the leader sequence) exists as a profile of oligomeric forms. There are 17 cysteines in the amino acid sequence with 8 likely disulfide pairs. Both N and O-linked glycosylation have been identified. The Table below provides the location of post-translational modifications (including glycosylation profile and disulfide bridges):

| Chain | Residue | Modification | Details if Appropriate |
|---|---|---|---|
| | N80 | G0F, non-glycosylated | Glycoforms detected |
| | N215 | G1FS1 | Glycoforms detected |
| | N240 | G0F-N, Man5 | Glycoforms detected |
| | N262 | G0F (major), Man5, | Glycoforms detected |
| | | G1SF1, G0F-N | |
| | N289 | G0F (major), Man5, | Glycoforms detected |
| | | G0F-N | |
| | N421 | G0F, G0F-N, Man5, G1F | Glycoforms detected |
| | N462 | G1F, Man5, G0F, G0F-N, | Glycoforms detected |
| | | also non-glycosylated | |
| | N771 | Man5 | Glycoforms detected |
| | N159 | Succinimide | Modification |
| | N283 | Succinimide | Modification |
| | N681 | Succinimide | Modification |
| | N682 | Succinimide | Modification |
| | M644 | Oxidation | Modification |
| | N688 | Succinimide | Modification |
| | N688 | Deamidation | Modification |

Glycosylation modifications along with oxidation, deamidation, and succinimidation were also detected.
Succinimide formation of Asni59 (2.6%) (Peptide T20)
Oxidation of Met644 (14.3%) (Peptide T71)
Succinimide formation of Asn283/Asn682/Asn688, residue (specific peptide could not be identified)
Deamidation of Asn688 (1.9% and 1.6%, respectively) (Peptide T74).

The Table below shows the peptide mapping via RP-UPLC-UV/MSE: summary of clipped peptides SL-172154 Reference Standard NB8670p33

| | | | Relative |
|---|---|---|---|
| Clip¹ | Peptide | Form¹ | Intensity (%) |
| W285/L286 clip | T30-31 G0F | unclipped | 90.5 |
| | | clipped (T30c9) | 9.5 |
| Y531/S532 clip | T53 | unclipped | 96.6 |
| | | clipped (T53c2) | 3.4 |
| Y701/1702 clip | T76-77 | unclipped | 97.0 |
| | | clipped (T77n5) | 3.0 |

¹A minimum intensity threshold of 3.0% of the most intense form of the unmodified peptide was set as a criterion for reporting clipped forms of the peptide.

The Table below shows de-mapping via RP-UPLC-UV/MSE: Summary of Modified Peptides SL-172154 Reference Standard N1B8670p33

| | | | Relative Intensity |
|---|---|---|---|
| Peptide | Modification¹ | Form | (%) |
| T10 | G0F (N80) | unmodified | 52.1 |
| | | modified | 47.9 |
| T20 | N-succ (N159) | unmodified | 97.4 |
| | | modified | 2.6 |
| T23 | G1FS1 (N215) | unmodified | — |
| | | modified | 100.0 |
| T25 | G0F-N (N240) | unmodified | — |
| | | modified | 100.0 |
| T25-26 | Man5 (N240) | unmodified | — |
| | | modified | 100.0 |
| T27-28 | — | unmodified | — |
| | G0F (N262) | modified | 81.4 |
| | Man5 (N262) | modified | 18.6 |
| T28 | — | unmodified | — |
| | G0F (N262) | modified | 71.4 |
| | G1FS1 (N262) | modified | 17.7 |
| | G0F-N (N262) | modified | 10.9 |
| T28-29 | — | unmodified | — |
| | G0F (N262) | modified | 61.3 |
| | G0F-N (N262) | modified | 22.9 |
| | Man5 (N262) | modified | 15.8 |
| T30 | — | unmodified | — |
| | G0F (N289) | modified | 73.2 |
| | Man5 (N289) | modified | 16.5 |
| | G0F-N (N289) | modified | 10.4 |
| T30-31 | — | unmodified | — |
| | G0F (N289) | modified | 60.8 |
| | G0F-N (N289) | modified | 25.6 |
| | Man5 (N289) | modified | 13.6 |
| T41-43 | — | unmodified | — |
| | G0F (N421) | modified | 74.4 |
| | G0F-N (N421) | modified | 16.2 |
| | Man5 (N421) | modified | 4.9 |
| | G1F (N421) | modified | 4.5 |
| T41-44 | G0F-N (N421) | unmodified | — |
| | | modified | 100.0 |
| T42-46 | Man5 (N421) | unmodified | — |
| | | modified | 100.0 |
| T43 | G0F (N421) | unmodified | — |
| | | modified | 100.0 |
| T43-44 | | unmodified | — |
| | G0F (N421) | modified | 92.3 |
| | Man5 (N421) | modified | 7.7 |
| T48-49 | — | unmodified | — |
| | G1F (N462) | modified | 64.5 |
| | Man5 (N462) | modified | 35.5 |
| T49 | — | unmodified (T49- | 64.9 |
| | | 50) | |
| | G0F (N462) | modified | 27.3 |
| | G0F-N (N462) | modified | 7.8 |
| T71 | M-ox (M644) | unmodified | 85.7 |
| | | modified | 14.3 |
| | — | unmodified | 96.5 |
| | N-deam | modified | 1.6 |
| T74 | (N688) | | |
| | N-succ (N681, | modified | 1.9 |
| | N682, | | |
| | N688) | | |
| T82-83c4 | Man5 (N771) | unmodified | — |
| | | modified | 100.0 |
| T82-83 | Man5 (N771) | unmodified | — |
| | | modified | 100.0 |
| T82-84 | Man5 (N771) | unmodified | — |
| | | modified | 100.0 |
| T83 | Man5 (N771) | unmodified | — |
| | | modified | 100.0 |

¹A minimum intensity threshold of 1.0% of the most intense form of the peptide was set as a criterion for reporting modified forms of the peptide.
²A dash (—) indicates a peptide that was not observed or was below the reporting threshold.

The Table below shows the summary of identified disulfide bonds in non-reduced digest:

Disulfide Bond(s)¹

| | | Scrambled | |
|---|---|---|---|
| Intrachain² | Interchain³ | Disulfide Bonds | Cysteinylation⁴ |
| C25 = C91 | C350 (chain1) = | C140 = C243 = | C749 = C |
| | C350 (chain2) | C709/C725 | |
| C140 = C198 | C353 (chain1) = | C615 (chain1) = | |
| | C353 (chain2) | C615 (chain2)³ | |
| C243 = C301 | | | |
| C385 = C445 | | | |
| C491 = C549 | | | |
| C603 = C615 | | | |
| C709 = C725 | | | |

¹Disulfide bond indicated by an equal sign (=).
²Disulfide bonds formed between Cys residues within the same protein chain.
³Disulfide bonds formed between Cys residues within different protein chains (chain1 and chain2).
⁴Cysteinylation due to the formation of a disulfide bond between a Cys residue of SL-172154 and a free Cys molecule.

The results from the reduced digest were searched for the presence of alkylated Cys residues, as a marker of free Cys. The alkylating reagent (IAM) was added to the sample prior to digestion to cap any free Cys residues. Alkylated Cys was observed on residues Cys709 (5.6%), C725 (4.3% and 3.4%) and Cys749 (49.9%). Two scrambled disulfide bonds were detected, {C140=C243=C709/C725} and {C615 chain1=C615 chain 2}

### Example 3: Functional Activity of the SIRPα Domain of the SIRPα-Fc-CD40L Chimeric Protein

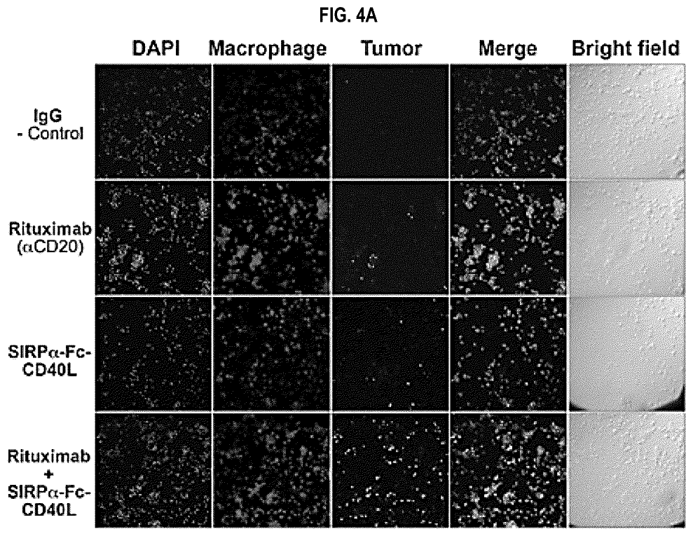

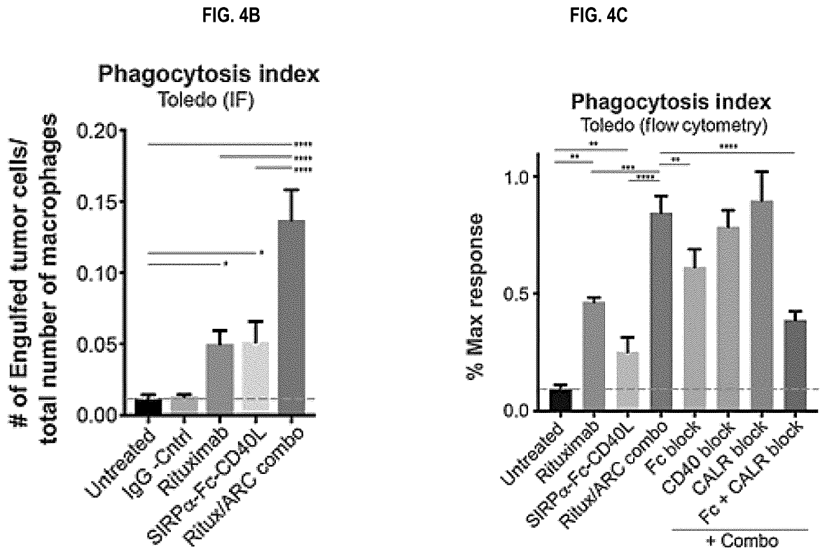

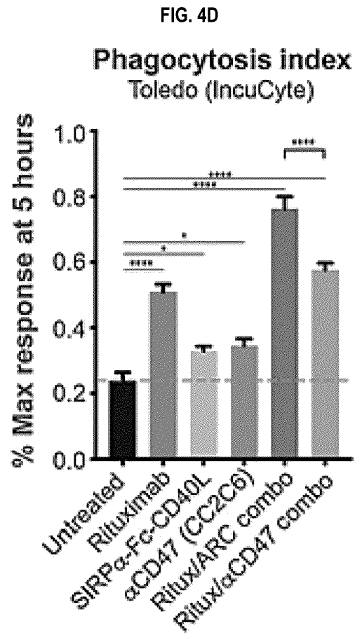

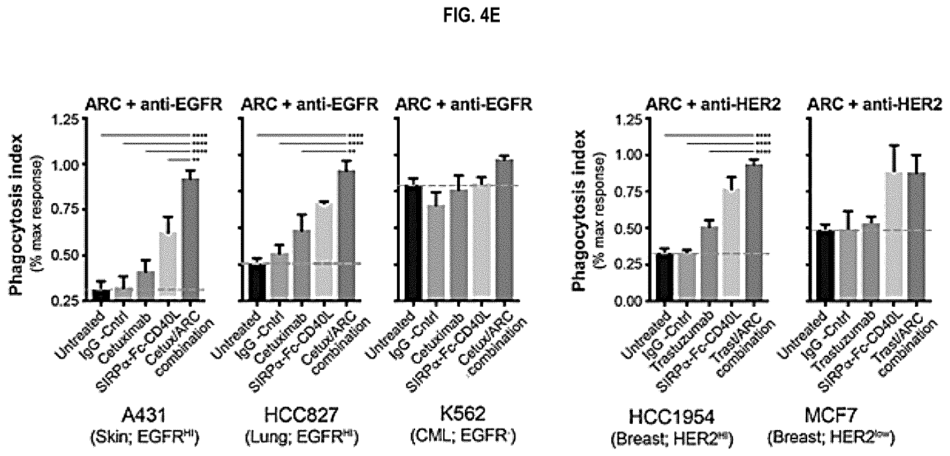

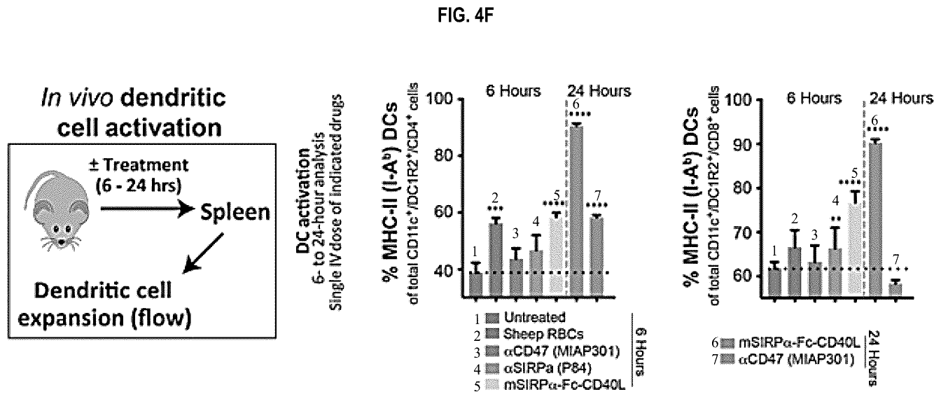

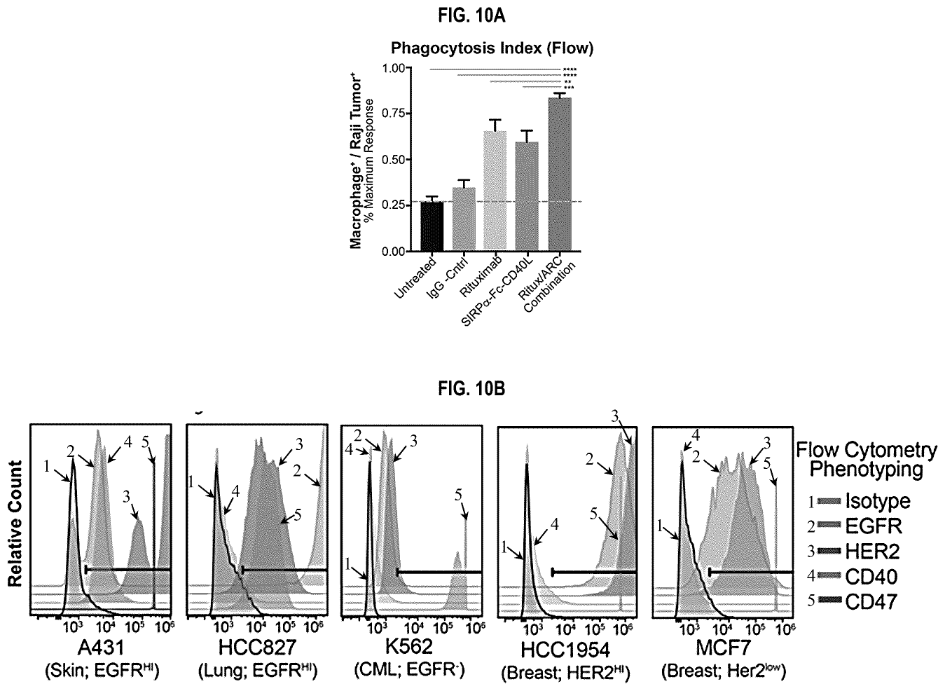

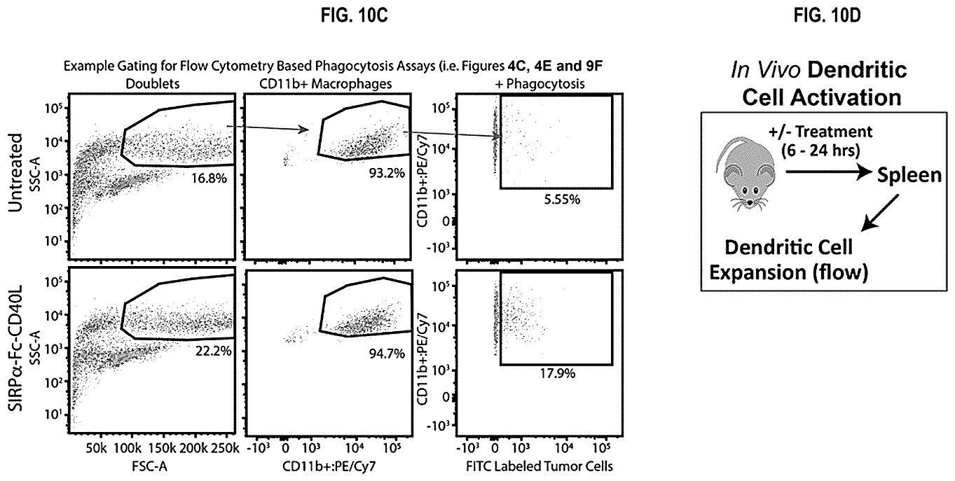

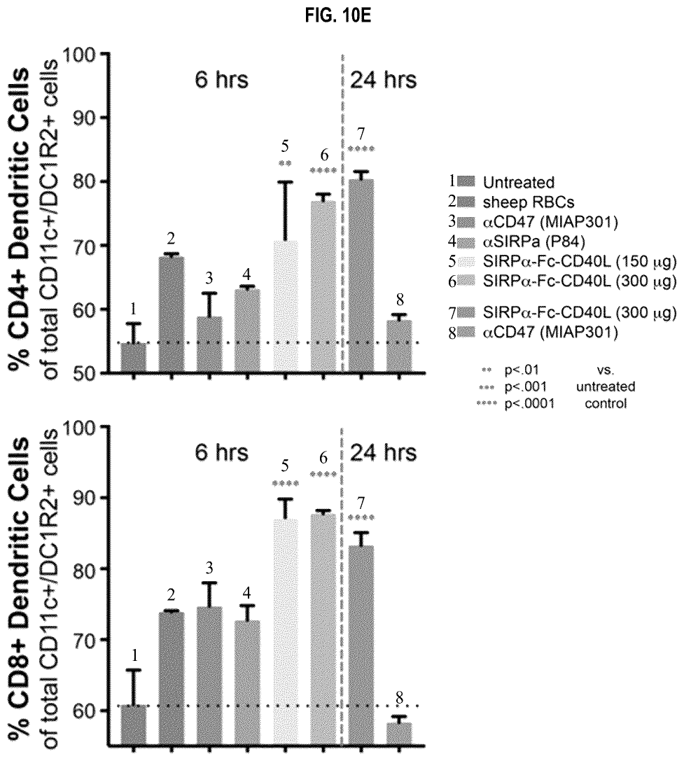

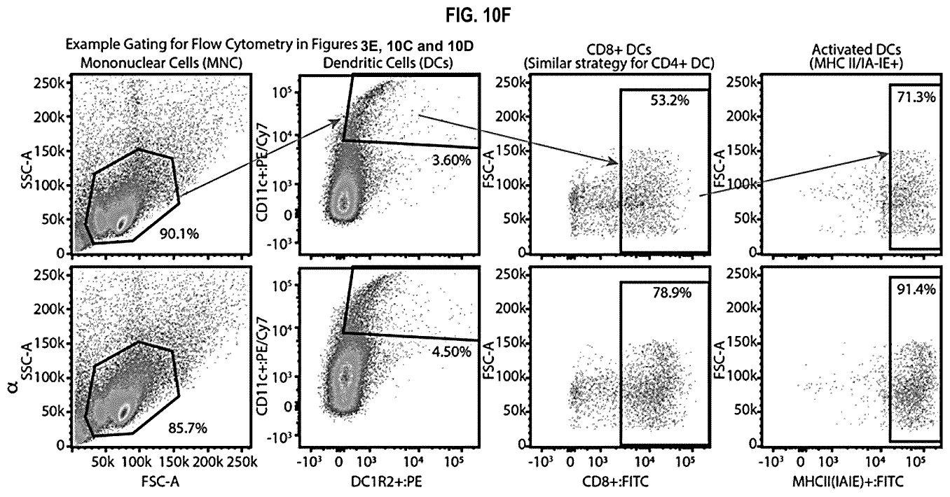

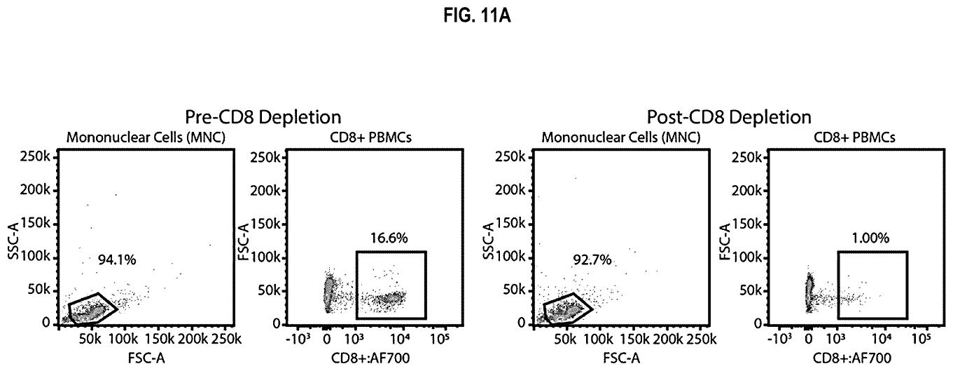

Given the oligomeric nature of SIRPα-Fc-CD40L, independently characterization of the functionality of both the SIRPα and CD40L domains was performed. Because of the bifunctionality of the SIRPα-Fc-CD40L chimeric protein, distinct functional assays were utilized to independently characterize the activity of both the SIRPα and CD40L domains. A common in vitro assay used to characterize the SIRPα/CD47 axis analyzes the ability of purified macrophages to phagocytose tumor cells (Oldenborg et al., Role of CD47 as a marker of self on red blood cells. Science 288:2051-2054 (2000); Gardai et al., Cell-surface calreticulin initiates clearance of viable or apoptotic cells through trans-activation of LRP on the phagocyte. Cell 123:321-334 (2005)), particularly in the presence of ADCP-competent therapeutic antibodies. Chao et al., Anti-CD47 antibody synergizes with rituximab to promote phagocytosis and eradicate non-Hodgkin lymphoma. Cell 142:699-713 (2010). Accordingly, in vitro tumor cell phagocytosis assays were established to determine whether the SIRPα-Fc-CD40L chimeric protein enhanced macrophage-mediated phagocytosis of various tumor cell lines both alone and in combination with targeted antibodies. Initially, several CD20+ lymphoma (Toledo, Raji, Ramos) cell lines were cocultured with human monocyte-derived macrophages to identify a suitable platform for assessing phagocytosis; using three analogous approaches; (i) immunofluorescence (IF); to visualize the overlap in signal between labeled macrophages and labeled tumor cells, (ii) flow cytometry; to quantitate distinct populations of dually-positive tumors and macrophages, and (iii) live-cell imaging using the IncuCyte platform; to both visualize and quantitate macrophage-mediated phagocytosis of tumor cells-using tumor cells that were precoated with a pH-sensitive marker (pHrodo) that only fluoresces when a tumor cell is engulfed by a macrophage and enters a low pH (pH 4.5-5.5) phagosome (FIG. 4A to FIG. 4E, and FIG. 10A). All three approaches demonstrate that the SIRPα-Fc-CD40L chimeric protein was capable of inducing phagocytosis as a monotherapy, and that this activity was significantly enhanced in CD20+ lymphoma cells lines when combined with the antibody-dependent cell-mediated phagocytosis (ADCP)-competent anti-CD20 agent, rituximab (FIG. 4A to FIG. 4D, and FIG. 10A). The phagocytosis-stimulating activity of the SIRPα-Fc-CD40L chimeric protein/rituximab combinations was partially inhibited when Fc receptors were blocked on macrophages; however, activity was not impacted when macrophages were pretreated with a CD40-blocking antibody indicating that binding of the SIRPα-Fc-CD40L chimeric protein to CD40 does not contribute to phagocytosis of tumor cells.

Calreticulin on tumor cells serves as a prophagocytic signal, facilitating tumor cell phagocytosis following blockade of the CD47/SIRPα pathway. Chao et al., Calreticulin is the dominant pro-phagocytic signal on multiple human cancers and is counterbalanced by CD47. Sci Transl Med 2:63ra94 (2010). It was demonstrated that both calreticulin and Fc receptor engagement was required for efficient phagocytosis of CD20+ B-cell lymphoma cells by the combination of the SIRPα-Fc-CD40L chimeric protein and rituximab using a calreticulin-blocking peptide (CALR), confirming the importance of Fc interactions for Fc-competent targeting antibodies and providing evidence that the initiation of phagocytosis by the SIRPα-Fc-CD40L chimeric protein was driven by the SIRPα domain (FIG. 4C). A similar monotherapy phagocytosis activity of the SIRPα-Fc-CD40L chimeric protein was observed to the CC2C6 clone of anti-CD47 (FIG. 4D). When both agents were combined with rituximab, the SIRPα-Fc-CD40L chimeric protein/rituximab combination stimulated significantly greater phagocytosis activity than the anti-CD47/rituximab combination suggesting competition by two separate therapeutics that require Fc receptor engagement. The phagocytic activity was examined in a range of human tumor cell lines using several ADCP-targeted antibodies; EGFR+ melanoma (A431 cells) and lung (HCC827 cells), EGFR-chronic myelogenous leukemia (CML; K562 cells), and HER2+ breast (HCC1954HER2 HI and MCF7HER2 LOW cells) were used to facilitate combinations with EGFR− (cetuximab) and HER2− (trastuzumab) targeted antibodies. Consistent with the lymphoma cell lines, monotherapy the SIRPα-Fc-CD40L chimeric protein-stimulated macrophage phagocytosis, which was enhanced in combination with targeted antibodies (FIG. 4E, FIG. 10B). Trastuzumab did not induce phagocytosis in the HER2^{LOW} cell line MCF7; however, the SIRPα-Fc-CD40L chimeric protein demonstrated modest monotherapy activity. A phagocytic activity was not observed with EGFR-negative K562 cells with monotherapy or combinations of the SIRPα-Fc-CD40L chimeric protein with cetuximab (FIG. 4E). Similar phagocytic activity was observed using the mouse surrogate, the mSIRPα-Fc-CD40L chimeric protein, to treat cocultures of BMDMs and either A20 or WEHI3 cells (FIG. 9F). Interestingly, a higher phagocytosis index was generated using the mSIRPα-Fc-CD40L chimeric protein, as compared with a mouse CD47-blocking antibody.

Finally, using an in vivo mouse assay that examines the activation status of splenic DCs in response to SIRPα/CD47 inhibitors or sheep RBCs (Keating, Rituximab: a review of its use in chronic lymphocytic leukaemia, low-grade or follicular lymphoma and diffuse large B-cell lymphoma. Drugs 70(11):1445-76) (2010)), it was observed that intravenous administration of sheep RBCs, CD47 blocking antibodies, or SIRPα blocking antibodies all stimulated upregulation of both activated CD4+ and CD8α+ DCs that were positive for MHC-II within 6 hours (FIG. 4F, FIG. 10D to FIG. 10F). Similarly, administration of the mouse SIRPα-Fc-CD40L chimeric protein also upregulated surface expression of MHC-II, CD80, and CD86 on splenic CD4+ and CD8α+ in a higher proportion of overall splenic DCs than was observed in the antibody-treated groups.

Collectively, these data demonstrated that the SIRPα domain of the SIRPα-Fc-CD40L chimeric protein functioned as expected by binding to CD47 with high affinity and potentiating macrophage-mediated phagocytosis alone and in the presence of multiple ADCP-competent antibodies.

### Example 4: Functional Activity of the CD40L Domain of the SIRPα-Fc-CD40L Chimeric Protein

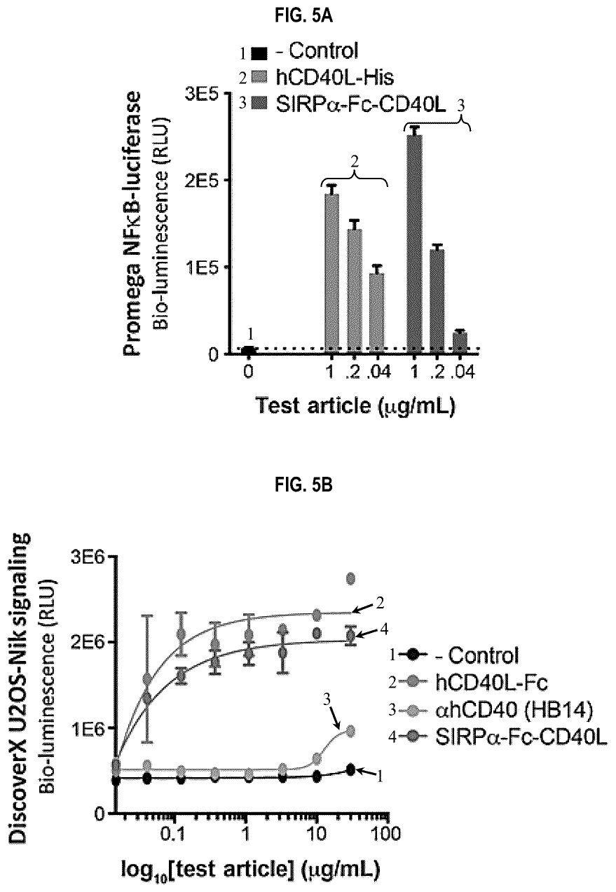

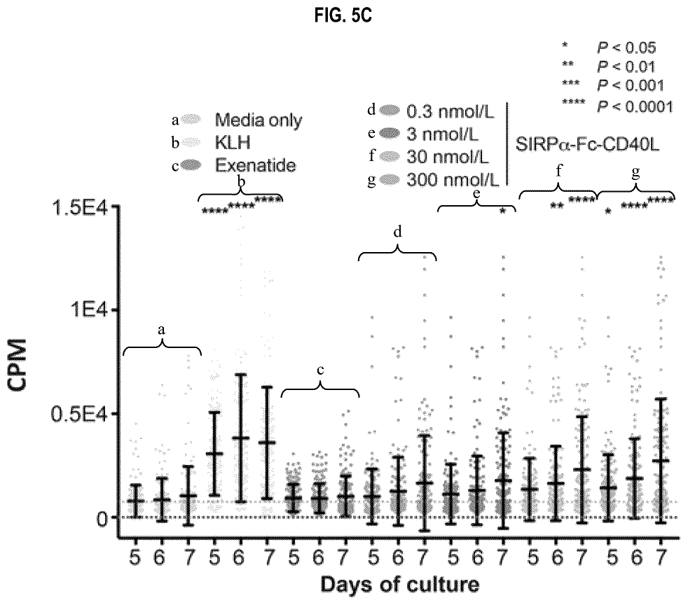

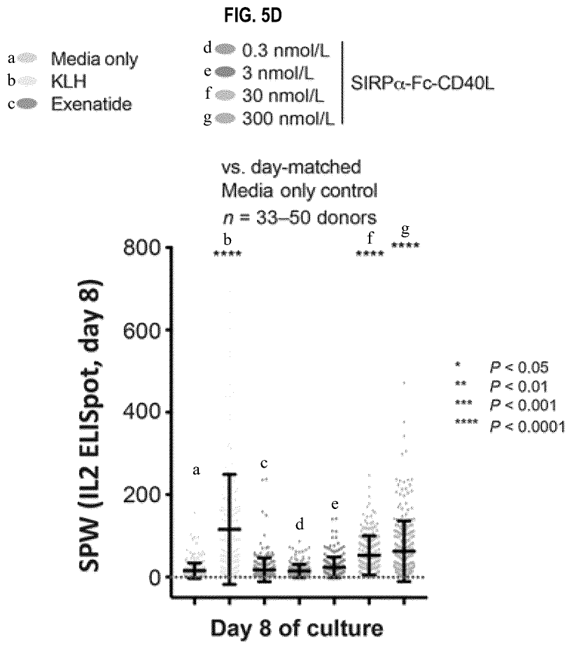

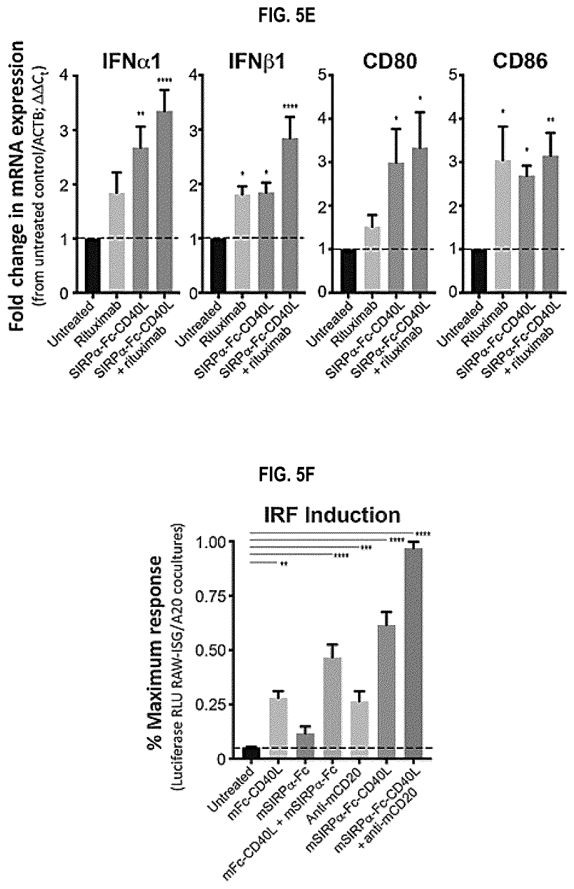

On the basis of the reported role of NIK signaling for CD40-dependent cross-priming, functionality of the CD40L domain of the SIRPα-Fc-CD40L chimeric protein was evaluated using two different CD40-dependent NFκB/NIK reporter systems (FIG. 5A and FIG. 5B). Senter and Sievers, The discovery and development of brentuximab vedotin for use in relapsed Hodgkin lymphoma and systemic anaplastic large cell lymphoma. Nat Biotechnol 30(7):631-637 (2012); de Weers et al., Daratumumab, a novel therapeutic human CD38 monoclonal antibody, induces killing of multiple myeloma and other hematological tumors. J Immunol 186(3):1840-1848 (2011). These data indicated that the SIRPα-Fc-CD40L chimeric protein had similar activity to a single-sided CD40L fusion protein in both reporter systems. The SIRPα-Fc-CD40L chimeric protein was present in a soluble form in both assays, and no Fc receptor or other cross-linking agents were present. Along these lines, a CD40 agonist antibody was unable to stimulate NIK/NFκB activity in the same system in the absence of an accessory cell that can provide Fc receptor engagement (FIG. 5B). These data indicated that the SIRPα-Fc-CD40L chimeric protein can stimulate CD40 signaling in the absence of cross-linking, likely due to its inherent hexameric configuration. Similar observations were made using the mSIRPα-Fc-CD40L chimeric protein in comparison with a mouse CD40 agonist antibody (FIG. 9G).

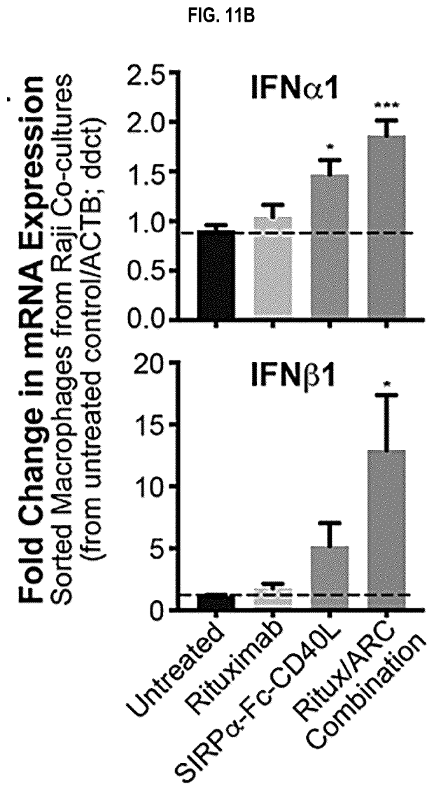

The observation that the SIRPα-Fc-CD40L chimeric protein stimulated CD40 signaling prompted investigation of other cellular functions that depend on CD40 signaling. CD40 stimulates proliferation of B cells and CD4+ T cells from human PBMCs in the presence of cross-linked anti-CD40 antibodies or CD40L. Valle et al., Activation of human B lymphocytes through CD40 and interleukin 4. Eur J Immunol 19:1463-1467 (1989). Cayabyab et al., CD40 preferentially costimulates activation of CD4+T lymphocytes. J Immunol 152:1523-1531 (1994). To investigate this readout, CD8+ T-cell-depleted PBMCs were isolated from a total of 33-50 different human donors and cultured in the presence of a dose titration of the SIRPα-Fc-CD40L chimeric protein (FIG. 5C, FIG. 5D and FIG. 11A). As compared with a media-only negative control and a Keyhole limpet hemocyanin (KLH)-positive control, soluble the SIRPα-Fc-CD40L chimeric protein stimulated dose-dependent proliferation of human PBMCs over a 7-day culture (FIG. 5C), and a dose-dependent increase in the number of IL2-secreting PBMC on day 8 of the culture (FIG. 5D), previously reported as a downstream event of CD40 activation. Kindler et al., Interleukin-2 secretion by human B lymphocytes occurs as a late event and requires additional stimulation after CD40 cross-linking. Eur J Immunol 25:1239-1243 (1995).

The activation status of macrophages from the SIRPα-Fc-CD40L chimeric protein-treated macrophage Toledo lymphoma cell cocultures was assessed by qRT-PCR for expression of the type I IFN-regulatory genes IFNα1 and IFNβ1, and the macrophage activation markers CD80 and CD86 (FIG. 5E). Monotherapy with the SIRPα-Fc-CD40L chimeric protein and rituximab (anti-CD20) induced macrophage activation and the expression of type I IFN genes in the isolated macrophages, which was enhanced when the two agents were combined. Similar results were observed in macrophages isolated after coculture with Raji cells in the presence of the SIRPα-Fc-CD40L chimeric protein, rituximab, or the combination (FIG. 11B).

A macrophage reporter system (RAW264.7 ISG) was utilized to determine activation of a type I IFN response by the mSIRPα-Fc-CD40L chimeric protein. When RAW264.7 ISG cells were cocultured with A20 lymphoma cells, the mSIRPα-Fc-CD40L chimeric protein stimulated an increase in IFN gene-driven luciferase activity (FIG. 5F). Commercially available recombinant murine Fc-CD40L was also able to stimulate IFN production; however, no significant signal was observed using a recombinant single-sided SIRPα-Fc protein indicating that type I IFN-activation acted downstream and independently from tumor cell phagocytosis, through CD40 engagement (FIG. 5F). A murine rituximab surrogate (anti-CD20) induced some monotherapy IFN response and significantly amplified the monotherapy signal seen with the mSIRPα-Fc-CD40L chimeric protein (FIG. 5F) providing additional rationale for the combination of the SIRPα-Fc-CD40L chimeric protein with targeted ADCP-competent antibodies. Such combinations may have had the ability to increase tumor cell phagocytosis and initiate pathways capable of activating APC and enhancing antigen processing/presentation. These data indicated that the magnitude of the type I IFN response could be enhanced when SIRPα and CD40L were physically linked to one another.

Collectively, these data demonstrate that the CD40L domain of the SIRPα-Fc-CD40L chimeric protein activated both canonical and noncanonical NFκB signaling, and stimulated human PBMC proliferation and IL2 secretion in vitro. CD47 blockade induces a type I IFN response following uptake of tumor mitochondrial DNA, leading to effective antitumor immunity.

While in vitro assays tend to favor the biology of either the SIRPα or CD40L domains (i.e., NFκB reporter assays primarily inform on CD40 activation, whereas macrophage phagocytosis assays primarily inform on SIRPα/CD47 blockade), in vivo studies provided a more complete view of the overall functionality of the construct. As demonstrated herein, the treatment with SIRPα-Fc-CD40L chimeric protein significantly improved rejection of both primary and secondary tumors as compared with individual antibodies targeting CD40 and CD47 used alone or in combination. The observation of enhanced antitumor immunity cannot be fully explained by the AH1-tetramer-positive CD8 T-cell population; however, it is possible that other clonotypes were activated that were not detected by the AH1 tetramer, which is consistent with the observation that CD8−depletion eliminated antitumor immunity. It is also possible that the cytolytic activity of individual tumor specific CD8+ T-cell clones was enhanced, which is under continued investigation.

Further, additional evidence for SIRPα-Fc-CD40L bridging an innate and adaptive immune response was provided by the observation that antitumor immunity was dependent both on type I IFNs and T cells in vivo. The T-cell-mediated immune responses downstream of CD47/SIRPα blockade may have been restrained by immune checkpoints. Accordingly, the sequencing of these antibodies with SIRPα-Fc-CD40L had a dramatic influence on the control and rejection of established tumors. Pretreatment with anti-CTLA4 or anti-PD-1 increased the proportion of CD40+ DCs and B cells within tumor-infiltrating leukocytes, providing possible mechanistic insight that could explain the antitumor benefit when CD40L was also present.

### Example 5: Antitumor Activity of the Murine SIRPα-Fc-CD40L Chimeric Protein

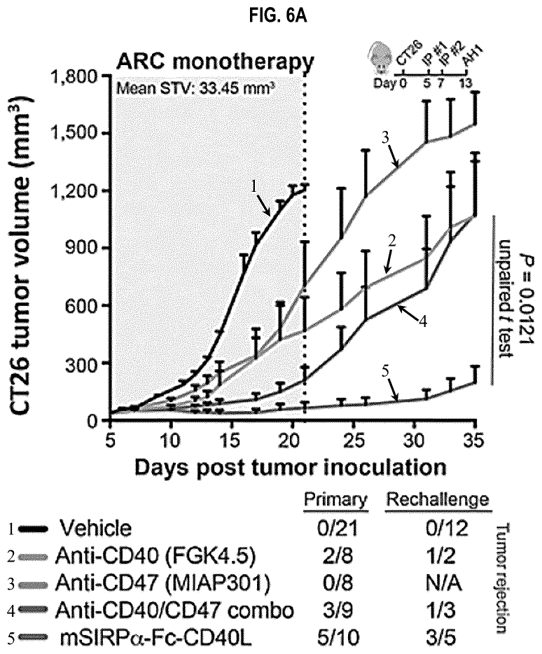

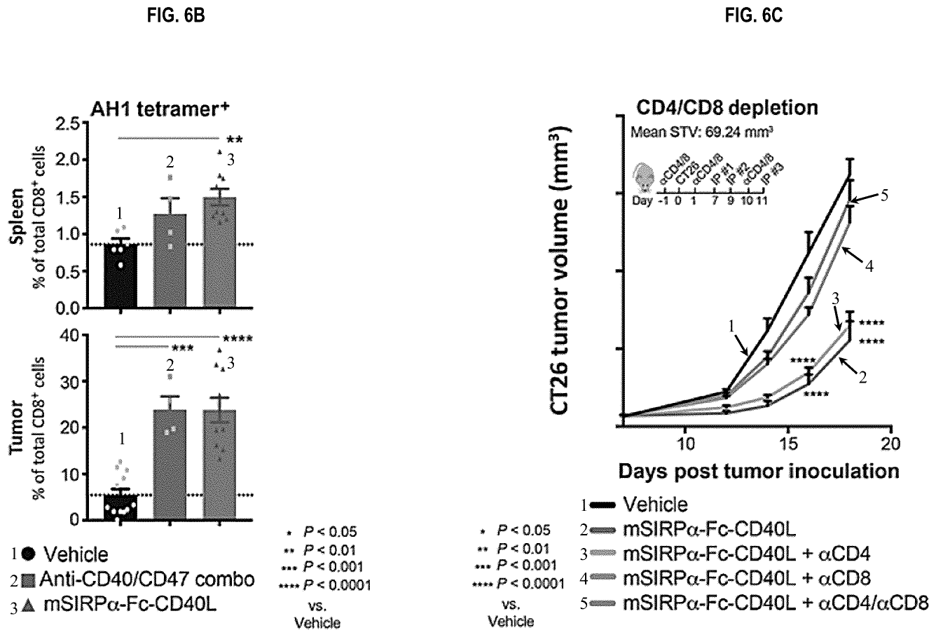

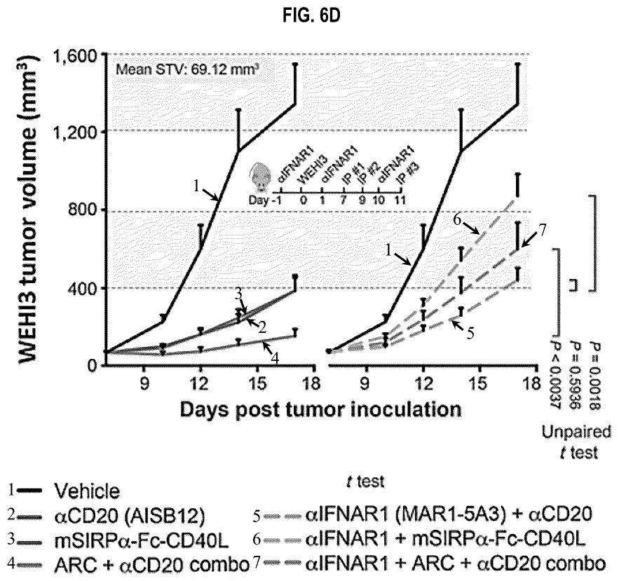

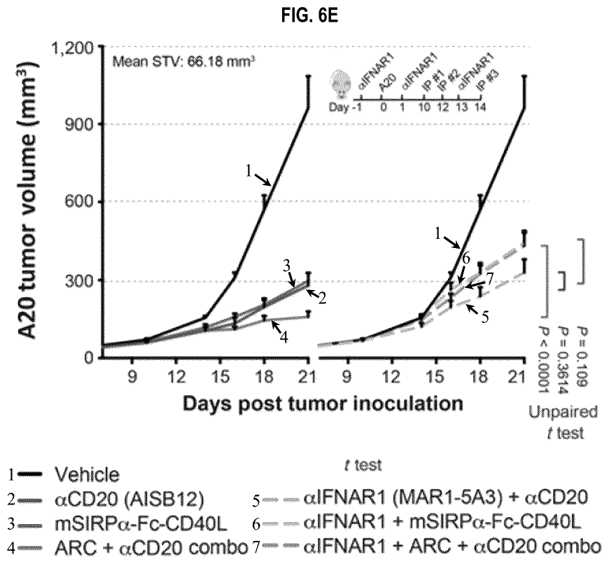

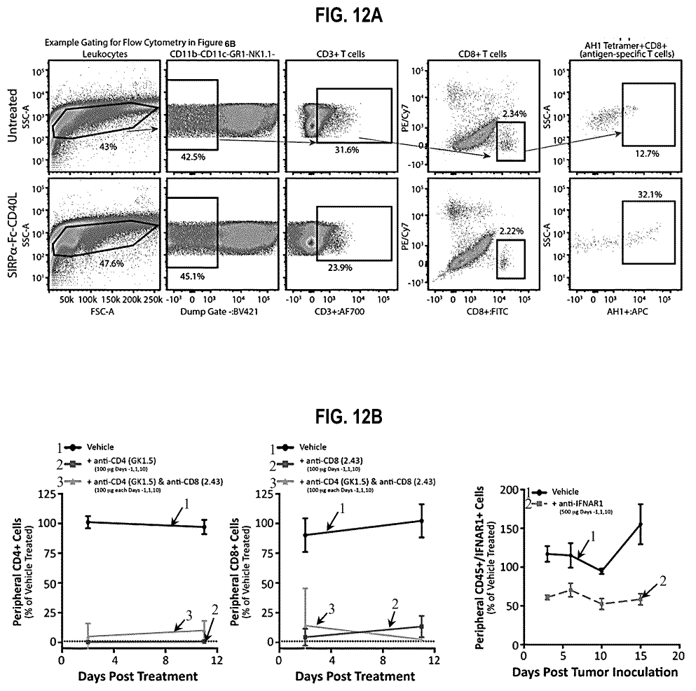

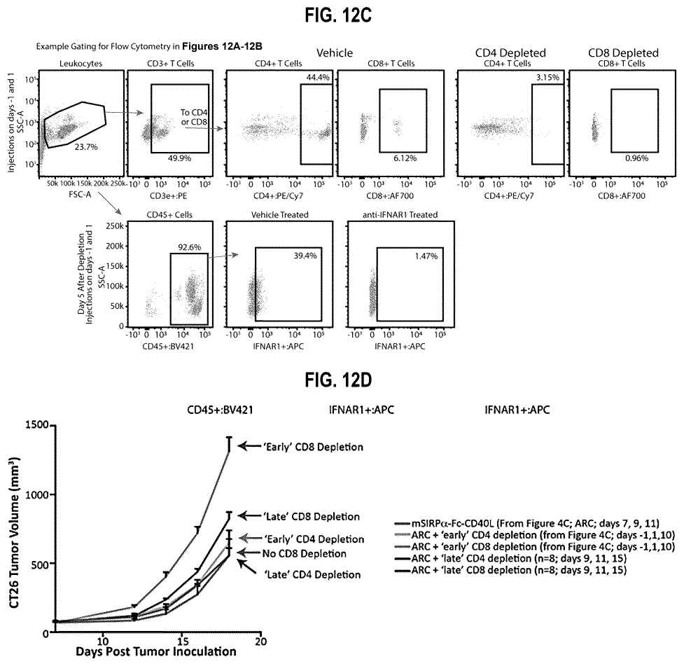

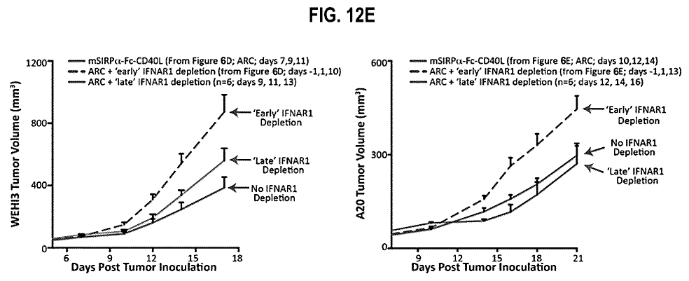

The syngeneic CT26 colon tumor model was used to provide an initial assessment of the antitumor activity of murine the SIRPα-Fc-CD40L chimeric protein in comparison with CD40 agonist and CD47-blocking antibodies. Implanted CT26 tumors were grown to approximately 30 mm³ before treatment was initiated with a fixed regimen of two doses of either CD40 agonist antibody (clone FGK4.5), CD47-blocking antibody (clone MIAP301), a combination of both antibodies or murine the SIRPα-Fc-CD40L chimeric protein. As compared with vehicle controls, both CD40 agonist and CD47-blocking antibodies provided moderate extensions in tumor growth, with 25% of mice completely rejecting primary tumors in the CD40 agonist monotherapy group (FIG. 6A). Mice treated with a combination of CD40 and CD47 antibodies were observed to have a longer delay in tumor outgrowth and 33% of mice rejected tumors. In comparison with the antibody groups, complete tumor rejection was observed in 50% of the mice treated with the mSIRPα-Fc-CD40L chimeric protein, along with significant tumor growth delay and prolonged survival (Mantel-Cox test, P=0.0364 vs. anti-CD40/anti-CD47 combination group) in the remaining mice. A majority of the mice that rejected the primary tumor rejected a secondary tumor challenge in the absence of additional treatment (60%; FIG. 6A). In both the antibody combination and the mSIRPα-Fc-CD40L chimeric protein-treated groups, there was an increase in the proportion of AH1-tetramer (specific for the MHC H-2Ld-restricted immunodominant epitope of gp70 expressed by the CT26 tumor cell line) CD8+ T cells in both the tumor and spleen (FIG. 6B and FIG. 12A). To determine whether these T-cell responses contributed to therapeutic efficacy, these studies were repeated in CD4+ and CD8+ T-cell antibody-depleted mice (FIG. 6C; FIG. 11A, and FIG. 12B to FIG. 12D). CD4+ T cells were partially required for therapeutic efficacy, whereas the loss of CD8+ cells completely eliminated the therapeutic benefit of the mSIRPα-Fc-CD40L chimeric protein, similar to CD47-blocking antibodies. Liu et al, CD47 blockade triggers T cell-mediated destruction of immunogenic tumors. Nat Med 21:1209-1215 (2015); Xu et al., Dendritic cells but not macrophages sense tumor mitochondrial DNA for cross-priming through signal regulatory protein alpha signaling. Immunity 47:363-373 (2017). CD8 depletion following the initiation of the mSIRPα-Fc-CD40L chimeric protein therapy partially abrogated the observed antitumor efficacy (FIG. 12D).

CD47-blocking antibodies plus rituximab potentiate macrophage-mediated phagocytosis, correlating to the combination's promising clinical efficacy in patients with late-stage diffuse large B-cell lymphoma. Advani et al., CD47 Blockade by Hu5F9-G4 and rituximab in non-Hodgkin's lymphoma. N Engl J Med 379:1711-1721 (2018); Sikic et al., First-in-human, first-in-class phase I trial of the anti-CD47 antibody Hu5F9-G4 in patients with advanced cancers. J Clin Oncol 37:946-53 (2019). Because the SIRPα-Fc-CD40L chimeric protein potentiated the activity of rituximab, the combination of the SIRPα-Fc-CD40L chimeric protein with a murine surrogate for rituximab (anti-mouse CD20; clone AISB12) was investigated in two CD20+ mouse tumor models, WEHI3 and A20. In both tumor models, similar control of established tumor growth was observed when anti-CD20 antibodies or the mSIRPα-Fc-CD40L chimeric protein were tested as monotherapies (FIG. 6D and FIG. 6E). An additive control of tumor growth was observed when anti-CD20 antibodies and the mSIRPα-Fc-CD40L chimeric protein were combined as compared with anti-CD20 monotherapy in A20 (P=0.0302) and WEHI (P=0.0166) or the mSIRPα-Fc-CD40L chimeric protein monotherapy in A20 (P=0.0017) and WEHI (0.0095). Next, it was sought to explore the previous in vitro findings implicating the SIRPα-Fc-CD40L chimeric protein in type I IFN activation in an in vivo setting and assessed the impact of an IFNα receptor 1 (IFNAR1) blocking antibody on antitumor efficacy. Antibody-mediated blockade of IFNAR1 significantly reduced the efficacy of the mSIRPα-Fc-CD40L chimeric protein both alone and in combination with anti-CD20 in mice bearing established WEHI3 tumors (FIG. 6D and FIG. 6E, FIG. 12C and FIG. 12E). IFNAR1 blockade most significantly impacted the mSIRPα-Fc-CD40L chimeric protein-treated groups, with less effect on anti-CD20 monotherapy-treated mice. Consistent with this observation, tumor control was similar between anti-CD20 monotherapy and in combination with the mSIRPα-Fc-CD40L chimeric protein, indicating that a majority of the combinatorial benefit required a functional type I IFN response. When depletion of IFNAR1 was initiated following initial treatment with the mSIRPα-Fc-CD40L chimeric protein, there was only marginal (WEHI3) or no (A20) acceleration in tumor growth, indicating that IFNAR1 signaling functions very early following treatment with the mSIRPα-Fc-CD40L chimeric protein (FIG. 12E).

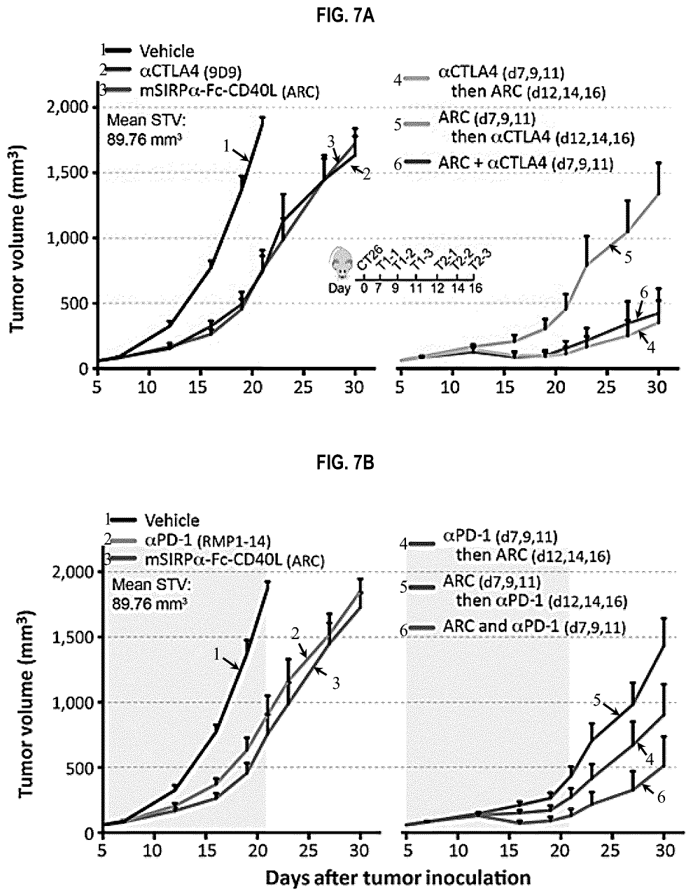

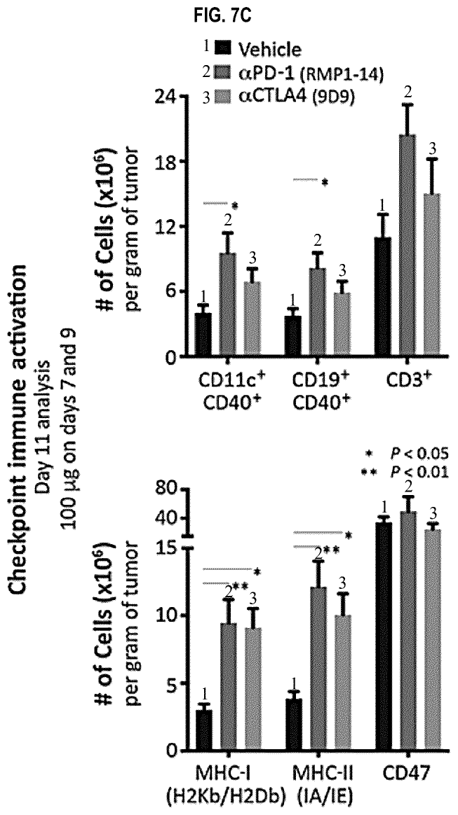

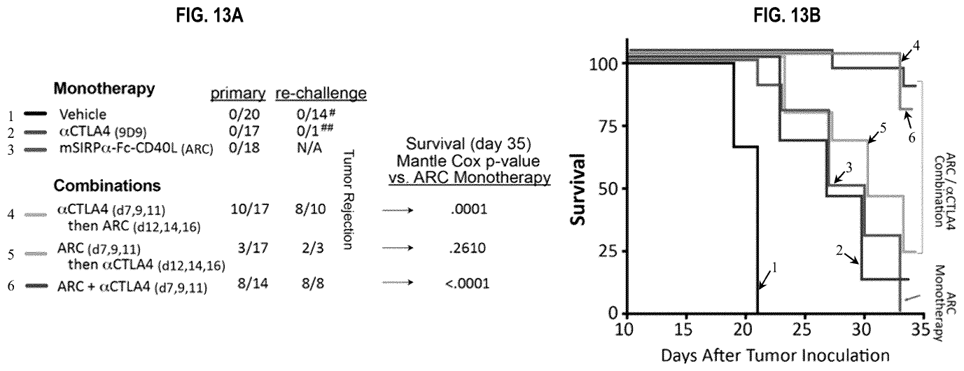

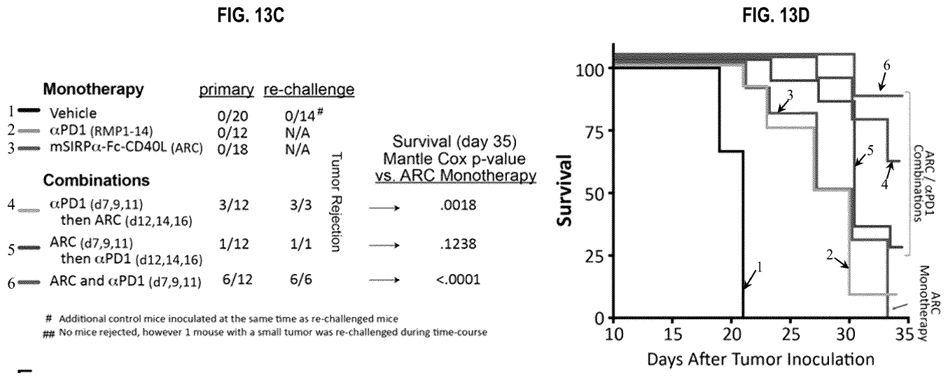

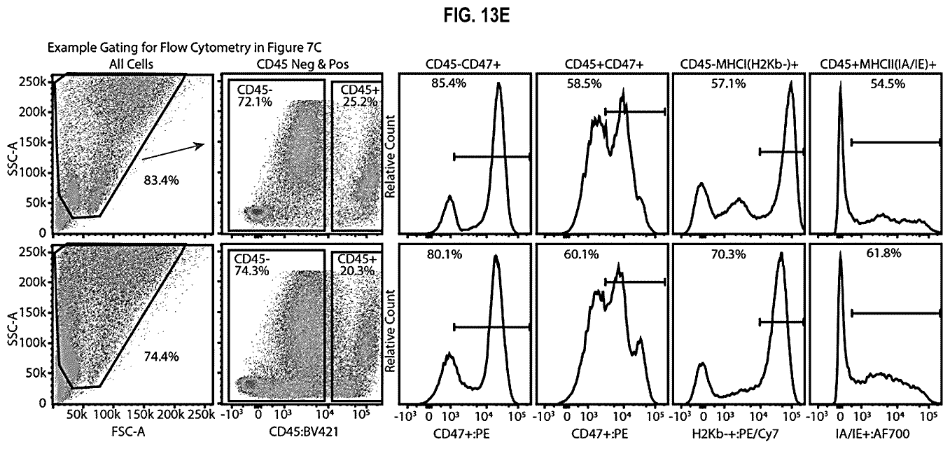

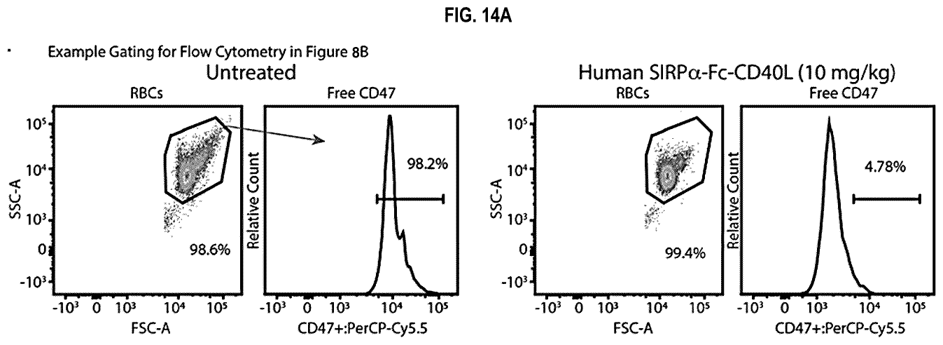

As the efficacy of the SIRPα-Fc-CD40L chimeric protein was immune-mediated, it was hypothesized, without being bound by theory, that CTLA4- and PD-1-blocking antibodies may improve the antitumor effects of the SIRPα-Fc-CD40L chimeric protein. To study these combinations, CT26 tumors grown to approximately 89 mm³ were treated with a fixed regimen of three doses of the mSIRPα-Fc-CD40L chimeric protein combined with three doses of CTLA4- or PD-1-blocking antibodies in different sequences (FIG. 7A and FIG. 7B). The larger starting tumor volumes were selected so that the monotherapy activity of both treatments would be reduced to observe additive or synergistic effects of the combination, the mSIRPα-Fc-CD40L chimeric protein had similar antitumor efficacy to anti-CTLA4 or anti-PD-1 antibodies (FIG. 7A and FIG. 7B); however the SIRPα-Fc-CD40L chimeric protein antitumor efficacy was significantly improved when given together with anti-CTLA4 (57% rejection), together with anti-PD-1 (50% rejection), following anti-CTLA4 (58.8% rejection), or following anti-PD-1 (25% rejection; FIG. 5A, FIG. 5B, and FIG. 13A to FIG. 13D). Nearly all of the mice that rejected the primary CT26 tumor, rejected a secondary tumor challenge compared with naïve mice (FIG. 13A to FIG. 13D).

Checkpoint blockade of PD-1 and CTLA4 can enhance the antitumor activity of immunotherapeutics with immune-priming activity, including CD40 agonists. To understand the mechanistic basis for synergy between PD-1/CTLA4 blockade and mSIRPα-Fc-CD40L, CT26 tumors were excised from anti-PD-1- or anti-CTLA4-treated mice 11 days after inoculation and performed phenotypic analysis of the tumor-infiltrating lymphocytes. Both agents expanded CD40+ dendritic cells/B cells and CD3+ T cells, and induced the upregulation of MHC-I and MHC-II (FIG. 7C). Thus, initial treatment with anti-PD-1 or anti-CTLA4 stimulated expansion of CD40-expressing immune cells potentially explaining the improved responsiveness with mSIRPα-Fc-CD40L. Checkpoint inhibitor blockade did not affect the tumor surface expression of CD47 (FIG. 7C and FIG. 13E), suggesting that checkpoint combination synergy functions independently of phagocytosis activity.

### Example 6: Safety and Activity of the SIRPα-Fc-CD40L Chimeric Protein in Nonhuman Primates

Enthusiasm surrounding the clinical utility of SIRPα/CD47 inhibition is somewhat tempered by expression of CD47 on erythrocytes and platelets, and the associated risk of hemolysis and thrombocytopenia. Lin et al., TTI-621 (SIRPalphaFc), a CD47-blocking cancer immunotherapeutic, triggers phagocytosis of lymphoma cells by multiple polarized macrophage subsets. PLoS One 12:e0187262 (2017); Advani et al., CD47 Blockade by Hu5F9-G4 and rituximab in non-Hodgkin's lymphoma. N Engl J Med 379:1711-1721 (2018); Brierley et al., The effects of monoclonal anti-CD47 on RBCs, compatibility testing, and transfusion requirements in refractory acute myeloid leukemia. Transfusion 59:2248-2254 (2019). As shown in the Table below, the Fc domain of the human SIRPα-Fc-CD40L chimeric protein does not bind effector Fc receptors:

### Maximum Fold-Change for Each Indicated Cytokine in NHP Serum Post-Infusion as Compared to Pre-Infusion

| | Vehicle | hSIRPα-Fc-CD40L |
|---|---|---|
| CCL2 | 2.89 | 31.04 |
| CXCL9 | 8.33 | 1482.36 |
| CXCL10 | 34.57 | 1830.25 |
| IFNα | Not Detected | 723.74 |
| IL-6 | 21.7 | 617.35 |
| IL-15 | Not Detected | 2381.65 |
| IL-23 | 20.67 | 420 |

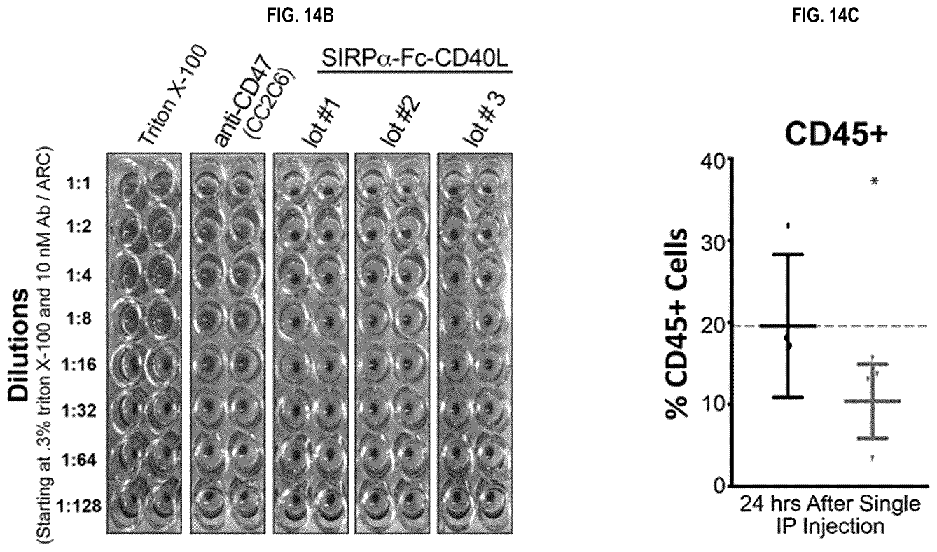

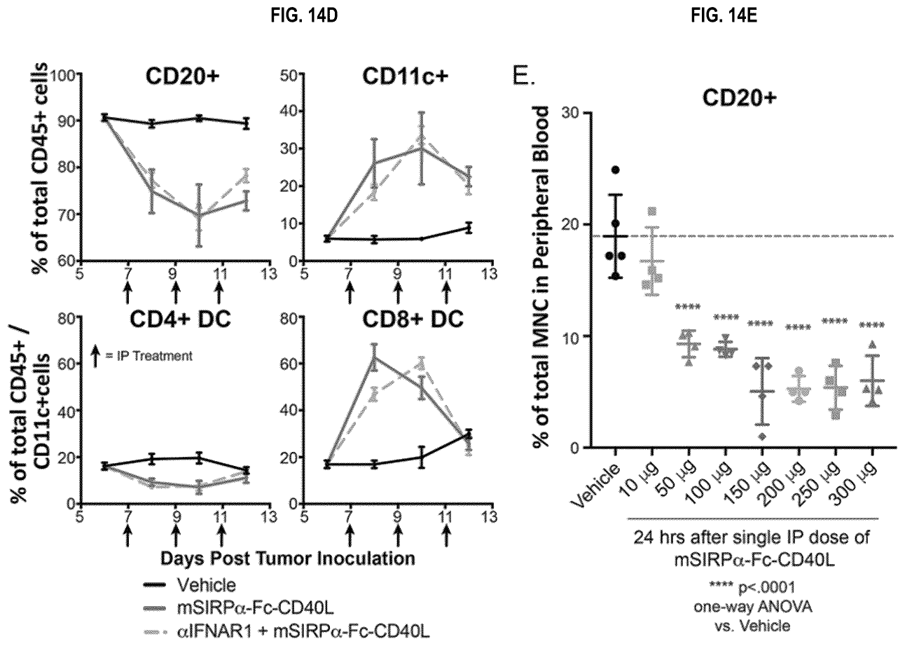

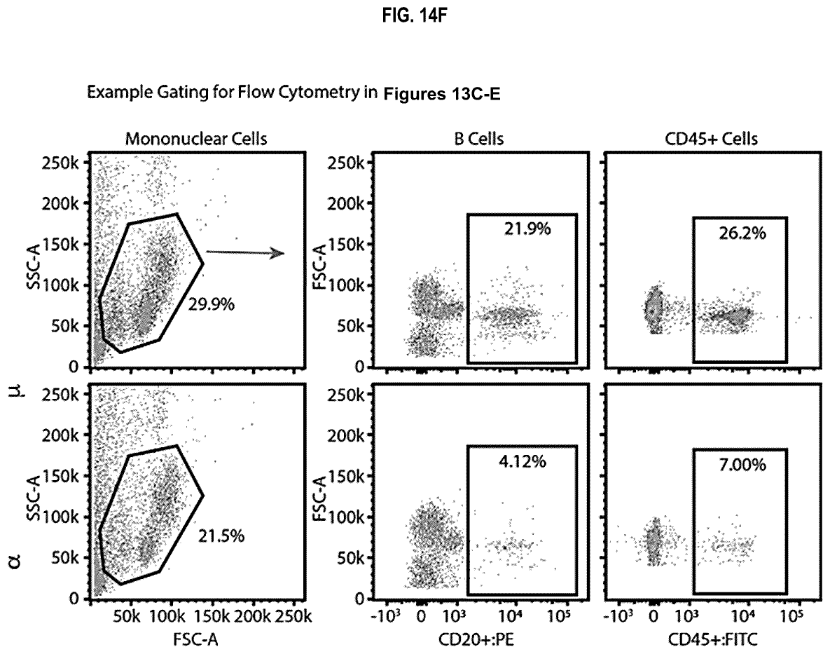

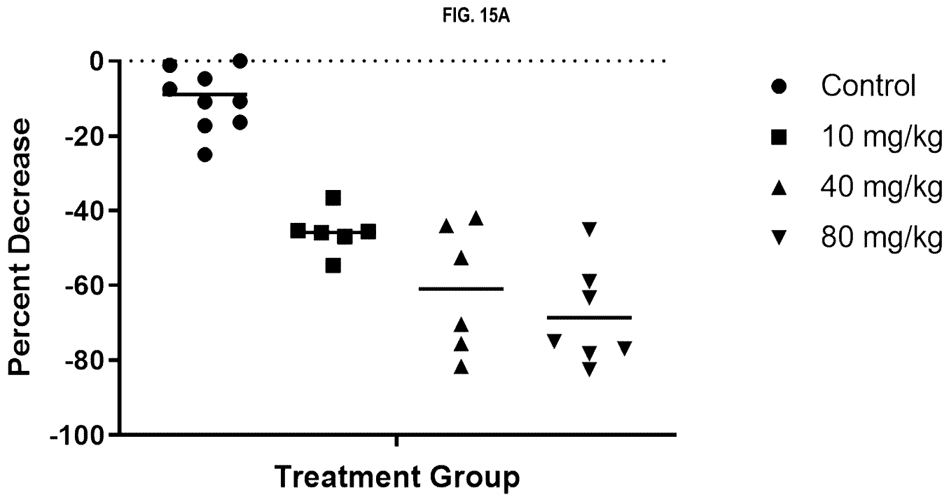

Interestingly, in vitro studies did not reveal evidence of hemolysis in human or cynomolgus macaque erythrocytes (FIG. 14B); however, the in vitro systems used to test this question had significant limitations, including a complete lack of macrophages. Thus, a more accurate measure of hematologic toxicities required in vivo dosing in a relevant animal model.

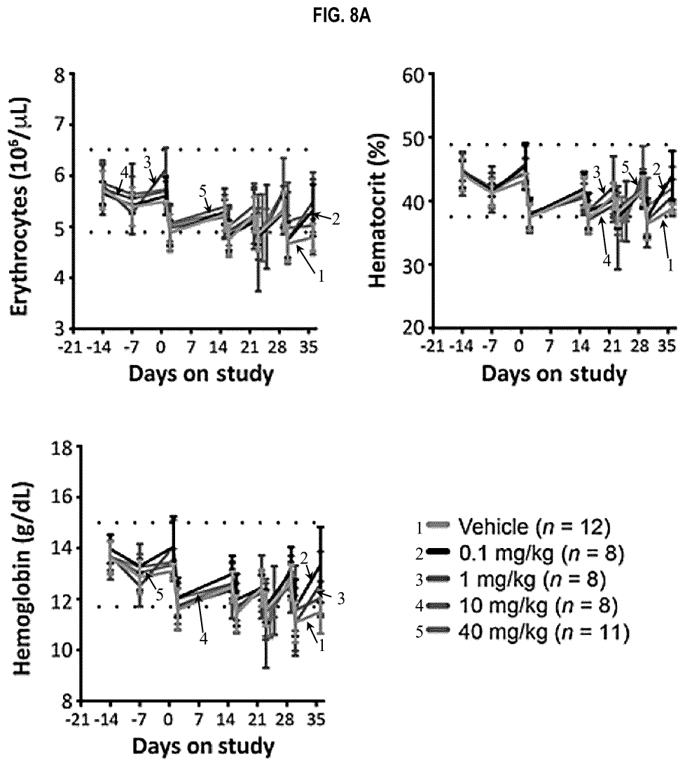

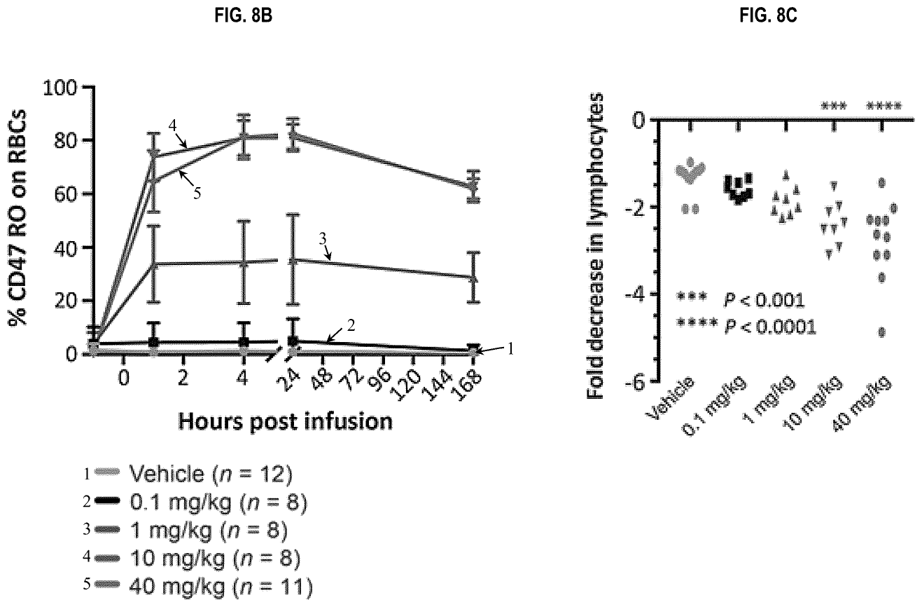

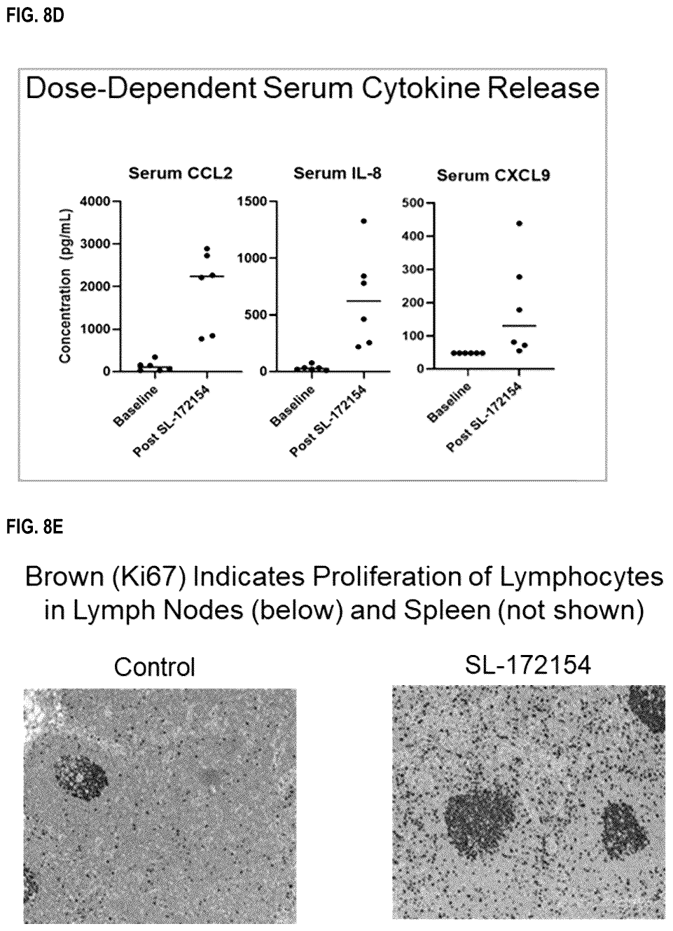

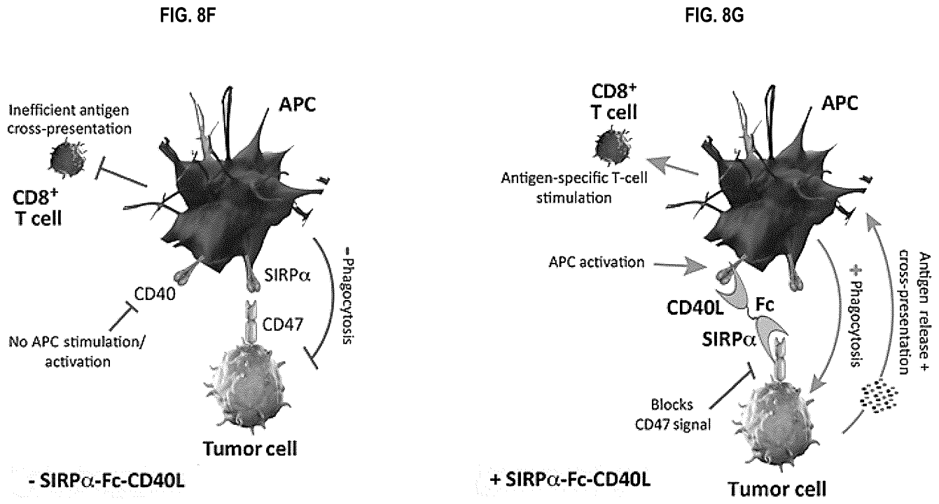

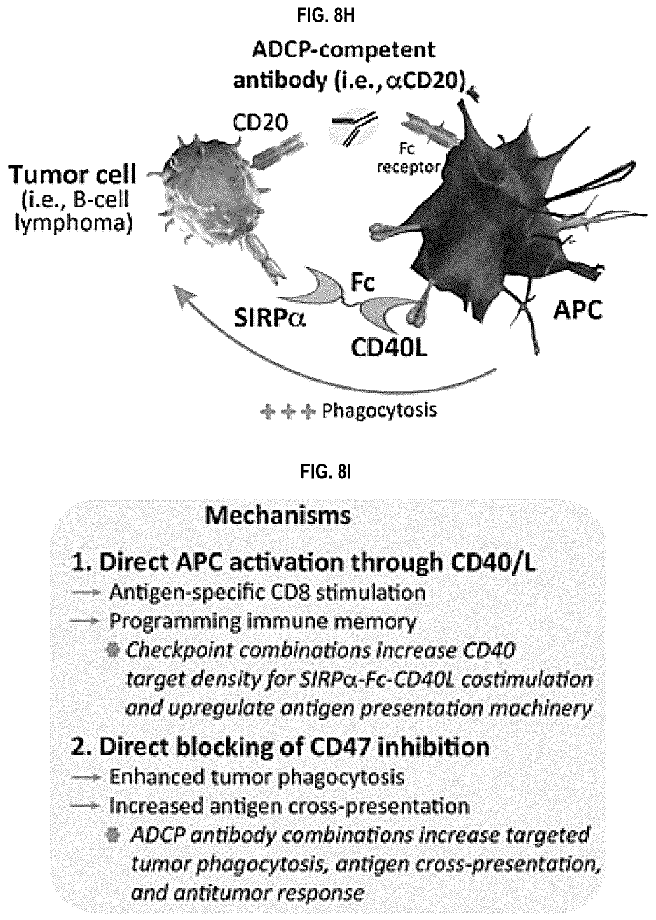

Cynomolgus macaques are a relevant species for evaluating SIRPα/CD47-related toxicity due to high homology of CD47 between human and cynomolgus macaque (98.69% identity), and the development of the priming dose strategy for the CD47-specific Hu5F9-G4 antibody. Liu et al., Pre-clinical development of a humanized anti-CD47 antibody with anti-cancer therapeutic potential. PLoS One 10:e0137345 (2015). A species cross-reactivity of the human SIRPα-Fc-CD40L chimeric protein with recombinant cynomolgus CD47 (1.7 nmol/L binding affinity) and CD40 (3.24 nmol/L binding affinity) was confirmed. Next, it was sought to test the safety and activity of the human SIRPα-Fc-CD40L chimeric protein following repeat doses in cynomolgus macaques. There was no evidence of hemolysis or thrombocytopenia following repeated infusion with the human SIRPα-Fc-CD40L chimeric protein at doses up to 40 mg/kg over the course of the study (FIG. 8A). Mild declines in hematology parameters were noted; however, these declines were also noted in the vehicle control group and were most likely related to procedural effects and repeated blood collections. Dose-dependent receptor occupancy was observed across the dosing groups which peaked 4 hours postinfusion at 80.96%±2.6%, and was roughly equivalent between the 10 and 40 mg/kg dose groups (FIG. 8B and FIG. 14A). CD47 occupancy was stable and remained at 62.78%±2.3% occupancy on RBC CD47 when evaluated 168 hours postinfusion (FIG. 8B). A dose-dependent episodic fluctuation in the total number of lymphocytes was also observed before and after each dose, which were of lower magnitude than the postdose reductions observed for circulating CD40+ lymphocytes (FIG. 8C). This peripheral decrease in CD40+ B cells was consistent with similar observations seen in the blood of mice treated with the mSIRPα-Fc-CD40L chimeric protein (FIG. 14C to FIG. 14F). In mice, the decrease in B cells was dose dependent, plateaued at a single intraperitoneal dose of 150 μg, and was accompanied by a significant increase in CD8+ DCs (FIG. 14C to FIG. 14F). Finally, as shown in the Table above, dose-dependent increases were observed in multiple serum cytokines/chemokines in cynomolgus macaques following each infusion of the human SIRPα-Fc-CD40L chimeric protein, including CCL2, CXCL9, CXCL10, IFNα, IL6, IL15, and IL23, suggestive of an on-target pharmacodynamic biology. FIG. 8D also shows the levels of cytokines CCL2, IL-8 and CXCL9 in serum after dosing compared to the background levels prior to dosing. FIG. 8E shows the staining of Ki67 positive cells in lymph nodes after dosing compared to the background levels prior to dosing. These data indicate that The overall mechanism through which the SIRPα-Fc-CD40L chimeric protein is proposed to bridge macrophage-mediated tumor cell phagocytosis to APC activation and antigen presentation is outlined in FIG. 8F to FIG. 8I.

Collectively, these data suggest that while CD47 blockade is an effective strategy to enhance macrophage mediated tumor cell phagocytosis, enhancing a type I IFN response via CD40 stimulation in a coordinated fashion with CD47/SIRPα blockade powerfully enhances antitumor immunity. The observation that SIRPα-Fc-CD40L stimulated dose-dependent elevation in multiple serum cytokines and CD40+ B-cell margination in cynomolgus macaques, without causing hemolysis or thrombocytopenia, provides justification to further explore this strategy in human patients with cancer.

### Example 7: Lymphocyte Margination by SL-172154

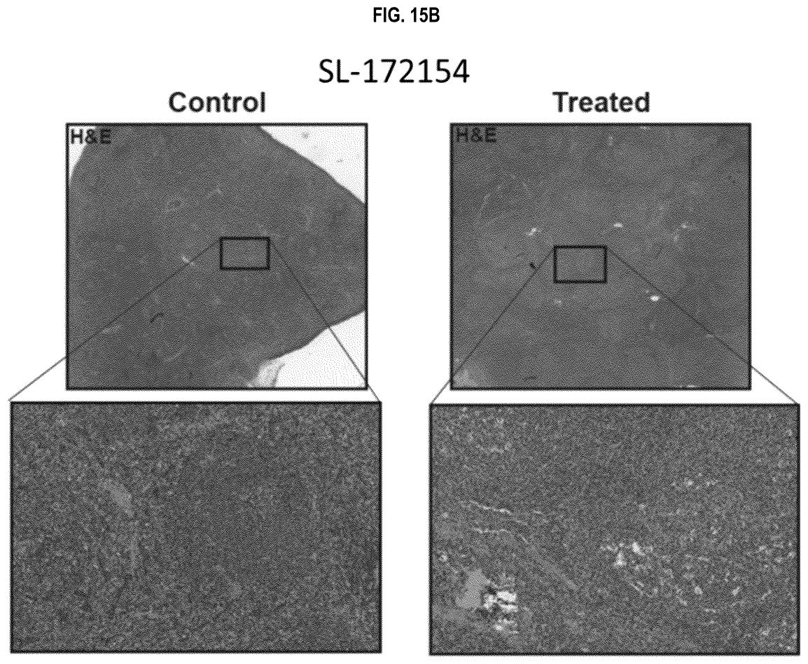

Next, the localization of lymphocytes following the treatment with SL-172154 was explored. Cynomolgus monkeys were treated with SL-172154 on Day 1, 8 and 15 at the indicated dose. Pre- and post-dose lymphocyte counts were obtained on day 15 prior to the third dose, and on day 16 approximately 24 hours after the third dose. FIG. 15A shows the post-dose lymphocyte margination from day 15 to day 16. The number of peripheral blood lymphocytes was observed to decrease in a dose-dependent manner following the Day 15 dose, and is plotted as the (100−((#of lymphocytes on Day 16)/(#of lymphocytes on Day 15)×100). Each data point indicates an individual animal. As shown in FIG. 15A, there was a dose-dependent decrease in the number of peripheral blood lymphocytes in SL-172154-treated monkeys compared to the control monkeys. These data illustrate the post-dose lymphocyte margination.

The effect of SL-172154 on CD40+ lymphocyte localization was further explored in cynomolgus monkeys. Cynomolgus monkeys were administered 5 consecutive weekly doses of SL-172154. Lymphocytes were stained in various tissue sections by immunohistochemistry and visualized microscopically. FIG. 15B shows the illustrative histochemical analysis of spleens of from untreated and SL-172154-treated cynomolgus monkeys. As shown in FIG. 15B, the spleen sections from SL-172154-treated cynomolgus monkeys showed higher levels of CD40+ lymphocytes compared to the lung sections from untreated cynomolgus monkeys. Similarly, the cynomolgus monkeys treated with SL-172154 were observed to have dose-dependent migration of CD40+ cells from the peripheral blood into secondary lymphoid organs including the lymph nodes and spleen (data not shown). These effects were seen in cynomolgus monkeys treated with SL-172154 across a dose range of 0.1-40 mg/kg for 5 consecutive weekly doses.

These data demonstrate that SL-171154 induced dose-dependent migration of lymphocytes from peripheral blood into secondary lymphoid organs including the lymph nodes and spleen. Collectively, these data provide evidence of on-target biology driven by CD40, and these effects were accompanied by distinct changes in multiple serum cytokines.

### Example 8: Phase 1[/2] Dose Escalation and Dose Expansion Trial Study Design of SL-172154 Administered Intravenously

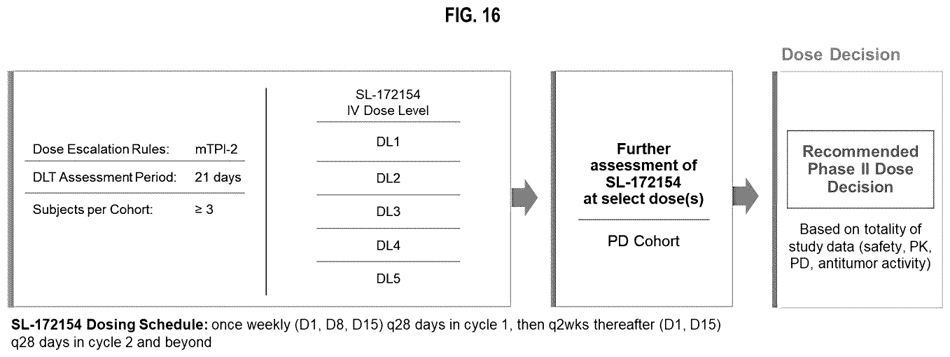

FIG. 16 shows a schematic of the design of the Phase 1 clinical trial of SL-172154. The Phase 1 clinical trial is a first in human, open label, multi-center, dose escalation and dose expansion study in subjects with advanced solid tumors or lymphomas. The primary objective of this study is to evaluate the safety, tolerability of SL-172154. The secondary objective of this study is to evaluate the recommended phase II dose (RP2D), pharmacokinetic (PK), anti-tumor activity and pharmacodynamic effects of SL-172154. A Phase 1 study of SL-172154 administered intratumorally in patients with locally advanced or metastatic cutaneous squamous cell carcinoma (CSCC) and squamous cell carcinoma of the head and neck (HNSCC) not amenable to further treatment with surgery, radiation, or standard systemic therapies was carried out. We anticipate enrolling patients in this study starting in November 2020. In the dose escalation portion of the study, three or more patients will be enrolled through each of four dose levels, ranging from 0.003 mg to 0.1 mg. Following the dose-escalation portion of the study, six patients are planned to be enrolled in a dose-expansion cohort to further evaluate pharmacodynamic endpoints (FIG. 16).

Following the identification of a monotherapy RP2D, SL-172154 will be evaluated in combination with cetuximab. Three or more patients will be enrolled through each of 4 dose levels in the dose escalation portion of the study. Following the dose-escalation portion of the study, 6 patients will be enrolled to further evaluate pharmacodynamic endpoints. Enrolling a total of ˜approximately 18 40 patients across the dose escalation and expansion portions of the study is anticipated. An overview of the clinical trial design is below: The study design consists of Dose Escalation Cohorts and PD Cohorts, shown in FIG. 16 (left and middle panel, respectively. The dose levels (DL) used in this study were DL1 through DL5, ranging from 0.1 mg/kg to 10.0 mg/kg. Specifically, DL1, DL2, DL3, DL4 and DL5 were 0.1 mg/kg, 0.3 mg/kg, 1.0 mg/kg, 3.0 mg/kg, and 10.0 mg/kg, respectively. Selection of the respective recommended phase 2 dose (RP2D) and schedule for SL-172154 will be based upon the totality of the data, including safety, PK, PD and anti-tumor activity, in patients treated in the dose escalation and the pharmacodynamic cohorts in each study (FIG. 16, right panel).

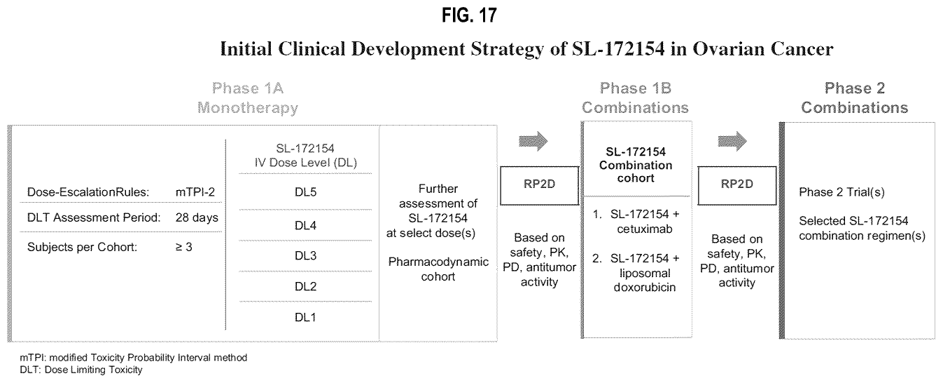

FIG. 17 shows a schematic of the initial clinical development strategy of SL-172154 in Ovarian Cancer. The dose levels (DL) used in this study were DL1 through DL5, ranging from 0.1 mg/kg to 10.0 mg/kg. Phase 1[/2]trial of SL-172154 administered intravenously in patients with advanced ovarian, fallopian tube and primary peritoneal cancers. Patients will include those who have failed platinum-based therapies and are ineligible for further platinum therapy. In the dose escalation portion of the study, three or more patients will be enrolled through each of five dose levels, ranging from 0.1 mg/kg to 10.0 mg/kg. Following the identification of a recommended Phase 2 dose, or RP2D, SL-172154 will be evaluated in two expansion cohorts in ovarian cancer: one in combination with cetuximab, an ADCC/ADCP competent antibody, and the other in combination with doxorubicin. We anticipate enrolling a total of approximately 70 patients across the dose escalation and expansion portions of the study.

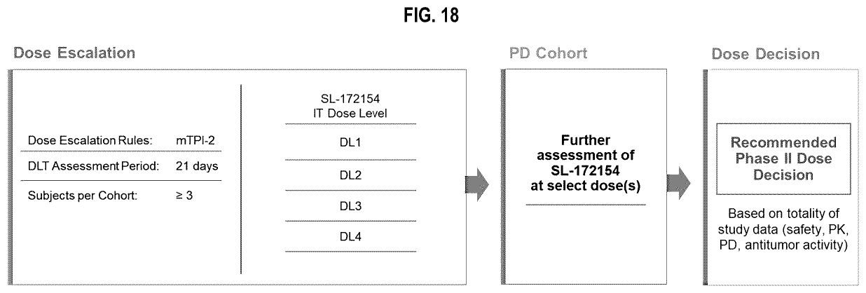

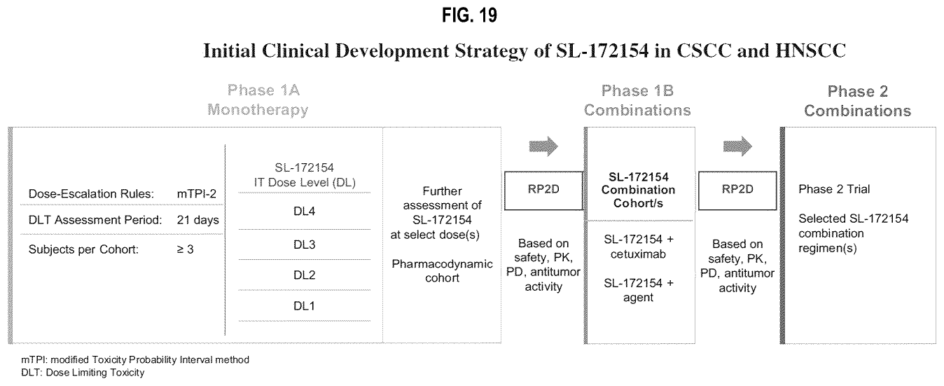

FIG. 18 and FIG. 19 show a schematic of the design of the Phase 1 clinical trial of SL-172154 in CSCC and HNSCC. In the dose escalation portion of the study, three or more patients will be enrolled through each of four dose levels, ranging from 0.003 mg to 0.1 mg. A Phase 1 study of SL-172154 administered intratumorally in patients with locally advanced or metastatic cutaneous squamous cell carcinoma (CSCC) and squamous cell carcinoma of the head and neck (HNSCC) will be carried out. Patients will include those that are not amenable to further treatment with surgery, radiation, or standard systemic therapies. In the dose escalation portion of the study, three or more patients will be enrolled through each of four dose levels, ranging from 0.003 mg to 0.1 mg. Following the dose-escalation portion of the study, six patients will be enrolled in a dose-expansion cohort to further evaluate pharmacodynamic endpoints. Following the identification of a monotherapy RP2D, SL-172154 will be evaluated in combination with cetuximab. Three or more patients will be enrolled through each of 4 dose levels in the dose escalation portion of the study. Following the dose-escalation portion of the study, we plan to enroll 6 patients to further evaluate pharmacodynamic endpoints. We anticipate enrolling a total of about 45 patients across the dose escalation and expansion portions of the study. The primary objective of each Phase 1[/2]trial is to assess the safety and tolerability of SL-172154 by intravenous infusion or intratumoral injection. The secondary objectives include evaluation of pharmacokinetic profile, immunogenicity, and anti-tumor activity. Exploratory objectives include assessment of pharmacodynamic activity in the blood, including receptor occupancy, immune phenotyping, serum cytokines, and in the tumor, including immunohistochemistry from pre- and post-treatment biopsies.

This dose escalation study was initiated. To date, no dose-limiting toxicities have been observed, and dosing in the higher dose-level cohorts will be continued. Since SL-172154 contains both CD47 inhibitory and CD40 agonist domains, the safety data can be considered in context with prior CD47 inhibitors and CD40 agonists. On the CD47 side any evidence of anemia or thrombocytopenia, was not observed. Without being bound by theory, it is believed that the observed lack of anemia or thrombocytopenia may be due to the mutated Fc region. Without being bound by theory, it is believed that the Fc domain lacking effector functions (e.g. reduced binding to Fc receptors (i.e. other than FcRn) with effector function) contributes to the safety profile, which differentiates SL-172154 from other CD47 inhibitors with active Fc domains that have reported anemia or thrombocytopenia in their clinical studies.

On the CD40 side, various CD40 agonist antibodies have been in clinical testing for well over 20 years, but progress has been repeatedly hampered by a combination of toxicities at low doses and evidence of a‘bell-shaped’ dose-response curve. These CD40 agonist antibodies have exhibited dose-limiting toxicities, including a combination of cytokine release syndrome and liver dysfunction, at doses of roughly 0.3 mg/kg. In contrast, as shown in the results discussed below, even a 3 mg/kg dose level of SL-172154-10 times higher than the CD40 agonist antibodies—did not produce dose-limiting toxicities. And further, from the results discussed below, the dose-response curve over the dose range tested to date has exhibited an escalating linear relationship between the dose of SL-172154 administered and the corresponding pharmacodynamic responses to SL-172154. Cytokine release syndrome or liver dysfunction were observed, yet unique evidence of CD40 engagement and pharmacodynamic activity was observed.

### Example 9: Phase 1 Dose Escalation Study of the Agonist Redirected Checkpoint, SL-172154 (SIRPα-Fc-CD40L) in Subjects with Platinum-Resistant Ovarian Cancer

### Methods

This first-in-human, Phase 1 dose escalation study is evaluating SL-172154 as monotherapy in patients with platinum resistant ovarian, fallopian tube and primary peritoneal cancers. Objectives include evaluation of safety, dose-limiting toxicity (DLT) and recommended phase 2 dose (RP2D), pharmacokinetic (PK) parameters, pharmacodynamic (PD) effects and antitumor activity based on Response Evaluation Criteria in Solid Tumors (RECIST).

### Results

14 heavily pretreated patients (median age, 67 years) were enrolled and treated with intravenous (IV) administration of SL-172154 across 4 dose levels on 2 schedules: schedule 1 (day 1, 8, 15, 29, Q2 weeks) at 0.1, 0.3 mg/kg and schedule 2 (weekly) at 0.3, 1.0, 3.0 mg/kg. The most common treatment-related (>20%) adverse events (AEs) of any grade (G) were fatigue (n=7, 50%), infusion-related reactions (IRR) (n=6, 43%), nausea (n=4, 29%), and decreased appetite (n=3, 21%). Treatment-related IRRs (G1/G2) generally occurred near the end of infusion or immediately post-infusion; the full dose was able to be delivered in each IRR event, and subsequent infusions in patients having IRRs were managed with pre-medications. No treatment related ≥G3 AEs or DLTs have occurred. CD47 receptor occupancy (RO) on leukocytes approached 90% at 1.0 and 3.0 mg/kg. Minimal binding to CD47+ red blood cells was observed at all dose levels. CD40 RO on B cells was >60% at doses ≥0.1 mg/kg and 75%-100% at 1.0 and 3.0 mg/kg. Rapid, transient B cell and monocyte margination was observed following infusion of SL-172154 and was consistent with dose-dependent increases in IL-12, MCP-1, MIP-1β, MIP-1α, and MDC. Interestingly, no appreciable increases in IL-6 or TNFα were noted and there was no correlation between IRRs and cytokine increases. Among 12 evaluable patients, stable disease was observed in 3 patients.

### Conclusions

SL-172154 has been well tolerated with no evidence of anemia, thrombocytopenia, liver dysfunction or cytokine release syndrome. A unique serum cytokine signature consistent with CD40 RO and activation has been observed and this signature is maintained following repeat dosing. Dose escalation will be continued to 6 mg/kg and 10 mg/kg doses of SL-172154. Surprisingly, the lack of IL-6 and TNFα increases indicate a lack of systemic inflammation.

As discussed above, the dose-response curve over the dose range tested to date has exhibited an escalating linear relationship between the dose of SL-172154 administered and the corresponding pharmacodynamic responses to SL-172154.

### Example 10: Phase 1 Dose Escalation Study of the Agonist Redirected Checkpoint, SL-172154 (SIRPα-Fc-CD40L) in Subjects with Acute Myeloid Leukemia (AML) and High-Risk Myelodysplastic Syndromes (HR-MDS)

### Methods

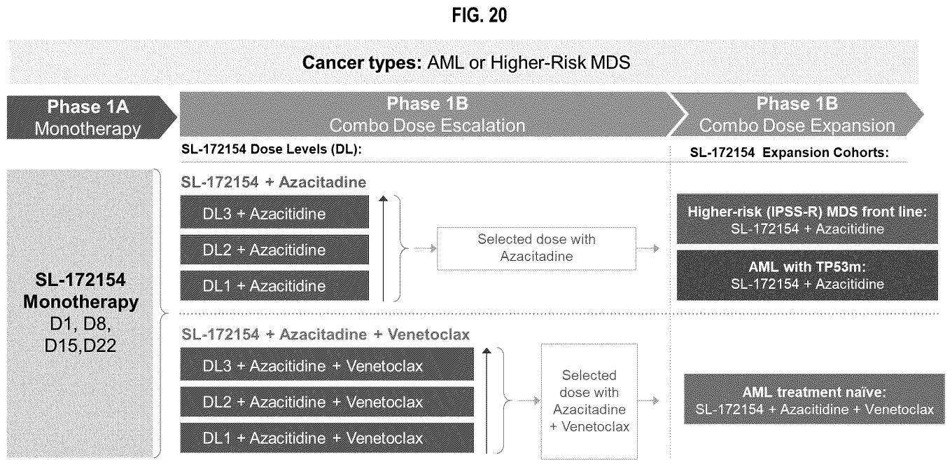

A Phase 1A/B clinical trial for evaluating SL-172154 as monotherapy in patients with acute myeloid leukemia (AML) and high-risk myelodysplastic syndromes (HR-MDS) will be carried out. Objectives for this study include evaluation of safety, tolerability, pharmacokinetics, anti-tumor activity, and pharmacodynamic effects of SL-172154, as both monotherapy and in combination. In AML, SL-172154 will be evaluated in combination with both azacitidine and venetoclax, as well as with azacitidine alone. In HR-MDS, SL-172154 will be evaluated in combination with azacitidine. Study design is shown in FIG. 20. Briefly, in Phase 1A (SL-172154 monotherapy), SL-172154 will be administered on days 1, 8, 15 and 22, and safety, tolerability, pharmacokinetics, anti-tumor activity, and pharmacodynamic effects will be assessed. During Phase 1B (combination therapy), a dose escalation of SL-172154 will be carried out with dose level DL1 through DL3 in patients administered. In this part of study, treatment naïve AML patients will be administered a combination of SL-172154 with azacitidine and venetoclax; patients having TP53 mutant AML will be administered a combination of SL-172154 with azacytidine; and patients having higher-risk (the International Prognostic Scoring System-Revised (IPSS-R) (IPSS-R)) MDS front line will be administered a combination of SL-172154 with venetoclax. After dose escalation, dose expansion will be carried out.

### INCORPORATION BY REFERENCE

All patents and publications referenced herein are hereby incorporated by reference in their entireties.

The publications discussed herein are provided solely for their disclosure prior to the filing date of the present application. Nothing herein is to be construed as an admission that the present disclosure is not entitled to antedate such publication by virtue of prior invention.

As used herein, all headings are simply for organization and are not intended to limit the disclosure in any manner. The content of any individual section may be equally applicable to all sections.

### EQUIVALENTS

While the invention has been disclosed in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

Those skilled in the art will recognize, or be able to ascertain, using no more than routine experimentation, numerous equivalents to the specific embodiments disclosed specifically herein. Such equivalents are intended to be encompassed in the scope of the following claims.

1.-151. (canceled)

152. A method for treating a cancer in a human subject comprising a step of administering to the human subject a chimeric protein having a general structure of: 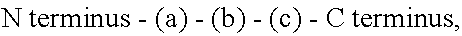
wherein:
(a) is a first domain comprising an extracellular domain of human Signal regulatory protein α (CD172a (SIRPα)),
(b) is a linker adjoining the first and second domains, wherein the linker comprises at least one cysteine residue capable of forming a disulfide bond and/or comprises a hinge-CH2-CH3 Fc domain, and
(c) is a second domain comprising an extracellular domain of human CD40 ligand (CD40L),
wherein the chimeric protein is administered at an initial dose of at least 1.0 mg/kg and optionally one or more subsequent administrations.

153. The method of claim 152, wherein the one or more subsequent administrations has a dose selected from about 0.0001, about 0.001, about 0.003, about 0.01, about 0.03, about 0.1, about 0.3, about 1, about 2, about 3, about 4, about 6.0, or about 10 mg/kg.

154. The method of claim 152, wherein the initial dose is less than the dose for at least one of the subsequent administrations.

155. The method of claim 152, wherein the initial dose is the same as the dose for at least one of the subsequent administrations.

156. The method of claim 152, wherein the chimeric protein is administered once a week, about two times a month, about three times a month, about once every three weeks, or about once every four weeks.

157. The method of claim 152, wherein the cancer comprises an advanced solid tumor or a lymphoma.

158. The method of claim 157, wherein the cancer is selected from ovarian cancer, fallopian tube cancer, peritoneal cancer, cutaneous squamous cell carcinoma (CSCC), and squamous cell carcinoma of the head and neck (SCCHN).

159. The method of claim 152, wherein the first domain is capable of binding a CD172a (SIRPα) ligand.

160. The method of claim 152, wherein the second domain is capable of binding a CD40 receptor.

161. The method of claim 152, wherein the linker comprises a hinge-CH2-CH3 Fc domain derived from IgG4, optionally wherein the linker comprises a hinge-CH2-CH3 Fc domain derived from human IgG4.

162. The method of claim 152, wherein the linker comprises an amino acid sequence that is at least 95% identical to the amino acid sequence of SEQ ID NO: 1, SEQ ID NO: 2, or SEQ ID NO: 3.

163. The method of claim 152, wherein the first domain comprises an amino acid sequence that is at least 95% identical to the amino acid sequence of SEQ ID NO: 57.

164. The method of claim 152, wherein the second domain comprises an amino acid sequence that is at least 95% identical to the amino acid sequence of SEQ ID NO: 58.

165. The method of claim 152, wherein
(a) the first domain comprises the amino acid sequence of SEQ ID NO: 57,
(b) the second domain comprises the amino acid sequence of SEQ ID NO: 58, and
(c) the linker comprises an amino acid sequence that is at least 95% identical to SEQ ID NO: 1, SEQ ID NO: 2, or SEQ ID NO: 3.

166. The method of claim 166, wherein the chimeric protein further comprises the amino acid sequence of SEQ ID NO: 5 and/or SEQ ID NO: 7.

167. The method of claim 152, wherein the chimeric protein comprises an amino acid sequence that is at least about 95% identical to SEQ ID NO: 59 or SEQ ID NO: 61.

168. The method of claim 167, wherein the chimeric protein comprises an amino acid sequence that is at least about 98% identical to SEQ ID NO: 59 or SEQ ID NO: 61.

169. The method of claim 152, wherein the human subject has failed platinum-based therapies, and optionally is ineligible for further platinum therapy.

170. The method of claim 152, wherein the human subject is not receiving a concurrent chemotherapy, immunotherapy, biologic or hormonal therapy, and/or wherein the human subject has received, been tolerant to, or is ineligible for standard therapy and/or the cancer has no approved therapy considered to be standard of care.

171. The method of claim 152, wherein the chimeric protein is administered at a dose of about 1 mg/kg, or about 3 mg/kg, or about 6 mg/kg, or about 10 mg/kg.
